SEQUENCE LISTING (99 sequences)

SEQ ID NO: 1 - protein, 797 aa, Artificial Sequence
  synthetic
MSKSFQQSSLSRDSQGHGRDLSAAGIGLLAAATQSLSMPASLGRMNQGTARLASLMNLGMSSSLNQQGAHSALSSASTSSHNLQSIFNIGSRGPLPLSSQHRGDADQASNILASFGLSARDLDELSRYPEDKITPENLPQILLQLKRRRTEEGPTLSYGRDGRSATREPPYRVPRDDWEEKRHFRRDSFDDRGPSLNPVLDYDHGSRSQESGYYDRMDYEDDRLRDGERCRDDSFFGETSHNYHKFDSEYERMGRGPGPLQERSLFEKKRGAPPSSNIEDFHGLLPKGYPHLCSICDLPVHSNKEWSQHINGASHSRRCQLLLEIYPEWNPDNDTGHTMGDPFMLQQSTNPAPGILGPPPPSFHLGGPAVGPRGNLGAGNGNLQGPRHMQKGRVETSRVVHIMDFQRGKNLRYQLLQLVEPFGVISNHLILNKINEAFIEMATTEDAQAAVDYYTTTPALVFGKPVRVHLSQKYKRIKKPEGKPDQKFDQKQELGRVIHLSNLPHSGYSDSAVLKLAEPYGKIKNYILMRMKSQAFIEMETREDAMAMVDHCLKKALWFQGRCVKVDLSEKYKKLVLRIPNRGIDLLKKDKSRKRSYSPDGKESPSDKKSKTDGSQKTESSTEGKEQEEKSGEDGEKDTKDDQTEQEPNMLLESEDELLVDEEEAAALLESGSSVGDETDLANLGDVASDGKKEPSDKAVKKDGSASAAAKKKLKKVDKIEELDQENEAALENGIKNEENTEPGAESSENADDPNKDTSENADGQSDENKDDYTIPDEYRIGPYQPNVPVGIDYVIP

SEQ ID NO: 2 - protein, 322 aa, Artificial Sequence
  synthetic
MSKSFQQSSLSRDSQGHGRDLSAAGIGLLAAATQSLSMPASLGRMNQGTARLASLMNLGMSSSLNQQGAHSALSSASTSSHNLQSIFNIGSRGPLPLSSQHRGDADQASNILASFGLSARDLDELSRYPEDKITPENLPQILLQLKRRRTEEGPTLSYGRDGRSATREPPYRVPRDDWEEKRHFRRDSFDDRGPSLNPVLDYDHGSRSQESGYYDRMDYEDDRLRDGERCRDDSFFGETSHNYHKFDSEYERMGRGPGPLQERSLFEKKRGAPPSSNIEDFHGLLPKGYPHLCSICDLPVHSNKEWSQHINGASHSRRCQLL

SEQ ID NO: 3 - protein, 287 aa, Artificial Sequence
  synthetic
MSKSFQQSSLSRDSQGHGRDLSAAGIGLLAAATQSLSMPASLGRMNQGTARLASLMNLGMSSSLNQQGAHSALSSASTSSHNLQSIFNIGSRGPLPLSSQHRGDADQASNILASFGLSARDLDELSRYPEDKITPENLPQILLQLKRRRTEEGPTLSYGRDGRSATREPPYRVPRDDWEEKRHFRRDSFDDRGPSLNPVLDYDHGSRSQESGYYDRMDYEDDRLRDGERCRDDSFFGETSHNYHKFDSEYERMGRGPGPLQERSLFEKKRGAPPSSNIEDFHGLLPK

SEQ ID NO: 4 - protein, 560 aa, Artificial Sequence
  synthetic
GYPHLCSICDLPVHSNKEWSQHINGASHSRRCQLLLEIYPEWNPDNDTGHTMGDPFMLQQSTNPAPGILGPPPPSFHLGGPAVGPRGNLGAGNGNLQGPRHMQKGRVETSRVVHIMDFQRGKNLRYQLLQLVEPFGVISNHLILNKINEAFIEMATTEDAQAAVDYYTTTPALVFGKPVRVHLSQKYKRIKKPEGKPDQKFDQKQELGRVIHLSNLPHSGYSDSAVLKLAEPYGKIKNYILMRMKSQAFIEMETREDAMAMVDHCLKKALWFQGRCVKVDLSEKYKKLVLRIPNRGIDLLKKDKSRKRSYSPDGKESPSDKKSKTDGSQKTESSTEGKEQEEKSGEDGEKDTKDDQTEQEPNMLLESEDELLVDEEEAAALLESGSSVGDETDLANLGDVASDGKKEPSDKAVKKDGSASAAAKKKLKKVDKIEELDQENEAALENGIKNEENTEPGAESSENADDPNKDTSENADGQSDENKDDYTIPDEYRIGPYQPNVPVGIDYVIPKTGFYCKLCSLFYTNEEVAKNTHCSSLPHYQKLKKFLNKLAEERRQKKET

SEQ ID NO: 5 - protein, 609 aa, Artificial Sequence [sequence omitted]

SEQ ID NO: 6 - protein, 585 aa, Artificial Sequence [sequence omitted]

SEQ ID NO: 7 - protein, 4 aa, Artificial Sequence
  synthetic
GGGG

SEQ ID NO: 8 - protein, 5 aa, Artificial Sequence
  synthetic
GGGGG

SEQ ID NO: 9 - protein, 5 aa, Artificial Sequence
  synthetic
GSGGG

SEQ ID NO: 10 - protein, 5 aa, Artificial Sequence
  synthetic
GSGGG

SEQ ID NO: 11 - protein, 4 aa, Artificial Sequence
  synthetic
GSGG

SEQ ID NO: 12 - protein, 4 aa, Artificial Sequence
  synthetic
SGGG

SEQ ID NO: 13 - protein, 5 aa, Artificial Sequence
  synthetic
GGGGS

SEQ ID NO: 14 - protein, 15 aa, Artificial Sequence
  synthetic
GGGGSGGGGSGGGGS

SEQ ID NO: 15 - protein, 5 aa, Artificial Sequence
  synthetic
GPPGS

SEQ ID NO: 16 - protein, 5 aa, Artificial Sequence
  synthetic
GPPGS

SEQ ID NO: 17 - protein, 4 aa, Artificial Sequence
  synthetic
GGGS

SEQ ID NO: 18 - protein, 3 aa, Artificial Sequence
  synthetic
GGS

SEQ ID NO: 19 - protein, 5 aa, Artificial Sequence
  synthetic
SGGGG

SEQ ID NO: 20 - protein, 4 aa, Artificial Sequence
  synthetic
SGGG

SEQ ID NO: 21 - protein, 3 aa, Artificial Sequence
  synthetic
SGG

SEQ ID NO: 22 - protein, 5 aa, Artificial Sequence
  synthetic
GGGGA

SEQ ID NO: 23 - protein, 5 aa, Artificial Sequence
  synthetic
EAAAK

SEQ ID NO: 24 - protein, 5 aa, Artificial Sequence
  synthetic
EAAAK

SEQ ID NO: 25 - protein, 112 aa, Artificial Sequence
  synthetic
ARNGDHCPLGPGRCCRLHTVRASLEDLGWADWVLSPREVQVTMCIGACPSQFRAANMHAQIKTSLHRLKPDTVPAPCCVPASYNPMVLIQKTDTGVSLQTYDDLLAKDCHCI

SEQ ID NO: 26 - protein, 23 aa, Artificial Sequence
  synthetic
GGSSEAAEAAEAAEAAEAAEAAE

SEQ ID NO: 27 - DNA, 21 bp, Artificial Sequence
  synthetic
tcaagaaggtggtgaagcagg

SEQ ID NO: 28 - DNA, 22 bp, Artificial Sequence
  synthetic
accaggaaatgagcttgacaaa

SEQ ID NO: 29 - DNA, 22 bp, Artificial Sequence
  synthetic
atcaatggagcaagtcacagtc

SEQ ID NO: 30 - DNA, 21 bp, Artificial Sequence
  synthetic
tgcaacatgaatggatcaccc

SEQ ID NO: 31 - DNA, 21 bp, Artificial Sequence
  synthetic
ctcagactctcgtccgaatcc

SEQ ID NO: 32 - DNA, 22 bp, Artificial Sequence
  synthetic
cagaagcaagatagctggcatc

SEQ ID NO: 33 - DNA, 20 bp, Artificial Sequence
  synthetic
gagaaggcggcttacctgag

SEQ ID NO: 34 - DNA, 20 bp, Artificial Sequence
  synthetic
cgaaggcccgctttaagaga

SEQ ID NO: 35 - DNA, 21 bp, Artificial Sequence
  synthetic
ggcatgtggcgtcatagtaga

SEQ ID NO: 36 - DNA, 21 bp, Artificial Sequence
  synthetic
cacggagttagctctgtgact

SEQ ID NO: 37 - DNA, 17 bp, Artificial Sequence
  synthetic
cgtgtgctgggctcctc

SEQ ID NO: 38 - DNA, 20 bp, Artificial Sequence
  synthetic
aaagctttgtctccgtcggt

SEQ ID NO: 39 - DNA, 20 bp, Artificial Sequence
  synthetic
ctgcgagtacctccatggtc

SEQ ID NO: 40 - DNA, 20 bp, Artificial Sequence
  synthetic
agagagaaagccaactccgc

SEQ ID NO: 41 - DNA, 20 bp, Artificial Sequence
  synthetic
tgctgacgtgtttcttgtcc

SEQ ID NO: 42 - DNA, 20 bp, Artificial Sequence
  synthetic
ttgcactgcctttcattctg

SEQ ID NO: 43 - DNA, 20 bp, Artificial Sequence
  synthetic
ccgtgacccagttcgacaac

SEQ ID NO: 44 - DNA, 19 bp, Artificial Sequence
  synthetic
cggcagtttagtgagcggt

SEQ ID NO: 45 - DNA, 20 bp, Artificial Sequence
  synthetic
gcttttcgagtcagtgctgc

SEQ ID NO: 46 - DNA, 21 bp, Artificial Sequence
  synthetic
ggggagaataactcagggttg

SEQ ID NO: 47 - DNA, 20 bp, Artificial Sequence
  synthetic
ttgattttgcccgtacccgt

SEQ ID NO: 48 - DNA, 20 bp, Artificial Sequence
  synthetic
ggatccggaagcattccctt

SEQ ID NO: 49 - DNA, 20 bp, Artificial Sequence
  synthetic
gcgcaacctctcctagaaac

SEQ ID NO: 50 - DNA, 20 bp, Artificial Sequence
  synthetic
agcagagcccggtattcttc

SEQ ID NO: 51 - DNA, 18 bp, Artificial Sequence
  synthetic
cccaggtaccagcagacc

SEQ ID NO: 52 - DNA, 24 bp, Artificial Sequence
  synthetic
tccaggagatgtaactctaatcca

SEQ ID NO: 53 - DNA, 20 bp, Artificial Sequence
  synthetic
gcgttcacctcttttccaag

SEQ ID NO: 54 - DNA, 20 bp, Artificial Sequence
  synthetic
gccatgtggatttctcgttt

SEQ ID NO: 55 - DNA, 20 bp, Artificial Sequence
  synthetic
cccacatcaaggaactggag

SEQ ID NO: 56 - DNA, 20 bp, Artificial Sequence
  synthetic
tgttggcatccaaggtcata

SEQ ID NO: 57 - DNA, 20 bp, Artificial Sequence
  synthetic
acccatcactggactggtgt

SEQ ID NO: 58 - DNA, 20 bp, Artificial Sequence
  synthetic
cacatcctcaaagagcctga

SEQ ID NO: 59 - DNA, 21 bp, Artificial Sequence
  synthetic
ggagctgtgttttggtgacct

SEQ ID NO: 60 - DNA, 21 bp, Artificial Sequence
  synthetic
gtagttcatgcagatggggca

SEQ ID NO: 61 - DNA, 21 bp, Artificial Sequence
  synthetic
agcaagagcacaacaatttgg

SEQ ID NO: 62 - DNA, 19 bp, Artificial Sequence
  synthetic
ccctgttcgtcccgtatca

SEQ ID NO: 63 - DNA, 18 bp, Artificial Sequence
  synthetic
gctcagctccctcaacca

SEQ ID NO: 64 - DNA, 19 bp, Artificial Sequence
  synthetic
gctgtgagagctgcattcg

SEQ ID NO: 65 - DNA, 39 bp, Artificial Sequence
  synthetic
catggactcttaccgaagtaatatccccatctgtgctct

SEQ ID NO: 66 - DNA, 39 bp, Artificial Sequence
  synthetic
agagcacagatggggatattacttcggtaagagtccatg

SEQ ID NO: 67 - DNA, 39 bp, Artificial Sequence
  synthetic
cgtcgatgccagcttctttaagaaatctacccagaatgg

SEQ ID NO: 68 - DNA, 39 bp, Artificial Sequence
  synthetic
ccattctgggtagatttcttaaagaagctggcatcgacg

SEQ ID NO: 69 - DNA, 39 bp, Artificial Sequence
  synthetic
gactatgtgataccttaaacagggttttactgtaagctg

SEQ ID NO: 70 - DNA, 39 bp, Artificial Sequence
  synthetic
cagcttacagtaaaaccctgtttaaggtatcacatagtc

SEQ ID NO: 71 - DNA, 34 bp, Artificial Sequence
  synthetic
aaaggatcctatccccatctgtgctctatatgtg

SEQ ID NO: 72 - DNA, 27 bp, Artificial Sequence
  synthetic
aaactcgagttaagtttccttcttctg

SEQ ID NO: 73 - DNA, 52 bp, Artificial Sequence
  synthetic
ggggacaagtttgtacaaaaaagcaggctccgccctcccgacaccctcggac

SEQ ID NO: 74 - DNA, 51 bp, Artificial Sequence
  synthetic
ggggaccactttgtacaagaaagctgggtctaaagctcctccagcagagcc

SEQ ID NO: 75 - DNA, 52 bp, Artificial Sequence; identical to SEQ ID NO: 73
  synthetic

SEQ ID NO: 76 - DNA, 49 bp, Artificial Sequence
  synthetic
ggggaccactttgtacaagaaagctgggtctagacctgcgcgggcgccc

SEQ ID NO: 77 - DNA, 31 bp, Artificial Sequence
  synthetic
aattgtagctataattcaatcatctaaattg

SEQ ID NO: 78 - DNA, 31 bp, Artificial Sequence
  synthetic
caatttagatgattgaattatagctacaatt

SEQ ID NO: 79 - DNA, 25 bp, Artificial Sequence
  synthetic
ccgagcctttgagaaggatcgcttt

SEQ ID NO: 80 - protein, 424 aa, Artificial Sequence
  synthetic
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHPGQGGRAPAQAGGLCSAAPGGGHPAPSWVAFAHTGAWGTGLPAPHVPCAPGALPQGAFVSQAARAAPALQPSQAAPAEGISQPAPARGDFAYAAPAPPDGALSHPQAPRWPPHPGKSREDRDPQRDGLPGPCAVAQPGPAQAGPQGQGVLAPPTSQGSPWWGWGRGPQVAGAAWEPQAGAAPPPQPAPPDASASARQGQMQGIPAPSQALQEPAPWSALPCGLLLDELLASPEFLQQAQPLLETEAPGELEASEEAASLEAPLSEEEYRALLEEL

SEQ ID NO: 81 - protein, 150 aa, Artificial Sequence
  synthetic
MALPTPSDSTLPAEARGRGRRRRLVWTPSQSEALRACFERNPYPGIATRERLAQAIGIPEPRVQIWFQNERSRQLRQHRRESRPWPGRRGPPEGRRKRTAVTGSQTALLLRAFEKDRFPGIAAREELARETGLPESRIQIWFQNRRARHP

SEQ ID NO: 82 - protein, 56 aa, Artificial Sequence
  synthetic
  misc_feature (1)..(1): Xaa can be any naturally occurring amino acid
XQYKLILNGKTLKGETTTEAVDAATAEKVFKQYANDNGVDGEWTYDDATKTFTVTE

SEQ ID NO: 83 - DNA, 6630 bp, Artificial Sequence
  synthetic
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatccactagtccagtgtggtggaattctgcagatatccagcacagtggcggccgctcgagaccatgtacccatacgatgttcctgactatgccggtaccgagctcggatccaccatggctagctggagccacccgcagttcgagaaaggtggaggttccggaggtggatcgggaggtggatcgtggagccacccgcagttcgaaaaagcggccgatatcacaagtttgtacaaaaaagcaggctccatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggcaggcggcctgtgcagcgcggcccccggcgggggtcaccctgctccctcgtgggtcgccttcgcccacaccggcgcgtggggaacggggcttcccgcaccccacgtgccctgcgcgcctggggctctcccacagggggctttcgtgagccaggcagcgagggccgcccccgcgctgcagcccagccaggccgcgccggcagaggggatctcccaacctgccccggcgcgcggggatttcgcctacgccgccccggctcctccggacggggcgctctcccaccctcaggctcctcggtggcctccgcacccgggcaaaagccgggaggaccgggacccgcagcgcgacggcctgccgggcccctgcgcggtggcacagcctgggcccgctcaagcggggccgcagggccaaggggtgcttgcgccacccacgtcccaggggagtccgtggtggggctggggccggggtccccaggtcgccggggcggcgtgggaaccccaagccggggcagctccacctccccagcccgcgcccccggacgcctccgcctccgcgcggcaggggcagatgcaaggcatcccggcgccctcccaggcgctccaggagccggcgccctggtctgcactcccctgcggcctgctgctggatgagctcctggcgagcccggagtttctgcagcaggcgcaacctctcctagaaacggaggccccgggggagctggaggcctcggaagaggccgcctcgctggaagcacccctcagcgaggaagaataccgggctctgctggaggagctttagaacccagctttcttgtacaaagtggtgacgtaagctaggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 84 - DNA, 5838 bp, Artificial Sequence
  synthetic
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagagatctccctatcagtgatagagatcgtcgacgagctcgtttagtgaaccgtcagatcgcctggagacgccatccacgctgttttgacctccatagaagacaccgggaccgatccagcctccggactctagcgtttaaacttaagcttggtaccgagctcggatccactagtccagtgtggtggaattctgcagatatccagcacagtggcggccgctcgagaccatgtacccatacgatgttcctgactatgccggtaccgagctcggatccaccatggctagctggagccacccgcagttcgagaaaggtggaggttccggaggtggatcgggaggtggatcgtggagccacccgcagttcgaaaaagcggccgatatcacaagtttgtacaaaaaagcaggctccatggccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggtctagaacccagctttcttgtacaaagtggtgacgtaagctaggggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtacctagaagttcctattccgaagttcctattctctagaaagtataggaacttccttggccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagcacgtactacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 85 - DNA, 6820 bp, Artificial Sequence
  synthetic
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctccaccgcggtggcggccgccaccatggattacaaggatgacgacgataagagcccgggcggatcctccaagtcattccagcagtcatctctcagtagggactcacagggtcatgggcgtgacctgtctgcggcaggaataggccttcttgctgctgctacccagtctttaagtatgccagcatctcttggaaggatgaaccagggtactgcacgccttgctagtttaatgaatcttggaatgagttcttcattgaatcaacaaggagctcatagtgcactgtcttctgctagtacttcttcccataatttgcagtctatatttaacattggaagtagaggtccactccctttatcttctcaacaccgtggagatgcagaccaggccagtaacattttggccagctttggtctgtctgctagagacttagatgaactgagtcgttatccagaggacaagattactcctgagaatttgccccaaatccttctacagcttaaaaggaggagaactgaagaaggccctaccttgagttatggtagagatggcagatctgctacacgggagccaccatacagagtacctagggatgattgggaagaaaaaaggcactttagaagagatagttttgatgatcgtggtcctagtctcaacccagtgcttgattatgaccatggaagtcgttctcaagaatctggttattatgacagaatggattatgaagatgacagattaagagatggagaaaggtgtagggatgattctttttttggtgagacctcgcataactatcataaatttgacagtgagtatgagagaatgggacgtggtcctggccccttacaagagagatctctctttgagaaaaagagaggcgctcctccaagtagcaatattgaagacttccatggactcttaccgaagggttatccccatctgtgctctatatgtgatttgccagttcattctaataaggagtggagtcaacatatcaatggagcaagtcacagtcgtcgatgccagcttcttcttgaaatctacccagaatggaatcctgacaatgatacaggacacacaatgggtgatccattcatgttgcagcagtctacaaatccagcaccaggaattctgggacctccacctccctcatttcatcttgggggaccagcagttggaccaagaggaaatctgggtgctggaaatggaaacctgcaaggacctagacacatgcagaaaggcagagtggaaactagcagagttgttcacatcatggattttcaacgagggaaaaacttgagataccagctattacagctggtagaaccatttggagtcatttcaaatcatctgattctaaataaaattaatgaggcatttattgaaatggcaaccacagaggatgctcaggccgcagtggattattacacaaccacaccagcgttagtatttggcaagccagtgagagttcatttatcccagaagtataaaagaataaagaaacctgaaggaaagccagatcagaagtttgatcaaaagcaagagcttggacgtgtgatacatctcagcaatttgccgcattctggctattctgatagtgctgttctcaagcttgctgagccttatgggaaaataaagaattacatattgatgaggatgaaaagtcaggcttttattgagatggagacaagagaagatgcaatggcaatggttgaccattgtttgaaaaaagccctttggtttcaggggagatgtgtgaaggttgacctgtctgagaaatataaaaaactggttctgaggattccaaacagaggcattgatttactgaaaaaagataaatcccgaaaaagatcttactctccagatggcaaagaatctccaagtgataagaaatccaaaactgatggttcccagaagactgagagttcaaccgaaggtaaagaacaagaagagaagtccggtgaagatggtgagaaagacacaaaggatgaccagacagagcaggaacctaatatgcttcttgaatctgaagatgagctacttgtagatgaagaagaagcagcagcactgctagaaagtggcagttcagtgggagacgagaccgatcttgctaatttaggtgatgtggcttctgatgggaaaaaggaaccatcagataaagctgtgaaaaaagatggaagtgcttcagcagcagcaaagaaaaagcttaaaaaggtggacaagatcgaggaacttgatcaagaaaacgaagcagcgttggaaaatggaattaaaaatgaggaaaacacagaaccaggtgctgaatcttctgagaacgctgatgatcccaacaaagatacaagtgaaaacgcagatggtcaaagtgatgagaacaaggacgactatacaatcccagatgagtatagaattggaccatatcagcccaatgttcctgttggtatagactatgtgatacctaaaacagggttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaactcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 86 - DNA, 6820 bp, Artificial Sequence
  synthetic
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctccaccgcggtggcggccgccaccatggattacaaggatgacgacgataagagcccgggcggatcctccaagtcattccagcagtcatctctcagtagggactcacagggtcatgggcgtgacctgtctgcggcaggaataggccttcttgctgctgctacccagtctttaagtatgccagcatctcttggaaggatgaaccagggtactgcacgccttgctagtttaatgaatcttggaatgagttcttcattgaatcaacaaggagctcatagtgcactgtcttctgctagtacttcttcccataatttgcagtctatatttaacattggaagtagaggtccactccctttatcttctcaacaccgtggagatgcagaccaggccagtaacattttggccagctttggtctgtctgctagagacttagatgaactgagtcgttatccagaggacaagattactcctgagaatttgccccaaatccttctacagcttaaaaggaggagaactgaagaaggccctaccttgagttatggtagagatggcagatctgctacacgggagccaccatacagagtacctagggatgattgggaagaaaaaaggcactttagaagagatagttttgatgatcgtggtcctagtctcaacccagtgcttgattatgaccatggaagtcgttctcaagaatctggttattatgacagaatggattatgaagatgacagattaagagatggagaaaggtgtagggatgattctttttttggtgagacctcgcataactatcataaatttgacagtgagtatgagagaatgggacgtggtcctggccccttacaagagagatctctctttgagaaaaagagaggcgctcctccaagtagcaatattgaagacttccatggactcttaccgaagggttaaccccatctgtgctctatatgtgatttgccagttcattctaataaggagtggagtcaacatatcaatggagcaagtcacagtcgtcgatgccagcttcttcttgaaatctacccagaatggaatcctgacaatgatacaggacacacaatgggtgatccattcatgttgcagcagtctacaaatccagcaccaggaattctgggacctccacctccctcatttcatcttgggggaccagcagttggaccaagaggaaatctgggtgctggaaatggaaacctgcaaggacctagacacatgcagaaaggcagagtggaaactagcagagttgttcacatcatggattttcaacgagggaaaaacttgagataccagctattacagctggtagaaccatttggagtcatttcaaatcatctgattctaaataaaattaatgaggcatttattgaaatggcaaccacagaggatgctcaggccgcagtggattattacacaaccacaccagcgttagtatttggcaagccagtgagagttcatttatcccagaagtataaaagaataaagaaacctgaaggaaagccagatcagaagtttgatcaaaagcaagagcttggacgtgtgatacatctcagcaatttgccgcattctggctattctgatagtgctgttctcaagcttgctgagccttatgggaaaataaagaattacatattgatgaggatgaaaagtcaggcttttattgagatggagacaagagaagatgcaatggcaatggttgaccattgtttgaaaaaagccctttggtttcaggggagatgtgtgaaggttgacctgtctgagaaatataaaaaactggttctgaggattccaaacagaggcattgatttactgaaaaaagataaatcccgaaaaagatcttactctccagatggcaaagaatctccaagtgataagaaatccaaaactgatggttcccagaagactgagagttcaaccgaaggtaaagaacaagaagagaagtccggtgaagatggtgagaaagacacaaaggatgaccagacagagcaggaacctaatatgcttcttgaatctgaagatgagctacttgtagatgaagaagaagcagcagcactgctagaaagtggcagttcagtgggagacgagaccgatcttgctaatttaggtgatgtggcttctgatgggaaaaaggaaccatcagataaagctgtgaaaaaagatggaagtgcttcagcagcagcaaagaaaaagcttaaaaaggtggacaagatcgaggaacttgatcaagaaaacgaagcagcgttggaaaatggaattaaaaatgaggaaaacacagaaccaggtgctgaatcttctgagaacgctgatgatcccaacaaagatacaagtgaaaacgcagatggtcaaagtgatgagaacaaggacgactatacaatcccagatgagtatagaattggaccatatcagcccaatgttcctgttggtatagactatgtgatacctaaaacagggttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaactcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 87 - DNA, 6820 bp, Artificial Sequence
  synthetic
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctccaccgcggtggcggccgccaccatggattacaaggatgacgacgataagagcccgggcggatcctccaagtcattccagcagtcatctctcagtagggactcacagggtcatgggcgtgacctgtctgcggcaggaataggccttcttgctgctgctacccagtctttaagtatgccagcatctcttggaaggatgaaccagggtactgcacgccttgctagtttaatgaatcttggaatgagttcttcattgaatcaacaaggagctcatagtgcactgtcttctgctagtacttcttcccataatttgcagtctatatttaacattggaagtagaggtccactccctttatcttctcaacaccgtggagatgcagaccaggccagtaacattttggccagctttggtctgtctgctagagacttagatgaactgagtcgttatccagaggacaagattactcctgagaatttgccccaaatccttctacagcttaaaaggaggagaactgaagaaggccctaccttgagttatggtagagatggcagatctgctacacgggagccaccatacagagtacctagggatgattgggaagaaaaaaggcactttagaagagatagttttgatgatcgtggtcctagtctcaacccagtgcttgattatgaccatggaagtcgttctcaagaatctggttattatgacagaatggattatgaagatgacagattaagagatggagaaaggtgtagggatgattctttttttggtgagacctcgcataactatcataaatttgacagtgagtatgagagaatgggacgtggtcctggccccttacaagagagatctctctttgagaaaaagagaggcgctcctccaagtagcaatattgaagacttccatggactcttaccgaagggttatccccatctgtgctctatatgtgatttgccagttcattctaataaggagtggagtcaacatatcaatggagcaagtcacagtcgtcgatgccagcttcttctttaaatctacccagaatggaatcctgacaatgatacaggacacacaatgggtgatccattcatgttgcagcagtctacaaatccagcaccaggaattctgggacctccacctccctcatttcatcttgggggaccagcagttggaccaagaggaaatctgggtgctggaaatggaaacctgcaaggacctagacacatgcagaaaggcagagtggaaactagcagagttgttcacatcatggattttcaacgagggaaaaacttgagataccagctattacagctggtagaaccatttggagtcatttcaaatcatctgattctaaataaaattaatgaggcatttattgaaatggcaaccacagaggatgctcaggccgcagtggattattacacaaccacaccagcgttagtatttggcaagccagtgagagttcatttatcccagaagtataaaagaataaagaaacctgaaggaaagccagatcagaagtttgatcaaaagcaagagcttggacgtgtgatacatctcagcaatttgccgcattctggctattctgatagtgctgttctcaagcttgctgagccttatgggaaaataaagaattacatattgatgaggatgaaaagtcaggcttttattgagatggagacaagagaagatgcaatggcaatggttgaccattgtttgaaaaaagccctttggtttcaggggagatgtgtgaaggttgacctgtctgagaaatataaaaaactggttctgaggattccaaacagaggcattgatttactgaaaaaagataaatcccgaaaaagatcttactctccagatggcaaagaatctccaagtgataagaaatccaaaactgatggttcccagaagactgagagttcaaccgaaggtaaagaacaagaagagaagtccggtgaagatggtgagaaagacacaaaggatgaccagacagagcaggaacctaatatgcttcttgaatctgaagatgagctacttgtagatgaagaagaagcagcagcactgctagaaagtggcagttcagtgggagacgagaccgatcttgctaatttaggtgatgtggcttctgatgggaaaaaggaaccatcagataaagctgtgaaaaaagatggaagtgcttcagcagcagcaaagaaaaagcttaaaaaggtggacaagatcgaggaacttgatcaagaaaacgaagcagcgttggaaaatggaattaaaaatgaggaaaacacagaaccaggtgctgaatcttctgagaacgctgatgatcccaacaaagatacaagtgaaaacgcagatggtcaaagtgatgagaacaaggacgactatacaatcccagatgagtatagaattggaccatatcagcccaatgttcctgttggtatagactatgtgatacctaaaacagggttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaactcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 88 - DNA, 6820 bp, Artificial Sequence
  synthetic
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctccaccgcggtggcggccgccaccatggattacaaggatgacgacgataagagcccgggcggatcctccaagtcattccagcagtcatctctcagtagggactcacagggtcatgggcgtgacctgtctgcggcaggaataggccttcttgctgctgctacccagtctttaagtatgccagcatctcttggaaggatgaaccagggtactgcacgccttgctagtttaatgaatcttggaatgagttcttcattgaatcaacaaggagctcatagtgcactgtcttctgctagtacttcttcccataatttgcagtctatatttaacattggaagtagaggtccactccctttatcttctcaacaccgtggagatgcagaccaggccagtaacattttggccagctttggtctgtctgctagagacttagatgaactgagtcgttatccagaggacaagattactcctgagaatttgccccaaatccttctacagcttaaaaggaggagaactgaagaaggccctaccttgagttatggtagagatggcagatctgctacacgggagccaccatacagagtacctagggatgattgggaagaaaaaaggcactttagaagagatagttttgatgatcgtggtcctagtctcaacccagtgcttgattatgaccatggaagtcgttctcaagaatctggttattatgacagaatggattatgaagatgacagattaagagatggagaaaggtgtagggatgattctttttttggtgagacctcgcataactatcataaatttgacagtgagtatgagagaatgggacgtggtcctggccccttacaagagagatctctctttgagaaaaagagaggcgctcctccaagtagcaatattgaagacttccatggactcttaccgaagggttatccccatctgtgctctatatgtgatttgccagttcattctaataaggagtggagtcaacatatcaatggagcaagtcacagtcgtcgatgccagcttcttcttgaaatctacccagaatggaatcctgacaatgatacaggacacacaatgggtgatccattcatgttgcagcagtctacaaatccagcaccaggaattctgggacctccacctccctcatttcatcttgggggaccagcagttggaccaagaggaaatctgggtgctggaaatggaaacctgcaaggacctagacacatgcagaaaggcagagtggaaactagcagagttgttcacatcatggattttcaacgagggaaaaacttgagataccagctattacagctggtagaaccatttggagtcatttcaaatcatctgattctaaataaaattaatgaggcatttattgaaatggcaaccacagaggatgctcaggccgcagtggattattacacaaccacaccagcgttagtatttggcaagccagtgagagttcatttatcccagaagtataaaagaataaagaaacctgaaggaaagccagatcagaagtttgatcaaaagcaagagcttggacgtgtgatacatctcagcaatttgccgcattctggctattctgatagtgctgttctcaagcttgctgagccttatgggaaaataaagaattacatattgatgaggatgaaaagtcaggcttttattgagatggagacaagagaagatgcaatggcaatggttgaccattgtttgaaaaaagccctttggtttcaggggagatgtgtgaaggttgacctgtctgagaaatataaaaaactggttctgaggattccaaacagaggcattgatttactgaaaaaagataaatcccgaaaaagatcttactctccagatggcaaagaatctccaagtgataagaaatccaaaactgatggttcccagaagactgagagttcaaccgaaggtaaagaacaagaagagaagtccggtgaagatggtgagaaagacacaaaggatgaccagacagagcaggaacctaatatgcttcttgaatctgaagatgagctacttgtagatgaagaagaagcagcagcactgctagaaagtggcagttcagtgggagacgagaccgatcttgctaatttaggtgatgtggcttctgatgggaaaaaggaaccatcagataaagctgtgaaaaaagatggaagtgcttcagcagcagcaaagaaaaagcttaaaaaggtggacaagatcgaggaacttgatcaagaaaacgaagcagcgttggaaaatggaattaaaaatgaggaaaacacagaaccaggtgctgaatcttctgagaacgctgatgatcccaacaaagatacaagtgaaaacgcagatggtcaaagtgatgagaacaaggacgactatacaatcccagatgagtatagaattggaccatatcagcccaatgttcctgttggtatagactatgtgatacctaaataagggttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaactcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 89 - DNA, 5959 bp, Artificial Sequence
  synthetic
atgcattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgattacgccaagctcgaaattaaccctcactaaagggaacaaaagctggagctccaccgcggtggcggccgccaccatggattacaaggatgacgacgataagagcccgggcggatcctatccccatctgtgctctatatgtgatttgccagttcattctaataaggagtggagtcaacatatcaatggagcaagtcacagtcgtcgatgccagcttcttcttgaaatctacccagaatggaatcctgacaatgatacaggacacacaatgggtgatccattcatgttgcagcagtctacaaatccagcaccaggaattctgggacctccacctccctcatttcatcttgggggaccagcagttggaccaagaggaaatctgggtgctggaaatggaaacctgcaaggacctagacacatgcagaaaggcagagtggaaactagcagagttgttcacatcatggattttcaacgagggaaaaacttgagataccagctattacagctggtagaaccatttggagtcatttcaaatcatctgattctaaataaaattaatgaggcatttattgaaatggcaaccacagaggatgctcaggccgcagtggattattacacaaccacaccagcgttagtatttggcaagccagtgagagttcatttatcccagaagtataaaagaataaagaaacctgaaggaaagccagatcagaagtttgatcaaaagcaagagcttggacgtgtgatacatctcagcaatttgccgcattctggctattctgatagtgctgttctcaagcttgctgagccttatgggaaaataaagaattacatattgatgaggatgaaaagtcaggcttttattgagatggagacaagagaagatgcaatggcaatggttgaccattgtttgaaaaaagccctttggtttcaggggagatgtgtgaaggttgacctgtctgagaaatataaaaaactggttctgaggattccaaacagaggcattgatttactgaaaaaagataaatcccgaaaaagatcttactctccagatggcaaagaatctccaagtgataagaaatccaaaactgatggttcccagaagactgagagttcaaccgaaggtaaagaacaagaagagaagtccggtgaagatggtgagaaagacacaaaggatgaccagacagagcaggaacctaatatgcttcttgaatctgaagatgagctacttgtagatgaagaagaagcagcagcactgctagaaagtggcagttcagtgggagacgagaccgatcttgctaatttaggtgatgtggcttctgatgggaaaaaggaaccatcagataaagctgtgaaaaaagatggaagtgcttcagcagcagcaaagaaaaagcttaaaaaggtggacaagatcgaggaacttgatcaagaaaacgaagcagcgttggaaaatggaattaaaaatgaggaaaacacagaaccaggtgctgaatcttctgagaacgctgatgatcccaacaaagatacaagtgaaaacgcagatggtcaaagtgatgagaacaaggacgactatacaatcccagatgagtatagaattggaccatatcagcccaatgttcctgttggtatagactatgtgatacctaaaacagggttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaactcgagggggggcccggtaccttaattaattaaggtaccaggtaagtgtacccaattcgccctatagtgagtcgtattacaattcactcgatcgcccttcccaacagttgcgcagcctgaatggcgaatggagatccaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagaatcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagaacatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagaacttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcc

SEQ ID NO: 90 - DNA, 5830 bp, Artificial Sequence
  synthetic
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatctcgtcgtgcatctgttggatcctccaagtcattccagcagtcatctctcagtagggactcacagggtcatgggcgtgacctgtctgcggcaggaataggccttcttgctgctgctacccagtctttaagtatgccagcatctcttggaaggatgaaccagggtactgcacgccttgctagtttaatgaatcttggaatgagttcttcattgaatcaacaaggagctcatagtgcactgtcttctgctagtacttcttcccataatttgcagtctatatttaacattggaagtagaggtccactccctttatcttctcaacaccgtggagatgcagaccaggccagtaacattttggccagctttggtctgtctgctagagacttagatgaactgagtcgttatccagaggacaagattactcctgagaatttgccccaaatccttctacagcttaaaaggaggagaactgaagaaggccctaccttgagttatggtagagatggcagatctgctacacgggagccaccatacagagtacctagggatgattgggaagaaaaaaggcactttagaagagatagttttgatgatcgtggtcctagtctcaacccagtgcttgattatgaccatggaagtcgttctcaagaatctggttattatgacagaatggattatgaagatgacagattaagagatggagaaaggtgtagggatgattctttttttggtgagacctcgcataactatcataaatttgacagtgagtatgagagaatgggacgtggtcctggccccttacaagagagatctctctttgagaaaaagagaggcgctcctccaagtagcaatattgaagacttccatggactcttaccgaagtaaccgggaattcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt

SEQ ID NO: 91 - DNA, 5959 bp, Artificial Sequence
  synthetic
gcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatataccatgaaacatcaccatcaccatcaccccatgaaacagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaaggatctggcagtggttctgagaatctttattttcagggcgccatggaagccctcccgacaccctcggacagcaccctccccgcggaagcccggggacgaggacggcgacggagactcgtttggaccccgagccaaagcgaggccctgcgagcctgctttgagcggaacccgtacccgggcatcgccaccagagaacggctggcccaggccatcggcattccggagcccagggtccagatttggtttcagaatgagaggtcacgccagctgaggcagcaccggcgggaatctcggccctggcccgggagacgcggcccgccagaaggccggcgaaagcggaccgccgtcaccggatcccagaccgccctgctcctccgagcctttgagaaggatcgctttccaggcatcgccgcccgggaggagctggccagagagacgggcctcccggagtccaggattcagatctggtttcagaatcgaagggccaggcacccgggacagggtggcagggcgcccgcgcaggtctagctcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgtt

SEQ ID NO: 92 - DNA, 9941 bp, Artificial Sequence
  synthetic
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaagggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagacaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 93 - DNA, 12484 bp, Artificial Sequence
  synthetic
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaagggtgcagcggcctccgcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagacaggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacatgtccaagtcattccagcagtcatctctcagtagggactcacagggtcatgggcgtgacctgtctgcggcaggaataggccttcttgctgctgctacccagtctttaagtatgccagcatctcttggaaggatgaaccagggtactgcacgccttgctagtttaatgaatcttggaatgagttcttcattgaatcaacaaggagctcatagtgcactgtcttctgctagtacttcttcccataatttgcagtctatatttaacattggaagtagaggtccactccctttatcttctcaacaccgtggagatgcagaccaggccagtaacattttggccagctttggtctgtctgctagagacttagatgaactgagtcgttatccagaggacaagattactcctgagaatttgccccaaatccttctacagcttaaaaggaggagaactgaagaaggccctaccttgagttatggtagagatggcagatctgctacacgggagccaccatacagagtacctagggatgattgggaagaaaaaaggcactttagaagagatagttttgatgatcgtggtcctagtctcaacccagtgcttgattatgaccatggaagtcgttctcaagaatctggttattatgacagaatggattatgaagatgacagattaagagatggagaaaggtgtagggatgattctttttttggtgagacctcgcataactatcataaatttgacagtgagtatgagagaatgggacgtggtcctggccccttacaagagagatctctctttgagaaaaagagaggcgctcctccaagtagcaatattgaagacttccatggactcttaccgaagggttatccccatctgtgctctatatgtgatttgccagttcattctaataaggagtggagtcaacatatcaatggagcaagtcacagtcgtcgatgccagcttcttcttgaaatctacccagaatggaatcctgacaatgatacaggacacacaatgggtgatccattcatgttgcagcagtctacaaatccagcaccaggaattctgggacctccacctccctcatttcatcttgggggaccagcagttggaccaagaggaaatctgggtgctggaaatggaaacctgcaaggacctagacacatgcagaaaggcagagtggaaactagcagagttgttcacatcatggattttcaacgagggaaaaacttgagataccagctattacagctggtagaaccatttggagtcatttcaaatcatctgattctaaataaaattaatgaggcatttattgaaatggcaaccacagaggatgctcaggccgcagtggattattacacaaccacaccagcgttagtatttggcaagccagtgagagttcatttatcccagaagtataaaagaataaagaaacctgaaggaaagccagatcagaagtttgatcaaaagcaagagcttggacgtgtgatacatctcagcaatttgccgcattctggctattctgatagtgctgttctcaagcttgctgagccttatgggaaaataaagaattacatattgatgaggatgaaaagtcaggcttttattgagatggagacaagagaagatgcaatggcaatggttgaccattgtttgaaaaaagccctttggtttcaggggagatgtgtgaaggttgacctgtctgagaaatataaaaaactggttctgaggattccaaacagaggcattgatttactgaaaaaagataaatcccgaaaaagatcttactctccagatggcaaagaatctccaagtgataagaaatccaaaactgatggttcccagaagactgagagttcaaccgaaggtaaagaacaagaagagaagtccggtgaagatggtgagaaagacacaaaggatgaccagacagagcaggaacctaatatgcttcttgaatctgaagatgagctacttgtagatgaagaagaagcagcagcactgctagaaagtggcagttcagtgggagacgagaccgatcttgctaatttaggtgatgtggcttctgatgggaaaaaggaaccatcagataaagctgtgaaaaaagatggaagtgcttcagcagcagcaaagaaaaagcttaaaaaggtggacaagatcgaggaacttgatcaagaaaacgaagcagcgttggaaaatggaattaaaaatgaggaaaacacagaaccaggtgctgaatcttctgagaacgctgatgatcccaacaaagatacaagtgaaaacgcagatggtcaaagtgatgagaacaaggacgactatacaatcccagatgagtatagaattggaccatatcagcccaatgttcctgttggtatagactatgtgatacctaaaacagggttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaaagtaaagcggccgcgactctagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 94 - DNA, 2544 bp, Artificial Sequence
  synthetic
atgtccaagtcattccagcagtcatctctcagtagggactcacagggtcatgggcgtgacctgtctgcggcaggaataggccttcttgctgctgctacccagtctttaagtatgccagcatctcttggaaggatgaaccagggtactgcacgccttgctagtttaatgaatcttggaatgagttcttcattgaatcaacaaggagctcatagtgcactgtcttctgctagtacttcttcccataatttgcagtctatatttaacattggaagtagaggtccactccctttatcttctcaacaccgtggagatgcagaccaggccagtaacattttggccagctttggtctgtctgctagagacttagatgaactgagtcgttatccagaggacaagattactcctgagaatttgccccaaatccttctacagcttaaaaggaggagaactgaagaaggccctaccttgagttatggtagagatggcagatctgctacacgggagccaccatacagagtacctagggatgattgggaagaaaaaaggcactttagaagagatagttttgatgatcgtggtcctagtctcaacccagtgcttgattatgaccatggaagtcgttctcaagaatctggttattatgacagaatggattatgaagatgacagattaagagatggagaaaggtgtagggatgattctttttttggtgagacctcgcataactatcataaatttgacagtgagtatgagagaatgggacgtggtcctggccccttacaagagagatctctctttgagaaaaagagaggcgctcctccaagtagcaatattgaagacttccatggactcttaccgaagggttatccccatctgtgctctatatgtgatttgccagttcattctaataaggagtggagtcaacatatcaatggagcaagtcacagtcgtcgatgccagcttcttcttgaaatctacccagaatggaatcctgacaatgatacaggacacacaatgggtgatccattcatgttgcagcagtctacaaatccagcaccaggaattctgggacctccacctccctcatttcatcttgggggaccagcagttggaccaagaggaaatctgggtgctggaaatggaaacctgcaaggacctagacacatgcagaaaggcagagtggaaactagcagagttgttcacatcatggattttcaacgagggaaaaacttgagataccagctattacagctggtagaaccatttggagtcatttcaaatcatctgattctaaataaaattaatgaggcatttattgaaatggcaaccacagaggatgctcaggccgcagtggattattacacaaccacaccagcgttagtatttggcaagccagtgagagttcatttatcccagaagtataaaagaataaagaaacctgaaggaaagccagatcagaagtttgatcaaaagcaagagcttggacgtgtgatacatctcagcaatttgccgcattctggctattctgatagtgctgttctcaagcttgctgagccttatgggaaaataaagaattacatattgatgaggatgaaaagtcaggcttttattgagatggagacaagagaagatgcaatggcaatggttgaccattgtttgaaaaaagccctttggtttcaggggagatgtgtgaaggttgacctgtctgagaaatataaaaaactggttctgaggattccaaacagaggcattgatttactgaaaaaagataaatcccgaaaaagatcttactctccagatggcaaagaatctccaagtgataagaaatccaaaactgatggttcccagaagactgagagttcaaccgaaggtaaagaacaagaagagaagtccggtgaagatggtgagaaagacacaaaggatgaccagacagagcaggaacctaatatgcttcttgaatctgaagatgagctacttgtagatgaagaagaagcagcagcactgctagaaagtggcagttcagtgggagacgagaccgatcttgctaatttaggtgatgtggcttctgatgggaaaaaggaaccatcagataaagctgtgaaaaaagatggaagtgcttcagcagcagcaaagaaaaagcttaaaaaggtggacaagatcgaggaacttgatcaagaaaacgaagcagcgttggaaaatggaattaaaaatgaggaaaacacagaaccaggtgctgaatcttctgagaacgctgatgatcccaacaaagatacaagtgaaaacgcagatggtcaaagtgatgagaacaaggacgactatacaatcccagatgagtatagaattggaccatatcagcccaatgttcctgttggtatagactatgtgatacctaaaacagggttttactgtaagctgtgttcactcttttatacaaatgaagaagttgcaaagaatactcattgcagcagccttcctcattatcagaaattaaagaaatttctgaataaattggcagaagaacgcagacagaagaaggaaacttaa

SEQ ID NO: 95 - protein, 239 aa, Artificial Sequence
  synthetic
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSRRASVGSPGIHRD

SEQ ID NO: 96 - protein, 35 aa, Artificial Sequence
  synthetic
GYPHLCSICDLPVHSNKEWSQHINGASHSRRCQLL

SEQ ID NO: 97 - protein, 76 aa, Artificial Sequence
  synthetic
RVVHIMDFQRGKNLRYQLLQLVEPFGVISNHLILNKINEAFIEMATTEDAQAAVDYYTTTPALVFGKPVRVHLSQK

SEQ ID NO: 98 - protein, 80 aa, Artificial Sequence
  synthetic
RVIHLSNLPHSGYSDSAVLKLAEPYGKIKNYILMRMKSQAFIEMETREDAMAMVDHCLKKALWFQGRCVKVDLSEKYKKL

SEQ ID NO: 99 - protein, 36 aa, Artificial Sequence
  synthetic
KTGFYCKLCSLFYTNEEVAKNTHCSSLPHYQKLKKF

(19) United States
(12) Patent Application Publication
(10) Pub. No.: US 2022/0098252 A1
(43) Pub. Date: Mar. 31, 2022

(54) INHIBITOR OF DUX4 AND USES THEREOF

(71) Applicants: OSPEDALE SAN RAFFAELE S.R.L., Milano (MI) (IT); FONDAZIONE CENTRO SAN RAFFAELE, Milano (MI) (IT)

(72) Inventors: Davide GABELLINI, Milano (MI) (IT); Roberto GIAMBRUNO, Milano (MI) (IT); Valeria RUNFOLA, Milano (MI) (IT); Claudia CARONNI, Milano (MI) (IT)

(73) Assignees: OSPEDALE SAN RAFFAELE S.R.L., Milano (MI) (IT); FONDAZIONE CENTRO SAN RAFFAELE, Milano (MI) (IT)

(21) Appl. No.: 17/425,359

(22) PCT Filed: Jan. 27, 2020
(86) PCT No.: PCT/EP2020/051910

§ 371 Date: Jul. 23, 2021

(30) Foreign Application Priority Data
Jan. 25, 2019 (EP) 19153786.9

(51) Int. Cl. C07K 14/47; C12N 15/63; A61P 35/02; A61P 25/00

(52) U.S. Cl. CPC C07K 14/47; A61P 25/00; A61P 35/02; C12N 15/63

(57) ABSTRACT

The present invention relates to an inhibitor of DUX4 and its use, in particular in the prevention and/or treatment of a condition associated with an aberrant expression and/or function of at least one DUX4 protein and/or of at least one DUX4 fusion protein. Preferably the inhibitor is MATRIN-3 (MATR3), fragment, variant, fusion, or conjugate thereof. The invention also relates to a pharmaceutical composition comprising such inhibitor, to vector and nucleic acids.

### TECHNICAL FIELD

The present invention relates to an inhibitor of DUX4 and its use, in particular in the prevention and/or treatment of a condition associated with an aberrant expression and/or function of at least one DUX4 protein and/or of at least one DUX4 fusion protein. Preferably the inhibitor is MATRIN-3 (MATR3), fragment, variant, fusion, or conjugate thereof. The invention also relates to a pharmaceutical composition comprising such inhibitor, to vector and nucleic acids.

### BACKGROUND ART

The double homeobox 4 (DUX4) gene encodes for a transcription factor with a key role in early development. In particular, DUX4 is transiently expressed from the zygote to the 4-cell stages in human embryos and is required to activate a cleavage-stage transcriptional program which is part of the zygotic genome activation (ZGA) (Nat. Genet. 2017, 49, 925-934; Nat. Genet. 2017, 49, 935-940; Nat. Genet. 2017, 49, 941-945). From the 8-cell stage onward, DUX4 gene is silenced by repeat-mediated epigenetic repression and remains silent in most tissues of the body with the exception of testis and thymus. The proper control of DUX4 expression and activity is vital, since its aberrant expression/activity is associated to several pathological conditions including facioscapulohumeral muscular dystrophy (FSHD) (Hum Mol Genet. 2018 Aug. 1; 27(R2):R153-R162), herpesvirus infection (Nat Microbiol. 2019 January; 4(1):164-176), acute lymphoblastic leukemia (ALL) (Nat Genet. 2016 December; 48(12):1481-1489; Nat Commun. 2016 Jun. 6; 7:11790; EBioMedicine. 2016 June; 8:173-183; Nat Genet. 2016 May; 48(5):569-74), undifferentiated small round blue cell sarcoma (Am J Case Rep 2015; 16: 87-94), rhabdomyosarcoma and several other human cancers (Cell Stem Cell. 2018 Dec. 6; 23(6):794-805.e4).

Facioscapulohumeral muscular dystrophy (FSHD) is one of the most prevalent neuromuscular disorders (1) and leads to significant lifetime morbidity, with up to 25% of patients requiring wheelchair. The disease is characterized by rostro-caudal progressive wasting in a specific subset of muscles. Symptoms typically appear as asymmetric weakness of the facial (facio), shoulder (scapulo), and upper arm (humeral) muscles, and might progress to affect other skeletal muscle groups. Extra-muscular manifestations can occur in severe cases, including retinal vasculopathy, hearing loss, respiratory defects, cardiac involvement, mental retardation and epilepsy (2). FSHD is not caused by a classical form of gene mutation that results in loss or altered protein function. Likewise, it differs from typical muscular dystrophies by the absence of sarcolemma defects (3). Instead, FSHD is linked to epigenetic alterations that affect the D4Z4 macrosatellite repeat array at 4q35 and cause chromatin relaxation leading to inappropriate gain of expression of the D4Z4-embedded double homeobox 4 (DUX4) gene (2).

Facioscapulohumeral muscular dystrophy (FSHD) is the most common neuromuscular disorder affecting all sexes and ages. Due to an unknown molecular mechanism, FSHD displays overlapping manifestations with amyotrophic lateral sclerosis (ALS). FSHD is caused by aberrant expression of the transcription factor double homeobox 4 (DUX4), which is toxic to skeletal muscle leading to disease.

DUX4 is a homeodomain-containing transcription factor and an important regulator of early development, as it plays an essential role in activating the embryonic genome during the 2- to 8-cell stage of development (4) (5) (6). As such, DUX4 is not typically expressed in somatic cells, and importantly it is silent in healthy skeletal muscle. While the exact pathways by which aberrant DUX4 expression leads to muscular dystrophy are incompletely known, ectopic expression of DUX4 in multiple cell lines as well as in skeletal muscle in vivo leads to apoptotic cell death (7) (8) (9) (10) (11) (12). Importantly, increased apoptosis and its dependence on DUX4 has been documented in FSHD cells and tissues (13) (14) (15) (16).

Despite several clinical trials (17) (18) (19) (20) (21), there continues to be no cure or therapeutic option available to FSHD patients. However, the consensus that ectopic DUX4 expression in skeletal muscle is the root cause of FSHD pathophysiology has opened the possibility of targeted therapies. Importantly, it has been shown that the ability of DUX4 to activate its direct transcriptional targets is required for DUX4-induced muscle toxicity (9) (22). Accordingly, DUX4 targets account for the majority of gene expression alterations in FSHD skeletal muscle (11) (23). Thus, blocking the ability of DUX4 to activate its transcriptional targets has strong therapeutic relevance.

Acute lymphoblastic leukemia (ALL) is the most common cancer in children and is the most frequent cause of death before 20 years of age (DOI: 10.1056/NEJMra1400972). During the last decades, the prognosis of childhood ALL has improved dramatically, but this has been obtained mainly by the use of more effective combination of existing chemotherapeutic agents, rather than the development of new therapies (DOI: 10.1056/NEJMra1400972). Moreover, the subgroup of patients with refractory/relapsed ALL still presents a dismal prognosis (doi: 10.1080/14656566.2017.1317746) indicating the need for innovative therapeutic approaches.

Approximately 85% of childhood ALL is due to defects in the B-cell precursor (BCP) lineage, where B-cells arrest at the precursor stage and do not differentiate into mature cells. B-progenitor ALL represents an heterogeneous disease, including multiple subtypes, commonly defined by structural chromosomal alterations (initiating lesions), followed by secondary somatic (tumor-acquired) DNA copy-number alterations and sequence mutations that contribute to leukemogenesis. Chromosomal alterations include aneuploidy and chromosomal rearrangements that result in oncogene deregulation or expression of chimeric fusion genes (doi: 10.11406/rinketsu.58.1031).

Recently, recurrent rearrangements affecting the double homeobox 4 gene (DUX4) gene have been described as the most frequent event detected in BCR-ABL1-negative ALL patients (doi: 10.1038/ng.3535; doi: 10.1038/ng.3691; doi: 10.1016/j.ebiom.2016.04.038; doi: 10.1038/ncomms11790). DUX4 is a primate-specific transcription factor, encoded by a repeat array in the subtelomeric region of human chromosome 4q. Its expression is normally restricted to germline and stem cells (doi: 10.1093/hmg/ddy162), while it is silent in somatic tissues. Aberrant expression of DUX4 in skeletal muscle, due to loss of epigenetic silencing, is the cause of facioscapulohumeral muscular dystrophy (FSHD) (doi: 10.1093/hmg/ddy162). Furthermore, DUX4 overexpression in somatic cells is extremely toxic, as it activates a pro-apoptotic transcriptional program, that is dependent on the presence of a proficient transactivation domain at the C-terminus of the protein (doi: 10.1093/hmg/ddy162). In ALL, the translocation places DUX4 under the control of the IGH enhancer and results in the disruption of the highly conserved C-terminus of DUX4, leading to pro-B cell expression of the fusion protein DUX4-IGH (doi: 10.1038/ng.3535; doi: 10.1038/ng.3691). Contrary to DUX4, DUX4-IGH does not trigger apoptosis, while it induces transformation of NIH-3T3 fibroblasts and is required for the proliferation of NALM-6 cells, which harbor DUX4-IGH fusion (doi: 10.1038/ng.3691). Moreover, expression of DUX4-IGH in mouse pro-B cells is sufficient to give rise to leukemia, while the expression of wild-type DUX4 in the same cells triggers cell death (doi: 10.1038/ng.3691).

DUX4-IGH expression is a universal feature of this subtype of leukemia occurring early in leukemogenesis and it is maintained in leukemia at relapse (doi: 10.1038/ng.3535; doi: 10.1038/ng.3691), strongly supporting the role of the fusion protein as oncogenic driver.

However, there is still the need for DUX4 inhibitors, in particular for the treatment of cancer, muscular dystrophy and infection.

### SUMMARY OF THE INVENTION

At present, no molecule able to directly control DUX4 function is currently known. The inventors identified Matrin 3 (MATR3), mutated in ALS, as the first cellular factor able to directly interfere with DUX4 and its toxicity. The inventors found that MATR3 binds to the DNA binding domain of DUX4, thereby opposing the activation of its genomic targets. Consequently, MATR3 expression blocks the amplification of DUX4 expression and rescues cell viability and myogenic differentiation of FSHD muscle cells. The present data promote MATR3 as a therapeutic molecule to develop a rational treatment for disease associated with an aberrant expression and/or function of at least one DUX4 protein and/or of at least one DUX4 fusion protein. The inventors have identified the first direct inhibitor of DUX4-induced toxicity. The inventors found that Matrin 3 (MATR3) directly binds to DUX4 and blocks its ability to activate target genes. Importantly, the inventors showed that expression of MATR3 increases survival and improves muscle differentiation of cellular models of FSHD. The present results point to MATR3 as a natural modulator of DUX4 activity that could be targeted for the development of novel therapeutic strategies to effectively treat a condition associated with an aberrant expression and/or function of at least one DUX4 protein and/or of at least one DUX4 fusion protein, such as muscular dystrophy, cancer or infection, more particularly FSHD, herpes infection or ALL. Therefore, the present invention provides a method of treating a condition associated with an aberrant expression and/or function of at least one DUX4 protein and/or of at least one DUX4 fusion protein comprising administering a therapeutically effective amount of MATRIN-3 (MATR3), fragment, variant, fusion, or conjugate thereof.

Preferably the MATRIN-3 (MATR3) variant is selected from Table 1.

Preferably the MATRIN-3 (MATR3) or a fragment thereof is an MCPP-MATRIN-3 (MATR3) fusion protein or an MCPP-Degrader-MATRIN-3 (MATR3) fusion protein.

Still preferably the MATRIN-3 (MATR3) or a fragment thereof is a fatty acid-MATRIN-3 (MATR3) conjugate or a PEG-MATRIN-3 (MATR3) conjugate.

The invention also provides a method of treating a condition associated with aberrant expression and/or function of DUX4 protein and/or of DUX4 fusion proteins comprising administering a therapeutically effective amount of a pharmaceutical composition comprising MATRIN-3 (MATR3) protein, variant, mutant, fusion, or conjugate thereof.

Preferably the pharmaceutical composition further comprises a therapeutic agent. The therapeutic agent is for example a FSHD: anti-inflammatory and/or anti-oxidant drugs, anti-cancer drugs (chemotherapy), radiation therapy and/or immune checkpoint blockade therapies. For example the anti-inflammatory drug may be aspirin, celecoxib, diclofenac, diflunisal, etodolac, ibuprofen, indomethacin, ketoprofen, ketorolac, nabumetone, naproxen, oxaprozin, piroxicam, salsalate, sulindac, tolmetin or as known in the art. The anti-oxidant drug may be vitamin E, vitamin C, zinc, selenium or as known in the art. The anti-cancer drug may be Bleomycin Sulfate, Cisplatin, Cosmegen (Dactinomycin), Dactinomycin, Etopophos (Etoposide Phosphate), Etoposide, Etoposide Phosphate, Ifex (Ifosfamide), Ifosfamide, Vinblastine Sulfate, Keytruda (Pembrolizumab), Lenvatinib Mesylate, Lenvima (Lenvatinib Mesylate), Megestrol Acetate, Pembrolizumab or as known in the art. The Immune checkpoint blockade therapy may be Ipilimumab, Nivolumab, Pembrolizumab, Atezolizumab, Avelumab, Durvalumab, Cemiplima, Spartalizumab or as known in the art. The radiation therapy may be X-rays, protons or as known in the art.

The present invention also provides a method of treating a condition associated with aberrant expression and/or function of at least one DUX4 protein and/or of at least one DUX4 fusion proteins comprising administering a therapeutically effective amount of a nucleic acid construct encoding the MATRIN-3 (MATR3) protein, fragment, variant, fusion, or conjugate thereof as defined above.

The present invention further provides a method of treating a condition associated with aberrant expression and/or function of at least one DUX4 protein and/or of at least one DUX4 fusion proteins comprising administering a therapeutically effective amount of an expression vector comprising the nucleic acid construct as defined above, preferably the expression vector comprises the nucleic acid construct as defined above and a promoter operatively linked thereto.

Preferably the promoter drives the expression of MATRIN-3 protein, fragment, variant, fusion, or conjugate thereof in the muscle or in the tumor or in the infected cell.

In a preferred embodiment the expression vector is an AVV vector.

Preferably the promoter is a muscle-specific promoter.

The present invention also provides a method of treating a condition associated with aberrant expression and/or function of DUX4 protein and/or of DUX4 fusion proteins comprising administering a therapeutically effective amount of a transformed cell comprising the vector as defined above, preferably the cell is a eukaryotic cell selected from the group consisting of a mammalian cell, an insect cell, a plant cell, a yeast cell and a protozoa cell. Still preferably the cell is a human cell or a bacterial cell.

Preferably the condition associated with aberrant expression and/or function of DUX4 protein and/or of DUX4 fusion proteins is selected from the group consisting of: muscular dystrophy, infection or cancer.

Preferably the cancer is selected from the group consisting of: acute lymphoblastic leukemia, undifferentiated small round blue cell sarcoma, rhabdomyosarcoma, breast, testis, kidney, stomach, lung, thymus, liver, uterus, larynx, esophagus, tongue, heart, connective, mouth, colon, mesothelioma, bladder, ovary, brain, tonsil, pancreas, peritoneum, prostatic or thyroid cancer (Cell Stem Cell. 2018 Dec. 6; 23(6):794-805.e4 incorporated by reference).

Preferably the infection is a herpes virus infection. Preferably the muscular dystrophy is FSHD. A condition associated with an aberrant expression and/or function of DUX4 protein and/or of DUX4 fusion protein means a condition in which the expression of the protein DUX4 itself or its fused forms (at the level of RNA or protein) is altered in comparison with a healthy subject, a subject not affected by such a condition.

DUX4 or its fused forms are normally not expressed in somatic tissues. As a result of genomic alterations, the expression of DUX4 or of its fused forms is aberrantly activated and/or the activity of DUX4 or of its fused form is altered in several conditions. Specifically, muscular dystrophy, infection or cancer such as FSHD, herpesvirus infection, rhabdomyosarcoma, ALL.

In these conditions, DUX4 or its fused forms are overexpressed. In ALL and undifferentiated small round blue cell sarcoma aberrant expression and activity of DUX4 fused forms is observed. Fused forms of DUX4 include the capicua locus (CIC-DUX4) or the immunoglobulin heavy locus (DUX4-IGH) fusion transcripts.

With the exception of 4-cell embryonic stage, testis and thymus, DUX4 is normally not expressed. Aberrant expression of DUX4 or its fused form is intended to be when expression (at the level of RNA or protein) is observed while it is not normally observed or overexpressed in respect to a proper control.

In the diseases or conditions of the invention, expression of DUX4 (RNA and protein) or its fused forms is observed and/or measured in tissues and cell types that normally do not expressed DUX4.

Due to genomic translocations, cancers such as ALL and undifferentiated small round blue cell sarcoma display expression of chimeric proteins in which DUX4 is fused to amino acids encoded by the immunoglobulin heavy locus (DUX4-IGH in ALL) or the capicua locus (CIC-DUX4). These DUX4 fusions display a different (aberrant) activity compared to normal DUX4, including the ability to regulate a different set of genes and to promote cell proliferation instead of cell death.

Aberrant DUX4 or its fused forms expression can be evaluated by quantitative RT-PCR, RNA sequencing, RNA-FISH, immunoblotting and immunofluorescence or any known method in the art.

Aberrant function of DUX4 or its fused forms can be evaluated by monitoring the expression of DUX4/DUX4-IGH/CIC-DUX4 target genes like for example MDB3L2, TRIM43, ERGalt, ETV4 or CCNE1 (Nat Commun. 2019 Jan. 21; 10(1):364; Elife. 2019 Jan. 15; 8. pii: e41740. doi: 10.7554/eLife.41740. [Epub ahead of print]; J Hematol Oncol. 2019 Jan. 14; 12(1):8; Haematologica. 2019 Jan. 10. pii: haematol.2018.204974. doi: 10.3324/haematol.2018.204974. [Epub ahead of print]; Haematologica. 2019 Jan. 10. pii: haematol.2018.204487. doi: 10.3324/haematol.2018.204487. [Epub ahead of print]; Nat Microbiol. 2019 January; 4(1):164-176; Proc Natl Acad Sci USA. 2018 Dec. 11; 115(50):E11711-E11720; Cell Stem Cell. 2018 Dec. 6; 23(6):794-805.e4; Hum Mol Genet. 2018 Dec. 6. doi: 10.1093/hmg/ddy405. [Epub ahead of print]; Hum Mol Genet. 2018 Nov. 16. doi: 10.1093/hmg/ddy400. [Epub ahead of print]; Sci Rep. 2018 Nov. 16; 8(1):16957; Haematologica. 2018 November; 103(11):e522-e526; Leukemia. 2018 Oct. 12. doi: 10.1038/s41375-018-0273-z. [Epub ahead of print]; Leukemia. 2018 June; 32(6):1466-1476; Hum Mol Genet. 2018 May 8. doi: 10.1093/hmg/ddy173 [Epub ahead of print]; J Pathol. 2018 May; 245(1):29-40; PLoS One. 2018 Feb. 7; 13(2):e0192657; Sci Rep. 2018 Jan. 12; 8(1):693; Nat Commun. 2017 Dec. 18; 8(1):2152; J Cell Sci. 2017 Nov. 1; 130(21):3685-3697; Nat Commun. 2017 Sep. 15; 8(1):550; J Hematol Oncol. 2017 Aug. 14; 10(1):148; PLoS Genet. 2017 Mar. 9; 13(3):e1006622; PLoS Genet. 2017 Mar. 8; 13(3):e1006658; Nat Genet. 2016 December; 48(12):1481-1489; Elife. 2016 Nov. 14; 5; Hum Mol Genet. 2016 Oct. 15; 25(20):4419-4431; J Cell Sci. 2016 Oct. 15; 129(20):3816-3831; Nat Commun. 2016 Jun. 6; 7:11790; EBioMedicine. 2016 June; 8:173-183; Hum Mol Genet. 2015 Oct. 15; 24(20):5901-14; Hum Mol Genet. 2015 Mar. 1; 24(5):1256-66; Ann Clin Transl Neurol. 2015 February; 2(2):151-66; Elife. 2015 Jan. 7; 4; J R Soc Interface. 2015 Jan. 6; 12(102):20140797; Mod Pathol. 2015 January; 28(1):57-68; Skelet Muscle. 2014 Oct. 24; 4:19; Hum Mol Genet. 2014 Oct. 15; 23(20):5342-52; Cell Rep. 2014 Sep. 11; 8(5):1484-96; Genes Chromosomes Cancer. 2014 July; 53(7):622-33; Biochem Biophys Res Commun. 2014 Mar. 28; 446(1):235-40; Hum Mol Genet. 2014 Jan. 1; 23(1):171-81; PLoS Genet. 2013 Nov.; 9(11):e1003947; PLoS One. 2013 May 22; 8(5):e64691; PLoS Genet. 2013 Apr.; 9(4):e1003415; Dev Cell. 2012 Jan. 17; 22(1):38-51) and by evaluating cell proliferation, cell differentiation, cell transformation, cell apoptosis, oxidative damage, tumor formation and tumor growth, according to any known method in the art.

MATR3 is an DNA- and RNA-binding component of the nuclear matrix involved in diverse processes, including the response to DNA damage (Cell Cycle 2010 9:1568-1576), mRNA stability (PLoS One 2011 6:e23882), RNA splicing (EMBO J 2015 34:653-668), nuclear retention of hyperedited RNA (Cell 2001 106:465-475) and restriction/latency of retroviruses (Retrovirology 2005 12:57; MBio. 2018 Nov. 13; 9(6). pii: e02158-18. doi: 10.1128/mBio.02158-18). MATR3 is 847 amino acids long and contains four known functional domains: Zinc finger 1 (aa 288-322), RNA recognition motif 1 (398-473), RNA recognition motif 2 (496-575) and Zinc finger 2 (798-833).

In the present invention Matrin 3 or a functional fragment thereof exhibits at least one of the following activities:
inhibits DUX4-induced toxicity in particular in HEK293 cells,
blocks induction of DUX4 targets, in particular in HEK293 cells,
interacts with the DNA-binding domain of DUX4,
inhibits DUX4 directly by blocking its ability to bind DNA,
inhibits the expression of DUX4 and DUX4 targets in particular in FSHD muscle cells,
rescues viability and myogenic differentiation in particular of FSHD muscle cells,
inhibits the expression of DUX4 and DUX4 targets in particular in FSHD muscle cells and
rescues viability and myogenic differentiation in particular of FSHD muscle cells.
the ability to treat, prevent, or ameliorate condition associated with an aberrant expression and/or function of at least one DUX4 protein and/or of at least one DUX4 fusion protein, such as muscular dystrophy, infection or cancer such as FSHD, herpes infection or ALL.

These activities may be measured as described herein or using known methods in the art.

The inventors have found that the first 287 amino acids of MATR3 are sufficient to bind DUX4 and inhibits its activity.

MATR3 full length, fragment, variant, fusion, or conjugate thereof or the minimal DUX4 binding MATR3 domain may be delivered to skeletal muscle by using recombinant adeno-associated viruses (rAAV), which are highly prevalent in musculoskeletal gene therapy due to their non-pathogenic nature, versatility, high transduction efficiency, natural muscle tropism and vector genome persistence for years (Curr Opin Pharmacol. 2017 June; 34:56-63). To this aim, the nucleotide coding sequence for MATR3 full length or the minimal DUX4 binding domain may be inserted in rAAV containing a muscle specific promoter such the CK8 regulatory cassette (Nat Commun. 2017 Feb. 14; 8:14454).

An alternative to the gene therapy-like approach for the delivery of MATR3 full length or fragment, variant, fusion, or conjugate thereof or the minimal DUX4 binding MATR3 domain can be the use of recombinant peptides. Peptides are highly selective and efficacious and, at the same time, relatively safe and well-tolerated. A particularly exciting application of peptides is the inhibition of protein-DNA interactions, which remain challenging targets for small molecules. Peptides are generally impermeable to the cell membrane. To allow cellular entry and drive selective uptake from skeletal muscle, MATR3-based peptides could be fused to muscle-targeting cell-penetrating peptides (MCPP) like B-MSP (Hum Mol Genet. 2009 Nov. 15; 18(22):4405-14), Pip6 (Mol Ther Nucleic Acids. 2012 Aug. 14; 1:e38), M12 (Mol Ther. 2014 Jul.; 22(7):1333-1341) or CyPep10 (Mol Ther. 2018 Jan. 3; 26(1):132-147). To increase potency of the MATR3-based fusion peptides, they could further be modified by appending an E3 ubiquitin ligase, which are factors driving attachment of ubiquitin molecules to a lysine on the target protein and triggering degradation of a protein of interest by the proteolytic activity mediated by the proteasome, a protein degradation “machine” within the cell that can digest a variety of proteins into short polypeptides and amino acids.

Pharmacologic protein degradation is a powerful approach with therapeutic relevance. This approach uses bifunctional small molecules (degrader) that engage both a target protein and an E3 ubiquitin ligase, like for example cereblon (CRBN) or Von-Hippel Lindau (VHL), which are expressed in skeletal muscle and have several already known and tested E3 ligase activators, for instance thalidomide, lenalidomide, and pomalidomide (Science 2015 348, 1376-1381; Molecular Cell 2017 67, 5-18). This allows potent and selective degradation of target proteins by enforcing proximity of the targeted protein and the E3 ligase, leading to ubiquitination and proteasomal degradation. After binding to DUX4, the MCPP-degrader-MATR3-based peptide will lead to the ubiquitination of DUX4 at lysin residues and proteasomal degradation. Moreover, cell permeability and metabolic stability of the MATR3-based fusion peptides could be increased by reversible bicyclization (Angew Chem Int Ed Engl. 2017 Feb. 1; 56(6):1525-1529, incorporated by reference).

In some embodiments, the methods of the invention comprise a portion of the wild type MATRIN-3 (MATR3) full length protein, e.g., having NCBI reference sequence number NM 199189.2, and encoded by the polynucleotide sequence which has NCBI reference sequence number NP 954659. By way of non-limiting example, in some embodiments, the methods of the invention comprise the mature MATRIN-3 (MATR3) protein, i.e., amino acid residues 1-847 of the wild type MATRIN-3 (MATR3) full length protein. In other embodiments, the methods of the invention comprise smaller fragments, domains, and/or regions of full length MATRIN-3 (MATR3) protein.

| MATR3 1-797 | |
|---|---|
| (SEQ ID No. 1) | |
| 1 | msksfqqssl srdsqghgrd lsaagiglla aatqslsmpa slgrmnqgta rlaslmnlgm |
| 61 | ssslnqqgah salssastss hnlqsifnig srgplplssq hrgdadqasn ilasfglsar |
| 121 | dldelsrype dkitpenlpq illqlkrrrt eegptlsygr dgrsatrepp yrvprddwee |
| 181 | krhfrrdsfd drgpslnpvl dydhgsrsqe sgyydrmdye ddrlrdgerc rddsffgets |
| 241 | hnyhkfdsey ermgrgpgpl gerslfekkr gappssnied fhgllpkgyp hlcsicdlpv |
| 301 | hsnkewsqhi ngashsrrcq llleiypewn pdndtghtmg dpfmlqqstn papgilgppp |
| 361 | psfhlggpav gprgnlgagn gnlqgprhmq kgrvetsrvv himdfqrgkn lryqllqlve |
| 421 | pfgvisnhli lnkineafie mattedaqaa vdyytttpal vfgkpvrvhl sqkykrikkp |
| 481 | egkpdqkfdq kqelgrvihl snlphsgysd savlklaepy gkiknyilmr mksqafieme |
| 541 | tredamamvd hclkkalwfq grcvkvdlse kykklvlrip nrgidllkkd ksrkrsyspd |
| 601 | gkespsdkks ktdgsqktes stegkeqeek sgedgekdtk ddqteqepnm llesedellv |
| 661 | deeeaaalle sgssvgdetd lanlgdvasd gkkepsdkav kkdgsasaaa kkklkkvdki |
| 721 | eeldqeneaa lengikneen tepgaessen addpnkdtse nadgqsdenk ddytipdeyr |
| 781 | igpyqpnvpv gidyvip |
| MATR3 1-322 | |
| (SEQ ID No. 2) | |
| 1 | msksfqqssl srdsqghgrd lsaagiglla aatqslsmpa slgrmnqgta rlaslmnlgm |
| 61 | ssslnqqgah salssastss hnlqsifnig srgplplssq hrgdadqasn ilasfglsar |
| 121 | dldelsrype dkitpenlpq illqlkrrrt eegptlsygr dgrsatrepp yrvprddwee |
| 181 | krhfrrdsfd drgpslnpvl dydhgsrsqe sgyydrmdye ddrlrdgerc rddsffgets |
| 241 | hnyhkfdsey ermgrgpgpl gerslfekkr gappssnied fhgllpkgyp hlcsicdlpv |
| 301 | hsnkewsqhi ngashsrrcq ll |
| MATR3 1-287 | |
| (SEQ ID No. 3) | |
| 1 | msksfqqssl srdsqghgrd lsaagiglla aatqslsmpa slgrmnqgta rlaslmnlgm |
| 61 | ssslnqqgah salssastss hnlqsifnig srgplplssq hrgdadqasn ilasfglsar |
| 121 | dldelsrype dkitpenlpq illqlkrrrt eegptlsygr dgrsatrepp yrvprddwee |
| 181 | krhfrrdsfd drgpslnpvl dydhgsrsqe sgyydrmdye ddrlrdgerc rddsffgets |
| 241 | hnyhkfdsey ermgrgpgpl gerslfekkr gappssnied fhgllpk |
| MATR3 288-847 | |
| (SEQ ID No. 4) | |
| 288 | gyp hlcsicdlpv |
| 301 | hsnkewsqhi ngashsrrcq llleiypewn pdndtghtmg dpfmlqqstn papgilgppp |
| 361 | psfhlggpav gprgnlgagn gnlqgprhmq kgrvetsrvv himdfqrgkn lryqllqlve |
| 421 | pfgvisnhli lnkineafie mattedaqaa vdyytttpal vfgkpvrvhl sqkykrikkp |
| 481 | egkpdqkfdq kqelgrvihl snlphsgysd savlklaepy gkiknyilmr mksqafieme |
| 541 | tredamamvd hclkkalwfq grcvkvdlse kykklvlrip nrgidllkkd ksrkrsyspd |
| 601 | gkespsdkks ktdgsqktes stegkeqeek sgedgekdtk ddqteqepnm llesedellv |
| 661 | deeeaaalle sgssvgdetd lanlgdvasd gkkepsdkav kkdgsasaaa kkklkkvdki |
| 721 | eeldqeneaa lengikneen tepgaessen addpnkdtse nadgqsdenk ddytipdeyr |
| 781 | igpyqpnvpv gidyvipktg fycklcslfy tneevaknth csslphyqkl kkflnklaee |
| 841 | rrqkket |

In some embodiments, the methods of the invention comprise variants or mutations of the MATRIN-3 (MATR3) protein sequence, e.g., biologically active MATRIN-3 (MATR3) variants, and can include truncated versions of the MATRIN-3 (MATR3) protein (in which residues from the C- and/or N-terminal regions have been eliminated, thereby shortening/truncating the protein), as well as variants with one or more point substitutions, deletions, and/or site-specific incorporation of amino acids at positions of interest (e.g., with conservative amino acid residues, with non-conservative residues, or with non-natural amino acid residues such as pyrrolysine). The terms “variant” and “mutant” are used interchangeably and are further defined herein. In some embodiments, the methods of the invention comprise MATRIN-3 (MATR3) fusion protein sequences, such as Fc fusions, or serum albumin (SA) fusion or fusion with muscle-targeting/cell-penetrating peptides, fusions with bifunctional small molecule degraders, or reversible bicyclization. The terms “fusion protein, “fusion polypeptide,” and “fusions” are used interchangeably and are further defined herein. In still other embodiments, the methods of the invention comprise conjugations of MATRIN-3 (MATR3) and fatty acids. Said conjugates and fusions may be intended to extend the half-life of the MATRIN-3 (MATR3) moiety, in addition to serving as therapeutic agents for the conditions listed herein. In some embodiments, the conjugates and fusions used in the methods of the inventions comprise wild type MATRIN-3 (MATR3); in other embodiments, the conjugates and fusions comprise variant MATRIN-3 (MATR3) sequences relative to the wild type full length or mature protein.

In some embodiments, the methods of the invention comprise MATRIN-3 (MATR3) fusion proteins, such Fc fusion, albumin fusion, fusion with muscle-targeting/cell-penetrating peptides, fusions with bifunctional small molecule degraders, or reversible bicyclization. Said fusions can comprise wild type MATRIN-3 (MATR3) or variants thereof. In some embodiments, the methods of the present invention comprise polypeptides which can be fused to a heterologous amino acid sequence, optionally via a linker, such as GS or (GGGGS)n, wherein n is one to about 20, and preferably 1, 2, 3 or 4. The heterologous amino acid sequence can be an IgG constant domain or fragment thereof (e.g., the Fc region), Human Serum Albumin (HSA), or albumin-binding polypeptides. In some embodiments, the heterologous amino acid sequence is derived from the human IgG4 Fc region because of its reduced ability to bind Fey receptors and complement factors compared to other IgG sub-types. The heterologous amino acid sequence can be a muscle-targeting/cell-penetrating peptide, a bifunctional small molecule degrader, or a reversible bicyclization. Such methods can comprise multimers of said fusion polypeptides. In some embodiments, the methods of the present invention comprise fusion proteins in which the heterologous amino acid sequence (e.g., MCPP, Degrader, etc.) is fused to the amino-terminal of the MATRIN-3 (MATR3) protein or variants as described herein; in other embodiments, the fusion occurs at the carboxyl-terminal of the MATRIN-3 (MATR3) protein or variants.

In some embodiments, the methods of the invention comprise MATRIN-3 (MATR3) conjugates, such as MATRIN-3 (MATR3) fatty acid (FA) conjugates, e.g., MATRIN-3 (MATR3) wild type protein (full length, mature, or fragment or truncation thereof) or variant covalently attached to a fatty acid moiety via a linker.

In some embodiments, the methods of the invention comprise MATRIN-3 (MATR3) fusion proteins or conjugates which are covalently linked to one or more polymers, such as polyethylene glycol (PEG) or polysialic acid. The PEG group is attached in such a way so as enhance, and/or not to interfere with, the biological function of the constituent portions of the fusion proteins or conjugates of the invention.

The invention also provides methods of treatment with a pharmaceutical composition comprising the MATRIN-3 (MATR3) fusion proteins or MATRIN-3 (MATR3) conjugates disclosed herein and a pharmaceutically acceptable formulation agent. Such pharmaceutical compositions can be used in a method for treating one or more of condition associated with an aberrant expression and/or function of DUX4 protein and/or of DUX4 fusion protein and the methods comprise administering to a human patient in need thereof a pharmaceutical composition of the invention.

The invention also provides methods of treatment with a pharmaceutical composition comprising the MATRIN-3 (MATR3) fusion proteins or MATRIN-3 (MATR3) conjugates disclosed herein and a pharmaceutically acceptable formulation agent. Such pharmaceutical compositions can be used in a method for treating one or more of condition associated with an aberrant expression and/or function of DUX4 protein and/or of DUX4 fusion protein and the methods comprise administering to a human patient in need thereof a pharmaceutical composition of the invention. The invention also provides MATRIN-3 (MATR3) fusion proteins or MATRIN-3 (MATR3) conjugates disclosed herein for the treatment of one or more condition associated with aberrant expression and/or function of DUX4 protein and/or of DUX4 fusion protein, such as muscular dystrophy, infection or cancer, in particular FSHD or ALL. The invention also provides pharmaceutical compositions comprising MATRIN-3 (MATR3) fusion proteins or MATRIN-3 (MATR3) conjugates disclosed herein for the treatment of one or more condition associated with aberrant expression and/or function of DUX4 protein and/or of DUX4 fusion protein, such as muscular dystrophy, infection or cancer, in particular FSHD or ALL.

In one embodiment, the methods of the invention comprise MATRIN-3 (MATR3) fusion proteins as described herein, e.g., serum albumin, the muscle-targeting cell penetrating peptide fusions ect. In some embodiments, said fusions can contain any suitable serum albumin, cell penetrating peptide (CPP) moiety, any suitable MATRIN-3 (MATR3) moiety, and if desired, any suitable linker. Generally, the CPP moiety, MATRIN-3 (MATR3) moiety and, if present, linker, are selected to provide a fusion polypeptide that would be predicted to have therapeutic efficacy in a condition associated with aberrant expression and/or function of DUX4 protein and/or of DUX4 fusion protein, such as muscular dystrophy, infection or cancer, in particular FSHD or ALL or other disorders described herein, and to be immunologically compatible with the species to which it is intended to be administered. For example, when the fusion polypeptide is intended to be administered to humans the CPP moiety can be B-MSP or a functional variant thereof, and the MATRIN-3 (MATR3) moiety can be human MATRIN-3 (MATR3) or a functional variant thereof. Similarly, CPP and functional variants thereof and MATRIN-3 (MATR3) and functional variants thereof that are derived from other species (e.g., pet or livestock animals) can be used when the fusion protein is intended for use in such species.

MATRIN-3 (MATR3) Moiety

The MATRIN-3 (MATR3) moiety used in the present methods of the invention, e.g., in any MATRIN-3 (MATR3) fusion protein or conjugate, such as fatty acid conjugate, can be any suitable MATRIN-3 (MATR3) polypeptide or functional variant thereof, for example a MATRIN-3 (MATR3) variant described in Table 1. Preferably, the MATRIN-3 (MATR3) moiety is human MATRIN-3 (MATR3) or a functional variant thereof. Human MATRIN-3 (MATR3) is 847 amino acids long and contains four known functional domains:

Zinc finger 1 (aa 288-322)
(SEQ ID NO: 96)
gyphlcsicdlpvhsnkewsqhingashsrrcqll
RNA recognition motif 1 (398-473)
(SEQ ID NO: 97)
rvvhimdfqrgknlryqllqlvepfgvisnhlilnkineafiemattedaq
aavdyytttpalvfgkpvrvhlsqk
RNA recognition motif 2 (496-575)
(SEQ ID NO: 98)
rvihlsnlphsgysdsavlklaepygkiknyilmrmksqafiemetredam
amvdhclkkalwfqgrcvkvdlsekykkl
Zinc finger 2 (798-833)
(SEQ ID NO: 99)
ktgfycklcslfytneevaknthcsslphyqklkkf

TABLE 1
MATR3 variants (Nat Neurosci. 2014
May; 17(5): 664-666; Neurobiol Aging. 2017
Jan; 49: 218.e1-218.e7, incorporated by reference)

| MATR3cDNA | MATR3 protein |
|---|---|
| variants | variation |
| c.48 + 1G > T | N.A. |
| c.196C > A | p.Q66K |
| c.214G > A | p.A72T |
| c.254C > G | p.S85C |
| c. −339 + 2T > A | N.A. |
| c.344T > G | p.F115C |
| c.439A > T | p.R147W |
| c.457G > T | p.G153C |
| c.460C > T | p.P154S |
| c.1180G > A | p.V394M |
| c.1829C > T | p.S610F |
| c.1864A > G | p.T622A |
| c.1991A > C | p.E664A |
| c.2120C > T | p.S707L |
| c.2360A > G | p.N787S |

Fusion proteins used in the present methods of the invention that contain a human MATRIN-3 (MATR3) moiety generally contain the 1-847, 1-287 or fewer amino acids of MATRIN-3 (MATR3) peptide or a functional variant thereof. The functional variant can include one or more amino acid deletions, additions or replacements in any desired combination, for example, a MATRIN-3 (MATR3) variant in Table 1. The amount of amino acid sequence variation (e.g., through amino acid deletions, additions or replacements) is limited to preserve weight loss activity of the mature MATRIN-3 (MATR3) peptide. In some embodiments, the functional variant of a mature MATRIN-3 (MATR3) peptide has from 1 to about 20, 1 to about 18, 1 to about 17, 1 to about 16, 1 to about 15, 1 to about 14, 1 to about 13, 1 to about 12, 1 to about 11, 1 to about 10, 1 to about 9, 1 to about 8, 1 to about 7, 1 to about 6, or 1 to about 5 amino acid deletions, additions or replacements, in any desired combination, relative to SEQ ID NO:1, 2, 3 or 4 or any of the four known functional domains as reported above. Alternatively, or in addition, the functional variant can have an amino acid sequence that has at least about 80%, at least about 85%, at least about 90%, or at least about 95%, 96%, 97%, 98%, or 99% amino acid sequence identity with SEQ ID NO:1, 2, 3 or 4 or any of the four known functional domains as reported above, preferably when measured over the full length of SEQ ID NO:1, 2, 3 or 4 or any of the four known functional domains as reported above. In a specific embodiment, a MATRIN-3 (MATR3) functional variant can have an amino acid sequence that has at least 90%, at least 95%, or at least 98% amino acid sequence identity with SEQ ID NO:1, 2, 3 or 4 or any of the four known functional domains as reported above, preferably when measured over the full length of SEQ ID NO:1, 2, 3 or 4 or any of the four known functional domains as reported above. Without wishing to be bound by any particular theory, it may be that MATRIN-3 (MATR3)'s therapeutic efficacy in a condition associated with aberrant expression and/or function of DUX4, such as FSHD or ALL, and related conditions mediated either through cellular signaling initiated by the binding of MATRIN-3 (MATR3) (and the fusion proteins and variants described herein) to DUX4 and/or co-factors, or by regulation of pathways utilized by other factors via direct competition or allosteric modulation. Amino acid substitutions, deletions, or additions are preferably at positions that are not involved in maintaining overall protein conformation.

Serum Albumin (SA) Moiety

The SA moiety is any suitable serum albumin (e.g., human serum albumin (HSA), or serum albumin from another species) or a functional variant thereof. Preferably, the SA moiety is an HSA or a functional variant thereof. The SA moiety prolongs the serum half-life of the fusion polypeptides to which it is added, in comparison to wild type MATRIN-3 (MATR3). Methods for pharmacokinetic analysis and determination of serum half-life will be familiar to those skilled in the art. Details may be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics,” M Gibaldi & D Perron, published by Marcel Dekker, 2^{nd} Rev. ex edition (1982), which describes pharmacokinetic parameters such as t alpha and t beta half-lives and area under the curve (AUC).

Human Serum Albumin (HSA) is a plasma protein of about 66,500 KDa and is comprised of 585 amino acids, including at least 17 disulfide bridges. (Peters, T., Jr. (1996), All about Albumin: Biochemistry, Genetics and Medical, Applications, pp 10, Academic Press, Inc., Orlando (ISBN 0-12-552110-3). HSA has a long half-life and is cleared very slowly by the liver. The plasma half-life of HSA is reported to be approximately 19 days (Peters, T., Jr. (1985) Adv. Protein Chem. 37, 161-245; Peters, T., Jr. (1996) All about Albumin, Academic Press, Inc., San Diego, Calif. (page 245-246)); Benotti P, Blackburn G L: Crit. Care Med (1979) 7:520-525).

HSA has been used to produce fusion proteins that have improved shelf and half-lifes. For example, PCT Publications WOO 1/79271 A and WO03/59934 A disclose (i) albumin fusion proteins comprising a variety of therapeutic protein (e.g., growth factors, scFvs); and (ii) HSAs that are reported to have longer shelf and half-lives than their therapeutic proteins alone.

HSA may comprise the full length sequence of 585 amino acids of mature naturally occurring HSA (following processing and removal of the signal and propeptides) or naturally occurring variants thereof, including allelic variants. Naturally occurring HSA and variants thereof are well-known in the art. (See, e.g., Meloun, et al, FEBS Letters 5S: 136 (1975); Behrens, et al., Fed. Proc. 34:591 (1975); Lawn, et al., Nucleic Acids Research 9:6102-6114 (1981); Minghetti, et al, J. Biol. Chem. 261:6747 (1986)); and Weitkamp, et al, Ann. Hum. Genet. 37:219 (1973).)

Full length HAS
(SEQ ID NO: 5)
MKWVTFISLLFLFSSAYSRGVFRRDAHKSEVAKRFKDLGEENFKALVLIAF
AQYLQQCPFEDHVKLVNEVTEFAKTCVADESAENCDKSLHTLFGDKLCTVA
TLRETYGEMADCCAKQEPERNECFLQHKDDNPNLPRLVRPEVDVMCTAFHD
NEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEV
SKLVTDLTKVHTECCHGDLLECADDRADLAKYICENQDSISSKLKECCEKP
LLEKSHCIAEVENDEMPADLPSLAADFVESKDVCKNYAEAKDVFLGMFLYE
YARRHPDYSVVLLLRLAKTYETTLEKCCAAADPHECYAKVFDEFKPLVEEP
QNLIKQNCELFEQLGEYKFQNALLVRYTKKVPQVSTPTLVEVSRNLGKVGS
KCCKHPEAKRMPCAEDYLSVVLNQLCVLHEKTPVSDRVTKCCTESLVNRRP
CFSALEVDETYVPKEFNAETFTFHADICTLSEKERQIKKQTALVELVKHKP
KATKEQLKAVMDDFAAFVEKCCKADDKETCFAEEGKKLVAASQAALGL
Mature HAS (25-609)
(SEQ ID NO: 6)
DAHKSE VAHRFKDLGE ENFKALVLIA FAQYLQQCPF EDHVKLVNEV
TEFAKTCVAD ESAENCDKSL HTLFGDKLCT VATLRETYGE
MADCCAKQEP ERNECFLQHK DDNPNLPRLV RPEVDVMCTA
FHDNEETFLK KYLYEIARRH PYFYAPELLF FAKRYKAAFT
ECCQAADKAA CLLPKLDELR DEGKASSAKQ RLKCASLQKF
GERAFKAWAV ARLSQRFPKA EFAEVSKLVT DLTKVHTECC
HGDLLECADD RADLAKYICE NQDSISSKLK ECCEKPLLEK
SHCIAEVEND EMFADLFSLA ADFVESKDVC KNYAEAKDVF
LGMFLYEYAR RHPDYSVVLL LRLAKTYETT LEKCCAAADP
HECYAKVFDE FKPLVEEPQN LIKQNCELFE QLGEYKFQNA
LLVRYTKKVP QVSTPTLVEV SRNLGKVGSK CCKHPEAKRM
PCAEDYLSVV LNQLCVLHEK TPVSDRVTKC CTESLVNRRF
CFSALEVDET YVPKEFNAET FTFHADICTL SEKERQIKKQ
TALVELVKHK PKATKEQLKA VMDDFAAFVE KCCKADDKET
CFAEEGKKLV AASQAALGL

Fusion proteins that contain a human serum albumin moiety generally contain the 585 amino acid HSA (amino acids 25-609 of SEQ ID NO:5, SEQ ID NO:6) or a functional variant thereof. The functional variant can include one or more amino acid deletions, additions or replacement in any desired combination, and includes functional fragments of HSA. The amount of amino acid sequence variation (e.g., through amino acid deletions, additions or replacements) is limited to preserve the serum half-life extending properties of HSA.

In some embodiments, the functional variant of HSA for use in the fusion proteins disclosed herein can have an amino acid sequence that has at least about 80%, at least about 85%, at least about 90%, or at least about 95% amino acid sequence identity with SEQ ID NO: 6, preferably when measured over the full length sequence of SEQ ID NO: 6. Alternatively or in addition, the functional variant of HSA can have from 1 to about 20, 1 to about 18, 1 to about 17, 1 to about 16, 1 to about 15, 1 to about 14, 1 to about 13, 1 to about 12, 1 to about 11, 1 to about 10, 1 to about 9, 1 to about 8, 1 to about 7, 1 to about 6, or 1 to about 5 amino acid deletions, additions or replacement, in any desired combination. In a specific embodiment, a functional variant of HSA for use in the fusion proteins disclosed herein comprises a C34A mutation.

Some functional variants of HSA for use in the fusion proteins disclosed herein may be at least 100 amino acids long, or at least 150 amino acids long, and may contain or consist of all or part of a domain of HSA, for example domain I (amino acids 1-194 of SEQ ID NO:6), II (amino acids 195-387 of SEQ ID NO:6), or III (amino acids 388-585 of SEQ ID NO:6). If desired, a functional variant of HSA may consist of or alternatively comprise any desired HSA domain combination, such as, domains I+II (amino acids 1-387 of SEQ ID NO:6), domains II+III (amino acids 195-585 of SEQ ID NO:6) or domains I+III (amino acids 1-194 of SEQ ID NO:6+ amino acids 388-585 of SEQ ID NO:6). As is well-known in the art, each domain of HSA is made up of two homologous subdomains, namely amino acids 1-105 and 120-194, 195-291 and 316-387, and 388-491 and 512-585 of domains I, II, and III respectively, with flexible inter-subdomain linker regions comprising residues Lys106 to Glul 19, Glu292 to Va1315 and Glu492 to Ala511. In certain embodiments, the SA moiety of the fusions proteins of the present invention contains at least one subdomain or domain of HSA.

Functional fragments of HSA suitable for use in the fusion proteins disclosed herein will contain at least about 5 or more contiguous amino acids of HSA, preferably at least about 10, at least about 15, at least about 20, at least about 25, at least about 30, at least about 50, or more contiguous amino acids of HSA sequence or may include part or all of specific domains of HSA.

In some embodiments, the functional variant (e.g., fragment) of HSA for use in the fusion proteins disclosed herein includes an N-terminal deletion, a C-terminal deletion or a combination of N-terminal and C-terminal deletions. Such variants are conveniently referred to using the amino acid number of the first and last amino acid in the sequence of the functional variant. For example, a functional variant with a C-terminal truncation can be amino acids 1-387 of HSA (SEQ ID NO:6).

Examples of HSA and HSA variants (including fragments) that are suitable for use in the MATRIN-3 (MATR3) fusion polypeptides described herein are known in the art. Suitable HSA and HSA variants include, for example full length mature HSA (SEQ ID NO:6) and fragments, such as amino acids 1-387, amino acids 54 to 61, amino acids 76 to 89, amino acids 92 to 100, amino acids 170 to 176, amino acids 247 to 252, amino acids 266 to 277, amino acids 280 to 288, amino acids 362 to 368, amino acids 439 to 447, amino acids 462 to 475, amino acids 478 to 486, and amino acids 560 to 566 of mature HSA. Such HSA polypeptides and functional variants are disclosed in PCT Publication WO 2005/077042A2, which is incorporated herein by reference in its entirety. Further variants of HSA, such as amino acids 1-373, 1-388, 1-389, 1-369, 1-419 and fragments that contain amino acid 1 through amino acid 369 to 419 of HSA are disclosed in European Published Application EP322094A1, and fragments that contain 1-177, 1-200 and amino acid 1 through amino acid 178 to 199 are disclosed in European Published Application EP399666A1.

Cell Penetrating Peptide (CPP) Moiety

Cell-penetrating peptides (CPPs) are short peptides that facilitate cellular intake/uptake of various cargo molecules (for example proteins or nucleic acids). CPPs typically have an amino acid composition that either contains a high relative abundance of positively charged amino acids such as lysine or arginine, has sequences that contain an alternating pattern of polar/charged amino acids and non-polar hydrophobic amino acids, or only apolar or hydrophobic amino acid groups. The cargo is associated with the CPP either through chemical linkage via covalent bonds or through non-covalent interactions.

One limitation of CPP use is the lack of cell specificity in CPP-mediated cargo delivery. Nevertheless, by mutagenesis or functional assays CPP variants with increased muscle-targeting have been discovered including B-MSP, Pip6, M12 or CyPep10 (Hum Mol Genet. 2009 Nov. 15; 18(22):4405-14; Mol Ther Nucleic Acids. 2012 Aug. 14; 1:e38; Mol Ther. 2014 Jul.; 22(7):1333-1341; Mol Ther. 2018 Jan. 3; 26(1):132-147).

Degraders

Proteolysis targeting chimera (PROTAC) is a strategy that utilizes the ubiquitin-proteasome system to target a specific protein and induce its degradation in the cell. Physiologically, the ubiquitin-proteasome system is responsible for clearing denatured, mutated, or harmful proteins in cells. PROTAC takes advantage of this protein destruction mechanism to remove specifically targeted proteins from cells. This technology takes advantage of bifunctional small molecules (degrader) in which a moiety target the protein of interest and a moiety of recognizes E3 ubiquitin ligase like for example cereblon (CRBN) or Von-Hippel Lindau (VHL) (Science 2015 348, 1376-1381; Molecular Cell 2017 67, 5-18). This allows potent and selective degradation of target proteins by enforcing proximity of the targeted protein and the E3 ligase, leading to ubiquitination and proteasomal degradation.

Reversible Bicyclization

Compared to small-molecule drugs, peptides are highly selective and efficacious and, at the same time, relatively safe and well-tolerated. However, peptides are inherently susceptible to proteolytic degradation. Additionally, peptides are generally impermeable to the cell membrane, largely limiting their applications to extracellular targets. Compared to their linear counterparts, cyclic peptides have reduced conformational freedom, which makes them more resistant to proteolysis and allows them to bind to their molecular targets with higher affinity and specificity. In particular, a short sequence motifs (FΦRRRR, where Φ is L-2-naphthylalanine) efficiently transport cyclic peptides inside cells and could be used as general transporters of cyclic peptides into mammalian cells (ACS Chem. Biol. 2013, 8:423-431). However, many peptide ligands must be in their extended conformations to be biologically active and are not compatible with the above cyclization approaches. To this end, a reversible bicyclization strategy, which allows the entire CPP-cargo fusion to be converted into a bicyclic structure by the formation of a pair of disulfide bonds, was recently described. When outside the cell, the peptide exists as a highly constrained bicycle, which possesses enhanced cell permeability and proteolytic stability. Upon entering the cytosol, the disulfide bonds are reduced by the intracellular glutathione (GSH) to produce the linear, biologically active peptide. The bicyclic system permits the formation of a small CPP ring for optimal cellular uptake11 and a separate cargo ring to accommodate peptides of different lengths (Angew Chem Int Ed Engl. 2017 Feb. 1; 56(6):1525-1529, incorporated by reference).

Linkers

Regarding the MATRIN-3 (MATR3) fusion proteins (e.g., SA, Fc, the cell-penetrating peptide (CPP), muscle-targeting cell-penetrating peptide (MCPP) MATRIN-3 (MATR3) fusion proteins) used in the present methods of the invention, the heterologous protein/peptide, e.g., SA, MCPP, and MATRIN-3 (MATR3) moieties can be directly bonded to each other in the contiguous polypeptide chain, or preferably indirectly bonded to each other through a suitable linker. The linker is preferably a peptide linker. Peptide linkers are commonly used in fusion polypeptides and methods for selecting or designing linkers are well-known. (See, e.g., Chen X et al. Adv. Drug Deliv. Rev. 65(10): 135701369 (2013) and Wriggers W et al., Biopolymers 80:736-746 (2005)).

Peptide linkers generally are categorized as i) flexible linkers, ii) helix forming linkers, and iii) cleavable linkers, and examples of each type are known in the art. Preferably, a flexible linker is included in the fusion polypeptides described herein. Flexible linkers may contain a majority of amino acids that are sterically unhindered, such as glycine and alanine. The hydrophilic amino acid Ser is also conventionally used in flexible linkers. Examples of flexible linkers include, polyglycines (e.g., (Gly)4 GGGG (SEQ ID NO: 7) and (Gly)5) GGGGG (SEQ ID NO: 8), polyalanines poly(Gly-Ala), and poly(Gly-Ser) (e.g., (Glyn-Sern)n or (Sern-Glyn)n, wherein each n is independent an integer equal to or greater than 1).

Peptide linkers can be of a suitable length. The peptide linker sequence may be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues in length. For example, a peptide linker can be from about 5 to about 50 amino acids in length; from about 10 to about 40 amino acids in length; from about 15 to about 30 amino acids in length; or from about 15 to about 20 amino acids in length. Variation in peptide linker length may retain or enhance activity, giving rise to superior efficacy in activity studies. The peptide linker sequence may be comprised of a naturally, or non-naturally, occurring amino acids.

In some aspects, the amino acids glycine and serine comprise the amino acids within the linker sequence. In certain aspects, the linker region comprises sets of glycine repeats (GSG3)n, where n is a positive integer equal to or greater than 1 (preferably 1 to about 20) (SEQ ID NO:9). More specifically, the linker sequence may be GSGGG (SEQ ID NO:10). The linker sequence may be GSGG (SEQ ID NO:11). In certain other aspects, the linker region orientation comprises sets of glycine repeats (SerGly3)n, where n is a positive integer equal to or greater than 1 (preferably 1 to about 20) (SEQ ID NO:12).

In more embodiments, a linker may contain glycine (G) and serine (S) in a random or preferably a repeated pattern. For example, the linker can be (GGGGS)n (SEQ ID NO:13), wherein n is an integer ranging from 1 to 20, preferably 1 to 4. In a particular example, n is 3 and the linker is GGGGSGGGGSGGGGS (SEQ ID NO:14).

In other embodiments, a linker may contain glycine (G), serine (S) and proline (P) in a random or preferably repeated pattern. For example, the linker can be (GPPGS)n (SEQ ID NO:15), wherein n is an integer ranging from 1 to 20, preferably 1-4. In a particular example, n is 1 and the linker is GPPGS (SEQ ID NO:16).

In general, the linker is not immunogenic when administered in a patient, such as a human. Thus, linkers may be chosen such that they have low immunogenicity or are thought to have low immunogenicity.

The linkers described herein are exemplary, and the linker can include other amino acids, such as Glu and Lys, if desired. The peptide linkers may include multiple repeats of, for example, (G45) (SEQ ID NO:13), (G3S) GGGS (SEQ ID NO:17), (G2S) GGS (SEQ ID NO:18) and/or (GlySer), if desired. In certain aspects, the peptide linkers may include multiple repeats of, for example, (SG4) SGGGG (SEQ ID NO:19), (SG3) SGGG (SEQ ID NO:20), (SG2) SGG (SEQ ID NO:21) or (SerGly). In other aspects, the peptide linkers may include combinations and multiples of repeating amino acid sequence units, such as (G3S)+(G4S)+(GlySer) (SEQ ID NO:17+SEQ ID NO:18+GlySer). In other aspects, Ser can be replaced with Ala e.g., (G4A, GGGGA) (SEQ ID NO:22) or (GA). In yet other aspects, the linker comprises the motif (EAAAK)n (SEQ ID NO:23), where n is a positive integer equal to or greater than 1, preferably 1 to about 20 (SEQ ID NO:24). In certain aspects, peptide linkers may also include cleavable linkers.

In a particular embodiment, a MATRIN-3 (MATR3) fusion or conjugate used in the present methods of the invention comprises a MATRIN-3 (MATR3) moiety (e.g., a MATRIN-3 (MATR3) polypeptide comprising an amino acid sequence that is at least 95% identical to

(SEQ ID NO: 25)
ARNGDHCPLGPGRCCRLHTVRASLEDLGWADWVLSPREVQVTMCIGACPSQ
FRAANMHAQIKTSLHRLKPDTVPAPCCVPASYNPMVLIQKTDTGVSLQTYD
DLLAKDCHCI

linked to a heterologous protein/peptide (e.g., MCPP or Degrader) or a conjugate moiety with a linker, wherein the linker has the amino acid sequence GGSSEAAEAAEAAEAAEAAEAAE (SEQ ID NO: 26). Additional non-limiting examples of linkers are described in PCT Publication No. WO2015/197446, which is incorporated herein by reference in its entirety, such as SEQ ID NOs: 4-13 and 24-38 disclosed therein.

Regarding the MATRIN-3 (MATR3) conjugates (e.g., the MATRIN-3 (MATR3) FA conjugates) used in the present methods of the invention, the MATRIN-3 (MATR3) moiety and conjugate moiety, e.g., fatty acid moiety, can be joined by a linker as follows: The linker separates the MATRIN-3 (MATR3) moiety and the conjugate moiety, e.g., fatty acid moiety. In particular embodiments, its chemical structure is not critical, since it serves primarily as a spacer. In a specific embodiment, the linker is a chemical moiety that contains two reactive groups/functional groups, one of which can react with the MATRIN-3 (MATR3) moiety and the other with the conjugate moiety, e.g., fatty acid moiety. The two reactive/functional groups of the linker are linked via a linking moiety or spacer, structure of which is not critical as long as it does not interfere with the coupling of the linker to the MATRIN-3 (MATR3) moiety and the conjugate moiety, e.g., fatty acid moiety, such as for example fatty acid moieties of Formula A1, A2 or A3.

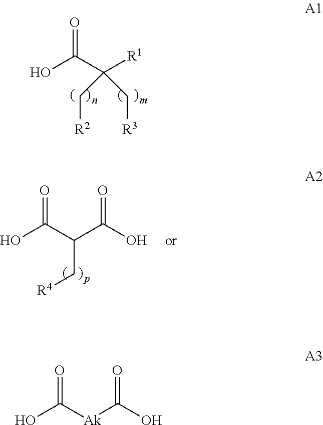

R¹ is CO₂H or H;
R², R³ and R⁴ are independently other H, OH, CO₂H, —CH═CH₂ or —C≡CH;
Ak is a branched C₆-C₁₀alkylene;
n, m and p are independently of each other an integer between 6 and 30; and which does not

The linker can be made up of amino acids linked together by peptide bonds. The amino acids can be natural or non-natural amino acids. In some embodiments of the present invention, the linker is made up of from 1 to 20 amino acids linked by peptide bonds, wherein the amino acids are selected from the 20 naturally occurring amino acids. In various embodiments, the 1 to 20 amino acids are selected from the amino acids glycine, serine, alanine, methionine, asparagine, glutamine, cysteine, glutamic acid and lysine, or amide derivatives thereof such as lysine amide.

In some embodiments, a linker is made up of a majority of amino acids that are sterically unhindered, such as glycine and alanine. In some embodiments, linkers are polyglycines, polyalanines, combinations of glycine and alanine (such as poly(Gly-Ala)), or combinations of glycine and serine (such as poly(Gly-Ser)). In some embodiments, a linker is made up of a majority of amino acids selected from histidine, alanine, methionine, glutamine, asparagine and glycine. In some embodiments, the linker contains a poly-histidine moiety. In other embodiments, the linker contains glutamic acid, glutamine, lysine or lysine amide or combination thereof.

In some embodiment, the linker may have more than two available reactive functional groups and can therefore serve as a way to link more than one fatty acid moiety. For example, amino acids such as Glutamine, Glutamic acid, Serine or Lysine can provide several points of attachment for a fatty acid moiety: the side chain of the amino acid and the functionality at the N-terminus or the C-terminus.

In some embodiments, the linker comprises 1 to 20 amino acids which are selected from non-natural amino acids. While a linker of 1-10 amino acid residues is preferred for conjugation with the fatty acid moiety, the present invention contemplates linkers of any length or composition. An example of non-natural amino acid linker is 8-Amino-3,6-dioxaoctanoic acid having the following formula:

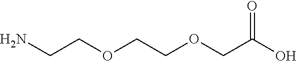 [SMILES: NCCOCCOCC(=O)O]

or its repeating units.

The linkers described herein are exemplary, and linkers that are much longer and which include other residues are contemplated by the present invention. Non-peptide linkers are also contemplated by the present invention.

In other embodiments, the linker comprise one or more alkyl groups, alkenyl groups, cycloalkyl groups, aryl groups, heteroaryl groups, heterocyclic groups, polyethylene glycol and/or one or more natural or unnatural amino acids, or combination thereof, wherein each of the alkyl, alkenyl, cycloalkyl, aryl, heteroaryl, heterocyclyl, polyethylene glycol and/or the natural or unnatural amino acids are optionally combined and linked together, or linked to the MATRIN-3 (MATR3) moiety and/or to the fatty acid moiety, via a chemical group selected from —C(O)O—, —OC(O)—, —NHC(O)—, —C(O)NH—, —O—, —NH—, —S—, —C(O)—, —OC(O)NH—, —NHC(O)—O—, ═NH—O—, ═NH—NH— or ═NH—N(alkyl)-.

Linkers containing alkyl spacer are for example —NH—(CH2)Z—C(O)— or —S—(CH2)Z— C(O)— or —O—(CH2)z-C(O)—, —NH—(CH2)Z—NH—, —O—C(O)—(CH2)z-C(O)—O—, —C(O)—(CH2)z-O—, —NHC(O)—(CH2)z-C(O)—NH— and the like wherein z is 2-20 can be used. These alkyl linkers can further be substituted by any non-sterically hindering group, including, but not limited to, a lower alkyl (e.g., Ci-C6), lower acyl, halogen (e.g., CI, Br), CN, NH2, or phenyl.

The linker can also be of polymeric nature. The linker may include polymer chains or units that are biostable or biodegradable. Polymers with repeat linkage may have varying degrees of stability under physiological conditions depending on bond lability. Polymers may contain bonds such as polycarbonates (—O—C(O)—O—), polyesters (—C(O)—O—), polyurethanes (—NH— C(O)—O—), polyamide (—C(O)—NH—). These bonds are provided by way of examples, and are not intended to limit the type of bonds employable in the polymer chains or linkers of the invention. Suitable polymers include, for example, polyethylene glycol (PEG), polyvinyl pyrrolidone, polyvinyl alcohol, polyamino acids, divinylether maleic anhydride, N-(2-hydroxypropyl)-methacrylicamide, dextran, dextran derivatives, polypropylene glycol, polyoxyethylated polyol, heparin, heparin fragments, polysaccharides, cellulose and cellulose derivatives, starch and starch derivatives, polyalkylene glycol and derivatives thereof, copolymers of polyalkylene glycols and derivatives thereof, polyvinyl ethyl ether, and the like and mixtures thereof. A polymer linker is for example polyethylene glycol (PEG). The PEG linker can be linear or branched. A molecular weight of the PEG linker in the present invention is not restricted to any particular size, but certain embodiments have a molecular weight between 100 to 5000 Dalton for example 500 to 1500 Dalton.

The linking moiety (or spacer) contains appropriate functional-reactive groups at both terminals that form a bridge between an amino group of the peptide or polypeptide/protein (e.g. N-terminus or side chain of a lysine) and a functional/reactive group on the fatty acid moiety (e.g the carboxylic acid functionality of the fatty acid moiety). Alternatively, the linking moiety (or spacer) contains appropriate functional-reactive groups at both terminals that form a bridge between an acid carboxylic group of the peptide or polypeptide/protein (e.g. C-terminus) and a functional/reactive group on the fatty acid moiety (e.g the carboxylic acid functionality of the fatty acid moiety of formula A1, A2 and A3 as above).

The linker may comprise several linking moieties (or spacer) of different nature (for example a combination of amino acids, heterocyclyl moiety, PEG and/or alkyl moieties). In this instance, each linking moiety contains appropriate functional-reactive groups at both terminals that form a bridge between an amino group of the peptide or polypeptide/protein (e.g. the N-terminus or the side chain of a lysine) and the next linking moiety of different nature and/or contains appropriate functional-reactive groups that form a bridge between the prior linking moiety of different nature and the fatty acid moiety. In other instance, each linking moiety contains appropriate functional-reactive groups at both terminals that form a bridge between an acid carboxylic group of the peptide or polypeptide/protein (e.g. the C-terminus) and the next linking moiety of different nature and/or contains appropriate functional-reactive groups that form a bridge between the prior linking moiety of different nature and the fatty acid moiety.

Additionally, a linking moiety may have more than 2 terminal functional groups and can therefore be linked to more than one fatty acid moiety. Example of these multi-functional group moieties are glutamic acid, lysine or serine. The side chain of the amino acid can also serve as a point of attachment for another fatty acid moiety.

The modified peptides or polypeptides and/or peptide-polypeptide partial construct (i.e. peptide/polypeptide attached to a partial linker) include reactive groups which can react with available reactive functionalities on the fatty acid moiety (or modified fatty acid moiety: i.e. already attached a partial linker) to form a covalent bond. Reactive groups are chemical groups capable of forming a covalent bond. Reactive groups are located at one site of conjugation and can generally be carboxy, phosphoryl, acyl group, ester or mixed anhydride, maleimide, N-hydroxysuccinimide, tetrazine, alkyne, imidate, pyridine-2-yl-disulfanyl, thereby capable of forming a covalent bond with functionalities like amino group, hydroxyl group, alkene group, hydrazine group, hydroxylamine group, an azide group or a thiol group at the other site of conjugation.

Reactive groups of particular interest for conjugating a MATRIN-3 (MATR3) moiety to a linker and/or a linker to the fatty acid moiety and/or to conjugate various linking moieties of different nature together are N-hydroxysuccinimide, alkyne (more particularly cyclooctyne).

Functionalities include: 1. thiol groups for reacting with maleimides, tosyl sulfone or pyridine-2-yldisulfanyl; 2. amino groups (for example amino functionality of an amino acid) for bonding to carboxylic acid or activated carboxylic acid (e.g. amide bond formation via N-hydroxysuccinamide chemistry), phosphoryl groups, acyl group or mixed anhydride; 3. Azide to undergo a Huisgen cycloaddition with a terminal alkyne and more particularly cyclooctyne (more commonly known as click chemistry); 4. carbonyl group to react with hydroxylamine or hydrazine to form oxime or hydrazine respectively; 5. Alkene and more particularly strained alkene to react with tetrazine in an aza [4+2] addition. While several examples of linkers and functionalities/reactive group are described herein, the methods of the present invention contemplate linkers of any length and composition.

MATRIN-3 (MATR3) Fusion Polypeptides

In specific aspects, MATRIN-3 (MATR3) fusion polypeptides described herein as useful for administration for the present methods of treatment of the invention may contain a MATRIN-3 (MATR3) moiety and a heterologous moiety, and optionally a linker. In a particular embodiment, a MATRIN-3 (MATR3) fusion polypeptide described herein as useful for administration for the present methods of treatment of the invention may contain a MATRIN-3 (MATR3) moiety and a heterologous moiety which is SA, a cell-penetrating peptide (CPP), a muscle-targeting cell-penetrating peptide (MCPP) or a variant thereof, and optionally a linker.

In specific aspects, MATRIN-3 (MATR3) fusion polypeptides described herein as useful for administration for the present methods of treatment of the invention may contain a MATRIN-3 (MATR3) moiety and SA, a cell-penetrating peptide (CPP) moiety, a muscle-targeting cell-penetrating peptide (MCPP) moiety or a variant thereof, and optionally a linker. In one embodiment, the fusion polypeptide is a contiguous amino acid chain in which the SA, CPP, MCPP moiety is located N-terminally to the MATRIN-3 (MATR3) moiety. The C-terminus of the SA, CPP or MCPP moiety can be directly bonded to the N-terminus of the MATRIN-3 (MATR3) moiety. Preferably, the C-terminus of the SA, CPP, MCPP moiety is indirectly bonded to the N-terminus of the MATRIN-3 (MATR3) moiety through a peptide linker.

The SA, CPP or MCPP moiety and MATRIN-3 (MATR3) moiety can be from any desired species. For example, the fusion protein can contain SA, CPP, MCPP and MATRIN-3 (MATR3) moieties that are from human, mouse, rat, dog, cat, horse or any other desired species. The SA, CPP, MCPP and MATRIN-3 (MATR3) moieties are generally from the same species, but fusion peptides in which the SA, CPP or MCPP moiety is from one species and the MATRIN-3 (MATR3) moiety is from another species (e.g., mouse SA, CPP or MCPP and human MATRIN-3 (MATR3)) are also encompassed by this disclosure.

In some embodiments, the fusion polypeptide comprises mouse serum albumin (SA), CPP or functional variant thereof and mature human MATRIN-3 (MATR3) peptide or functional variant thereof.

In preferred embodiments, the SA moiety is HAS, CPP moiety is B-MSP or a functional variant thereof and the MATRIN-3 (MATR3) moiety is the mature human MATR3 peptide or a functional variant thereof. When present, the optional linker is preferably a flexible peptide linker.

In particular embodiments, the fusion polypeptide comprises

A) an SA moiety selected from the group consisting of HSA (25-609) (SEQ ID NO: 6), and HSA (25-609) in which Cys34 is replaced with Ser and Asn503 is replaced with Gin; and

B) a MATRIN-3 moiety selected from the group consisting of sequences as indicated in Table 1.

If desired, the fusion polypeptide can further comprise a linker that links the C-terminus of the SA moiety to the N-terminus of the MATRIN-3 moiety. Preferably, the linker is selected from (GGGGS)n (SEQ ID NO:13) and (GPPGS)n (SEQ ID NO:15), wherein n is one to about 20. Preferred linkers include ((GGGGS)n (SEQ ID NO:13) and (GPPGS)n (SEQ ID NO:15), wherein n is 1, 2, 3 or 4.

If desired, the fusion polypeptide can contain additional amino acid sequence. For example, an affinity tag can be included to facilitate detecting and/or purifying the fusion polypeptide.

MATRIN-3 (MATR3) Conjugates

Various embodiments of the MATRIN-3 (MATR3) conjugates, e.g., MATRIN-3 (MATR3) fatty acid conjugates, that can be used in the present methods of treatment of the invention are described herein. It will be recognized that features specified in each embodiment may be combined with other specified features to provide further embodiments.

In a specific embodiment, a MATRIN-3 (MATR3) conjugate for the methods provided here comprises a MATRIN-3 (MATR3) polypeptide or a functional variant thereof conjugated to a moiety, such as a fatty acid moiety, optionally comprising a linker. In some embodiment of the invention, the fatty acid residue is a lipophilic residue.

In another embodiment the fatty acid residue is negatively charged at physiological pH. In another embodiment the fatty acid residue comprises a group which can be negatively charged. One preferred group which can be negatively charged is a carboxylic acid group.

In another embodiment of the invention, the fatty acid residue binds non-covalently to albumin or other plasma proteins. In yet another embodiment of the invention the fatty acid residue is selected from a straight chain alkyl group, a branched alkyl group, a group which has an (O-carboxylic acid group, a partially or completely hydrogenated cyclopentanophenanthrene skeleton.

In another embodiment the fatty acid residue is a cibacronyl residue. In another embodiment the fatty acid residue has from 6 to 40 carbon atoms, from 8 to 26 carbon atoms or from 8 to 20 carbon atoms.

In another embodiment, the fatty acid residue is an acyl group selected from the group comprising R—C(O)— wherein R is a C4-38 linear or branched alkyl or a C4-38 linear or branched alkenyl where each said alkyl and alkenyl are optionally substituted with one ore more substituents selected from —CO2H, hydroxyl, —SO3H, halo and —NHC(O)C(O)OH. The acyl group (R—C(O)—) derives from the reaction of the corresponding carboxylic acid R—C(O)OH with an amino group on the MATRIN-3 (MATR3) polypeptide.

In another embodiment the fatty acid residue is an acyl group selected from the group comprising CH3(CH2)r-CO, wherein r is an integer from 4 to 38, preferably an integer from 4 to 24, more preferred selected from the group comprising CH3(CH2)6CO—, CH3(CH2)s-CO—, CH3(CH2)10-CO—, CH3 (CH2)12-CO—, CH3 (CH2)14-CO—, CH3 (CH2)16-CO—, CH3 (CH2)18-CO—, CH3(CH2)20-CO and CH3(CH2)22-CO—.

In another embodiment the fatty acid residue is an acyl group of a straight-chain or branched alkane.

In another embodiment the fatty acid residue is an acyl group selected from the group comprising HOOC—(CH2)sCO—, wherein s is an integer from 4 to 38, preferably an integer from 4 to 24, more preferred selected from the group comprising HOOC(CH2)i4-CO—, HOOC(CH2)16-CO—, HOOC(CH2)18-CO—, HOOC(CH2)20-CO— and HOOC(CH2)22-CO—.

In another embodiment the fatty acid residue is a group of the formula CH3-(CH2)X— CO—NH—CH(CH2CO2H)—C(O)— wherein x is an integer of from 8 to 24.

In yet another embodiment the fatty acid residue is selected from the group consisting of: CH3-(CH2)6_24-CO2H; CF3-(CF2)4_9-CH2CH2-CO2H; CF3-(CF2)4.9-CH2CH2-O-CH2-CO2H; CO2H-(CH2)6.24-CO2H; SO2H-(CH2)6.24-CO2H; wherein the fatty acid is linked to an amino group on MATRIN-3 (MATR3) polypeptide (N-terminus or side chain of a lysine) or to an amino group on a linker via one of its carboxylic functionalities.

Specific examples of fatty acid are:

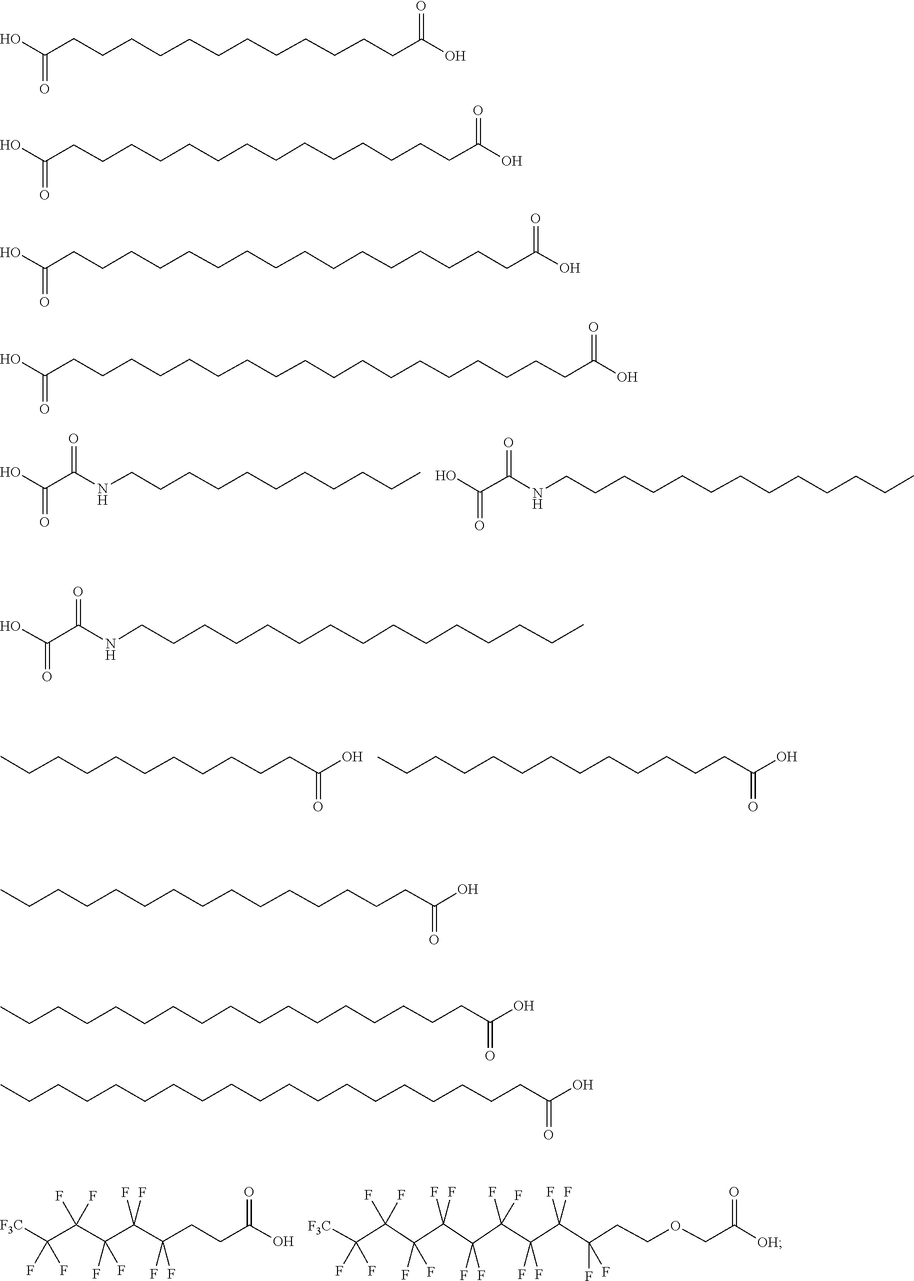

wherein the fatty acid is linked to the N-terminus of MATRIN-3 (MATR3) or to an amino group on the side chain of MATRIN-3 (MATR3) or to an amino group on a linker via one of its carboxylic acid functionalities.

Of particular interest, the linker between the above mentioned fatty acids and the MATRIN-3 (MATR3) comprises lysine, glutamic acid, repeating units of:

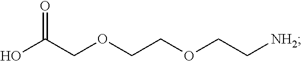

preferably 1 to 3; or mixture thereof.

More preferably, the linker comprises one or more glutamic acid amino acids and one or more repeating unit of CO2H-CH2-O-CH2-CH2-O-CH2-CH2-NH2.

Examples of fatty acid linked to one or two glutamic acid amino acids are:

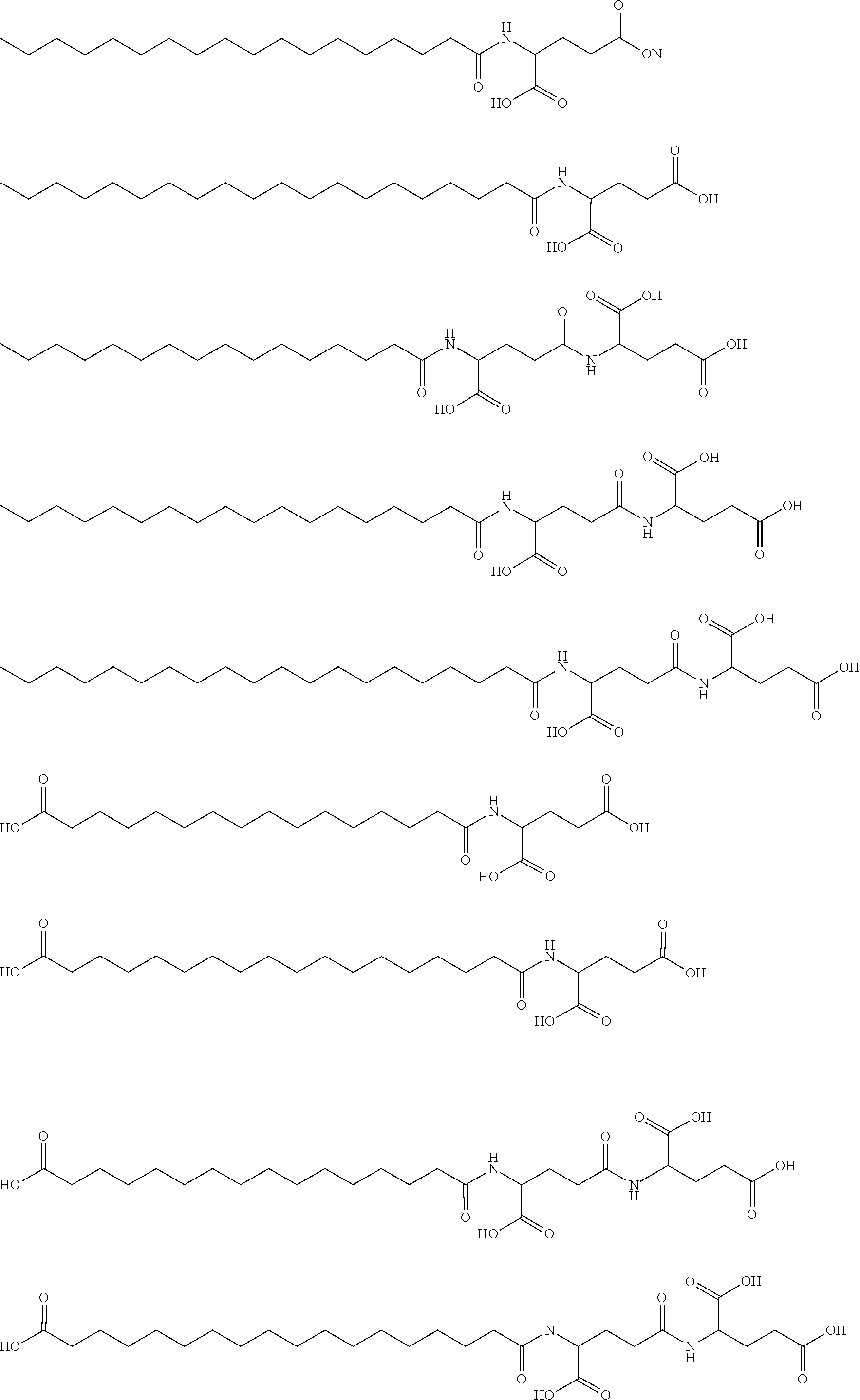

wherein the chiral carbon atoms independently are either R or S and wherein the fatty acid-linker moiety is linked to the N-terminus of MATRIN-3 (MATR3) or to an amino group on the side chain of MATRIN-3 (MATR3) or to an amino group on another linking moiety via one of the Glutamic acid's carboxylic acid functionalities.

Also, of particular interest, the linker comprises one or more Lysine or Lysine amide amino acids, and one or more repeating unit of CO2H-CH2-O-CH2-CH2-O-CH2-CH2-NH2.

Example of fatty acid moity(ies) linked to a Lysine or/and a Lysine amide amino acids are:

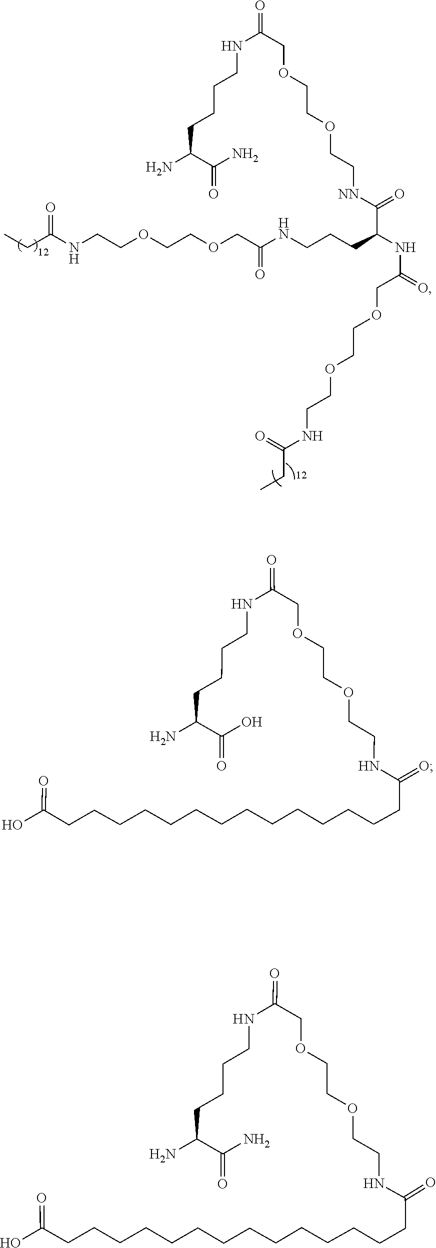

wherein the primary amino group of the lysine is attached the C-terminus of MATRIN-3 (MATR3) or to a carboxylic acid functionality on a side chain of MATRIN-3 (MATR3); or to a carboxylic acid functionality on another linking moiety.

Another specific example of linkers to be used with above fatty acids is 4-sulfamoylbutanoic acid:

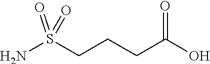

Examples of fatty acids linked to the above linker are:

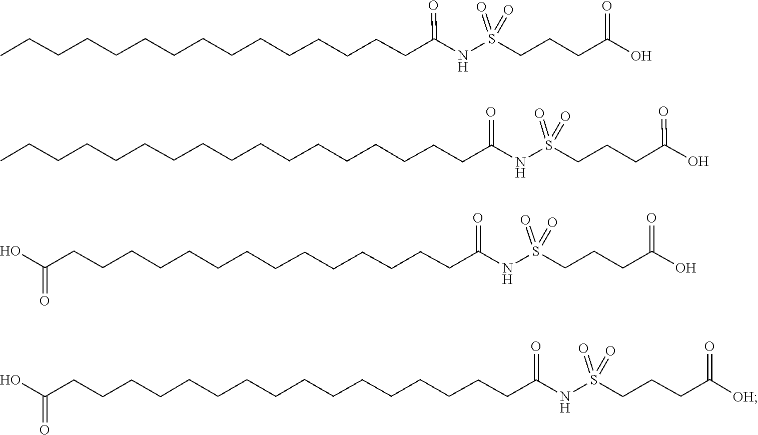 [SMILES: CCCCCCCCCCCCCCCC(=O)NS(=O)(=O)CCCC(=O)O ; CCCCCCCCCCCCCCCCCC(=O)NS(=O)(=O)CCCC(=O)O ; O=C(O)CCCCCCCCCCCCCCC(=O)NS(=O)(=O)CCCC(=O)O ; O=C(O)CCCCCCCCCCCCCCCCC(=O)NS(=O)(=O)CCCC(=O)O]

wherein the fatty acid-linker moiety is linked to the N-terminus of MATRIN-3 (MATR3) or to an amino group on the side chain of MATRIN-3 (MATR3) or to an amino group on another linking moiety via the carboxylic acid functionality on the sulfamoyl butanoic acid moiety.

Additionally, such fatty acid linker construct can further comprise repeating units of:

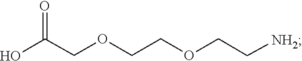

preferably 1 to 4.

Other examples of fatty acid-linker constructs are further disclosed in US 2013/0040884, Albumin-binding conjugates comprising fatty acid and PEG (Novo Nordisk) which is incorporated by reference.

Such constructs are preferably linked to the N-terminus of MATRIN-3 (MATR3) via a carboxylic acid functionality.

In embodiment 1, the invention pertains to a conjugate comprising a MATRIN-3 (MATR3) moiety linked to a fatty acid moiety via a linker wherein the fatty acid moiety has the following Formulae A1, A2 or A3:

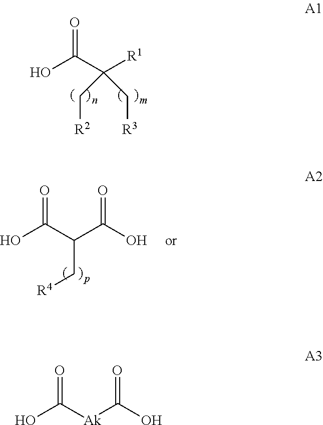

R¹ is CO2H, H;

R², R³ and R⁴ are independently of each other H, OH, CO2H, —CH═CH2 or —C≡CH;

Ak is a branched C6-C3 alkylene;

n, m and p are independently of each other an integer between 6 and 30; or an amide, an ester or a pharmaceutically acceptable salt thereof.

Preferred embodiments are also disclosed in WO2017/109706 incorporated by reference.

The invention pertains to conjugate according to any of the preceding conjugate's embodiments wherein the linker comprises an oligo ethylene glycol moiety as disclosed in WO2017/109706, incorporated by reference.

The invention pertains to conjugate according to any of the preceding conjugate's embodiments wherein the linker comprises (or further comprises) a heterocyclic moiety as disclosed in WO2017/109706, incorporated by reference.

Such heterocyclyl containing linkers are obtained for example by azide-alkyne Huisgen cycloaddition, which more commonly known as click chemistry. More particularly, some of the heterocyclyl depicted supra result from the reaction of a cycloalkyne with an azide—containing moiety.

Cycloalkyne are readily available from commercial sources and can therefore be functionalized via cycloaddition with a moiety containing an azide functionality (e.g. a linker containing a terminal azide functionality). Examples of the use of cyclic alkyne click chemistry in protein labeling has been described in US 2009/0068738 which is herein incorporated by reference.

These reagents which are readily available and/or commercially available are attached directly or via a linker as described supra to the peptide or polypeptide of interest. The alkyne, maleimide or tetrazine reactive groups are reacted with a functional group (azide, thiol and alkene respectively) which is present on the fatty acid moiety or on a linker-fatty acid construct (such as for example a PEG-fatty acid construct).

In a further embodiment, the invention pertains to a conjugate according to any of the preceding conjugate's embodiments wherein the linker comprises or further comprises one or more amino acids independently selected from histidine, methionine, alanine, glutamine, asparagine and glycine. In one particular aspect of this embodiment, the linker comprises 1 to 6 amino acid selected from histidine, alanine and methionine.

The invention also pertains to a conjugate according to any one of the preceding conjugate's embodiments wherein the MATRIN-3 (MATR3) moiety is MATRIN-3 (MATR3)), or related proteins and homologs, variants, fragments and other modified forms thereof. In a further embodiment, the invention pertains to a conjugate according to any one of the preceding conjugate's embodiments wherein the MATRIN-3 (MATR3) moiety is a MATRIN-3 (MATR3) variant.

Nucleic Acids and Host Cells

The invention also relates to nucleic acids that encode MATR3, MATR3 fragments, or fusion polypeptides containing MATR3 or MATR3 fragments described herein as useful for administration for the present methods of treatment of the invention, including vectors that can be used to produce the polypeptides. The nucleic acids are isolated and/or recombinant. In certain embodiments, the nucleic acid encodes a fusion polypeptide in which HSA, CPP or MCPP or a functional variant thereof is located N-terminally to human mature MATRIN-3 (MATR3) or a functional variant thereof. If desired the nucleic acid can further encode a linker (e.g., a flexible peptide linker) that bonds the C-terminus of the SA, CPP, MCPP or a functional variant thereof to the N-terminus of human mature MATRIN-3 (MATR3) or a functional variant thereof. If desired, the nucleic acid can also encode a leader, or signal, sequence to direct cellular processing and secretion of the fusion polypeptide.

In preferred embodiments, the nucleic acid encodes a fusion polypeptide in which the SA moiety is HSA or a functional variant thereof and the MATRIN-3 (MATR3) moiety is the mature human MATRIN-3 peptide or a functional variant thereof. When present, the optional linker is preferably a flexible peptide linker. In particular embodiments, the nucleic acid encodes a fusion polypeptide that comprises A) an SA moiety selected from the group consisting of HSA (25-609) (SEQ ID NO:6), and HSA (25-609) in which Cys34 is replaced with Ser and Asn503 is replaced with Gin; and B) a MATRIN-3 moiety selected from the group consisting of sequences of SEQ ID No. 4 or 6 or as disclosed in Table 1.

If desired, the encoded fusion polypeptide can further comprise a linker that links the C-terminus of the SA, CPP or MCPP moiety to the N-terminus of the MATRIN-3 (MATR3) moiety. Preferably, the linker is selected from (GGGGS)n (SEQ ID NO: 13) and (GPPGS)n (SEQ ID NO:16) and (GPPGS)n (SEQ ID NO:15), wherein n is one to about 20. Preferred linkers include ((GGGGS)n (SEQ ID NO:13) and (GPPGS)n (SEQ ID NO:15), wherein n is 1, 2, 3 or 4.

For expression in host cells, the nucleic acid encoding a fusion polypeptide can be present in a suitable vector and after introduction into a suitable host, the sequence can be expressed to produce the encoded fusion polypeptide according to standard cloning and expression techniques, which are known in the art (e.g., as described in Sambrook, J., Fritsh, E. F., and Maniatis, T. Molecular Cloning: A Laboratory Manual 2^{nd} ed., Cold Spring Harbor Laboratory, Cold Spring Harbor Laboratory Press, Cold Spring Harbor, N.Y., 1989). The invention also relates to such vectors comprising a nucleic acid sequence according to the invention.

A recombinant expression vector can be designed for expression of a MATRIN-3 (MATR3) fusion polypeptide in prokaryotic (e.g., E. coli) or eukaryotic cells (e.g., insect cells, yeast cells, or mammalian cells). Representative host cells include many E. coli strains, mammalian cell lines, such as CHO, CHO-K1, and HEK293; insect cells, such as Sf9 cells; and yeast cells, such as S. cerevisiae and P. pastoris. Alternatively, the recombinant expression vector can be transcribed and translated in vitro, for example using T7 promoter regulatory sequences and T7 polymerase and an in vitro translation system. Vectors suitable for expression in host cells and cell-free in vitro systems are well known in the art. Generally, such a vector contains one or more expression control elements that are operably linked to the sequence encoding the fusion polypeptide.

Expression control elements include, for example, promoters, enhancers, splice sites, poly adenylation signals and the like. Usually a promoter is located upstream and operably linked to the nucleic acid sequence encoding the fusion polypeptide. The vector can comprise or be associated with any suitable promoter, enhancer, and other expression-control elements.

Examples of such elements include strong expression promoters (e.g., a human CMV IE promoter/enhancer, an RSV promoter, SV40 promoter, SL3-3 promoter, MMTV promoter, or HIV LTR promoter, EF1 alpha promoter, CAG promoter) and effective poly (A) termination sequences. Additional elements that can be present in a vector to facilitate cloning and propagation include, for example, an origin of replication for plasmid product in E. coli, an antibiotic resistance gene as a selectable marker, and/or a convenient cloning site (e.g., a polylinker).

In another aspect of the instant disclosure, host cells comprising the nucleic acids and vectors disclosed herein are provided. In various embodiments, the vector or nucleic acid is integrated into the host cell genome, which in other embodiments the vector or nucleic acid is extra-chromosomal. If desired the host cells can be isolated.

Recombinant cells, such as yeast, bacterial (e.g., E. coli), and mammalian cells (e.g., immortalized mammalian cells) comprising such a nucleic acid, vector, or combinations of either or both thereof are provided. In various embodiments, cells comprising a non-integrated nucleic acid, such as a plasmid, cosmid, phagemid, or linear expression element, which comprises a sequence coding for expression of a fusion polypeptide comprising the human MATRIN-3 (MATR3) protein or a functional variant thereof fused or not with SA, CPP or a MCPP or the functional variant thereof and, are provided.

A vector comprising a nucleic acid sequence encoding a MATRIN-3 (MATR3) fusion polypeptide provided herein can be introduced into a host cell using any suitable method, such as by transformation, transfection or transduction. Suitable methods are well known in the art. In one example, a nucleic acid encoding a fusion polypeptide comprising the SA, CPP or MCPP or the functional variant thereof and human MATRIN-3 (MATR3) protein or the functional variant thereof can be positioned in and/or delivered to a host cell or host animal via a viral vector. Any suitable viral vector can be used in this capacity.

The invention also provides a method for producing a fusion polypeptide as described herein, comprising maintaining a recombinant host cell comprising a recombinant nucleic acid of the invention under conditions suitable for expression of the recombinant nucleic acid, whereby the recombinant nucleic acid is expressed, and a fusion polypeptide is produced. In some embodiments, the method further comprises isolating the fusion polypeptide.

In the present invention a preferred mode of treatment is by a gene therapy-type approach in which MATR3 or fragments, variant, fusion thereof will be delivered using vectors, preferably AAV derived vectors, preferably with a muscle-specific promoter, preferably the vector is administered intramuscularly or systemically.

Therapeutic Methods and Pharmaceutical Compositions

DUX4 is a homeodomain-containing transcription factor and an important regulator of early human development as it plays an essential role in activating the embryonic genome during the 2- to 8-cell stage of development (Nat. Genet. 49, 925-934 (2017); Nat. Genet. 49, 935-940 (2017); Nat. Genet. 49, 941-945 (2017). As such, it is not typically expressed in healthy somatic cells, and importantly it is silent in healthy skeletal muscle or B-cells.

The present invention refers to the treatment of a condition associated with an aberrant expression and/or function of DUX4 protein and/or of DUX4 fusion protein (such as CIC-DUX4 or DUX4-IGH). Such condition includes muscular dystrophy, infection and cancer.

For instance, facioscapulohumeral muscular dystrophy (FSHD) is one of the most prevalent neuromuscular disorders (Neurology 83, 1056-9 (2014) and leads to significant lifetime morbidity, with up to 25% of patients requiring wheelchair. The disease is characterized by rostro-caudal progressive and asymmetric weakness in a specific subset of muscles. Symptoms typically appear as asymmetric weakness of the facial (facio), shoulder (scapulo), and upper arm (humeral) muscles, and progress to affect nearly all skeletal muscle groups. Extra-muscular manifestations can occur in severe cases, including retinal vasculopathy, hearing loss, respiratory defects, cardiac involvement, mental retardation and epilepsy (Curr. Neurol. Neurosci. Rep. 16, 66 (2016). FSHD is not caused by a classical form of gene mutation that results in loss or altered protein function. Likewise, it differs from typical muscular dystrophies by the absence of sarcolemma defects (J. Cell Biol. 191, 1049-1060 (2010). Instead, FSHD is linked to epigenetic alterations affecting the D4Z4 macrosatellite repeat array in 4q35 and causing chromatin relaxation leading to inappropriate gain of expression of the D4Z4-embedded double homeobox 4 (DUX4) gene (Curr. Neurol. Neurosci. Rep. 16, 66 (2016).

Acute lymphoblastic leukemia (ALL) is the most common cancer among children and the most frequent cause of death from cancer before 20 years of age. Approximately 80-85% of pediatric ALL is of B cell origin and results from arrest at an immature B-precursor cell stage (N. Engl. J Med. 373, 1541-52 (2015). The underlying etiology of most cases of childhood ALL remains largely unknown. Nevertheless, sentinel chromosomal translocations occur frequently and recurrent ALL-associated translocations can be initiating events that drive leukemogenesis (J. Clin. Oncol. 33, 2938-48 (2015). Importantly, the characterization of gene expression, biochemical and functional consequences of these mutations may provide a window of therapeutic opportunity. Indeed, therapeutic strategies tailored to target ALL-associated driver lesions and pathways may increase anti-leukemia efficacy and decrease relapse, as well as reduce undesirable off-target toxicities (J. Clin. Oncol. 33, 2938-48 (2015). Recently, recurrent DUX4 rearrangements were reported in up to 7% of B-ALL patients (Nat. Genet. 48, 569-74 (2016); EBioMedicine 8, 173-83 (2016); Nat. Commun. 7, 11790 (2016); Nat. Genet. 48, 1481-1489 (2016). Nearly all cases exhibit rearrangement of DUX4 to the immunoglobulin heavy chain (IGH) enhancer region resulting in truncation of DUX4 C terminus and addition of amino acids from read-through into the IGH locus. The rearrangement has two functional consequences. First, the translocation hijacks the IGH enhancer resulting in overexpression of DUX4 in the B cell lineage. Second, the truncation of DUX4 C terminus and the appendage of amino acids encoded by the IGH locus changes the biology of the resulting DUX4-IGH fusion protein. While DUX4 is pro-apoptotic, DUX4-IGH induces transformation in NIH-3T3 fibroblasts and is required for the proliferation of DUX4-IGH expressing NALM6 B-ALL cells (Nat. Genet. 48, 569-74 (2016); Nat. Genet. 48, 1481-1489 (2016). Moreover, expression of DUX4-IGH in mouse pro-B cells is sufficient to give rise to leukemia. In contrast, mouse pro-B cells expressing wild-type DUX4 undergo cell death (Nat. Genet. 48, 569-74 (2016). The DUX4 rearrangement is a clonal event acquired early in leukemogenesis and the expression of DUX4-IGH is maintained in leukemias at relapse (Nat. Genet. 48, 569-74 (2016); Nat. Genet. 48, 1481-1489 (2016), strongly supporting DUX4-IGH as an oncogenic driver.

There are no drugs currently approved to prevent or treat a condition associated with an aberrant expression and/or function of DUX4 protein and/or of DUX4 fusion protein (CIC-DUX4 or DUX4-IGH), such as FSHD or DUX-IGH associated ALL. For the first time, the inventors identified a molecule (MATR3) able to inhibit the activity of both DUX4 and DUX4-IGH/CIC-DUX4 for the treatment of muscular dystrophies, infection or cancer such as FSHD and DUX-IGH associated ALL.

An effective amount of the therapeutic vector or the fusion polypeptide, usually in the form of a pharmaceutical composition, is administered to a subject in need thereof. The therapeutic vector or the fusion polypeptide can be administered in a single dose or multiple doses, and the amount administered, and dosing regimen will depend upon the particular therapeutic vector or fusion protein selected, the severity of the subject's condition and other factors. A clinician of ordinary skill can determine appropriate dosing and dosage regimen based on a number of other factors, for example, the individual's age, sensitivity, tolerance and overall well-being.

The administration can be performed by any suitable route using suitable methods, such as parenterally (e.g., intravenous, subcutaneous, intraperitoneal, intramuscular, intrathecal injections or infusion), orally, topically, intranasally or by inhalation. Parental administration is generally preferred. Intravenous administration is preferred.

MATRIN-3 (MATR3) therapeutic vectors or MATRIN-3 (MATR3) fusion polypeptides of the present invention can be administered to the subject in need thereof alone or with one or more other agents. When the therapeutic vector or fusion polypeptide is administered with another agent, the agents can be administered concurrently or sequentially to provide overlap in the therapeutic effects of the agents. Examples of other agents that can be administered in combination with the therapeutic vector or the fusion polypeptide include: anti-inflammatory agents, anti-oxidants, chemotherapy, radiotherapy.

The invention also relates to pharmaceutical compositions comprising a MATRIN-3 (MATR3) conjugate or a MATRIN-3 (MATR3) fusion polypeptide as described herein (e.g., comprising a fusion polypeptide comprising SA, CPP, MCPP or a functional variant thereof and human MATRIN-3 (MATR3) protein or a functional variant thereof). Such pharmaceutical compositions can comprise a therapeutically effective amount of the fusion polypeptide and a pharmaceutically or physiologically acceptable carrier. The carrier is generally selected to be suitable for the intended mode of administration and can include agents for modifying, maintaining, or preserving, for example, the pH, osmolarity, viscosity, clarity, color, isotonicity, odor, sterility, stability, rate of dissolution or release, adsorption, or penetration of the composition. Typically, these carriers include aqueous or alcoholic/aqueous solutions, emulsions or suspensions, including saline and/or buffered media.

Suitable agents for inclusion in the pharmaceutical compositions include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, diluents, excipients and/or pharmaceutical adjuvants.

Parenteral vehicles include sodium chloride solution, Ringer's dextrose, dextrose and sodium chloride and lactated Ringer's. Suitable physiologically-acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some case it will be preferable to include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents and inert gases, may also be present. The precise formulation will depend on the route of administration. Additional relevant principle, methods and components for pharmaceutical formulations are well known. (See, e.g., Allen, Loyd V. Ed, (2012) Remington's Pharmaceutical Sciences, 22th Edition).

When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

In certain embodiments, the pharmaceutical composition is for subcutaneous administration. Suitable formulation components and methods for subcutaneous administration of polypeptide therapeutics (e.g., antibodies, fusion proteins and the like) are known in the art. See, e.g., Published United States Patent Application No 2011/0044977 and U.S. Pat. Nos. 8,465,739 and 8,476,239. Typically, the pharmaceutical compositions for subcutaneous administration contain suitable stabilizers (e.g, amino acids, such as methionine, and or saccharides such as sucrose), buffering agents and tonicifying agents.

### Definitions

The term “amino acid mimetic,” as used herein, refers to chemical compounds that have a structure that is different from the general chemical structure of an amino acid, but functions in a manner similar to a naturally occurring amino acid.

“Conservative” amino acid replacements or substitutions refer to replacing one amino acid with another that has a side chain with similar size, shape and/or chemical characteristics. Examples of conservative amino acid replacements include replacing one amino acid with another amino acid within the following groups: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M).

The term “effective amount” refers to an amount sufficient to achieve the desired therapeutic effect, under the conditions of administration, such as an amount sufficient to bind DUX4 or DUX4-IGH or CIC-DUX4, prevent the interaction with DNA of DUX4 or DUX4-IGH or CIC-DUX4, inhibit the activation of specific target genes by DUX4 or DUX4-IGH or CIC-DUX4, reduce the toxic effects of DUX4, DUX4-IGH or CIC-DUX4 or reduce the cancer activity of DUX4-IGH or CIC-DUX4. For example, a “therapeutically-effective amount” of a MATRIN-3 (MATR3) therapeutic agent administered to a patient exhibiting, suffering, or prone to suffer from a condition associated with aberrant expression and/or function of DUX4, such as FSHD or ALL is such an amount which causes an improvement in the pathological symptoms, disease progression, physiological conditions associated with or induces resistance to succumbing to the afore mentioned disorders.

“Functional variant” and “biologically active variant” refer to a polypeptide that contains an amino acid sequence that differs from a reference polypeptide (e.g., HAS, human IgFc, CPP, MCPP, Degrader, human wild type mature MATRIN-3 (MATR3) peptide) by sequence replacement, deletion, or addition (e.g. HAS, human IgFc, CPP, MCPP or Degrader fusion polypeptide), and/or addition of non-polypeptide moieties (e.g. PEG, fatty acids) but retains desired functional activity of the reference polypeptide. The amino acid sequence of a functional variant can include one or more amino acid replacements, additions or deletions relative to the reference polypeptide, and include fragments of the reference polypeptide that retain the desired activity.

For example, a functional variant of HAS, human IgFc, CPP, MCPP (e.g., reversible bicyclization) prolongs the serum half-life of the fusion polypeptides described herein in comparison to the half-life of MATRIN-3 (MATR3), while retaining the reference MATRIN-3 (MATR3) (e.g., human MATRIN-3 (MATR3)) polypeptide's activity (e.g., reduced expression of DUX4 or DUX 4 fused form (CIC-DUX4 or DUX4-IGH) or target genes) activity. Polypeptide variants possessing a somewhat decreased level of activity relative to their wild-type versions can nonetheless be considered to be functional or biologically active polypeptide variants, although ideally a biologically active polypeptide possesses similar or enhanced biological properties relative to its wild-type protein counterpart (a protein that contains the reference amino acid sequence).

“Identity” means, in relation to nucleotide or amino acid sequence of a nucleic acid or polypeptide molecule, the overall relatedness between two such molecules. Calculation of the percent sequence identity (nucleotide or amino acid sequence identity) of two sequences, for example, can be performed by aligning the two sequences for optimal comparison purposes (e.g., gaps can be introduced in one or both of a first and a second nucleic acid or amino acid sequence for optimal alignment). The nucleotides or amino acids at corresponding positions are then compared. When a position in the first sequence is occupied by the same nucleotide or amino acid as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two sequences can be determined using methods such as those described by the National Center for Biotechnology Information (http://www.ncbi.nlm.nih.gov/). For example, the percent identity between two sequences can be determined using Clustal 2.0 multiple sequence alignment program and default parameters. Larkin M A et al. (2007) “Clustal W and Clustal X version 2.0.” Bioinformatics 23(21): 2947-2948.

The term “moiety,” as used herein, refers to a portion of a fusion polypeptide (e.g., SA-MATRIN3, CPP-MATRIN3, MCPP-MATRIN-3 (MATR3)) or fatty acid-conjugate described herein (e.g., AHA-(200-308)-hMATRIN-3 (MATR3)). The fusion proteins used in the methods of the present invention include, e.g., a MATRIN-3 (MATR3) moiety, which contains an amino acid sequence derived from MATRIN-3 (MATR3), and a SA, CPP or MCPP moiety, which contains an amino acid sequence derived from SA, CPP or MCPP. The fatty acid conjugates used in the methods of the present invention include, e.g., a MATRIN-3 (MATR3) moiety, which contains an amino acid sequence derived from MATRIN-3 (MATR3), and an fatty acid moiety, e.g., a fatty acid comprising one of the Formulae further described herein. The term “moiety” can also refer to a linker or functional molecule (e.g., PEG) comprising a fatty acid conjugate or fusion protein used in the methods of the present invention. The fusion protein optionally contains a linker moiety, which links the MATRIN-3 (MATR3) moiety and the SA, CPP or MCPP moiety, in the fusion polypeptide.

Without wishing to be bound by any particular theory, it is believed that the MATRIN-3 (MATR3) moiety confers biological function of bind DUX4 or DUX4-IGH or CIC-DUX4, prevent the interaction with DNA of DUX4 or DUX4-IGH or CIC-DUX4, inhibit the activation of specific target genes by DUX4 or DUX4-IGH or CIC-DUX4, reduce the toxic effects of DUX4, or reduce the cancer activity of DUX4-IGH or CIC-DUX4, while the SA, CPP or MCPP moiety prolongs the serum half-life, improves expression and stability, and increase delivery to skeletal muscle of the fusion polypeptides described herein.

The term “naturally occurring” when used in connection with biological materials such as nucleic acid molecules, polypeptides, host cells, and the like, refers to materials that are found in nature and are not manipulated by man. Similarly, “non-naturally occurring” as used herein refers to a material that is not found in nature or that has been structurally modified or synthesized by man. When used in connection with nucleotides, the term “naturally occurring” refers to the bases adenine (A), cytosine (C), guanine (G), thymine (T), and uracil (U). When used in connection with amino acids, the term “naturally occurring” refers to the 20 conventional amino acids (i.e., alanine (A), cysteine (C), aspartic acid (D), glutamic acid (E), phenylalanine (F), glycine (G), histidine (H), isoleucine (I), lysine (K), leucine (L), methionine (M), asparagine (N), proline (P), glutamine (Q), arginine (R), serine (S), threonine (T), valine (V), tryptophan (W), and tyrosine (Y)), as well as selenocysteine, pyrrolysine (PYL), and pyrroline-carboxy-lysine (PCL).

As used herein, the terms “variant,” “mutant,” as well as any like terms, when used in reference to MATRIN-3 (MATR3) or MCPP or specific versions thereof (e.g., “MATRIN-3 (MATR3) variant,” “human MATRIN-3 (MATR3) variant,” etc.) define protein or polypeptide sequences that comprise modifications, truncations, deletions, or other variants of naturally occurring (i.e., wild-type) protein or polypeptide counterparts or corresponding native sequences. “MATRIN-3 (MATR3) variant,” for instance, is described relative to the wild-type (i.e., naturally occurring) MATRIN-3 (MATR3) protein as described herein and known in the literature.

A “subject” is an individual to whom a MATRIN-3 (MATR3) fusion polypeptide or MATRIN-3 (MATR3) conjugate (e.g., usually in the form of a pharmaceutical composition) is administered. The subject is preferably a human, but “subject” includes animals, mammals, pet and livestock animals, such as cows, sheep, goats, horses, dogs, cats, rabbits, guinea pigs, rats, mice or other bovine, ovine, equine, canine, feline, rodent or murine species, poultry and fish.

The term “MATRIN-3 (MATR3) therapeutic agent” as used herein means a MATRIN-3 (MATR3) polypeptide, MATRIN-3 (MATR3) variant, MATRIN-3 (MATR3) fusion protein, or MATRIN-3 (MATR3) conjugate (e.g., a MATRIN-3 (MATR3) fatty acid conjugate), or a pharmaceutical composition comprising one or more of the same, that is administered to a subject in order to treat in a condition associated with aberrant expression and/or function of DUX4, such as FSHD or ALL

The terms “conjugate” and “fatty acid conjugate” are used interchangeably and are intended to refer to the entity formed as a result of a covalent attachment of a polypeptide or protein (or fragment and/or variant thereof) and a fatty acid moiety, optionally via a linker.

One of ordinary skill in the art will appreciate that various amino acid substitutions, e.g, conservative amino acid substitutions, may be made in the sequence of any of the polypeptide or protein described herein, without necessarily decreasing its activity. As used herein, “amino acid commonly used as a substitute thereof includes conservative substitutions (i.e., substitutions with amino acids of comparable chemical characteristics). For the purposes of conservative substitution, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Examples of amino acid substitutions include substituting an L-amino acid for its corresponding D-amino acid, substituting cysteine for homocysteine or other non-natural amino acids having a thiol-containing side chain, substituting a lysine for homolysine, diaminobutyric acid, diaminopropionic acid, ornithine or other non-natural amino acids having an amino containing side chain, or substituting an alanine for norvaline or the like.

The term “amino acid,” as used herein, refers to naturally occurring amino acids, unnatural amino acids, amino acid analogues and amino acid mimetics that function in a manner similar to the naturally occurring amino acids, all in their D and L stereoisomers if their structure allows such stereoisomeric forms. Amino acids are referred to herein by either their name, their commonly known three letter symbols or by the one-letter symbols recommended by the IUPAC-IUB Biochemical Nomenclature Commission.

The term “naturally occurring” refers to materials which are found in nature and are not manipulated by man. Similarly, “non-naturally occurring,” “un-natural,” and the like, as used herein, refers to a material that is not found in nature or that has been structurally modified or synthesized by man. When used in connection with amino acids, the term “naturally occurring” refers to the 20 conventional amino acids (i.e., alanine (A or Ala), cysteine (C or Cys), aspartic acid (D or Asp), glutamic acid (E or Glu), phenylalanine (F or Phe), glycine (G or Gly), histidine (H or His), isoleucine (I or He), lysine (K or Lys), leucine (L or Leu), methionine (M or Met), asparagine (N or Asn), proline (P or Pro), glutamine (Q or Gin), arginine (R or Arg), serine (S or Ser), threonine (T or Thr), valine (V or Val), tryptophan (W or Trp), and tyrosine (Y or Tyr)).

The terms “non-natural amino acid” and “unnatural amino acid,” as used herein, are interchangeably intended to represent amino acid structures that cannot be generated biosynthetically in any organism using unmodified or modified genes from any organism, whether the same or different. The terms refer to an amino acid residue that is not present in the naturally occurring (wild-type) protein sequence or the sequences of the present invention. These include, but are not limited to, modified amino acids and/or amino acid analogues that are not one of the 20 naturally occurring amino acids, selenocysteine, pyrrolysine (Pyl), or pyrroline-carboxy-lysine (Pel, e.g., as described in PCT patent publication WO2010/48582). Such non-natural amino acid residues can be introduced by substitution of naturally occurring amino acids, and/or by insertion of non-natural amino acids into the naturally occurring (wild-type) protein sequence or the sequences of the invention. The non-natural amino acid residue also can be incorporated such that a desired functionality is imparted to the molecule, for example, the ability to link a functional moiety (e.g., PEG). When used in connection with amino acids, the symbol “U” shall mean “non-natural amino acid” and “unnatural amino acid,” as used herein.

The term “analogue” as used herein referring to a polypeptide or protein means a modified peptide or protein wherein one or more amino acid residues of the peptide/protein have been substituted by other amino acid residues and/or wherein one or more amino acid residues have been deleted from the peptide/protein and/or wherein one or more amino acid residues have been added the peptide/protein. Such addition or deletion of amino acid residues can take place at the N-terminal of the peptide and/or at the C-terminal of the peptide.

The terms “MATRIN-3 (MATR3) polypeptide” and “MATRIN-3 (MATR3) protein” are used interchangeably and mean a naturally-occurring wild-type polypeptide expressed in a mammal, such as a human or a mouse. For purposes of this disclosure, the term “MATRIN-3 (MATR3) protein” can be used interchangeably to refer to any full-length MATRIN-3 (MATR3) polypeptide, which consists of 847 amino acid residues; (NCBI Ref. Seq. NP 954659) contains four known functional domains: Zinc finger 1 (aa 288-322), RNA recognition motif 1 (398-473), RNA recognition motif 2 (496-575) and Zinc finger 2 (798-833).

The term “MATRIN-3 (MATR3) variant” encompasses a MATRIN-3 (MATR3) polypeptide in which a naturally occurring MATRIN-3 (MATR3) polypeptide sequence has been modified. Such modifications include, but are not limited to, one or more amino acid substitutions, including substitutions with non-naturally occurring amino acids non-naturally-occurring amino acid analogs and amino acid mimetics.

In one aspect, the term “MATRIN-3 (MATR3) variant” refers to a MATRIN-3 (MATR3) protein sequence in which at least one residue normally found at a given position of a native MATRIN-3 (MATR3) polypeptide is deleted or is replaced by a residue not normally found at that position in the native MATRIN-3 (MATR3) sequence. In some cases it will be desirable to replace a single residue normally found at a given position of a native MATRIN-3 (MATR3) polypeptide with more than one residue that is not normally found at the position; in still other cases it may be desirable to maintain the native MATRIN-3 (MATR3) polypeptide sequence and insert one or more residues at a given position in the protein; in still other cases it may be desirable to delete a given residue entirely; all of these constructs are encompassed by the term “MATRIN-3 (MATR3) variant. The methods of the present invention also encompass nucleic acid molecules encoding such MATRIN-3 (MATR3) variant polypeptide sequences.

In various embodiments, a MATRIN-3 (MATR3) variant comprises an amino acid sequence that is at least about 85 percent identical to a naturally-occurring MATRIN-3 (MATR3) protein. In other embodiments, a MATRIN-3 (MATR3) polypeptide comprises an amino acid sequence that is at least about 90%, or about 95%, 96%, 97%, 98%, or 99% identical to a naturally-occurring MATRIN-3 (MATR3) polypeptide amino acid sequence. Such MATRIN-3 (MATR3) mutant polypeptides preferably, but need not, possess at least one activity of a wild-type MATRIN-3 (MATR3) mutant polypeptide, such as:
inhibits DUX4-induced toxicity in particular in HEK293 cells,
blocks induction of DUX4 targets, in particular in HEK293 cells,
interacts with the DNA-binding domain of DUX4,
inhibits DUX4 directly by blocking its ability to bind DNA,
inhibits the expression of DUX4 and DUX4 targets in particular in FSHD muscle cells,
rescues viability and myogenic differentiation in particular of FSHD muscle cells,
inhibits the expression of DUX4 and DUX4 targets in particular in FSHD muscle cells and
rescues viability and myogenic differentiation in particular of FSHD muscle cells;
the ability to treat, prevent, or ameliorate condition associated with an aberrant expression and/or function of at least one DUX4 protein and/or of at least one DUX4 fusion protein, such as muscular dystrophy, infection or cancer such as FSHD, herpes infection or ALL.

Although the MATRIN-3 (MATR3) polypeptides and MATRIN-3 (MATR3) mutant polypeptides, and the constructs comprising such polypeptides are primarily disclosed in terms of human MATRIN-3 (MATR3), the invention is not so limited and extends to MATRIN-3 (MATR3) polypeptides and MATRIN-3 (MATR3) mutant polypeptides and the constructs comprising such polypeptides where the MATRIN-3 (MATR3) polypeptides and MATRIN-3 (MATR3) mutant polypeptides are derived from other species (e.g., cynomolgous monkeys, mice and rats). In some instances, a MATRIN-3 (MATR3) polypeptide or a MATRIN-3 (MATR3) mutant polypeptide can be used to treat or ameliorate a disorder in a subject in a mature form of a MATRIN-3 (MATR3) mutant polypeptide that is derived from the same species as the subject.

A MATRIN-3 (MATR3) mutant polypeptide is preferably biologically active. In various respective embodiments, a MATRIN-3 (MATR3) polypeptide or a MATRIN-3 (MATR3) mutant polypeptide has a biological activity that is equivalent to, greater to or less than that of the naturally occurring form of the mature MATRIN-3 (MATR3) protein. Examples of biological activities include the ability to bind DUX4 or DUX4-IGH or CIC-DUX4, prevent the interaction with DNA of DUX4 or DUX4-IGH or CIC-DUX4, inhibit the activation of specific target genes by DUX4 or DUX4-IGH or CIC-DUX4, reduce the toxic effects of DUX4, or reduce the cancer activity of DUX4-IGH or CIC-DUX4. As used herein in the context of the structure of a polypeptide or protein, the term “N-terminus” (or “amino terminus”) and “C-terminus” (or “carboxyl terminus”) refer to the extreme amino and carboxyl ends of the polypeptide, respectively.

The term “therapeutic polypeptide” or “therapeutic protein” as used herein means a polypeptide or protein which is being developed for therapeutic use, or which has been developed for therapeutic use.

The present invention will be illustrated by means of non-limiting examples in reference to the following figures.

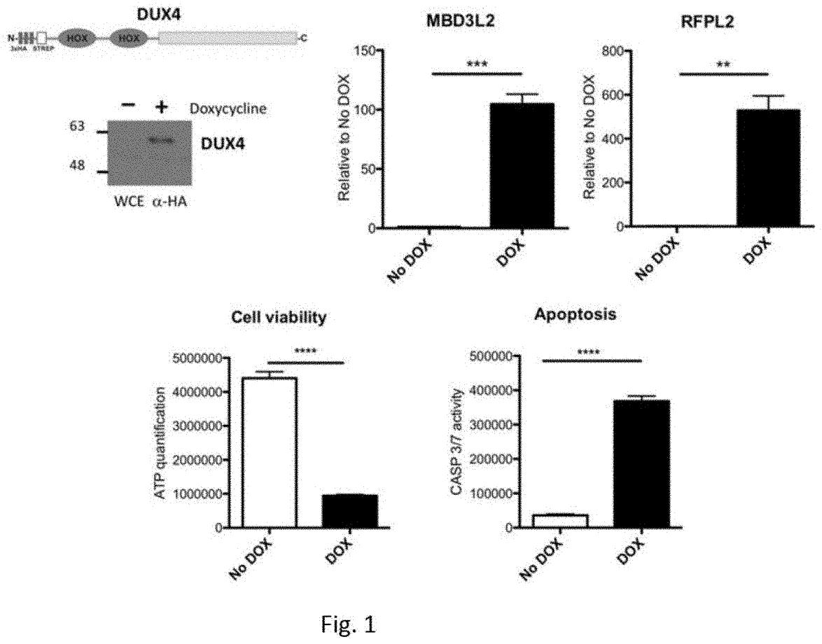

FIG. 1. Characterization of iSH-DUX4 cells.

After doxycycline administration, DUX4 protein expression is detectable after 4 h (top left), DUX4 target genes are upregulated after 8 h (top right), and significant apoptosis is detectable within 24 h (bottom) (unpaired two-tailed Student's t test, **p<0.01, ***p<0.001, ****p<0.0001, n=3, mean±SEM).

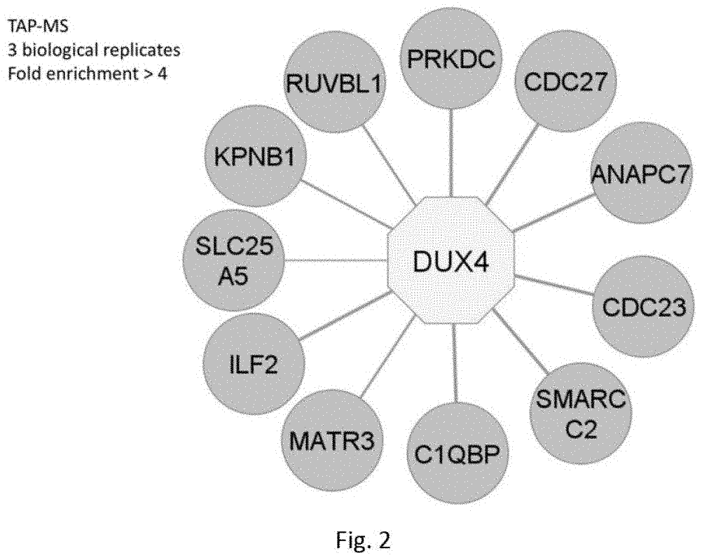

FIG. 2. DUX4 nuclear interactome.

Graphical representation of DUX4 and its interacting proteins in the nucleus of mammalian cells. In the figure, proteins identified in all the STREP-HA affinity purifications with a spectral count average of DUX4/EV control ratio >4 are displayed. DUX4 is highlighted in the center and the interactors are displayed on the side. The thickness of the edges is proportional to the spectral count average of DUX4/EV control ratio.

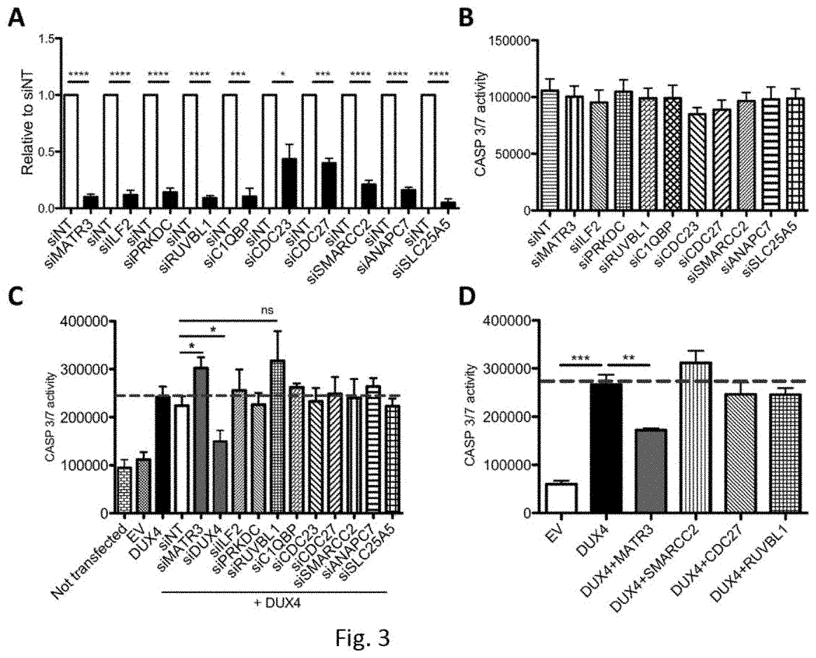

FIG. 3. MATR3 protects from DUX4-induced apoptosis in HEK293 cells.

A. Real-time quantitative PCR (RT-qPCR) showing the efficiency of knockdown for the indicated DUX4 interactors in HEK293 cells. Values are expressed relative to cells transfected with control siRNAs (siNT) (unpaired two-tailed Student's t test, *p<0.05; ***p<0.001; ****p<0.0001. n=3, mean±SEM).

B. Knockdown of DUX4 interactors does not affect cell viability in the absence of DUX4 expression. Caspase 3/7 activity assays performed upon knockdown of the indicated targets in HEK293 cells not expressing DUX4 (paired two-tailed Student's t test, n=4, mean±SEM).

C. MATR3 knockdown increases DUX4 toxicity. HEK293 cells transfected with empty vector (EV), DUX4 or DUX4 in combination with siRNAs specific for the indicated targets. Cells were collected 48 h after transfection following by caspase 3/7 activity assay (paired two-tailed Student's t test, *p<0.05, n=5, mean±SEM).

D. MATR3 overexpression reduces DUX4-induced apoptosis. HEK293 cells transfected with empty vector (EV), DUX4 or DUX4 in combination with expression vectors for the indicated factors. Cells were collected 48 h after transfection followed by caspase 3/7 activity assay (paired two-tailed Student's t test, **p<0.01, ***p<0.001, n=4, mean±SEM).

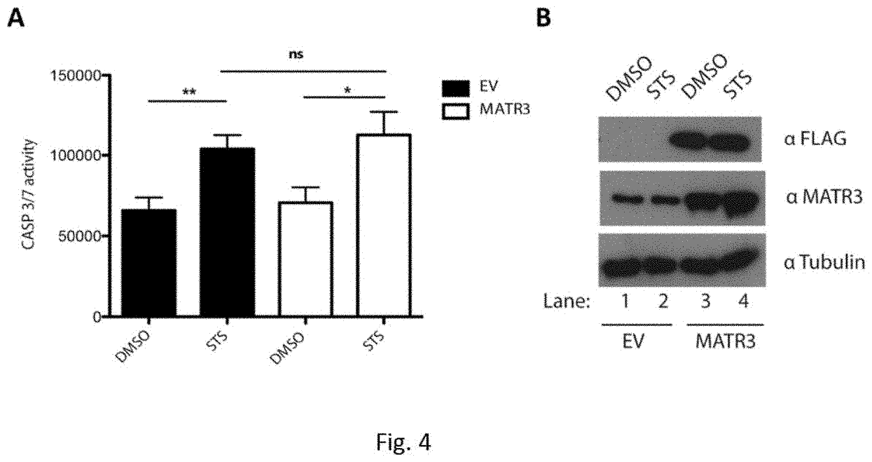

FIG. 4. MATR3 does not protect from Staurosporine-induced apoptosis.

A. HEK293 cells were transfected with empty vector (EV) or MATR3. 24 h after transfection, cells were treated with Staurosporine or DMSO (as negative control) for 6 h followed by caspase 3/7 activity assay (unpaired two-tailed Student's t test, *p<0.05, **p<0.01, n=8, mean±SEM).

B. Immunoblotting with anti-FLAG (recognizing transfected MATR3), anti-MATR3 (recognizing endogenous as well as transfected MATR3) and anti-tubulin (as loading control) on total proteins extracts from HEK293 cells transfected with EV (lanes 1 and 2) or MATR3 (lanes 3 and 4) and treated with DMSO (lanes 1 and 3) or Staurosporine (lanes 2 and 4).

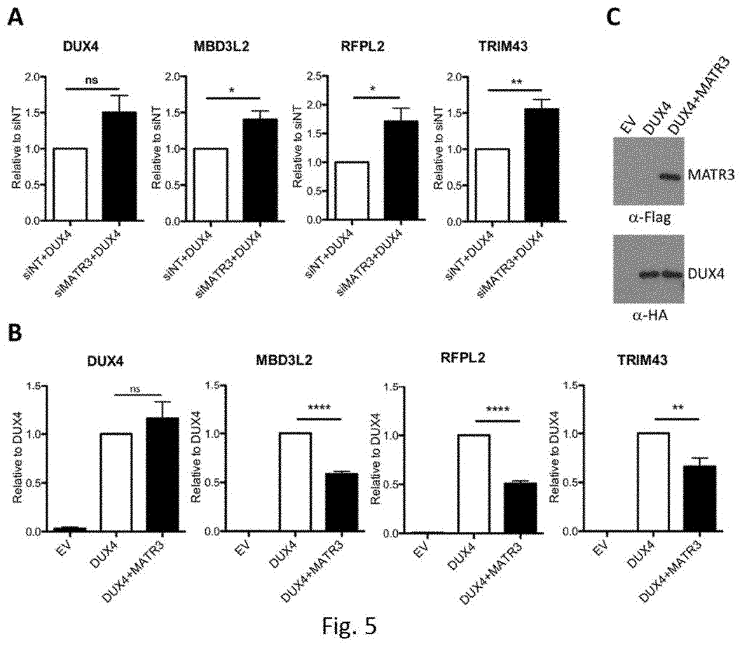

FIG. 5. MATR3 blocks DUX4-transcriptional activity in HEK293 cells.

A. HEK293 cells were transfected with DUX4 in combination with control (siNT) or MATR3 siRNAs and the expression levels of the indicated transcripts were measured by RT-qPCR. Data are represented relative to siNT (unpaired two-tailed Student's t test, *p<0.05, **p<0.01, n=4, mean±SEM).

B. HEK293 cells were transfected with empty vector (EV), DUX4 or DUX4 in combination with MATR3 and the expression levels of the indicated transcripts were measured by RT-qPCR. Data are expressed relative to DUX4 (unpaired two-tailed Student's t test, **p<0.01, ****p<0.0001, n=4, mean±SEM).

C. Immunoblotting with anti-Flag (for MATR3, top) or anti-HA (for DUX4, bottom) antibodies on whole cell extracts from HEK293 cells transfected with empty vector (EV), HA-tagged DUX4, or HA-DUX4 and Flag-tagged MATR3. One representative experiment is shown.

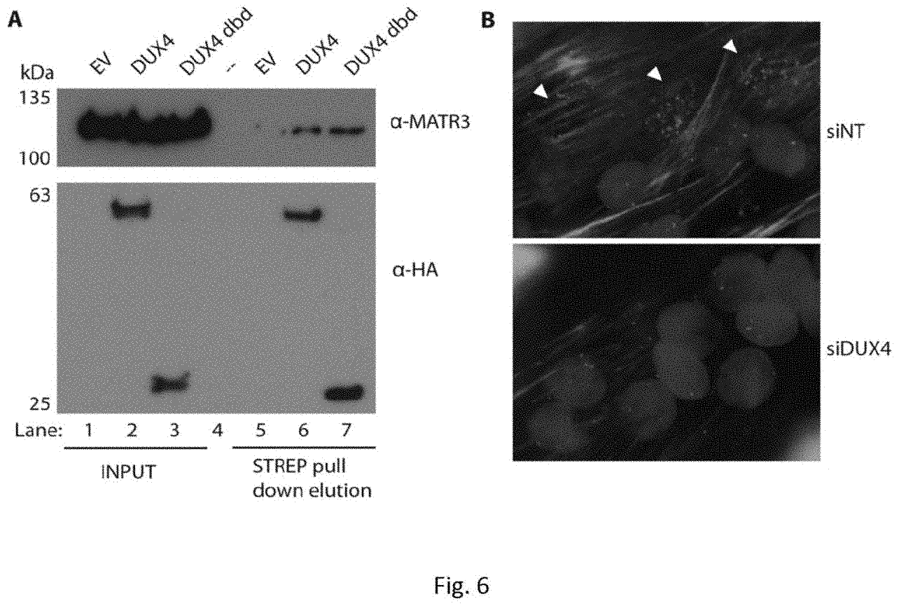

FIG. 6. The endogenous DUX4 and MATR3 interact in primary FSHD muscle cells.

A. Strep-Tactin pull-down of transfected DUX4 full length or DUX4 DNA-binding domain (dbd) with endogenous MATR3. HEK293 cells were transfected with empty vector (EV), DUX4 or DUX4 dbd. Nuclear proteins were incubated with Strep-Tactin beads, pull-down complexes were specifically eluted with D-Biotin-excess. Immunoblotting was performed with antibodies against MATR3 or HA (detecting DUX4 and DUX4 dbd), which showed interaction of the endogenous MATR3 with both DUX4 full length (lane 6) and DUX4 dbd (lane 7).

B. Proximity ligation assay (PLA) supports the interaction between endogenous DUX4 and MATR3 in primary FSHD muscle cells. Terminally differentiated FSHD muscle cells treated with control (siNT) or DUX4 siRNAs were incubated with anti-DUX4 and anti-MATR3 antibodies followed by PLA staining. Positive PLA signals (white arrows) are present in nuclei of FSHD cells treated with siNT, while they are absent in cells treated with siDUX4.

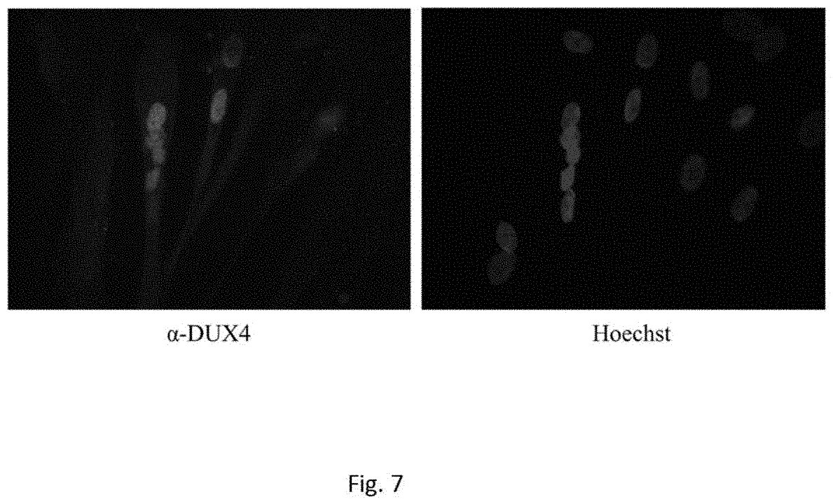

FIG. 7. Endogenous DUX4 is expressed only in a fraction of FSHD myonuclei.

Representative immunofluorescence of DUX4 (red, left) performed with anti-DUX4 E5-5 antibody in primary FSHD myotubes. Hoechst 33342 was used to stain nuclei (blue, right).

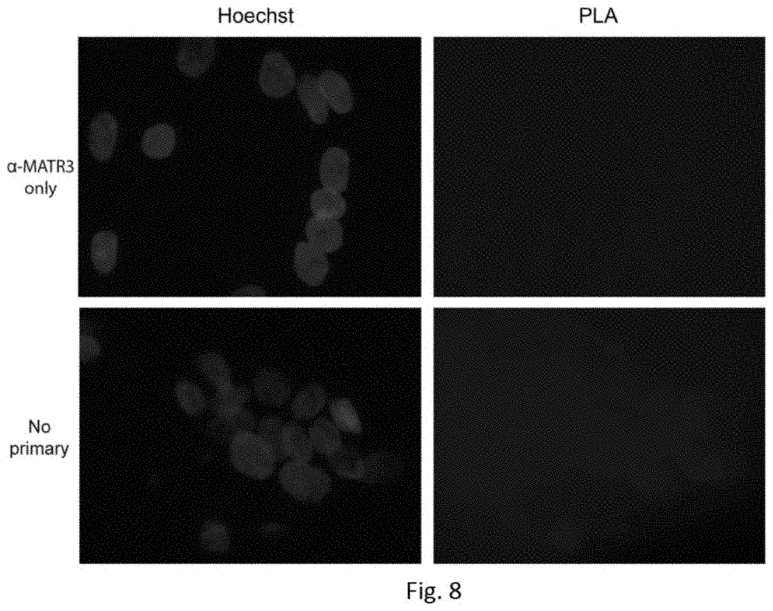

FIG. 8. PLA signal is specific for DUX4-MATR3 interaction.

Proximity ligation assay (PLA) performed in primary FSHD myotubes with only one (anti-MATR3, top) or without any (bottom) primary antibody as negative controls, to assess the specificity of the interaction between endogenous MATR3 and DUX4 shown in FIG. 6B. Hoechst 33342 was used to stain nuclei (blue, right).

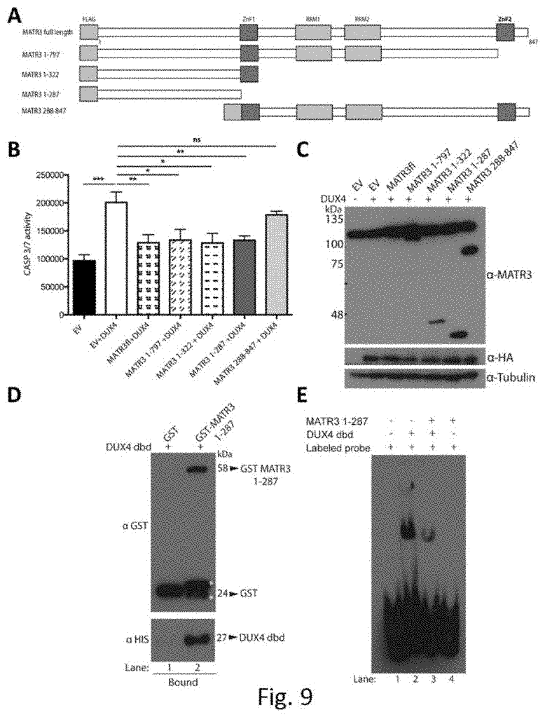

FIG. 9. MATR3 directly inhibits DNA binding by DUX4.

A. Schematic representation illustrating the principal domains of MATR3 full length (1-847). Deletion mutants 1-797, 1-322, 1-287 and 288-847 are also depicted. The N-terminal grey box indicates the FLAG-tag.

B. Caspase 3/7 activity assay in HEK293 cells transfected with empty vector (EV), DUX4 and DUX4 in combination with the indicated MATR3 constructs (unpaired two-tailed Student's t test, *p<0.05, **p<0.01, ***p<0.001, n=10, mean±SEM).

C. Immunoblotting with anti-MATR3 (recognizing endogenous as well as transfected MATR3), anti-HA (recognizing transfected DUX4) and anti-tubulin (as loading control) antibodies on total proteins extracts from HEK293 cells transfected with empty vector (EV), DUX4 or DUX4 in combination with the indicated MATR3 constructs.

D. Pull-down assay with purified His-DUX4 dbd and purified GST-MATR3 1-287 or GST (as negative control) analyzed by immunoblotting with antibodies against GST or His tag. Asterisks (*) indicate the position of two degradation products of GST-MATR3 1-287. One representative experiment out of three independent experiments is shown.

E. Electromobility shift assay with a labeled probe containing DUX4 binding sites and purified DUX4 dbd. The addition of purified MATR3 1-287 reduces DUX4 binding to the probe (lane 3). MATR3 1-287 alone is not able to bind DNA (lane 4). One representative experiment out of three independent experiments is shown.

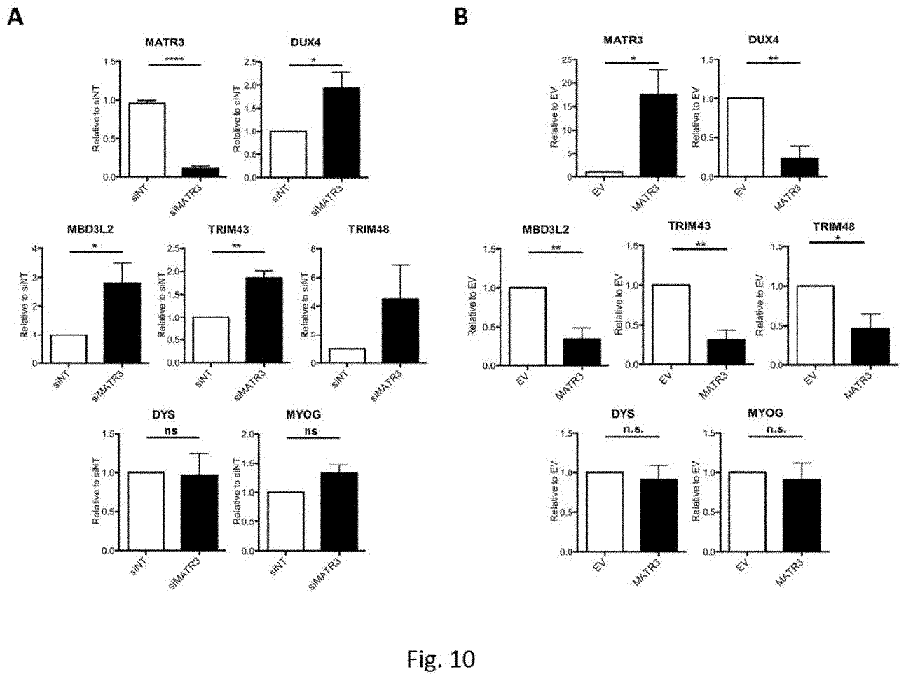

FIG. 10. MATR3 inhibits DUX4-transcriptional activity in primary FSHD muscle cells.

A. Primary FSHD muscle cells were transfected with either control (siNT) or MATR3 siRNAs and the expression levels of the indicated transcripts were measured by real-time quantitative PCR (RT-qPCR). Data are represented relative to siNT.

B. Primary FSHD muscle cells were transduced with empty vector (EV) or MATR3 lentiviruses and the expression levels of the indicated transcripts were measured by RT-qPCR. Data are represented relative to EV.

(unpaired two-tailed Student's t test, *p<0.05, **p<0.01, ****p<0.0001, n=4, mean±SEM).

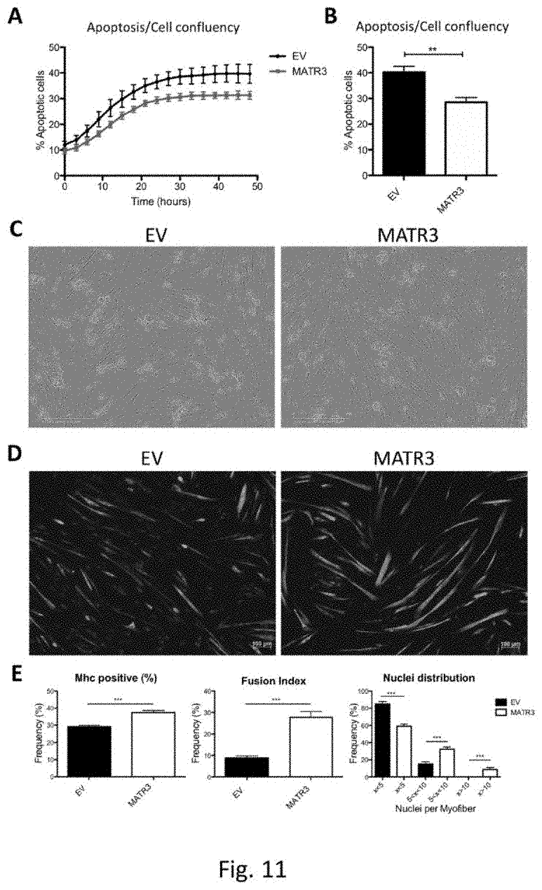

FIG. 11. MATR3 overexpression rescues DUX4 toxicity in FSHD muscle cells.

A. Real-time apoptotic levels in FSHD muscle cells transduced with empty vector (EV) or MATR3 lentiviruses. Results are reported as percentage (%) of apoptotic cells upon normalization of the apoptotic signal over cell confluence (n=3).

B. Percentage of apoptotic cells extracted at a single time-point (48 h) during the apoptosis quantification time course in FSHD muscle cells transduced with EV or MATR3 lentiviruses (unpaired two-tailed Student's t test, **p<0.01, n=3, mean±SEM).

C. Representative images of FSHD muscle cells transduced with EV or MATR3 lentiviruses in combination with the apoptosis detection reagent. A merge of the phase contrast and fluorescence signals is shown.

D. Representative images of immunofluorescence for Myosin Heavy Chain and nuclear staining in terminally differentiated FSHD muscle cells treated with EV or MATR3 lentiviruses.

E. Differentiation index expressed as percentage of MHC-positive cells, calculated in comparison with the total number of nuclei. Fusion index, calculated as the number of nuclei present in myotubes (at least 3 nuclei) in comparison with the total number of nuclei. Nuclei distribution, calculated as the frequency of MHC+ cells containing the indicated number of nuclei (unpaired two-tailed Student's t test, ***p<0.001, n=3, mean±SEM).

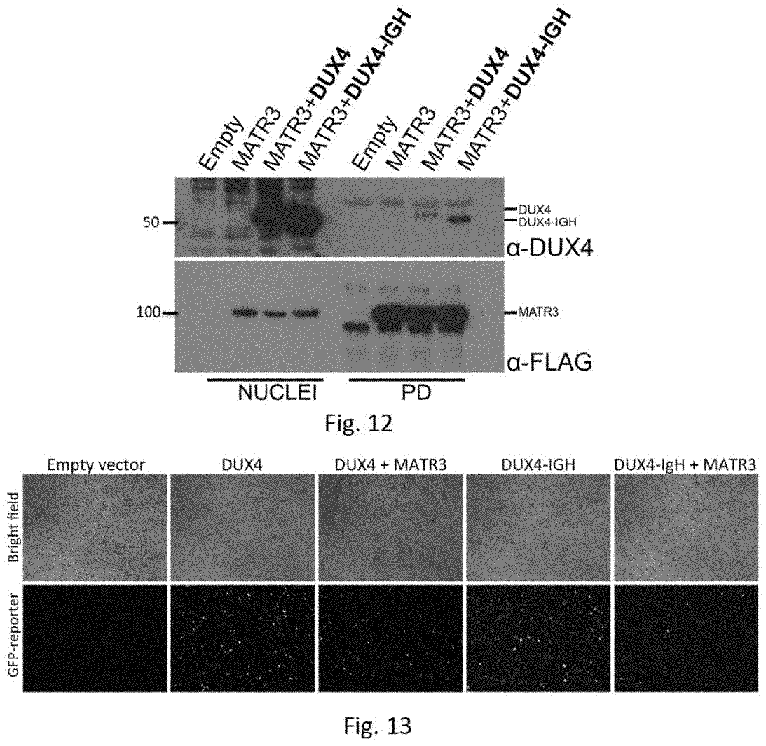

FIG. 12. MATR3 interacts with DUX4-IGH. Cell were transfected with empty vector, FLAG-MATR3 alone, FLAG-MATR3 plus DUX4 (as positive control) or FLAG-MATR3 plus DUX4-IGH. Nuclear pulldowns of FLAG-tagged MATR3 were followed by immunoblotting with antibodies specific for DUX4 (top) or FLAG-MATR3 (bottom).

FIG. 13. MATR3 inhibits DUX4-IGH. Representative GFP fluorescence images of HEK293 cells (20×) transfected with a DUX4-/DUX4-IGH-dependent GFP reporter together with EV, DUX4 or DUX4 plus MATR3, DUX4-IGH or DUX4-IGH plus MATR3, as indicated.

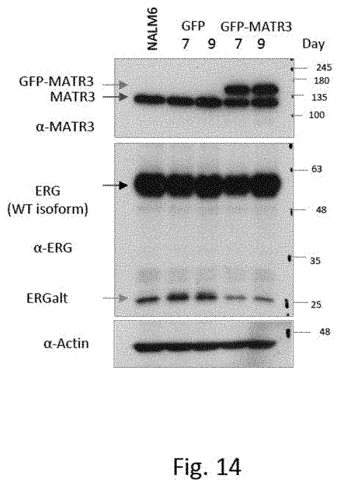

FIG. 14. MATR3 inhibits ERGalt expression in leukemic cells. Western blot analysis of DUX4-IGH expressing NALM6 B-ALL cells un-transduced or transduced with GFP or GFP-MATR3 lentiviral vectors. The GFP-MATR3 transgene is indicated by a green arrow, while a red arrow indicates the endogenous MATR3. The full length (WT) and ERGalt are indicated by black and blue arrows, respectively. Actin is used as loading control.

### DETAILED DESCRIPTION OF THE INVENTION

Material and Methods

Study Design

The primary objective of this study was to identify specific DUX4 interactors and determine their ability to regulate DUX4 activity. The inventors used a proteomic approach to isolate proteins interacting with DUX4 and controlled laboratory experiments to test if the identified factors could affect DUX4-induced toxicity. Biochemical, gene expression, apoptosis and differentiation assays were used to dissect how MATR3 inhibits DUX4. Determination of sample sizes was based on previous experiences with apoptosis and gene expression studies. At least three biological replicates were used in all the experiments. Because of the conspicuous effect of MATR3 overexpression on survival of DUX4 expressing cells, these studies could not be blinded. All procedures involving human samples were approved by IRCCS San Raffaele Scientific Institute Ethical Committee.

Constructs and Cloning Procedures

FLAG MATR3 C-terminal truncation mutants 1-287, 1-322 and 1-797 were generated though mutagenic PCR by the introduction of termination codons into the expression vector pCMV-Tag2B N-terminal FLAG MATR3 full-length (Addgene #32880), using the QuickChange Lightning site-directed mutagenesis kit (Agilent Technologies).

Primers used for replacing specific amino acids with the termination codon are listed in Table 2.

TABLE 2
List of antibodies, primer and siRNA
Antibodies
Western Blot Antibodies
Primary antobodies
Rabbit anti-Matrin 3 (#PA5-57720; Thermo Fisher Scientific) 1:1000
Mouse anti-Tubulin (#T9026; Sigma-Aldrich) 1:10000
Mouse anti-GST (#G1160; Sigma-Aldrich) 1:10000
Mouse anti-6xHis (#631212; Clontech) 1:5000
Rabbit anti-Matrin 3 (#A300-591A; Bethyl) 1:1000
Mouse anti-FLAG-M2 (#F1804; Sigma-Aldrich) 1:1000
Mouse anti-HA.11 (#MMS-101R; Covance) 1:1000
Secondary antobodies
Peroxidase-AffiniPure Donkey Anti-Mouse IgG
(H + L) (#JI715035150; Jackson
ImmunoResearch) 1:10000
Peroxidase-AffiniPure Donkey Anti-Goat IgG
(H + L) (#705-035-003; Jackson
ImmunoResearch) 1:10000
PLA Antibodies
Rabbit anti-Matrin 3 (#A300-591A; Bethyl) 1:200
Mouse anti-DUX4 P2B1 (#SAB5200019; Sigma Aldrich) 1:50
IF antobodies
Primary antobodies
Rabbit anti-DUX4 E55 (#ab124699, Abcam) 1:100
Mouse anti-myosin, sarcomere (MHC) (MF 20;
Developmental Studies Hybridoma Bank) 1:2
Secondary antobodies
Alexa Fluor 555 goat anti-rabbit (#A-27039, Molecular Probes)
Alexa Fluor 488 goat anti-mouse (#A-11001, Molecular Probes) 1: 500

Primer Oligonucleotides

| Target gene | Sequence | |
|---|---|---|
| Primers used | | |
| in RT-qPCR | | |
| GAPDH | Fw | TCAAGAAGGTGGTGAAGCAGG (SEQ ID NO: 27) |
| | Rv | ACCAGGAAATGAGCTTGACAAA (SEQ ID NO: 28) |
| MATR3 | Fw | ATCAATGGAGCAAGTCACAGTC (SEQ ID NO: 29) |
| | Rv | TGCAACATGAATGGATCACCC (SEQ ID NO: 30) |
| ILF2 | Fw | CTCAGACTCTCGTCCGAATCC (SEQ ID NO: 31) |
| | Rv | CAGAAGCAAGATAGCTGGCATC (SEQ ID NO: 32) |
| PRKDC | Fw | GAGAAGGCGGCTTACCTGAG (SEQ ID NO: 33) |
| | Rv | CGAAGGCCCGCTTTAAGAGA (SEQ ID NO: 34) |
| RUVBL1 | Fw | GGCATGTGGCGTCATAGTAGA (SEQ ID NO: 35) |
| | Rv | CACGGAGTTAGCTCTGTGACT (SEQ ID NO: 36) |
| C1QBP | Fw | CGTGTGCTGGGCTCCTC (SEQ ID NO: 37) |
| | Rv | AAAGCTTTGTCTCCGTCGGT (SEQ ID NO: 38) |
| CDC23 | Fw | CTGCGAGTACCTCCATGGTC (SEQ ID NO: 39) |
| | Rv | AGAGAGAAAGCCAACTCCGC (SEQ ID NO: 40) |
| CDC27 | Fw | TGCTGACGTGTTTCTTGTCC (SEQ ID NO: 41) |
| | Rv | TTGCACTGCCTTTCATTCTG (SEQ ID NO: 42) |
| SMARCC2 | Fw | CCGTGACCCAGTTCGACAAC (SEQ ID NO: 43) |
| | Rv | CGGCAGTTTAGTGAGCGGT (SEQ ID NO: 44) |
| ANAPC7 | Fw | GCTTTTCGAGTCAGTGCTGC (SEQ ID NO: 45) |
| | Rv | GGGGAGAATAACTCAGGGTTG (SEQ ID NO: 46) |
| SLC25A5 | Fw | TTGATTTTGCCCGTACCCGT (SEQ ID NO: 47) |
| | Rv | GGATCCGGAAGCATTCCCTT (SEQ ID NO: 48) |
| DUX4 | Fw | GCGCAACCTCTCCTAGAAAC (SEQ ID NO: 49) |
| (overexpressed) | Rv | AGCAGAGCCCGGTATTCTTC (SEQ ID NO: 50) |
| DUX4 | Fw | CCCAGGTACCAGCAGACC (SEQ ID NO: 51) |
| (endogenous) | Rv | TCCAGGAGATGTAACTCTAATCCA (SEQ ID NO: 52) |
| MBD3L2 | Fw | GCGTTCACCTCTTTTCCAAG (SEQ ID NO: 53) |
| | Rv | GCCATGTGGATTTCTCGTTT (SEQ ID NO: 54) |
| RFPL2 | Fw | CCCACATCAAGGAACTGGAG (SEQ ID NO: 55) |
| | Rv | TGTTGGCATCCAAGGTCATA (SEQ ID NO: 56) |
| TRIM43 | Fw | ACCCATCACTGGACTGGTGT (SEQ ID NO: 57) |
| | Rv | CACATCCTCAAAGAGCCTGA (SEQ ID NO: 58) |
| TRIM48 | Fw | GGAGCTGTGTTTTGGTGACCT (SEQ ID NO: 59) |
| | Rv | GTAGTTCATGCAGATGGGGCA (SEQ ID NO: 60) |
| DYSTROPHIN | Fw | AGCAAGAGCACAACAATTTGG (SEQ ID NO: 61) |
| | Rv | CCCTGTTCGTCCCGTATCA (SEQ ID NO: 62) |
| MYOGENIN | Fw | GCTCAGCTCCCTCAACCA (SEQ ID NO: 63) |
| | Rv | GCTGTGAGAGCTGCATTCG (SEQ ID NO: 64) |
| Primers used | | |
| for cloning | | |
| FLAG MATR3 | Fw | CATGGACTCTTACCGAAGTAATATCCCCATCTGTGCTCT (SEQ ID NO: 65) |
| 1-287 | Rv | AGAGCACAGATGGGGATATTACTTCGGTAAGAGTCCATG (SEQ ID NO: 66) |
| FLAG MATR3 | Fw | CGTCGATGCCAGCTTCTTTAAGAAATCTACCCAGAATGG (SEQ ID NO: 67) |
| 1-322 | Rv | CCATTCTGGGTAGATTTCTTAAAGAAGCTGGCATCGACG (SEQ ID NO: 68) |
| FLAG MATR3 | Fw | GACTATGTGATACCTTAAACAGGGTTTTACTGTAAGCTG (SEQ ID NO: 69) |
| 1-797 | Rv | CAGCTTACAGTAAAACCCTGTTTAAGGTATCACATAGTC (SEQ ID NO: 70) |
| FLAG MATR3 | Fw | AAAGGATCCTATCCCCATCTGTGCTCTATATGTG (SEQ ID NO: 71) |
| 288-end | Rv | AAACTCGAGTTAAGTTTCCTTCTTCTG (SEQ ID NO: 72) |
| DUX4 full- | Fw | GGGGACAAGTTTGTACAAAAAAGCAGGCTCCGCCCTCCCGACACCCTCGG |
| length (attB) | | AC (SEQ ID NO: 73) |
| | Rv | GGGGACCACTTTGTACAA |
| | | GAAAGCTGGGTCTAAAGCTCCTCCAGCAGAGCC (SEQ ID NO: 74) |
| DUX4 dbd | Fw | GGGGACAAGTTTGTACAAAAAAGCAGGCTCCGCCCTCCCGACACCCTCGG |
| (attB) | | AC (SEQ ID NO: 75) |
| | Rv | GGGGACCACTTTGTACAAGAAAGCTGGGTCTAGACCTGCGCGGGCGCCC |
| | | (SEQ ID NO: 76) |
| EMSA probe | | |
| FRG1 Peak 1 | Fw | AATTGTAGCTATAATTCAATCATCTAAATTG (SEQ ID NO: 77) |
| | Rv | CAATTTAGATGATTGAATTATAGCTACAATT (SEQ ID NO: 78) |

List of siRNAs

| Target | Species | Description | Sequence/Catalogue number |
|---|---|---|---|
| MATR3 | Human | ON-TARGETplus SMARTpool - | FE5L017382000005 |
| | | Dharmacon | |
| DUX4 | Human | Stealth RNA (custom) - Life | CCGAGCCTTTGAGAAGGATCGCTTT |
| | | Technologies | (SEQ ID NO: 79) |
| ILF2 | Human | ON-TARGETplus SMARTpool - | FE5L017599000005 |
| | | Dharmacon | |
| PRKDC | Human | ON-TARGETplus SMARTpool - | FE5L005030000005 |
| | | Dharmacon | |
| RUVBL1 | Human | ON-TARGETplus SMARTpool - | FE5L008312000005 |
| | | Dharmacon | |
| C1QBP | Human | ON-TARGETplus SMARTpool - | FE5L011225010005 |
| | | Dharmacon | |
| CDC23 | Human | ON-TARGETplus SMARTpool - | FE5L009523000005 |
| | | Dharmacon | |
| CDC27 | Human | ON-TARGETplus SMARTpool - | FE5L003229000005 |
| | | Dharmacon | |
| SMARCC2 | Human | ON-TARGETplus SMARTpool - | FE5L008977000005 |
| | | Dharmacon | |
| ANAPC7 | Human | ON-TARGETplus SMARTpool - | FE5L021035000005 |
| | | Dharmacon | |
| SLC25A5 | Human | ON-TARGETplus SMARTpool - | FE5L007486020005 |
| | | Dharmacon | |

FLAG MATR3 288-end were cloned into a pCMV-Tag2B vector (Addgene), previously XhoI and BamHI (New England BioLabs) digested and dephosphorylated (Antarctic Phosphatase; New England Biolabs). The coding sequence of interest was amplified by PCR (GoTaq flexi DNA polymerase; Promega) employing MATR3 full-length as template and 5′-end overhang primers containing restriction enzyme sites listed in Table 2.

DUX4 full-length (NP_001292997.1)
(SEQ ID NO: 80)

| | |
|---|---|
| 1 | malptpsdst lpaeargrgr rrrlvwtpsq sealracfer |
| | npypgiatre rlaqaigipe |
| 61 | prvqiwfqne rsrqlrqhrr esrpwpgrrg ppegrrkrta |
| | vtgsqtalll rafekdrfpg |
| 121 | iaareelare tglpesriqi wfqnrrarhp gqggrapaqa |
| | gglcsaapgg ghpapswvaf |
| 181 | ahtgawgtgl paphvpcapg alpqgafvsq aaraapalqp |
| | sqaapaegis qpapargdfa |
| 241 | yaapappdga lshpqaprwp phpgksredr dpqrdglpgp |
| | cavaqpgpaq agpqgqgvla |
| 301 | pptsqgspww gwgrgpqvag aawepqagaa pppqpappda |
| | sasarqgqmq gipapsgalq |
| 361 | epapwsalpc gllldellas peflqqaqpl leteapgele |
| | aseeaaslea plseeeyral |
| 421 | leel |

and dbd
(SEQ ID NO: 81)

| | |
|---|---|
| 1 | malptpsdst lpaeargrgr rrrlvwtpsq sealracfer |
| | npypgiatre rlaqaigipe |
| 61 | prvqiwfqne rsrqlrqhrr esrpwpgrrg ppegrrkrta |
| | vtgsqtalll rafekdrfpg |
| 121 | iaareelare tglpesriqi wfqnrrarhp |

vectors were generated through the Gateway Technology, employing as DNA template pCS2-mkgDUX4 vector (Addgene #21156) as DNA template, the primers listed in Table S2 and plasmid pcDNA FRT/TO strep-HA as destination vector, kindly provided by Dr. Giulio Superti-Furga (CeMM Research Center for Molecular Medicine of the Austrian Academy of Sciences, 1090 Vienna, Austria).

For recombinant protein purification, DUX4 dbd insert was cloned into the bacteria expression vector pET-GB1, which contains the GB1 peptide (SEQ ID NO:82):

| | |
|---|---|
| 1 | xqyklilngk tlkgetttea vdaataekvf kqyandngvd |
| | gewtyddatk tftvte |

to improve the protein solubility and 6xHis as tag (82).

The vector was digested with NcoI and XhoI (New England BioLabs) and purified using QIAquick PCR purification kit (QIAGEN).

DUX4 dbd insert was amplified by PCR (GoTaq flexi DNA polymerase; Promega) employing pTO-STREP HA DUX4 vector as template and 5′-end overhang primers containing the restriction enzyme sites.

pGEX-2tk vector digested with BamHI and EcoRI (New England BioLabs) and purified was used for the cloning of MATR3 1-287. MATR3 1-287 insert was amplified by PCR (GoTaq flexi DNA polymerase; Promega) using pCMV FLAG-MATR3 vector as template and 5′-end overhang primers containing the restriction enzyme sites.

pTO STREP-HA DUX4 full length
(SEQ ID NO: 83)
gacggatcgggagatctcccgatcccctatggtgcactctcagtacaatctgctctgatgccgcatagttaagccagtatctgctccctgctt
gtgtgttggaggtcgctgagtagtgcgcgagcaaaatttaagctacaacaaggcaaggcttgaccgacaattgcatgaagaatctgcttag
ggttaggcgttttgcgctgcttcgcgatgtacgggccagatatacgcgttgacattgattattgactagttattaatagtaatcaattacggggt
cattagttcatagcccatatatggagttccgcgttacataacttacggtaaatggcccgcctggctgaccgcccaacgacccccgcccattg
acgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggat
agcggtttgactcacggggatttccaagtctccaccccattgacgtcaatgggagtttgttttggcaccaaaatcaacgggactttccaaaat
gtcgtaacaactccgccccattgacgcaaatgggcggtaggcgtgtacggtgggaggtctatataagcagagctctccctatcagtgata
gagatctccctatcagtgatagagatcgtcgacgagctcgtttagtgaaccgtcagatcgcctggagacgccatccacgctgttttgacctc
catagaagacaccgggaccgatccagcctccggactctagcgtttaaacttaagcttggtaccgagctcggatccactagtccagtgtggt
ggaattctgcagatatccagcacagtggcggccgctcgagaccatgtacccatacgatgttcctgactatgccggtaccgagctcggatc
caccatggctagctggagccacccgcagttcgagaaaggtggaggttccggaggtggatcgggaggtggatcgtggagccacccgca
gttcgaaaaagcggccgatatcacaagtttGTACAAAAAAGCAGGCTCCATGGCCCTCCCGACACCC
TCGGACAGCACCCTCCCCGCGGAAGCCCGGGGACGAGGACGGCGACGGAGACTCG
TTTGGACCCCGAGCCAAAGCGAGGCCCTGCGAGCCTGCTTTGAGCGGAACCCGTAC
CCGGGCATCGCCACCAGAGAACGGCTGGCCCAGGCCATCGGCATTCCGGAGCCCA
GGGTCCAGATTTGGTTTCAGAATGAGAGGTCACGCCAGCTGAGGCAGCACCGGCG
GGAATCTCGGCCCTGGCCCGGGAGACGCGGCCCGCCAGAAGGCCGGCGAAAGCGG
ACCGCCGTCACCGGATCCCAGACCGCCCTGCTCCTCCGAGCCTTTGAGAAGGATCG
CTTTCCAGGCATCGCCGCCCGGGAGGAGCTGGCCAGAGAGACGGGCCTCCCGGAG
TCCAGGATTCAGATCTGGTTTCAGAATCGAAGGGCCAGGCACCCGGGACAGGGTG
GCAGGGCGCCCGCGCAGGCAGGCGGCCTGTGCAGCGCGGCCCCCGGCGGGGGTCA
CCCTGCTCCCTCGTGGGTCGCCTTCGCCCACACCGGCGCGTGGGGAACGGGGCTTC
CCGCACCCCACGTGCCCTGCGCGCCTGGGGCTCTCCCACAGGGGGCTTTCGTGAGC
CAGGCAGCGAGGGCCGCCCCCGCGCTGCAGCCCAGCCAGGCCGCGCCGGCAGAGG
GGATCTCCCAACCTGCCCCGGCGCGCGGGGATTTCGCCTACGCCGCCCCGGCTCCT
CCGGACGGGGCGCTCTCCCACCCTCAGGCTCCTCGGTGGCCTCCGCACCCGGGCAA
AAGCCGGGAGGACCGGGACCCGCAGCGCGACGGCCTGCCGGGCCCCTGCGCGGTG
GCACAGCCTGGGCCCGCTCAAGCGGGGCCGCAGGGCCAAGGGGTGCTTGCGCCAC
CCACGTCCCAGGGGAGTCCGTGGTGGGGCTGGGGCCGGGGTCCCCAGGTCGCCGG
GGCGGCGTGGGAACCCCAAGCCGGGGCAGCTCCACCTCCCCAGCCCGCGCCCCCG
GACGCCTCCGCCTCCGCGCGGCAGGGGCAGATGCAAGGCATCCCGGCGCCCTCCC
AGGCGCTCCAGGAGCCGGCGCCCTGGTCTGCACTCCCCTGCGGCCTGCTGCTGGAT
GAGCTCCTGGCGAGCCCGGAGTTTCTGCAGCAGGCGCAACCTCTCCTAGAAACGGA
GGCCCCGGGGGAGCTGGAGGCCTCGGAAGAGGCCGCCTCGCTGGAAGCACCCCTC
AGCGAGGAAGAATACCGGGCTCTGCTGGAGGAGCTTTAGAACCCAGCTTTcttgtacaaa
gtggtgacgtaagctaggggcccgtttaaacccgctgatcagcctcgactgtgccttctagttgccagccatctgttgtttgcccctcccccg
tgccttccttgaccctggaaggtgccactcccactgtcctttcctaataaaatgaggaaattgcatcgcattgtctgagtaggtgtcattctatt
ctggggggtggggtggggcaggacagcaagggggaggattgggaagacaatagcaggcatgctggggatgcggtgggctctatgg
cttctgaggcggaaagaaccagctggggctctagggggtatccccacgcgccctgtagcggcgcattaagcgcggcgggtgtggtggt
tacgcgcagcgtgaccgctacacttgccagcgccctagcgcccgctcctttcgctttcttcccttcctttctcgccacgttcgccggctttccc
cgtcaagctctaaatcgggggctccctttagggttccgatttagtgctttacggcacctcgaccccaaaaaacttgattagggtgatggttca
cgtacctagaagttcctattccgaagttcctattctctagaaagtataggaacttccttggccaaaaagcctgaactcaccgcgacgtctgtc
gagaagtttctgatcgaaaagttcgacagcgtctccgacctgatgcagctctcggagggcgaagaatctcgtgctttcagcttcgatgtag
gagggcgtggatatgtcctgcgggtaaatagctgcgccgatggtttctacaaagatcgttatgtttatcggcactttgcatcggccgcgctc
ccgattccggaagtgcttgacattggggaattcagcgagagcctgacctattgcatctcccgccgtgcacagggtgtcacgttgcaagac
ctgcctgaaaccgaactgcccgctgttctgcagccggtcgcggaggccatggatgcgatcgctgcggccgatcttagccagacgagcg
ggttcggcccattcggaccgcaaggaatcggtcaatacactacatggcgtgatttcatatgcgcgattgctgatccccatgtgtatcactgg
caaactgtgatggacgacaccgtcagtgcgtccgtcgcgcaggctctcgatgagctgatgctttgggccgaggactgccccgaagtccg
gcacctcgtgcacgcggatttcggctccaacaatgtcctgacggacaatggccgcataacagcggtcattgactggagcgaggcgatgtt
cggggattcccaatacgaggtcgccaacatcttcttctggaggccgtggttggcttgtatggagcagcagacgcgctacttcgagcggag
gcatccggagcttgcaggatcgccgcggctccgggcgtatatgctccgcattggtcttgaccaactctatcagagcttggttgacggcaat
ttcgatgatgcagcttgggcgcagggtcgatgcgacgcaatcgtccgatccggagccgggactgtcgggcgtacacaaatcgcccgca
gaagcgcggccgtctggaccgatggctgtgtagaagtactcgccgatagtggaaaccgacgccccagcactcgtccgagggcaaagg
aatagcacgtactacgagatttcgattccaccgccgccttctatgaaaggttgggcttcggaatcgttttccgggacgccggctggatgatc
ctccagcgcggggatctcatgctggagttcttcgcccaccccaacttgtttattgcagcttataatggttacaaataaagcaatagcatcaca
aatttcacaaataaagcatttttttcactgcattctagttgtggtttgtccaaactcatcaatgtatcttatcatgtctgtataccgtcgacctctagc
tagagcttggcgtaatcatggtcatagctgtttcctgtgtgaaattgttatccgctcacaattccacacaacatacgagccggaagcataaag
tgtaaagcctggggtgcctaatgagtgagctaactcacattaattgcgttgcgctcactgcccgctttccagtcgggaaacctgtcgtgcca
gctgcattaatgaatcggccaacgcgcggggagaggcggtttgcgtattgggcgctcttccgcttcctcgctcactgactcgctgcgctcg
gtcgttcggctgcggcgagcggtatcagctcactcaaaggcggtaatacggttatccacagaatcaggggataacgcaggaaagaacat
gtgagcaaaaggccagcaaaaggccaggaaccgtaaaaaggccgcgttgctggcgtttttccataggctccgcccccctgacgagcat
cacaaaaatcgacgctcaagtcagaggtggcgaaacccgacaggactataaagataccaggcgtttccccctggaagctccctcgtgcg
ctctcctgttccgaccctgccgcttaccggatacctgtccgcctttctcccttcgggaagcgtggcgctttctcatagctcacgctgtaggtat
ctcagttcggtgtaggtcgttcgctccaagctgggctgtgtgcacgaaccccccgttcagcccgaccgctgcgccttatccggtaactatc
gtcttgagtccaacccggtaagacacgacttatcgccactggcagcagccactggtaacaggattagcagagcgaggtatgtaggcggt
gctacagagttcttgaagtggtggcctaactacggctacactagaagaacagtatttggtatctgcgctctgctgaagccagttaccttcgga
aaaagagttggtagctcttgatccggcaaacaaaccaccgctggtagcggtggtttttttgtttgcaagcagcagattacgcgcagaaaaa
aaggatctcaagaagatcctttgatcttttctacggggtctgacgctcagtggaacgaaaactcacgttaagggattttggtcatgagattatc
aaaaaggatcttcacctagatccttttaaattaaaaatgaagttttaaatcaatctaaagtatatatgagtaaacttggtctgacagttaccaatg
cttaatcagtgaggcacctatctcagcgatctgtctatttcgttcatccatagttgcctgactccccgtcgtgtagataactacgatacgggag
ggcttaccatctggccccagtgctgcaatgataccgcgagacccacgctcaccggctccagatttatcagcaataaaccagccagccgg
aagggccgagcgcagaagtggtcctgcaactttatccgcctccatccagtctattaattgttgccgggaagctagagtaagtagttcgcca
gttaatagtttgcgcaacgttgttgccattgctacaggcatcgtggtgtcacgctcgtcgtttggtatggcttcattcagctccggttcccaacg
atcaaggcgagttacatgatcccccatgttgtgcaaaaaagcggttagctccttcggtcctccgatcgttgtcagaagtaagttggccgcag
tgttatcactcatggttatggcagcactgcataattctatactgtcatgccatccgtaagatgcttttctgtgactggtgagtactcaaccaagt
cattctgagaatagtgtatgcggcgaccgagttgctcttgcccggcgtcaatacgggataataccgcgccacatagcagaactttaaaagt
gctcatcattggaaaacgttcttcggggcgaaaactctcaaggatcttaccgctgttgagatccagttcgatgtaacccactcgtgcaccca
actgatcttcagcatcttttactttcaccagcgtttctgggtgagcaaaaacaggaaggcaaaatgccgcaaaaaagggaataagggcgac
acggaaatgttgaatactcatactcttcctttttcaatattattgaagcatttatcagggttattgtctcatgagcggatacatatttgaatgtattta
gaaaaataaacaaataggggttccgcgcacatttccccgaaaagtgccacctgacgtc
pTO STREP-HA DUX4 DNA BINDING DOMAIN (dbd)
(SEQ ID NO: 84)
gacggatcgggagatctcccgatcccctatggtgcactctcagtacaatctgctctgatgccgcatagttaagccagtatctgctccctgctt
gtgtgttggaggtcgctgagtagtgcgcgagcaaaatttaagctacaacaaggcaaggcttgaccgacaattgcatgaagaatctgcttag
ggttaggcgttttgcgctgcttcgcgatgtacgggccagatatacgcgttgacattgattattgactagttattaatagtaatcaattacggggt
cattagttcatagcccatatatggagttccgcgttacataacttacggtaaatggcccgcctggctgaccgcccaacgacccccgcccattg
acgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggat
agcggtttgactcacggggatttccaagtctccaccccattgacgtcaatgggagtttgttttggcaccaaaatcaacgggactttccaaaat
gtcgtaacaactccgccccattgacgcaaatgggcggtaggcgtgtacggtgggaggtctatataagcagagctctccctatcagtgata
gagatctccctatcagtgatagagatcgtcgacgagctcgtttagtgaaccgtcagatcgcctggagacgccatccacgctgttttgacctc
catagaagacaccgggaccgatccagcctccggactctagcgtttaaacttaagcttggtaccgagctcggatccactagtccagtgtggt
ggaattctgcagatatccagcacagtggcggccgctcgagaccatgtacccatacgatgttcctgactatgccggtaccgagctcggatc
caccatggctagctggagccacccgcagttcgagaaaggtggaggttccggaggtggatcgggaggtggatcgtggagccacccgca
gttcgaaaaagcggccgatatcacaagtttGTACAAAAAAGCAGGCTCCATGGCCCTCCCGACACCC
TCGGACAGCACCCTCCCCGCGGAAGCCCGGGGACGAGGACGGCGACGGAGACTCG
TTTGGACCCCGAGCCAAAGCGAGGCCCTGCGAGCCTGCTTTGAGCGGAACCCGTAC
CCGGGCATCGCCACCAGAGAACGGCTGGCCCAGGCCATCGGCATTCCGGAGCCCA
GGGTCCAGATTTGGTTTCAGAATGAGAGGTCACGCCAGCTGAGGCAGCACCGGCG
GGAATCTCGGCCCTGGCCCGGGAGACGCGGCCCGCCAGAAGGCCGGCGAAAGCGG
ACCGCCGTCACCGGATCCCAGACCGCCCTGCTCCTCCGAGCCTTTGAGAAGGATCG
CTTTCCAGGCATCGCCGCCCGGGAGGAGCTGGCCAGAGAGACGGGCCTCCCGGAG
TCCAGGATTCAGATCTGGTTTCAGAATCGAAGGGCCAGGCACCCGGGACAGGGTG
GCAGGGCGCCCGCGCAGGTCTAGAACCCAGCTTTcttgtacaaagtggtgacgtaagctaggggcccgt
ttaaacccgctgatcagcctcgactgtgccttctagttgccagccatctgttgtttgcccctcccccgtgccttccttgaccctggaaggtgcc
actcccactgtcctttcctaataaaatgaggaaattgcatcgcattgtctgagtaggtgtcattctattctggggggtggggtggggcaggac
agcaagggggaggattgggaagacaatagcaggcatgctggggatgcggtgggctctatggcttctgaggcggaaagaaccagctgg
ggctctagggggtatccccacgcgccctgtagcggcgcattaagcgcggcgggtgtggtggttacgcgcagcgtgaccgctacacttg
ccagcgccctagcgcccgctcctttcgctttatccatcctttctcgccacgttcgccggattccccgtcaagctctaaatcgggggctccc
tttagggttccgatttagtgctttacggcacctcgaccccaaaaaacttgattagggtgatggttcacgtacctagaagttcctattccgaagtt
cctattctctagaaagtataggaacttccttggccaaaaagcctgaactcaccgcgacgtctgtcgagaagtttctgatcgaaaagttcgac
agcgtctccgacctgatgcagctctcggagggcgaagaatctcgtgctttcagcttcgatgtaggagggcgtggatatgtcctgcgggtaa
atagctgcgccgatggtttctacaaagatcgttatgtttatcggcactttgcatcggccgcgctcccgattccggaagtgcttgacattgggg
aattcagcgagagcctgacctattgcatctcccgccgtgcacagggtgtcacgttgcaagacctgcctgaaaccgaactgcccgctgttct
gcagccggtcgcggaggccatggatgcgatcgctgcggccgatcttagccagacgagcgggttcggcccattcggaccgcaaggaat
cggtcaatacactacatggcgtgatttcatatgcgcgattgctgatccccatgtgtatcactggcaaactgtgatggacgacaccgtcagtg
cgtccgtcgcgcaggctctcgatgagctgatgctttgggccgaggactgccccgaagtccggcacctcgtgcacgcggatttcggctcc
aacaatgtcctgacggacaatggccgcataacagcggtcattgactggagcgaggcgatgttcggggattcccaatacgaggtcgccaa
catcttcttctggaggccgtggttggcttgtatggagcagcagacgcgctacttcgagcggaggcatccggagcttgcaggatcgccgcg
gctccgggcgtatatgctccgcattggtatgaccaactctatcagagcttggttgacggcaatttcgatgatgcagcttgggcgcagggtc
gatgcgacgcaatcgtccgatccggagccgggactgtcgggcgtacacaaatcgcccgcagaagcgcggccgtctggaccgatggct
gtgtagaagtactcgccgatagtggaaaccgacgccccagcactcgtccgagggcaaaggaatagcacgtactacgagatttcgattcc
accgccgccttctatgaaaggttgggatcggaatcgttttccgggacgccggctggatgatcctccagcgcggggatctcatgctggagt
tatcgcccaccccaacttgtttattgcagatataatggttacaaataaagcaatagcatcacaaatttcacaaataaagcatttttttcactgca
ttctagttgtggtttgtccaaactcatcaatgtatcttatcatgtctgtataccgtcgacctctagctagagcttggcgtaatcatggtcatagctg
tttcctgtgtgaaattgttatccgctcacaattccacacaacatacgagccggaagcataaagtgtaaagcctggggtgcctaatgagtgag
ctaactcacattaattgcgttgcgctcactgcccgctttccagtcgggaaacctgtcgtgccagctgcattaatgaatcggccaacgcgcgg
ggagaggcggtttgcgtattgggcgctcttccgcttcctcgctcactgactcgctgcgctcggtcgttcggctgcggcgagcggtatcagc
tcactcaaaggcggtaatacggttatccacagaatcaggggataacgcaggaaagaacatgtgagcaaaaggccagcaaaaggccag
gaaccgtaaaaaggccgcgttgctggcgtttttccataggctccgcccccctgacgagcatcacaaaaatcgacgctcaagtcagaggtg
gcgaaacccgacaggactataaagataccaggcgtttccccctggaagctccctcgtgcgctctcctgttccgaccctgccgcttaccgg
atacctgtccgcctttctcccttcgggaagcgtggcgctttctcatagctcacgctgtaggtatctcagttcggtgtaggtcgttcgctccaag
ctgggctgtgtgcacgaaccccccgttcagcccgaccgctgcgccttatccggtaactatcgtcttgagtccaacccggtaagacacgact
tatcgccactggcagcagccactggtaacaggattagcagagcgaggtatgtaggcggtgctacagagttcttgaagtggtggcctaact
acggctacactagaagaacagtatttggtatctgcgctctgctgaagccagttaccttcggaaaaagagttggtagctcttgatccggcaaa
caaaccaccgctggtagcggtggtttttttgtttgcaagcagcagattacgcgcagaaaaaaaggatctcaagaagatcctttgatcttttcta
cggggtctgacgctcagtggaacgaaaactcacgttaagggattttggtcatgagattatcaaaaaggatcttcacctagatccttttaaatta
aaaatgaagttttaaatcaatctaaagtatatatgagtaaacttggtctgacagttaccaatgcttaatcagtgaggcacctatctcagcgatct
gtctatttcgttcatccatagttgcctgactccccgtcgtgtagataactacgatacgggagggcttaccatctggccccagtgctgcaatgat
accgcgagacccacgctcaccggctccagatttatcagcaataaaccagccagccggaagggccgagcgcagaagtggtcctgcaac
tttatccgcctccatccagtctattaattgttgccgggaagctagagtaagtagttcgccagttaatagtttgcgcaacgttgttgccattgctac
aggcatcgtggtgtcacgctcgtcgtttggtatggcttcattcagctccggttcccaacgatcaaggcgagttacatgatcccccatgttgtg
caaaaaagcggttagctccttcggtcctccgatcgttgtcagaagtaagttggccgcagtgttatcactcatggttatggcagcactgcataa
ttctatactgtcatgccatccgtaagatgcttttctgtgactggtgagtactcaaccaagtcattctgagaatagtgtatgcggcgaccgagtt
gctcttgcccggcgtcaatacgggataataccgcgccacatagcagaactttaaaagtgctcatcattggaaaacgttcttcggggcgaaa
actctcaaggatcttaccgctgttgagatccagttcgatgtaacccactcgtgcacccaactgatcttcagcatcttttactttcaccagcgtttc
tgggtgagcaaaaacaggaaggcaaaatgccgcaaaaaagggaataagggcgacacggaaatgttgaatactcatactatcattttca
atattattgaagcatttatcagggttattgtctcatgagcggatacatatttgaatgtatttagaaaaataaacaaataggggttccgcgcacatt
tccccgaaaagtgccacctgacgtc
pCMV-FLAG MATR3 full lcngth
(SEQ ID NO: 85)
ATGCATTAGTTATTAATAGTAATCAATTACGGGGTCATTAGTTCATAGCCCATATAT
GGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGT
ACATCTACGTATTAGTCATCGCTATTACCATGGTGATGCGGTTTTGGCAGTACATCA
ATGGGCGTGGATAGCGGTTTGACTCACGGGGATTTCCAAGTCTCCACCCCATTGAC
GTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATA
TAAGCAGAGCTGGTTTAGTGAACCGTCAGATCCGCTAGCGATTACGCCAAGCTCGA
AATTAACCCTCACTAAAGGGAACAAAAGCTGGAGCTCCACCGCGGTGGCGGCCGC
CACCATGGATTACAAGGATGACGACGATAAGAGCCCGGGCggatccTCCAAGTCATTC
CAGCAGTCATCTCTCAGTAGGGACTCACAGGGTCATGGGCGTGACCTGTCTGCGGC
AGGAATAGGCCTTCTTGCTGCTGCTACCCAGTCTTTAAGTATGCCAGCATCTCTTGG
AAGGATGAACCAGGGTACTGCACGCCTTGCTAGTTTAATGAATCTTGGAATGAGTT
CTTCATTGAATCAACAAGGAGCTCATAGTGCACTGTCTTCTGCTAGTACTTCTTCCC
ATAATTTGCAGTCTATATTTAACATTGGAAGTAGAGGTCCACTCCCTTTATCTTCTC
AACACCGTGGAGATGCAGACCAGGCCAGTAACATTTTGGCCAGCTTTGGTCTGTCT
GCTAGAGACTTAGATGAACTGAGTCGTTATCCAGAGGACAAGATTACTCCTGAGAA
TTTGCCCCAAATCCTTCTACAGCTTAAAAGGAGGAGAACTGAAGAAGGCCCTACCT
TGAGTTATGGTAGAGATGGCAGATCTGCTACACGGGAGCCACCATACAGAGTACCT
AGGGATGATTGGGAAGAAAAAAGGCACTTTAGAAGAGATAGTTTTGATGATCGTG
GTCCTAGTCTCAACCCAGTGCTTGATTATGACCATGGAAGTCGTTCTCAAGAATCT
GGTTATTATGACAGAATGGATTATGAAGATGACAGATTAAGAGATGGAGAAAGGT
GTAGGGATGATTCTTTTTTTGGTGAGACCTCGCATAACTATCATAAATTTGACAGTG
AGTATGAGAGAATGGGACGTGGTCCTGGCCCCTTACAAGAGAGATCTCTCTTTGAG
AAAAAGAGAGGCGCTCCTCCAAGTAGCAATATTGAAGACTTCCATGGACTCTTACC
GAAGGGTTATCCCCATCTGTGCTCTATATGTGATTTGCCAGTTCATTCTAATAAGGA
GTGGAGTCAACATATCAATGGAGCAAGTCACAGTCGTCGATGCCAGCTTCTTCTTG
AAATCTACCCAGAATGGAATCCTGACAATGATACAGGACACACAATGGGTGATCC
ATTCATGTTGCAGCAGTCTACAAATCCAGCACCAGGAATTCTGGGACCTCCACCTC
CCTCATTTCATCTTGGGGGACCAGCAGTTGGACCAAGAGGAAATCTGGGTGCTGGA
AATGGAAACCTGCAAGGACCTAGACACATGCAGAAAGGCAGAGTGGAAACTAGCA
GAGTTGTTCACATCATGGATTTTCAACGAGGGAAAAACTTGAGATACCAGCTATTA
CAGCTGGTAGAACCATTTGGAGTCATTTCAAATCATCTGATTCTAAATAAAATTAA
TGAGGCATTTATTGAAATGGCAACCACAGAGGATGCTCAGGCCGCAGTGGATTATT
ACACAACCACACCAGCGTTAGTATTTGGCAAGCCAGTGAGAGTTCATTTATCCCAG
AAGTATAAAAGAATAAAGAAACCTGAAGGAAAGCCAGATCAGAAGTTTGATCAAA
AGCAAGAGCTTGGACGTGTGATACATCTCAGCAATTTGCCGCATTCTGGCTATTCT
GATAGTGCTGTTCTCAAGCTTGCTGAGCCTTATGGGAAAATAAAGAATTACATATT
GATGAGGATGAAAAGTCAGGCTTTTATTGAGATGGAGACAAGAGAAGATGCAATG
GCAATGGTTGACCATTGTTTGAAAAAAGCCCTTTGGTTTCAGGGGAGATGTGTGAA
GGTTGACCTGTCTGAGAAATATAAAAAACTGGTTCTGAGGATTCCAAACAGAGGC
ATTGATTTACTGAAAAAAGATAAATCCCGAAAAAGATCTTACTCTCCAGATGGCAA
AGAATCTCCAAGTGATAAGAAATCCAAAACTGATGGTTCCCAGAAGACTGAGAGT
TCAACCGAAGGTAAAGAACAAGAAGAGAAGTCCGGTGAAGATGGTGAGAAAGAC
ACAAAGGATGACCAGACAGAGCAGGAACCTAATATGCTTCTTGAATCTGAAGATG
AGCTACTTGTAGATGAAGAAGAAGCAGCAGCACTGCTAGAAAGTGGCAGTTCAGT
GGGAGACGAGACCGATCTTGCTAATTTAGGTGATGTGGCTTCTGATGGGAAAAAG
GAACCATCAGATAAAGCTGTGAAAAAAGATGGAAGTGCTTCAGCAGCAGCAAAGA
AAAAGCTTAAAAAGGTGGACAAGATCGAGGAACTTGATCAAGAAAACGAAGCAG
CGTTGGAAAATGGAATTAAAAATGAGGAAAACACAGAACCAGGTGCTGAATCTTC
TGAGAACGCTGATGATCCCAACAAAGATACAAGTGAAAACGCAGATGGTCAAAGT
GATGAGAACAAGGACGACTATACAATCCCAGATGAGTATAGAATTGGACCATATC
AGCCCAATGTTCCTGTTGGTATAGACTATGTGATACCTAAAACAGGGTTTTACTGT
AAGCTGTGTTCACTCTTTTATACAAATGAAGAAGTTGCAAAGAATACTCATTGCAG
CAGCCTTCCTCATTATCAGAAATTAAAGAAATTTCTGAATAAATTGGCAGAAGAAC
GCAGACAGAAGAAGGAAACTTAActcgagGGGGGGCCCGGTACCTTAATTAATTAAG
GTACCAGGTAAGTGTACCCAATTCGCCCTATAGTGAGTCGTATTACAATTCACTCG
ATCGCCCTTCCCAACAGTTGCGCAGCCTGAATGGCGAATGGAGATCCAATTTTTAA
GTGTATAATGTGTTAAACTACTGATTCTAATTGTTTGTGTATTTTAGATTCACAGTC
CCAAGGCTCATTTCAGGCCCCTCAGTCCTCACAGTCTGTTCATGATCATAATCAGCC
ATACCACATTTGTAGAGGTTTTACTTGCTTTAAAAAACCTCCCACACCTCCCCCTGA
ACCTGAAACATAAAATGAATGCAATTGTTGTTGTTAACTTGTTTATTGCAGCTTATA
ATGGTTACAAATAAAGCAATAGCATCACAAATTTCACAAATAAAGCATTTTTTTCA
CTGCATTCTAGTTGTGGTTTGTCCAAACTCATCAATGTATCTTAACGCGTAAATTGT
AAGCGTTAATATTTTGTTAAAATTCGCGTTAAATTTTTGTTAAATCAGCTCATTTTTT
AACCAATAGGCCGAAATCGGCAAAATCCCTTATAAATCAAAAGAATAGACCGAGA
TAGGGTTGAGTGTTGTTCCAGTTTGGAACAAGAGTCCACTATTAAAGAACGTGGAC
TCCAACGTCAAAGGGCGAAAAACCGTCTATCAGGGCGATGGCCCACTACGTGAAC
CATCACCCTAATCAAGTTTTTTGGGGTCGAGGTGCCGTAAAGCACTAAATCGGAAC
CCTAAAGGGAGCCCCCGATTTAGAGCTTGACGGGGAAAGCCGGCGAACGTGGCGA
GAAAGGAAGGGAAGAAAGCGAAAGGAGCGGGCGCTAGGGCGCTGGCAAGTGTAG
CGGTCACGCTGCGCGTAACCACCACACCCGCCGCGCTTAATGCGCCGCTACAGGGC
GCGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCA
ATAATATTGAAAAAGGAAGAATCCTGAGGCGGAAAGAACCAGCTGTGGAATGTGT
GTCAGTTAGGGTGTGGAAAGTCCCCAGGCTCCCCAGCAGGCAGAAGTATGCAAAG
CATGCATCTCAATTAGTCAGCAACCAGGTGTGGAAAGTCCCCAGGCTCCCCAGCAG
GCAGAAGTATGCAAAGCATGCATCTCAATTAGTCAGCAACCATAGTCCCGCCCCTA
ACTCCGCCCATCCCGCCCCTAACTCCGCCCAGTTCCGCCCATTCTCCGCCCCATGGC
TGACTAATTTTTTTTATTTATGCAGAGGCCGAGGCCGCCTCGGCCTCTGAGCTATTC
CAGAAGTAGTGAGGAGGCTTTTTTGGAGGCCTAGGCTTTTGCAAAGATCGATCAAG
AGACAGGATGAGGATCGTTTCGCATGATTGAACAAGATGGATTGCACGCAGGTTCT
CCGGCCGCTTGGGTGGAGAGGCTATTCGGCTATGACTGGGCACAACAGACAATCG
GCTGCTCTGATGCCGCCGTGTTCCGGCTGTCAGCGCAGGGGCGCCCGGTTCTTTTTG
TCAAGACCGACCTGTCCGGTGCCCTGAATGAACTGCAAGACGAGGCAGCGCGGCT
ATCGTGGCTGGCCACGACGGGCGTTCCTTGCGCAGCTGTGCTCGACGTTGTCACTG
AAGCGGGAAGGGACTGGCTGCTATTGGGCGAAGTGCCGGGGCAGGATCTCCTGTC
ATCTCACCTTGCTCCTGCCGAGAAAGTATCCATCATGGCTGATGCAATGCGGCGGC
TGCATACGCTTGATCCGGCTACCTGCCCATTCGACCACCAAGCGAAACATCGCATC
GAGCGAGCACGTACTCGGATGGAAGCCGGTCTTGTCGATCAGGATGATCTGGACG
AAGAACATCAGGGGCTCGCGCCAGCCGAACTGTTCGCCAGGCTCAAGGCGAGCAT
GCCCGACGGCGAGGATCTCGTCGTGACCCATGGCGATGCCTGCTTGCCGAATATCA
TGGTGGAAAATGGCCGCTTTTCTGGATTCATCGACTGTGGCCGGCTGGGTGTGGCG
GACCGCTATCAGGACATAGCGTTGGCTACCCGTGATATTGCTGAAGAACTTGGCGG
CGAATGGGCTGACCGCTTCCTCGTGCTTTACGGTATCGCCGCTCCCGATTCGCAGC
GCATCGCCTTCTATCGCCTTCTTGACGAGTTCTTCTGAGCGGGACTCTGGGGTTCGA
AATGACCGACCAAGCGACGCCCAACCTGCCATCACGAGATTTCGATTCCACCGCCG
CCTTCTATGAAAGGTTGGGCTTCGGAATCGTTTTCCGGGACGCCGGCTGGATGATC
CTCCAGCGCGGGGATCTCATGCTGGAGTTCTTCGCCCACCCTAGGGGGAGGCTAAC
TGAAACACGGAAGGAGACAATACCGGAAGGAACCCGCGCTATGACGGCAATAAA
AAGACAGAATAAAACGCACGGTGTTGGGTCGTTTGTTCATAAACGCGGGGTTCGGT
CCCAGGGCTGGCACTCTGTCGATACCCCACCGAGACCCCATTGGGGCCAATACGCC
CGCGTTTCTTCCTTTTCCCCACCCCACCCCCCAAGTTCGGGTGAAGGCCCAGGGCTC
GCAGCCAACGTCGGGGCGGCAGGCCCTGCCATAGCCTCAGGTTACTCATATATACT
TTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAG
ACCCCGTAGAAAAGATCAAAGGATCTTCTTGAGATCCTTTTTTTCTGCGCGTAATCT
GCTGCTTGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCC
pCMV-FLAG MATR3 1-287
(SEQ ID NO: 86)
ATGCATTAGTTATTAATAGTAATCAATTACGGGGTCATTAGTTCATAGCCCATATAT
GGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGT
ACATCTACGTATTAGTCATCGCTATTACCATGGTGATGCGGTTTTGGCAGTACATCA
ATGGGCGTGGATAGCGGTTTGACTCACGGGGATTTCCAAGTCTCCACCCCATTGAC
GTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATA
TAAGCAGAGCTGGTTTAGTGAACCGTCAGATCCGCTAGCGATTACGCCAAGCTCGA
AATTAACCCTCACTAAAGGGAACAAAAGCTGGAGCTCCACCGCGGTGGCGGCCGC
CACCATGGATTACAAGGATGACGACGATAAGAGCCCGGGCggatccTCCAAGTCATTC
CAGCAGTCATCTCTCAGTAGGGACTCACAGGGTCATGGGCGTGACCTGTCTGCGGC
AGGAATAGGCCTTCTTGCTGCTGCTACCCAGTCTTTAAGTATGCCAGCATCTCTTGG
AAGGATGAACCAGGGTACTGCACGCCTTGCTAGTTTAATGAATCTTGGAATGAGTT
CTTCATTGAATCAACAAGGAGCTCATAGTGCACTGTCTTCTGCTAGTACTTCTTCCC
ATAATTTGCAGTCTATATTTAACATTGGAAGTAGAGGTCCACTCCCTTTATCTTCTC
AACACCGTGGAGATGCAGACCAGGCCAGTAACATTTTGGCCAGCTTTGGTCTGTCT
GCTAGAGACTTAGATGAACTGAGTCGTTATCCAGAGGACAAGATTACTCCTGAGAA
TTTGCCCCAAATCCTTCTACAGCTTAAAAGGAGGAGAACTGAAGAAGGCCCTACCT
TGAGTTATGGTAGAGATGGCAGATCTGCTACACGGGAGCCACCATACAGAGTACCT
AGGGATGATTGGGAAGAAAAAAGGCACTTTAGAAGAGATAGTTTTGATGATCGTG
GTCCTAGTCTCAACCCAGTGCTTGATTATGACCATGGAAGTCGTTCTCAAGAATCT
GGTTATTATGACAGAATGGATTATGAAGATGACAGATTAAGAGATGGAGAAAGGT
GTAGGGATGATTCTTTTTTTGGTGAGACCTCGCATAACTATCATAAATTTGACAGTG
AGTATGAGAGAATGGGACGTGGTCCTGGCCCCTTACAAGAGAGATCTCTCTTTGAG
AAAAAGAGAGGCGCTCCTCCAAGTAGCAATATTGAAGACTTCCATGGACTCTTACC
GAAGGGTTAACCCCATCTGTGCTCTATATGTGATTTGCCAGTTCATTCTAATAAGGA
GTGGAGTCAACATATCAATGGAGCAAGTCACAGTCGTCGATGCCAGCTTCTTCTTG
AAATCTACCCAGAATGGAATCCTGACAATGATACAGGACACACAATGGGTGATCC
ATTCATGTTGCAGCAGTCTACAAATCCAGCACCAGGAATTCTGGGACCTCCACCTC
CCTCATTTCATCTTGGGGGACCAGCAGTTGGACCAAGAGGAAATCTGGGTGCTGGA
AATGGAAACCTGCAAGGACCTAGACACATGCAGAAAGGCAGAGTGGAAACTAGCA
GAGTTGTTCACATCATGGATTTTCAACGAGGGAAAAACTTGAGATACCAGCTATTA
CAGCTGGTAGAACCATTTGGAGTCATTTCAAATCATCTGATTCTAAATAAAATTAA
TGAGGCATTTATTGAAATGGCAACCACAGAGGATGCTCAGGCCGCAGTGGATTATT
ACACAACCACACCAGCGTTAGTATTTGGCAAGCCAGTGAGAGTTCATTTATCCCAG
AAGTATAAAAGAATAAAGAAACCTGAAGGAAAGCCAGATCAGAAGTTTGATCAAA
AGCAAGAGCTTGGACGTGTGATACATCTCAGCAATTTGCCGCATTCTGGCTATTCT
GATAGTGCTGTTCTCAAGCTTGCTGAGCCTTATGGGAAAATAAAGAATTACATATT
GATGAGGATGAAAAGTCAGGCTTTTATTGAGATGGAGACAAGAGAAGATGCAATG
GCAATGGTTGACCATTGTTTGAAAAAAGCCCTTTGGTTTCAGGGGAGATGTGTGAA
GGTTGACCTGTCTGAGAAATATAAAAAACTGGTTCTGAGGATTCCAAACAGAGGC
ATTGATTTACTGAAAAAAGATAAATCCCGAAAAAGATCTTACTCTCCAGATGGCAA
AGAATCTCCAAGTGATAAGAAATCCAAAACTGATGGTTCCCAGAAGACTGAGAGT
TCAACCGAAGGTAAAGAACAAGAAGAGAAGTCCGGTGAAGATGGTGAGAAAGAC
ACAAAGGATGACCAGACAGAGCAGGAACCTAATATGCTTCTTGAATCTGAAGATG
AGCTACTTGTAGATGAAGAAGAAGCAGCAGCACTGCTAGAAAGTGGCAGTTCAGT
GGGAGACGAGACCGATCTTGCTAATTTAGGTGATGTGGCTTCTGATGGGAAAAAG
GAACCATCAGATAAAGCTGTGAAAAAAGATGGAAGTGCTTCAGCAGCAGCAAAGA
AAAAGCTTAAAAAGGTGGACAAGATCGAGGAACTTGATCAAGAAAACGAAGCAG
CGTTGGAAAATGGAATTAAAAATGAGGAAAACACAGAACCAGGTGCTGAATCTTC
TGAGAACGCTGATGATCCCAACAAAGATACAAGTGAAAACGCAGATGGTCAAAGT
GATGAGAACAAGGACGACTATACAATCCCAGATGAGTATAGAATTGGACCATATC
AGCCCAATGTTCCTGTTGGTATAGACTATGTGATACCTAAAACAGGGTTTTACTGT
AAGCTGTGTTCACTCTTTTATACAAATGAAGAAGTTGCAAAGAATACTCATTGCAG
CAGCCTTCCTCATTATCAGAAATTAAAGAAATTTCTGAATAAATTGGCAGAAGAAC
GCAGACAGAAGAAGGAAACTTAActcgagGGGGGGCCCGGTACCTTAATTAATTAAG
GTACCAGGTAAGTGTACCCAATTCGCCCTATAGTGAGTCGTATTACAATTCACTCG
ATCGCCCTTCCCAACAGTTGCGCAGCCTGAATGGCGAATGGAGATCCAATTTTTAA
GTGTATAATGTGTTAAACTACTGATTCTAATTGTTTGTGTATTTTAGATTCACAGTC
CCAAGGCTCATTTCAGGCCCCTCAGTCCTCACAGTCTGTTCATGATCATAATCAGCC
ATACCACATTTGTAGAGGTTTTACTTGCTTTAAAAAACCTCCCACACCTCCCCCTGA
ACCTGAAACATAAAATGAATGCAATTGTTGTTGTTAACTTGTTTATTGCAGCTTATA
ATGGTTACAAATAAAGCAATAGCATCACAAATTTCACAAATAAAGCATTTTTTTCA
CTGCATTCTAGTTGTGGTTTGTCCAAACTCATCAATGTATCTTAACGCGTAAATTGT
AAGCGTTAATATTTTGTTAAAATTCGCGTTAAATTTTTGTTAAATCAGCTCATTTTTT
AACCAATAGGCCGAAATCGGCAAAATCCCTTATAAATCAAAAGAATAGACCGAGA
TAGGGTTGAGTGTTGTTCCAGTTTGGAACAAGAGTCCACTATTAAAGAACGTGGAC
TCCAACGTCAAAGGGCGAAAAACCGTCTATCAGGGCGATGGCCCACTACGTGAAC
CATCACCCTAATCAAGTTTTTTGGGGTCGAGGTGCCGTAAAGCACTAAATCGGAAC
CCTAAAGGGAGCCCCCGATTTAGAGCTTGACGGGGAAAGCCGGCGAACGTGGCGA
GAAAGGAAGGGAAGAAAGCGAAAGGAGCGGGCGCTAGGGCGCTGGCAAGTGTAG
CGGTCACGCTGCGCGTAACCACCACACCCGCCGCGCTTAATGCGCCGCTACAGGGC
GCGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCA
ATAATATTGAAAAAGGAAGAATCCTGAGGCGGAAAGAACCAGCTGTGGAATGTGT
GTCAGTTAGGGTGTGGAAAGTCCCCAGGCTCCCCAGCAGGCAGAAGTATGCAAAG
CATGCATCTCAATTAGTCAGCAACCAGGTGTGGAAAGTCCCCAGGCTCCCCAGCAG
GCAGAAGTATGCAAAGCATGCATCTCAATTAGTCAGCAACCATAGTCCCGCCCCTA
ACTCCGCCCATCCCGCCCCTAACTCCGCCCAGTTCCGCCCATTCTCCGCCCCATGGC
TGACTAATTTTTTTTATTTATGCAGAGGCCGAGGCCGCCTCGGCCTCTGAGCTATTC
CAGAAGTAGTGAGGAGGCTTTTTTGGAGGCCTAGGCTTTTGCAAAGATCGATCAAG
AGACAGGATGAGGATCGTTTCGCATGATTGAACAAGATGGATTGCACGCAGGTTCT
CCGGCCGCTTGGGTGGAGAGGCTATTCGGCTATGACTGGGCACAACAGACAATCG
GCTGCTCTGATGCCGCCGTGTTCCGGCTGTCAGCGCAGGGGCGCCCGGTTCTTTTTG
TCAAGACCGACCTGTCCGGTGCCCTGAATGAACTGCAAGACGAGGCAGCGCGGCT
ATCGTGGCTGGCCACGACGGGCGTTCCTTGCGCAGCTGTGCTCGACGTTGTCACTG
AAGCGGGAAGGGACTGGCTGCTATTGGGCGAAGTGCCGGGGCAGGATCTCCTGTC
ATCTCACCTTGCTCCTGCCGAGAAAGTATCCATCATGGCTGATGCAATGCGGCGGC
TGCATACGCTTGATCCGGCTACCTGCCCATTCGACCACCAAGCGAAACATCGCATC
GAGCGAGCACGTACTCGGATGGAAGCCGGTCTTGTCGATCAGGATGATCTGGACG
AAGAACATCAGGGGCTCGCGCCAGCCGAACTGTTCGCCAGGCTCAAGGCGAGCAT
GCCCGACGGCGAGGATCTCGTCGTGACCCATGGCGATGCCTGCTTGCCGAATATCA
TGGTGGAAAATGGCCGCTTTTCTGGATTCATCGACTGTGGCCGGCTGGGTGTGGCG
GACCGCTATCAGGACATAGCGTTGGCTACCCGTGATATTGCTGAAGAACTTGGCGG
CGAATGGGCTGACCGCTTCCTCGTGCTTTACGGTATCGCCGCTCCCGATTCGCAGC
GCATCGCCTTCTATCGCCTTCTTGACGAGTTCTTCTGAGCGGGACTCTGGGGTTCGA
AATGACCGACCAAGCGACGCCCAACCTGCCATCACGAGATTTCGATTCCACCGCCG
CCTTCTATGAAAGGTTGGGCTTCGGAATCGTTTTCCGGGACGCCGGCTGGATGATC
CTCCAGCGCGGGGATCTCATGCTGGAGTTCTTCGCCCACCCTAGGGGGAGGCTAAC
TGAAACACGGAAGGAGACAATACCGGAAGGAACCCGCGCTATGACGGCAATAAA
AAGACAGAATAAAACGCACGGTGTTGGGTCGTTTGTTCATAAACGCGGGGTTCGGT
CCCAGGGCTGGCACTCTGTCGATACCCCACCGAGACCCCATTGGGGCCAATACGCC
CGCGTTTCTTCCTTTTCCCCACCCCACCCCCCAAGTTCGGGTGAAGGCCCAGGGCTC
GCAGCCAACGTCGGGGCGGCAGGCCCTGCCATAGCCTCAGGTTACTCATATATACT
TTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAG
ACCCCGTAGAAAAGATCAAAGGATCTTCTTGAGATCCTTTTTTTCTGCGCGTAATCT
GCTGCTTGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCC
pCMV-FLAG MATR3 1-322
(SEQ ID NO: 87)
ATGCATTAGTTATTAATAGTAATCAATTACGGGGTCATTAGTTCATAGCCCATATAT
GGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGT
ACATCTACGTATTAGTCATCGCTATTACCATGGTGATGCGGTTTTGGCAGTACATCA
ATGGGCGTGGATAGCGGTTTGACTCACGGGGATTTCCAAGTCTCCACCCCATTGAC
GTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATA
TAAGCAGAGCTGGTTTAGTGAACCGTCAGATCCGCTAGCGATTACGCCAAGCTCGA
AATTAACCCTCACTAAAGGGAACAAAAGCTGGAGCTCCACCGCGGTGGCGGCCGC
CACCATGGATTACAAGGATGACGACGATAAGAGCCCGGGCggatccTCCAAGTCATTC
CAGCAGTCATCTCTCAGTAGGGACTCACAGGGTCATGGGCGTGACCTGTCTGCGGC
AGGAATAGGCCTTCTTGCTGCTGCTACCCAGTCTTTAAGTATGCCAGCATCTCTTGG
AAGGATGAACCAGGGTACTGCACGCCTTGCTAGTTTAATGAATCTTGGAATGAGTT
CTTCATTGAATCAACAAGGAGCTCATAGTGCACTGTCTTCTGCTAGTACTTCTTCCC
ATAATTTGCAGTCTATATTTAACATTGGAAGTAGAGGTCCACTCCCTTTATCTTCTC
AACACCGTGGAGATGCAGACCAGGCCAGTAACATTTTGGCCAGCTTTGGTCTGTCT
GCTAGAGACTTAGATGAACTGAGTCGTTATCCAGAGGACAAGATTACTCCTGAGAA
TTTGCCCCAAATCCTTCTACAGCTTAAAAGGAGGAGAACTGAAGAAGGCCCTACCT
TGAGTTATGGTAGAGATGGCAGATCTGCTACACGGGAGCCACCATACAGAGTACCT
AGGGATGATTGGGAAGAAAAAAGGCACTTTAGAAGAGATAGTTTTGATGATCGTG
GTCCTAGTCTCAACCCAGTGCTTGATTATGACCATGGAAGTCGTTCTCAAGAATCT
GGTTATTATGACAGAATGGATTATGAAGATGACAGATTAAGAGATGGAGAAAGGT
GTAGGGATGATTCTTTTTTTGGTGAGACCTCGCATAACTATCATAAATTTGACAGTG
AGTATGAGAGAATGGGACGTGGTCCTGGCCCCTTACAAGAGAGATCTCTCTTTGAG
AAAAAGAGAGGCGCTCCTCCAAGTAGCAATATTGAAGACTTCCATGGACTCTTACC
GAAGGGTTATCCCCATCTGTGCTCTATATGTGATTTGCCAGTTCATTCTAATAAGGA
GTGGAGTCAACATATCAATGGAGCAAGTCACAGTCGTCGATGCCAGCTTCTTCTTT
AAATCTACCCAGAATGGAATCCTGACAATGATACAGGACACACAATGGGTGATCC
ATTCATGTTGCAGCAGTCTACAAATCCAGCACCAGGAATTCTGGGACCTCCACCTC
CCTCATTTCATCTTGGGGGACCAGCAGTTGGACCAAGAGGAAATCTGGGTGCTGGA
AATGGAAACCTGCAAGGACCTAGACACATGCAGAAAGGCAGAGTGGAAACTAGCA
GAGTTGTTCACATCATGGATTTTCAACGAGGGAAAAACTTGAGATACCAGCTATTA
CAGCTGGTAGAACCATTTGGAGTCATTTCAAATCATCTGATTCTAAATAAAATTAA
TGAGGCATTTATTGAAATGGCAACCACAGAGGATGCTCAGGCCGCAGTGGATTATT
ACACAACCACACCAGCGTTAGTATTTGGCAAGCCAGTGAGAGTTCATTTATCCCAG
AAGTATAAAAGAATAAAGAAACCTGAAGGAAAGCCAGATCAGAAGTTTGATCAAA
AGCAAGAGCTTGGACGTGTGATACATCTCAGCAATTTGCCGCATTCTGGCTATTCT
GATAGTGCTGTTCTCAAGCTTGCTGAGCCTTATGGGAAAATAAAGAATTACATATT
GATGAGGATGAAAAGTCAGGCTTTTATTGAGATGGAGACAAGAGAAGATGCAATG
GCAATGGTTGACCATTGTTTGAAAAAAGCCCTTTGGTTTCAGGGGAGATGTGTGAA
GGTTGACCTGTCTGAGAAATATAAAAAACTGGTTCTGAGGATTCCAAACAGAGGC
ATTGATTTACTGAAAAAAGATAAATCCCGAAAAAGATCTTACTCTCCAGATGGCAA
AGAATCTCCAAGTGATAAGAAATCCAAAACTGATGGTTCCCAGAAGACTGAGAGT
TCAACCGAAGGTAAAGAACAAGAAGAGAAGTCCGGTGAAGATGGTGAGAAAGAC
ACAAAGGATGACCAGACAGAGCAGGAACCTAATATGCTTCTTGAATCTGAAGATG
AGCTACTTGTAGATGAAGAAGAAGCAGCAGCACTGCTAGAAAGTGGCAGTTCAGT
GGGAGACGAGACCGATCTTGCTAATTTAGGTGATGTGGCTTCTGATGGGAAAAAG
GAACCATCAGATAAAGCTGTGAAAAAAGATGGAAGTGCTTCAGCAGCAGCAAAGA
AAAAGCTTAAAAAGGTGGACAAGATCGAGGAACTTGATCAAGAAAACGAAGCAG
CGTTGGAAAATGGAATTAAAAATGAGGAAAACACAGAACCAGGTGCTGAATCTTC
TGAGAACGCTGATGATCCCAACAAAGATACAAGTGAAAACGCAGATGGTCAAAGT
GATGAGAACAAGGACGACTATACAATCCCAGATGAGTATAGAATTGGACCATATC
AGCCCAATGTTCCTGTTGGTATAGACTATGTGATACCTAAAACAGGGTTTTACTGT
AAGCTGTGTTCACTCTTTTATACAAATGAAGAAGTTGCAAAGAATACTCATTGCAG
CAGCCTTCCTCATTATCAGAAATTAAAGAAATTTCTGAATAAATTGGCAGAAGAAC
GCAGACAGAAGAAGGAAACTTAActcgagGGGGGGCCCGGTACCTTAATTAATTAAG
GTACCAGGTAAGTGTACCCAATTCGCCCTATAGTGAGTCGTATTACAATTCACTCG
ATCGCCCTTCCCAACAGTTGCGCAGCCTGAATGGCGAATGGAGATCCAATTTTTAA
GTGTATAATGTGTTAAACTACTGATTCTAATTGTTTGTGTATTTTAGATTCACAGTC
CCAAGGCTCATTTCAGGCCCCTCAGTCCTCACAGTCTGTTCATGATCATAATCAGCC
ATACCACATTTGTAGAGGTTTTACTTGCTTTAAAAAACCTCCCACACCTCCCCCTGA
ACCTGAAACATAAAATGAATGCAATTGTTGTTGTTAACTTGTTTATTGCAGCTTATA
ATGGTTACAAATAAAGCAATAGCATCACAAATTTCACAAATAAAGCATTTTTTTCA
CTGCATTCTAGTTGTGGTTTGTCCAAACTCATCAATGTATCTTAACGCGTAAATTGT
AAGCGTTAATATTTTGTTAAAATTCGCGTTAAATTTTTGTTAAATCAGCTCATTTTTT
AACCAATAGGCCGAAATCGGCAAAATCCCTTATAAATCAAAAGAATAGACCGAGA
TAGGGTTGAGTGTTGTTCCAGTTTGGAACAAGAGTCCACTATTAAAGAACGTGGAC
TCCAACGTCAAAGGGCGAAAAACCGTCTATCAGGGCGATGGCCCACTACGTGAAC
CATCACCCTAATCAAGTTTTTTGGGGTCGAGGTGCCGTAAAGCACTAAATCGGAAC
CCTAAAGGGAGCCCCCGATTTAGAGCTTGACGGGGAAAGCCGGCGAACGTGGCGA
GAAAGGAAGGGAAGAAAGCGAAAGGAGCGGGCGCTAGGGCGCTGGCAAGTGTAG
CGGTCACGCTGCGCGTAACCACCACACCCGCCGCGCTTAATGCGCCGCTACAGGGC
GCGTCAGGTGGCACTTTTCGGGGAAATGTGCGCGGAACCCCTATTTGTTTATTTTTC
TAAATACATTCAAATATGTATCCGCTCATGAGACAATAACCCTGATAAATGCTTCA
ATAATATTGAAAAAGGAAGAATCCTGAGGCGGAAAGAACCAGCTGTGGAATGTGT
GTCAGTTAGGGTGTGGAAAGTCCCCAGGCTCCCCAGCAGGCAGAAGTATGCAAAG
CATGCATCTCAATTAGTCAGCAACCAGGTGTGGAAAGTCCCCAGGCTCCCCAGCAG
GCAGAAGTATGCAAAGCATGCATCTCAATTAGTCAGCAACCATAGTCCCGCCCCTA
ACTCCGCCCATCCCGCCCCTAACTCCGCCCAGTTCCGCCCATTCTCCGCCCCATGGC
TGACTAATTTTTTTTATTTATGCAGAGGCCGAGGCCGCCTCGGCCTCTGAGCTATTC
CAGAAGTAGTGAGGAGGCTTTTTTGGAGGCCTAGGCTTTTGCAAAGATCGATCAAG
AGACAGGATGAGGATCGTTTCGCATGATTGAACAAGATGGATTGCACGCAGGTTCT
CCGGCCGCTTGGGTGGAGAGGCTATTCGGCTATGACTGGGCACAACAGACAATCG
GCTGCTCTGATGCCGCCGTGTTCCGGCTGTCAGCGCAGGGGCGCCCGGTTCTTTTTG
TCAAGACCGACCTGTCCGGTGCCCTGAATGAACTGCAAGACGAGGCAGCGCGGCT
ATCGTGGCTGGCCACGACGGGCGTTCCTTGCGCAGCTGTGCTCGACGTTGTCACTG
AAGCGGGAAGGGACTGGCTGCTATTGGGCGAAGTGCCGGGGCAGGATCTCCTGTC
ATCTCACCTTGCTCCTGCCGAGAAAGTATCCATCATGGCTGATGCAATGCGGCGGC
TGCATACGCTTGATCCGGCTACCTGCCCATTCGACCACCAAGCGAAACATCGCATC
GAGCGAGCACGTACTCGGATGGAAGCCGGTCTTGTCGATCAGGATGATCTGGACG
AAGAACATCAGGGGCTCGCGCCAGCCGAACTGTTCGCCAGGCTCAAGGCGAGCAT
GCCCGACGGCGAGGATCTCGTCGTGACCCATGGCGATGCCTGCTTGCCGAATATCA
TGGTGGAAAATGGCCGCTTTTCTGGATTCATCGACTGTGGCCGGCTGGGTGTGGCG
GACCGCTATCAGGACATAGCGTTGGCTACCCGTGATATTGCTGAAGAACTTGGCGG
CGAATGGGCTGACCGCTTCCTCGTGCTTTACGGTATCGCCGCTCCCGATTCGCAGC
GCATCGCCTTCTATCGCCTTCTTGACGAGTTCTTCTGAGCGGGACTCTGGGGTTCGA
AATGACCGACCAAGCGACGCCCAACCTGCCATCACGAGATTTCGATTCCACCGCCG
CCTTCTATGAAAGGTTGGGCTTCGGAATCGTTTTCCGGGACGCCGGCTGGATGATC
CTCCAGCGCGGGGATCTCATGCTGGAGTTCTTCGCCCACCCTAGGGGGAGGCTAAC
TGAAACACGGAAGGAGACAATACCGGAAGGAACCCGCGCTATGACGGCAATAAA
AAGACAGAATAAAACGCACGGTGTTGGGTCGTTTGTTCATAAACGCGGGGTTCGGT
CCCAGGGCTGGCACTCTGTCGATACCCCACCGAGACCCCATTGGGGCCAATACGCC
CGCGTTTCTTCCTTTTCCCCACCCCACCCCCCAAGTTCGGGTGAAGGCCCAGGGCTC
GCAGCCAACGTCGGGGCGGCAGGCCCTGCCATAGCCTCAGGTTACTCATATATACT
TTAGATTGATTTAAAACTTCATTTTTAATTTAAAAGGATCTAGGTGAAGATCCTTTT
TGATAATCTCATGACCAAAATCCCTTAACGTGAGTTTTCGTTCCACTGAGCGTCAG
ACCCCGTAGAAAAGATCAAAGGATCTTCTTGAGATCCTTTTTTTCTGCGCGTAATCT
GCTGCTTGCAAACAAAAAAACCACCGCTACCAGCGGTGGTTTGTTTGCCGGATCAA
GAGCTACCAACTCTTTTTCCGAAGGTAACTGGCTTCAGCAGAGCGCAGATACCAAA
TACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCC
pCMV-FLAG MATR3 1-797
(SEQ ID NO: 88)
ATGCATTAGTTATTAATAGTAATCAATTACGGGGTCATTAGTTCATAGCCCATATAT
GGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGT
ACATCTACGTATTAGTCATCGCTATTACCATGGTGATGCGGTTTTGGCAGTACATCA
ATGGGCGTGGATAGCGGTTTGACTCACGGGGATTTCCAAGTCTCCACCCCATTGAC
GTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATA
TAAGCAGAGCTGGTTTAGTGAACCGTCAGATCCGCTAGCGATTACGCCAAGCTCGA
AATTAACCCTCACTAAAGGGAACAAAAGCTGGAGCTCCACCGCGGTGGCGGCCGC
CACCATGGATTACAAGGATGACGACGATAAGAGCCCGGGCggatccTCCAAGTCATTC
CAGCAGTCATCTCTCAGTAGGGACTCACAGGGTCATGGGCGTGACCTGTCTGCGGC
AGGAATAGGCCTTCTTGCTGCTGCTACCCAGTCTTTAAGTATGCCAGCATCTCTTGG
AAGGATGAACCAGGGTACTGCACGCCTTGCTAGTTTAATGAATCTTGGAATGAGTT
CTTCATTGAATCAACAAGGAGCTCATAGTGCACTGTCTTCTGCTAGTACTTCTTCCC
ATAATTTGCAGTCTATATTTAACATTGGAAGTAGAGGTCCACTCCCTTTATCTTCTC
AACACCGTGGAGATGCAGACCAGGCCAGTAACATTTTGGCCAGCTTTGGTCTGTCT
GCTAGAGACTTAGATGAACTGAGTCGTTATCCAGAGGACAAGATTACTCCTGAGAA
TTTGCCCCAAATCCTTCTACAGCTTAAAAGGAGGAGAACTGAAGAAGGCCCTACCT
TGAGTTATGGTAGAGATGGCAGATCTGCTACACGGGAGCCACCATACAGAGTACCT
AGGGATGATTGGGAAGAAAAAAGGCACTTTAGAAGAGATAGTTTTGATGATCGTG
GTCCTAGTCTCAACCCAGTGCTTGATTATGACCATGGAAGTCGTTCTCAAGAATCT
GGTTATTATGACAGAATGGATTATGAAGATGACAGATTAAGAGATGGAGAAAGGT
GTAGGGATGATTCTTTTTTTGGTGAGACCTCGCATAACTATCATAAATTTGACAGTG
AGTATGAGAGAATGGGACGTGGTCCTGGCCCCTTACAAGAGAGATCTCTCTTTGAG
AAAAAGAGAGGCGCTCCTCCAAGTAGCAATATTGAAGACTTCCATGGACTCTTACC
GAAGGGTTATCCCCATCTGTGCTCTATATGTGATTTGCCAGTTCATTCTAATAAGGA
GTGGAGTCAACATATCAATGGAGCAAGTCACAGTCGTCGATGCCAGCTTCTTCTTG
AAATCTACCCAGAATGGAATCCTGACAATGATACAGGACACACAATGGGTGATCC
ATTCATGTTGCAGCAGTCTACAAATCCAGCACCAGGAATTCTGGGACCTCCACCTC
CCTCATTTCATCTTGGGGGACCAGCAGTTGGACCAAGAGGAAATCTGGGTGCTGGA
AATGGAAACCTGCAAGGACCTAGACACATGCAGAAAGGCAGAGTGGAAACTAGCA
GAGTTGTTCACATCATGGATTTTCAACGAGGGAAAAACTTGAGATACCAGCTATTA
CAGCTGGTAGAACCATTTGGAGTCATTTCAAATCATCTGATTCTAAATAAAATTAA
TGAGGCATTTATTGAAATGGCAACCACAGAGGATGCTCAGGCCGCAGTGGATTATT
ACACAACCACACCAGCGTTAGTATTTGGCAAGCCAGTGAGAGTTCATTTATCCCAG
AAGTATAAAAGAATAAAGAAACCTGAAGGAAAGCCAGATCAGAAGTTTGATCAAA
AGCAAGAGCTTGGACGTGTGATACATCTCAGCAATTTGCCGCATTCTGGCTATTCT
GATAGTGCTGTTCTCAAGCTTGCTGAGCCTTATGGGAAAATAAAGAATTACATATT
GATGAGGATGAAAAGTCAGGCTTTTATTGAGATGGAGACAAGAGAAGATGCAATG
GCAATGGTTGACCATTGTTTGAAAAAAGCCCTTTGGTTTCAGGGGAGATGTGTGAA
GGTTGACCTGTCTGAGAAATATAAAAAACTGGTTCTGAGGATTCCAAACAGAGGC
ATTGATTTACTGAAAAAAGATAAATCCCGAAAAAGATCTTACTCTCCAGATGGCAA
AGAATCTCCAAGTGATAAGAAATCCAAAACTGATGGTTCCCAGAAGACTGAGAGT
TCAACCGAAGGTAAAGAACAAGAAGAGAAGTCCGGTGAAGATGGTGAGAAAGAC
ACAAAGGATGACCAGACAGAGCAGGAACCTAATATGCTTCTTGAATCTGAAGATG
AGCTACTTGTAGATGAAGAAGAAGCAGCAGCACTGCTAGAAAGTGGCAGTTCAGT
GGGAGACGAGACCGATCTTGCTAATTTAGGTGATGTGGCTTCTGATGGGAAAAAG
GAACCATCAGATAAAGCTGTGAAAAAAGATGGAAGTGCTTCAGCAGCAGCAAAGA
AAAAGCTTAAAAAGGTGGACAAGATCGAGGAACTTGATCAAGAAAACGAAGCAG
CGTTGGAAAATGGAATTAAAAATGAGGAAAACACAGAACCAGGTGCTGAATCTTC
TGAGAACGCTGATGATCCCAACAAAGATACAAGTGAAAACGCAGATGGTCAAAGT
GATGAGAACAAGGACGACTATACAATCCCAGATGAGTATAGAATTGGACCATATC
AGCCCAATGTTCCTGTTGGTATAGACTATGTGATACCTAAATAAGGGTTTTACTGTA
AGCTGTGTTCACTCTTTTATACAAATGAAGAAGTTGCAAAGAATACTCATTGCAGC
AGCCTTCCTCATTATCAGAAATTAAAGAAATTTCTGAATAAATTGGCAGAAGAACG
CAGACAGAAGAAGGAAACTTAActcgagGGGGGGCCCGGTACCTTAATTAATTAAGG
TACCAGGTAAGTGTACCCAATTCGCCCTATAGTGAGTCGTATTACAATTCACTCGA
TCGCCCTTCCCAACAGTTGCGCAGCCTGAATGGCGAATGGAGATCCAATTTTTAAG
TGTATAATGTGTTAAACTACTGATTCTAATTGTTTGTGTATTTTAGATTCACAGTCC
CAAGGCTCATTTCAGGCCCCTCAGTCCTCACAGTCTGTTCATGATCATAATCAGCCA
TACCACATTTGTAGAGGTTTTACTTGCTTTAAAAAACCTCCCACACCTCCCCCTGAA
CCTGAAACATAAAATGAATGCAATTGTTGTTGTTAACTTGTTTATTGCAGCTTATAA
TGGTTACAAATAAAGCAATAGCATCACAAATTTCACAAATAAAGCATTTTTTTCAC
TGCATTCTAGTTGTGGTTTGTCCAAACTCATCAATGTATCTTAACGCGTAAATTGTA
AGCGTTAATATTTTGTTAAAATTCGCGTTAAATTTTTGTTAAATCAGCTCATTTTTTA
ACCAATAGGCCGAAATCGGCAAAATCCCTTATAAATCAAAAGAATAGACCGAGAT
AGGGTTGAGTGTTGTTCCAGTTTGGAACAAGAGTCCACTATTAAAGAACGTGGACT
CCAACGTCAAAGGGCGAAAAACCGTCTATCAGGGCGATGGCCCACTACGTGAACC
ATCACCCTAATCAAGTTTTTTGGGGTCGAGGTGCCGTAAAGCACTAAATCGGAACC
CTAAAGGGAGCCCCCGATTTAGAGCTTGACGGGGAAAGCCGGCGAACGTGGCGAG
AAAGGAAGGGAAGAAAGCGAAAGGAGCGGGCGCTAGGGCGCTGGCAAGTGTAGC
GGTCACGCTGCGCGTAACCACCACACCCGCCGCGCTTAATGCGCCGCTACAGGGCG
CGTCAGGTGGCACTTTTCGGGGAAATGTGCGCGGAACCCCTATTTGTTTATTTTTCT
AAATACATTCAAATATGTATCCGCTCATGAGACAATAACCCTGATAAATGCTTCAA
TAATATTGAAAAAGGAAGAATCCTGAGGCGGAAAGAACCAGCTGTGGAATGTGTG
TCAGTTAGGGTGTGGAAAGTCCCCAGGCTCCCCAGCAGGCAGAAGTATGCAAAGC
ATGCATCTCAATTAGTCAGCAACCAGGTGTGGAAAGTCCCCAGGCTCCCCAGCAGG
CAGAAGTATGCAAAGCATGCATCTCAATTAGTCAGCAACCATAGTCCCGCCCCTAA
CTCCGCCCATCCCGCCCCTAACTCCGCCCAGTTCCGCCCATTCTCCGCCCCATGGCT
GACTAATTTTTTTTATTTATGCAGAGGCCGAGGCCGCCTCGGCCTCTGAGCTATTCC
AGAAGTAGTGAGGAGGCTTTTTTGGAGGCCTAGGCTTTTGCAAAGATCGATCAAGA
GACAGGATGAGGATCGTTTCGCATGATTGAACAAGATGGATTGCACGCAGGTTCTC
CGGCCGCTTGGGTGGAGAGGCTATTCGGCTATGACTGGGCACAACAGACAATCGG
CTGCTCTGATGCCGCCGTGTTCCGGCTGTCAGCGCAGGGGCGCCCGGTTCTTTTTGT
CAAGACCGACCTGTCCGGTGCCCTGAATGAACTGCAAGACGAGGCAGCGCGGCTA
TCGTGGCTGGCCACGACGGGCGTTCCTTGCGCAGCTGTGCTCGACGTTGTCACTGA
AGCGGGAAGGGACTGGCTGCTATTGGGCGAAGTGCCGGGGCAGGATCTCCTGTCA
TCTCACCTTGCTCCTGCCGAGAAAGTATCCATCATGGCTGATGCAATGCGGCGGCT
GCATACGCTTGATCCGGCTACCTGCCCATTCGACCACCAAGCGAAACATCGCATCG
AGCGAGCACGTACTCGGATGGAAGCCGGTCTTGTCGATCAGGATGATCTGGACGA
AGAACATCAGGGGCTCGCGCCAGCCGAACTGTTCGCCAGGCTCAAGGCGAGCATG
CCCGACGGCGAGGATCTCGTCGTGACCCATGGCGATGCCTGCTTGCCGAATATCAT
GGTGGAAAATGGCCGCTTTTCTGGATTCATCGACTGTGGCCGGCTGGGTGTGGCGG
ACCGCTATCAGGACATAGCGTTGGCTACCCGTGATATTGCTGAAGAACTTGGCGGC
GAATGGGCTGACCGCTTCCTCGTGCTTTACGGTATCGCCGCTCCCGATTCGCAGCG
CATCGCCTTCTATCGCCTTCTTGACGAGTTCTTCTGAGCGGGACTCTGGGGTTCGAA
ATGACCGACCAAGCGACGCCCAACCTGCCATCACGAGATTTCGATTCCACCGCCGC
CTTCTATGAAAGGTTGGGCTTCGGAATCGTTTTCCGGGACGCCGGCTGGATGATCC
TCCAGCGCGGGGATCTCATGCTGGAGTTCTTCGCCCACCCTAGGGGGAGGCTAACT
GAAACACGGAAGGAGACAATACCGGAAGGAACCCGCGCTATGACGGCAATAAAA
AGACAGAATAAAACGCACGGTGTTGGGTCGTTTGTTCATAAACGCGGGGTTCGGTC
CCAGGGCTGGCACTCTGTCGATACCCCACCGAGACCCCATTGGGGCCAATACGCCC
GCGTTTCTTCCTTTTCCCCACCCCACCCCCCAAGTTCGGGTGAAGGCCCAGGGCTCG
CAGCCAACGTCGGGGCGGCAGGCCCTGCCATAGCCTCAGGTTACTCATATATACTT
TAGATTGATTTAAAACTTCATTTTTAATTTAAAAGGATCTAGGTGAAGATCCTTTTT
GATAATCTCATGACCAAAATCCCTTAACGTGAGTTTTCGTTCCACTGAGCGTCAGA
CCCCGTAGAAAAGATCAAAGGATCTTCTTGAGATCCTTTTTTTCTGCGCGTAATCTG
CTGCTTGCAAACAAAAAAACCACCGCTACCAGCGGTGGTTTGTTTGCCGGATCAAG
AGCTACCAACTCTTTTTCCGAAGGTAACTGGCTTCAGCAGAGCGCAGATACCAAAT
ACTGTCCTTCTAGTGTAGCCGTAGTTAGGCCACCACTTCAAGAACTCTGTAGCACC
GCCTACATACCTCGCTCTGCTAATCCTGTTACCAGTGGCTGCTGCCAGTGGCGATA
AGTCGTGTCTTACCGGGTTGGACTCAAGACGATAGTTACCGGATAAGGCGCAGCGG
TCGGGCTGAACGGGGGGTTCGTGCACACAGCCCAGCTTGGAGCGAACGACCTACA
CCGAACTGAGATACCTACAGCGTGAGCTATGAGAAAGCGCCACGCTTCCCGAAGG
GAGAAAGGCGGACAGGTATCCGGTAAGCGGCAGGGTCGGAACAGGAGAGCGCAC
GAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGG
AAAAACGCCAGCAACGCGGCCTTTTTACGGTTCCTGGCCTTTTGCTGGCCTTTTGCT
CACATGTTCTTTCCTGCGTTATCCCCTGATTCTGTGGATAACCGTATTACCGCC
pCMV-FLAG MATR3 288-847
(SEQ ID NO: 89)
ATGCATTAGTTATTAATAGTAATCAATTACGGGGTCATTAGTTCATAGCCCATATAT
GGAGTTCCGCGTTACATAACTTACGGTAAATGGCCCGCCTGGCTGACCGCCCAACG
ACCCCCGCCCATTGACGTCAATAATGACGTATGTTCCCATAGTAACGCCAATAGGG
ACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAAT
GGCCCGCCTGGCATTATGCCCAGTACATGACCTTATGGGACTTTCCTACTTGGCAGT
ACATCTACGTATTAGTCATCGCTATTACCATGGTGATGCGGTTTTGGCAGTACATCA
ATGGGCGTGGATAGCGGTTTGACTCACGGGGATTTCCAAGTCTCCACCCCATTGAC
GTCAATGGGAGTTTGTTTTGGCACCAAAATCAACGGGACTTTCCAAAATGTCGTAA
CAACTCCGCCCCATTGACGCAAATGGGCGGTAGGCGTGTACGGTGGGAGGTCTATA
TAAGCAGAGCTGGTTTAGTGAACCGTCAGATCCGCTAGCGATTACGCCAAGCTCGA
AATTAACCCTCACTAAAGGGAACAAAAGCTGGAGCTCCACCGCGGTGGCGGCCGC
CACCATGGATTACAAGGATGACGACGATAAGAGCCCGGGCggatccTATCCCCATCTG
TGCTCTATATGTGATTTGCCAGTTCATTCTAATAAGGAGTGGAGTCAACATATCAAT
GGAGCAAGTCACAGTCGTCGATGCCAGCTTCTTCTTGAAATCTACCCAGAATGGAA
TCCTGACAATGATACAGGACACACAATGGGTGATCCATTCATGTTGCAGCAGTCTA
CAAATCCAGCACCAGGAATTCTGGGACCTCCACCTCCCTCATTTCATCTTGGGGGA
CCAGCAGTTGGACCAAGAGGAAATCTGGGTGCTGGAAATGGAAACCTGCAAGGAC
CTAGACACATGCAGAAAGGCAGAGTGGAAACTAGCAGAGTTGTTCACATCATGGA
TTTTCAACGAGGGAAAAACTTGAGATACCAGCTATTACAGCTGGTAGAACCATTTG
GAGTCATTTCAAATCATCTGATTCTAAATAAAATTAATGAGGCATTTATTGAAATG
GCAACCACAGAGGATGCTCAGGCCGCAGTGGATTATTACACAACCACACCAGCGT
TAGTATTTGGCAAGCCAGTGAGAGTTCATTTATCCCAGAAGTATAAAAGAATAAAG
AAACCTGAAGGAAAGCCAGATCAGAAGTTTGATCAAAAGCAAGAGCTTGGACGTG
TGATACATCTCAGCAATTTGCCGCATTCTGGCTATTCTGATAGTGCTGTTCTCAAGC
TTGCTGAGCCTTATGGGAAAATAAAGAATTACATATTGATGAGGATGAAAAGTCA
GGCTTTTATTGAGATGGAGACAAGAGAAGATGCAATGGCAATGGTTGACCATTGTT
TGAAAAAAGCCCTTTGGTTTCAGGGGAGATGTGTGAAGGTTGACCTGTCTGAGAAA
TATAAAAAACTGGTTCTGAGGATTCCAAACAGAGGCATTGATTTACTGAAAAAAG
ATAAATCCCGAAAAAGATCTTACTCTCCAGATGGCAAAGAATCTCCAAGTGATAAG
AAATCCAAAACTGATGGTTCCCAGAAGACTGAGAGTTCAACCGAAGGTAAAGAAC
AAGAAGAGAAGTCCGGTGAAGATGGTGAGAAAGACACAAAGGATGACCAGACAG
AGCAGGAACCTAATATGCTTCTTGAATCTGAAGATGAGCTACTTGTAGATGAAGAA
GAAGCAGCAGCACTGCTAGAAAGTGGCAGTTCAGTGGGAGACGAGACCGATCTTG
CTAATTTAGGTGATGTGGCTTCTGATGGGAAAAAGGAACCATCAGATAAAGCTGTG
AAAAAAGATGGAAGTGCTTCAGCAGCAGCAAAGAAAAAGCTTAAAAAGGTGGAC
AAGATCGAGGAACTTGATCAAGAAAACGAAGCAGCGTTGGAAAATGGAATTAAAA
ATGAGGAAAACACAGAACCAGGTGCTGAATCTTCTGAGAACGCTGATGATCCCAA
CAAAGATACAAGTGAAAACGCAGATGGTCAAAGTGATGAGAACAAGGACGACTAT
ACAATCCCAGATGAGTATAGAATTGGACCATATCAGCCCAATGTTCCTGTTGGTAT
AGACTATGTGATACCTAAAACAGGGTTTTACTGTAAGCTGTGTTCACTCTTTTATAC
AAATGAAGAAGTTGCAAAGAATACTCATTGCAGCAGCCTTCCTCATTATCAGAAAT
TAAAGAAATTTCTGAATAAATTGGCAGAAGAACGCAGACAGAAGAAGGAAACTTA
ActcgagGGGGGGCCCGGTACCTTAATTAATTAAGGTACCAGGTAAGTGTACCCAATT
CGCCCTATAGTGAGTCGTATTACAATTCACTCGATCGCCCTTCCCAACAGTTGCGCA
GCCTGAATGGCGAATGGAGATCCAATTTTTAAGTGTATAATGTGTTAAACTACTGA
TTCTAATTGTTTGTGTATTTTAGATTCACAGTCCCAAGGCTCATTTCAGGCCCCTCA
GTCCTCACAGTCTGTTCATGATCATAATCAGCCATACCACATTTGTAGAGGTTTTAC
TTGCTTTAAAAAACCTCCCACACCTCCCCCTGAACCTGAAACATAAAATGAATGCA
ATTGTTGTTGTTAACTTGTTTATTGCAGCTTATAATGGTTACAAATAAAGCAATAGC
ATCACAAATTTCACAAATAAAGCATTTTTTTCACTGCATTCTAGTTGTGGTTTGTCC
AAACTCATCAATGTATCTTAACGCGTAAATTGTAAGCGTTAATATTTTGTTAAAATT
CGCGTTAAATTTTTGTTAAATCAGCTCATTTTTTAACCAATAGGCCGAAATCGGCAA
AATCCCTTATAAATCAAAAGAATAGACCGAGATAGGGTTGAGTGTTGTTCCAGTTT
GGAACAAGAGTCCACTATTAAAGAACGTGGACTCCAACGTCAAAGGGCGAAAAAC
CGTCTATCAGGGCGATGGCCCACTACGTGAACCATCACCCTAATCAAGTTTTTTGG
GGTCGAGGTGCCGTAAAGCACTAAATCGGAACCCTAAAGGGAGCCCCCGATTTAG
AGCTTGACGGGGAAAGCCGGCGAACGTGGCGAGAAAGGAAGGGAAGAAAGCGAA
AGGAGCGGGCGCTAGGGCGCTGGCAAGTGTAGCGGTCACGCTGCGCGTAACCACC
ACACCCGCCGCGCTTAATGCGCCGCTACAGGGCGCGTCAGGTGGCACTTTTCGGGG
AAATGTGCGCGGAACCCCTATTTGTTTATTTTTCTAAATACATTCAAATATGTATCC
GCTCATGAGACAATAACCCTGATAAATGCTTCAATAATATTGAAAAAGGAAGAAT
CCTGAGGCGGAAAGAACCAGCTGTGGAATGTGTGTCAGTTAGGGTGTGGAAAGTC
CCCAGGCTCCCCAGCAGGCAGAAGTATGCAAAGCATGCATCTCAATTAGTCAGCA
ACCAGGTGTGGAAAGTCCCCAGGCTCCCCAGCAGGCAGAAGTATGCAAAGCATGC
ATCTCAATTAGTCAGCAACCATAGTCCCGCCCCTAACTCCGCCCATCCCGCCCCTA
ACTCCGCCCAGTTCCGCCCATTCTCCGCCCCATGGCTGACTAATTTTTTTTATTTATG
CAGAGGCCGAGGCCGCCTCGGCCTCTGAGCTATTCCAGAAGTAGTGAGGAGGCTTT
TTTGGAGGCCTAGGCTTTTGCAAAGATCGATCAAGAGACAGGATGAGGATCGTTTC
GCATGATTGAACAAGATGGATTGCACGCAGGTTCTCCGGCCGCTTGGGTGGAGAG
GCTATTCGGCTATGACTGGGCACAACAGACAATCGGCTGCTCTGATGCCGCCGTGT
TCCGGCTGTCAGCGCAGGGGCGCCCGGTTCTTTTTGTCAAGACCGACCTGTCCGGT
GCCCTGAATGAACTGCAAGACGAGGCAGCGCGGCTATCGTGGCTGGCCACGACGG
GCGTTCCTTGCGCAGCTGTGCTCGACGTTGTCACTGAAGCGGGAAGGGACTGGCTG
CTATTGGGCGAAGTGCCGGGGCAGGATCTCCTGTCATCTCACCTTGCTCCTGCCGA
GAAAGTATCCATCATGGCTGATGCAATGCGGCGGCTGCATACGCTTGATCCGGCTA
CCTGCCCATTCGACCACCAAGCGAAACATCGCATCGAGCGAGCACGTACTCGGATG
GAAGCCGGTCTTGTCGATCAGGATGATCTGGACGAAGAACATCAGGGGCTCGCGC
CAGCCGAACTGTTCGCCAGGCTCAAGGCGAGCATGCCCGACGGCGAGGATCTCGT
CGTGACCCATGGCGATGCCTGCTTGCCGAATATCATGGTGGAAAATGGCCGCTTTT
CTGGATTCATCGACTGTGGCCGGCTGGGTGTGGCGGACCGCTATCAGGACATAGCG
TTGGCTACCCGTGATATTGCTGAAGAACTTGGCGGCGAATGGGCTGACCGCTTCCT
CGTGCTTTACGGTATCGCCGCTCCCGATTCGCAGCGCATCGCCTTCTATCGCCTTCT
TGACGAGTTCTTCTGAGCGGGACTCTGGGGTTCGAAATGACCGACCAAGCGACGCC
CAACCTGCCATCACGAGATTTCGATTCCACCGCCGCCTTCTATGAAAGGTTGGGCT
TCGGAATCGTTTTCCGGGACGCCGGCTGGATGATCCTCCAGCGCGGGGATCTCATG
CTGGAGTTCTTCGCCCACCCTAGGGGGAGGCTAACTGAAACACGGAAGGAGACAA
TACCGGAAGGAACCCGCGCTATGACGGCAATAAAAAGACAGAATAAAACGCACGG
TGTTGGGTCGTTTGTTCATAAACGCGGGGTTCGGTCCCAGGGCTGGCACTCTGTCG
ATACCCCACCGAGACCCCATTGGGGCCAATACGCCCGCGTTTCTTCCTTTTCCCCAC
CCCACCCCCCAAGTTCGGGTGAAGGCCCAGGGCTCGCAGCCAACGTCGGGGCGGC
AGGCCCTGCCATAGCCTCAGGTTACTCATATATACTTTAGATTGATTTAAAACTTCA
TTTTTAATTTAAAAGGATCTAGGTGAAGATCCTTTTTGATAATCTCATGACCAAAAT
CCCTTAACGTGAGTTTTCGTTCCACTGAGCGTCAGACCCCGTAGAAAAGATCAAAG
GATCTTCTTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTTTTTCCGA
AGGTAACTGGCTTCAGCAGAGCGCAGATACCAAATACTGTCCTTCTAGTGTAGCCG
TAGTTAGGCCACCACTTCAAGAACTCTGTAGCACCGCCTACATACCTCGCTCTGCT
AATCCTGTTACCAGTGGCTGCTGCCAGTGGCGATAAGTCGTGTCTTACCGGGTTGG
ACTCAAGACGATAGTTACCGGATAAGGCGCAGCGGTCGGGCTGAACGGGGGGTTC
GTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATC
CGGTAAGCGGCAGGGTCGGAACAGGAGAGCGCACGAGGGAGCTTCCAGGGGGAA
ACGCCTGGTATCTTTATAGTCCTGTCGGGTTTCGCCACCTCTGACTTGAGCGTCGAT
TTTTGTGATGCTCGTCAGGGGGGCGGAGCCTATGGAAAAACGCCAGCAACGCGGC
CTTTTTACGGTTCCTGGCCTTTTGCTGGCCTTTTGCTCACATGTTCTTTCCTGCGTTA
TCCCCTGATTCTGTGGATAACCGTATTACCGCC
pGEX2tk-GST-MATR3 1-287
(SEQ ID NO: 90)
ACGTTATCGACTGCACGGTGCACCAATGCTTCTGGCGTCAGGCAGCCATCGGAAGC
TGTGGTATGGCTGTGCAGGTCGTAAATCACTGCATAATTCGTGTCGCTCAAGGCGC
ACTCCCGTTCTGGATAATGTTTTTTGCGCCGACATCATAACGGTTCTGGCAAATATT
CTGAAATGAGCTGTTGACAATTAATCATCGGCTCGTATAATGTGTGGAATTGTGAG
CGGATAACAATTTCACACAGGAAACAGTATTCATGTCCCCTATACTAGGTTATTGG
AAAATTAAGGGCCTTGTGCAACCCACTCGACTTCTTTTGGAATATCTTGAAGAAAA
ATATGAAGAGCATTTGTATGAGCGCGATGAAGGTGATAAATGGCGAAACAAAAAG
TTTGAATTGGGTTTGGAGTTTCCCAATCTTCCTTATTATATTGATGGTGATGTTAAA
TTAACACAGTCTATGGCCATCATACGTTATATAGCTGACAAGCACAACATGTTGGG
TGGTTGTCCAAAAGAGCGTGCAGAGATTTCAATGCTTGAAGGAGCGGTTTTGGATA
TTAGATACGGTGTTTCGAGAATTGCATATAGTAAAGACTTTGAAACTCTCAAAGTT
GATTTTCTTAGCAAGCTACCTGAAATGCTGAAAATGTTCGAAGATCGTTTATGTCAT
AAAACATATTTAAATGGTGATCATGTAACCCATCCTGACTTCATGTTGTATGACGCT
CTTGATGTTGTTTTATACATGGACCCAATGTGCCTGGATGCGTTCCCAAAATTAGTT
TGTTTTAAAAAACGTATTGAAGCTATCCCACAAATTGATAAGTACTTGAAATCCAG
CAAGTATATAGCATGGCCTTTGCAGGGCTGGCAAGCCACGTTTGGTGGTGGCGACC
ATCCTCCAAAATCGGATCTGGTTCCGCGTGGATCTCGTCGTGCATCTGTTGGATCCT
CCAAGTCATTCCAGCAGTCATCTCTCAGTAGGGACTCACAGGGTCATGGGCGTGAC
CTGTCTGCGGCAGGAATAGGCCTTCTTGCTGCTGCTACCCAGTCTTTAAGTATGCCA
GCATCTCTTGGAAGGATGAACCAGGGTACTGCACGCCTTGCTAGTTTAATGAATCT
TGGAATGAGTTCTTCATTGAATCAACAAGGAGCTCATAGTGCACTGTCTTCTGCTA
GTACTTCTTCCCATAATTTGCAGTCTATATTTAACATTGGAAGTAGAGGTCCACTCC
CTTTATCTTCTCAACACCGTGGAGATGCAGACCAGGCCAGTAACATTTTGGCCAGC
TTTGGTCTGTCTGCTAGAGACTTAGATGAACTGAGTCGTTATCCAGAGGACAAGAT
TACTCCTGAGAATTTGCCCCAAATCCTTCTACAGCTTAAAAGGAGGAGAACTGAAG
AAGGCCCTACCTTGAGTTATGGTAGAGATGGCAGATCTGCTACACGGGAGCCACCA
TACAGAGTACCTAGGGATGATTGGGAAGAAAAAAGGCACTTTAGAAGAGATAGTT
TTGATGATCGTGGTCCTAGTCTCAACCCAGTGCTTGATTATGACCATGGAAGTCGTT
CTCAAGAATCTGGTTATTATGACAGAATGGATTATGAAGATGACAGATTAAGAGAT
GGAGAAAGGTGTAGGGATGATTCTTTTTTTGGTGAGACCTCGCATAACTATCATAA
ATTTGACAGTGAGTATGAGAGAATGGGACGTGGTCCTGGCCCCTTACAAGAGAGA
TCTCTCTTTGAGAAAAAGAGAGGCGCTCCTCCAAGTAGCAATATTGAAGACTTCCA
TGGACTCTTACCGAAGTAACCGGGAATTCATCGTGACTGACTGACGATCTGCCTCG
CGCGTTTCGGTGATGACGGTGAAAACCTCTGACACATGCAGCTCCCGGAGACGGTC
ACAGCTTGTCTGTAAGCGGATGCCGGGAGCAGACAAGCCCGTCAGGGCGCGTCAG
CGGGTGTTGGCGGGTGTCGGGGCGCAGCCATGACCCAGTCACGTAGCGATAGCGG
AGTGTATAATTCTTGAAGACGAAAGGGCCTCGTGATACGCCTATTTTTATAGGTTA
ATGTCATGATAATAATGGTTTCTTAGACGTCAGGTGGCACTTTTCGGGGAAATGTG
CGCGGAACCCCTATTTGTTTATTTTTCTAAATACATTCAAATATGTATCCGCTCATG
AGACAATAACCCTGATAAATGCTTCAATAATATTGAAAAAGGAAGAGTATGAGTA
TTCAACATTTCCGTGTCGCCCTTATTCCCTTTTTTGCGGCATTTTGCCTTCCTGTTTTT
GCTCACCCAGAAACGCTGGTGAAAGTAAAAGATGCTGAAGATCAGTTGGGTGCAC
GAGTGGGTTACATCGAACTGGATCTCAACAGCGGTAAGATCCTTGAGAGTTTTCGC
CCCGAAGAACGTTTTCCAATGATGAGCACTTTTAAAGTTCTGCTATGTGGCGCGGT
ATTATCCCGTGTTGACGCCGGGCAAGAGCAACTCGGTCGCCGCATACACTATTCTC
AGAATGACTTGGTTGAGTACTCACCAGTCACAGAAAAGCATCTTACGGATGGCATG
ACAGTAAGAGAATTATGCAGTGCTGCCATAACCATGAGTGATAACACTGCGGCCA
ACTTACTTCTGACAACGATCGGAGGACCGAAGGAGCTAACCGCTTTTTTGCACAAC
ATGGGGGATCATGTAACTCGCCTTGATCGTTGGGAACCGGAGCTGAATGAAGCCAT
ACCAAACGACGAGCGTGACACCACGATGCCTGCAGCAATGGCAACAACGTTGCGC
AAACTATTAACTGGCGAACTACTTACTCTAGCTTCCCGGCAACAATTAATAGACTG
GATGGAGGCGGATAAAGTTGCAGGACCACTTCTGCGCTCGGCCCTTCCGGCTGGCT
GGTTTATTGCTGATAAATCTGGAGCCGGTGAGCGTGGGTCTCGCGGTATCATTGCA
GCACTGGGGCCAGATGGTAAGCCCTCCCGTATCGTAGTTATCTACACGACGGGGAG
TCAGGCAACTATGGATGAACGAAATAGACAGATCGCTGAGATAGGTGCCTCACTG
ATTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCATG
ACCAAAATCCCTTAACGTGAGTTTTCGTTCCACTGAGCGTCAGACCCCGTAGAAAA
GATCAAAGGATCTTCTTGAGATCCTTTTTTTCTGCGCGTAATCTGCTGCTTGCAAAC
AAAAAAACCACCGCTACCAGCGGTGGTTTGTTTGCCGGATCAAGAGCTACCAACTC
TTTTTCCGAAGGTAACTGGCTTCAGCAGAGCGCAGATACCAAATACTGTCCTTCTA
GTGTAGCCGTAGTTAGGCCACCACTTCAAGAACTCTGTAGCACCGCCTACATACCT
CGCTCTGCTAATCCTGTTACCAGTGGCTGCTGCCAGTGGCGATAAGTCGTGTCTTAC
CGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGAT
ACCTACAGCGTGAGCTATGAGAAAGCGCCACGCTTCCCGAAGGGAGAAAGGCGGA
CAGGTATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTGACTTGA
GCGTCGATTTTTGTGATGCTCGTCAGGGGGGCGGAGCCTATGGAAAAACGCCAGCA
ACGCGGCCTTTTTACGGTTCCTGGCCTTTTGCTGGCCTTTTGCTCACATGTTCTTTCC
TGCGTTATCCCCTGATTCTGTGGATAACCGTATTACCGCCTTTGAGTGAGCTGATAC
CGCTCGCCGCAGCCGAACGACCGAGCGCAGCGAGTCAGTGAGCGAGGAAGCGGAA
GAGCGCCTGATGCGGTATTTTCTCCTTACGCATCTGTGCGGTATTTCACACCGCATA
AATTCCGACACCATCGAATGGTGCAAAACCTTTCGCGGTATGGCATGATAGCGCCC
GGAAGAGAGTCAATTCAGGGTGGTGAATGTGAAACCAGTAACGTTATACGATGTC
GCAGAGTATGCCGGTGTCTCTTATCAGACCGTTTCCCGCGTGGTGAACCAGGCCAG
CCACGTTTCTGCGAAAACGCGGGAAAAAGTGGAAGCGGCGATGGCGGAGCTGAAT
TACATTCCCAACCGCGTGGCACAACAACTGGCGGGCAAACAGTCGTTGCTGATTGG
CGTTGCCACCTCCAGTCTGGCCCTGCACGCGCCGTCGCAAATTGTCGCGGCGATTA
AATCTCGCGCCGATCAACTGGGTGCCAGCGTGGTGGTGTCGATGGTAGAACGAAG
CGGCGTCGAAGCCTGTAAAGCGGCGGTGCACAATCTTCTCGCGCAACGCGTCAGTG
GGCTGATCATTAACTATCCGCTGGATGACCAGGATGCCATTGCTGTGGAAGCTGCC
TGCACTAATGTTCCGGCGTTATTTCTTGATGTCTCTGACCAGACACCCATCAACAGT
ATTATTTTCTCCCATGAAGACGGTACGCGACTGGGCGTGGAGCATCTGGTCGCATT
GGGTCACCAGCAAATCGCGCTGTTAGCGGGCCCATTAAGTTCTGTCTCGGCGCGTC
TGCGTCTGGCTGGCTGGCATAAATATCTCACTCGCAATCAAATTCAGCCGATAGCG
GAACGGGAAGGCGACTGGAGTGCCATGTCCGGTTTTCAACAAACCATGCAAATGC
TGAATGAGGGCATCGTTCCCACTGCGATGCTGGTTGCCAACGATCAGATGGCGCTG
GGCGCAATGCGCGCCATTACCGAGTCCGGGCTGCGCGTTGGTGCGGATATCTCGGT
AGTGGGATACGACGATACCGAAGACAGCTCATGTTATATCCCGCCGTTAACCACCA
TCAAACAGGATTTTCGCCTGCTGGGGCAAACCAGCGTGGACCGCTTGCTGCAACTC
TCTCAGGGCCAGGCGGTGAAGGGCAATCAGCTGTTGCCCGTCTCACTGGTGAAAAG
AAAAACCACCCTGGCGCCCAATACGCAAACCGCCTCTCCCCGCGCGTTGGCCGATT
CATTAATGCAGCTGGCACGACAGGTTTCCCGACTGGAAAGCGGGCAGTGAGCGCA
ACGCAATTAATGTGAGTTAGCTCACTCATTAGGCACCCCAGGCTTTACACTTTATGC
TTCCGGCTCGTATGTTGTGTGGAATTGTGAGCGGATAACAATTTCACACAGGAAAC
AGCTATGACCATGATTACGGATTCACTGGCCGTCGTTTTACAACGTCGTGACTGGG
AAAACCCTGGCGTTACCCAACTTAATCGCCTTGCAGCACATCCCCCTTTCGCCAGCT
GGCGTAATAGCGAAGAGGCCCGCACCGATCGCCCTTCCCAACAGTTGCGCAGCCTG
AATGGCGAATGGCGCTTTGCCTGGTTTCCGGCACCAGAAGCGGTGCCGGAAAGCTG
GCTGGAGTGCGATCTTCCTGAGGCCGATACTGTCGTCGTCCCCTCAAACTGGCAGA
TGCACGGTTACGATGCGCCCATCTACACCAACGTAACCTATCCCATTACGGTCAAT
CCGCCGTTTGTTCCCACGGAGAATCCGACGGGTTGTTACTCGCTCACATTTAATGTT
GATGAAAGCTGGCTACAGGAAGGCCAGACGCGAATTATTTTTGATGGCGTTGGAAT
T
pETGB1a-His- DUX4 DNA binding domain (dbd)
(SEQ ID NO: 91)
GCGCTCACTGCCCGCTTTCCAGTCGGGAAACCTGTCGTGCCAGCTGCATTAATGAA
TCGGCCAACGCGCGGGGAGAGGCGGTTTGCGTATTGGGCGCCAGGGTGGTTTTTCT
TTTCACCAGTGAGACGGGCAACAGCTGATTGCCCTTCACCGCCTGGCCCTGAGAGA
GTTGCAGCAAGCGGTCCACGCTGGTTTGCCCCAGCAGGCGAAAATCCTGTTTGATG
GTGGTTAACGGCGGGATATAACATGAGCTGTCTTCGGTATCGTCGTATCCCACTAC
CGAGATATCCGCACCAACGCGCAGCCCGGACTCGGTAATGGCGCGCATTGCGCCC
AGCGCCATCTGATCGTTGGCAACCAGCATCGCAGTGGGAACGATGCCCTCATTCAG
CATTTGCATGGTTTGTTGAAAACCGGACATGGCACTCCAGTCGCCTTCCCGTTCCGC
TATCGGCTGAATTTGATTGCGAGTGAGATATTTATGCCAGCCAGCCAGACGCAGAC
GCGCCGAGACAGAACTTAATGGGCCCGCTAACAGCGCGATTTGCTGGTGACCCAAT
GCGACCAGATGCTCCACGCCCAGTCGCGTACCGTCTTCATGGGAGAAAATAATACT
GTTGATGGGTGTCTGGTCAGAGACATCAAGAAATAACGCCGGAACATTAGTGCAG
GCAGCTTCCACAGCAATGGCATCCTGGTCATCCAGCGGATAGTTAATGATCAGCCC
ACTGACGCGTTGCGCGAGAAGATTGTGCACCGCCGCTTTACAGGCTTCGACGCCGC
TTCGTTCTACCATCGACACCACCACGCTGGCACCCAGTTGATCGGCGCGAGATTTA
ATCGCCGCGACAATTTGCGACGGCGCGTGCAGGGCCAGACTGGAGGTGGCAACGC
CAATCAGCAACGACTGTTTGCCCGCCAGTTGTTGTGCCACGCGGTTGGGAATGTAA
TTCAGCTCCGCCATCGCCGCTTCCACTTTTTCCCGCGTTTTCGCAGAAACGTGGCTG
GCCTGGTTCACCACGCGGGAAACGGTCTGATAAGAGACACCGGCATACTCTGCGA
CATCGTATAACGTTACTGGTTTCACATTCACCACCCTGAATTGACTCTCTTCCGGGC
GCTATCATGCCATACCGCGAAAGGTTTTGCGCCATTCGATGGTGTCCGGGATCTCG
ACGCTCTCCCTTATGCGACTCCTGCATTAGGAAGCAGCCCAGTAGTAGGTTGAGGC
CGTTGAGCACCGCCGCCGCAAGGAATGGTGCATGCAAGGAGATGGCGCCCAACAG
TCCCCCGGCCACGGGGCCTGCCACCATACCCACGCCGAAACAAGCGCTCATGAGCC
CGAAGTGGCGAGCCCGATCTTCCCCATCGGTGATGTCGGCGATATAGGCGCCAGCA
ACCGCACCTGTGGCGCCGGTGATGCCGGCCACGATGCGTCCGGCGTAGAGGATCG
AGATCTCGATCCCGCGAAATTAATACGACTCACTATAGGGGAATTGTGAGCGGATA
ACAATTCCCCTCTAGAAATAATTTTGTTTAACTTTAAGAAGGAGATATACCATGAA
ACATCACCATCACCATCACCCCATGAAACAGTACAAGCTTATCCTGAACGGTAAAA
CCCTGAAAGGTGAAACCACCACCGAAGCTGTTGACGCTGCTACCGCGGAAAAAGT
TTTCAAACAGTACGCTAACGACAACGGTGTTGACGGTGAATGGACCTACGACGAC
GCTACCAAAACCTTCACGGTAACCGAAGGATCTGGCAGTGGTTCTGAGAATCTTTA
TTTTCAGGGCGCCATGGaaGCCCTCCCGACACCCTCGGACAGCACCCTCCCCGCGGA
AGCCCGGGGACGAGGACGGCGACGGAGACTCGTTTGGACCCCGAGCCAAAGCGAG
GCCCTGCGAGCCTGCTTTGAGCGGAACCCGTACCCGGGCATCGCCACCAGAGAAC
GGCTGGCCCAGGCCATCGGCATTCCGGAGCCCAGGGTCCAGATTTGGTTTCAGAAT
GAGAGGTCACGCCAGCTGAGGCAGCACCGGCGGGAATCTCGGCCCTGGCCCGGGA
GACGCGGCCCGCCAGAAGGCCGGCGAAAGCGGACCGCCGTCACCGGATCCCAGAC
CGCCCTGCTCCTCCGAGCCTTTGAGAAGGATCGCTTTCCAGGCATCGCCGCCCGGG
AGGAGCTGGCCAGAGAGACGGGCCTCCCGGAGTCCAGGATTCAGATCTGGTTTCA
GAATCGAAGGGCCAGGCACCCGGGACAGGGTGGCAGGGCGCCCGCGCAGGTCTAG
CTCGAGCACCACCACCACCACCACTGAGATCCGGCTGCTAACAAAGCCCGAAAGG
AAGCTGAGTTGGCTGCTGCCACCGCTGAGCAATAACTAGCATAACCCCTTGGGGCC
TCTAAACGGGTCTTGAGGGGTTTTTTGCTGAAAGGAGGAACTATATCCGGATTGGC
GAATGGGACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGCTTTCTTCC
CTTCCTTTCTCGCCACGTTCGCCGGCTTTCCCCGTCAAGCTCTAAATCGGGGGCTCC
CTTTAGGGTTCCGATTTAGTGCTTTACGGCACCTCGACCCCAAAAAACTTGATTAG
GGTGATGGTTCACGTAGTGGGCCATCGCCCTGATAGACGGTTTTTCGCCCTTTGAC
GTTGGAGTCCACGTTCTTTAATAGTGGACTCTTGTTCCAAACTGGAACAACACTCA
ACCCTATCTCGGTCTATTCTTTTGATTTATAAGGGATTTTGCCGATTTCGGCCTATTG
GTTAAAAAATGAGCTGATTTAACAAAAATTTAACGCGAATTTTAACAAAATATTAA
CGTTTACAATTTCAGGTGGCACTTTTCGGGGAAATGTGCGCGGAACCCCTATTTGTT
TATTTTTCTAAATACATTCAAATATGTATCCGCTCATGAATTAATTCTTAGAAAAAC
TCATCGAGCATCAAATGAAACTGCAATTTATTCATATCAGGATTATCAATACCATA
TTTTTGAAAAAGCCGTTTCTGTAATGAAGGAGAAAACTCACCGAGGCAGTTCCATA
GGATGGCAAGATCCTGGTATCGGTCTGCGATTCCGACTCGTCCAACATCAATACAA
CCTATTAATTTCCCCTCGTCAAAAATAAGGTTATCAAGTGAGAAATCACCATGAGT
GACGACTGAATCCGGTGAGAATGGCAAAAGTTTATGCATTTCTTTCCAGACTTGTT
CAACAGGCCAGCCATTACGCTCGTCATCAAAATCACTCGCATCAACCAAACCGTTA
TTCATTCGTGATTGCGCCTGAGCGAGACGAAATACGCGATCGCTGTTAAAAGGACA
ATTACAAACAGGAATCGAATGCAACCGGCGCAGGAACACTGCCAGCGCATCAACA
ATATTTTCACCTGAATCAGGATATTCTTCTAATACCTGGAATGCTGTTTTCCCGGGG
ATCGCAGTGGTGAGTAACCATGCATCATCAGGAGTACGGATAAAATGCTTGATGGT
CGGAAGAGGCATAAATTCCGTCAGCCAGTTTAGTCTGACCATCTCATCTGTAACAT
CATTGGCAACGCTACCTTTGCCATGTTTCAGAAACAACTCTGGCGCATCGGGCTTC
CCATACAATCGATAGATTGTCGCACCTGATTGCCCGACATTATCGCGAGCCCATTT
ATACCCATATAAATCAGCATCCATGTTGGAATTTAATCGCGGCCTAGAGCAAGACG
TTTCCCGTTGAATATGGCTCATAACACCCCTTGTATTACTGTTTATGTAAGCAGACA
GTTTTATTGTTCATGACCAAAATCCCTTAACGTGAGTTTTCGTTCCACTGAGCGTCA
GACCCCGTAGAAAAGATCAAAGGATCTTCTTGAGATCCTTTTTTTCTGCGCGTAATC
TGCTGCTTGCAAACAAAAAAACCACCGCTACCAGCGGTGGTTTGTTTGCCGGATCA
AGAGCTACCAACTCTTTTTCCGAAGGTAACTGGCTTCAGCAGAGCGCAGATACCAA
ATACTGTCCTTCTAGTGTAGCCGTAGTTAGGCCACCACTTCAAGAACTCTGTAGCA
CCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCG
GTCGGGCTGAACGGGGGGTTCGTGCACACAGCCCAGCTTGGAGCGAACGACCTAC
ACCGAACTGAGATACCTACAGCGTGAGCTATGAGAAAGCGCCACGCTTCCCGAAG
GGAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCGC
CACCTCTGACTTGAGCGTCGATTTTTGTGATGCTCGTCAGGGGGGCGGAGCCTATG
GAAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCTTT
GAGTGAGCTGATACCGCTCGCCGCAGCCGAACGACCGAGCGCAGCGAGTCAGTGA
GCGAGGAAGCGGAAGAGCGCCTGATGCGGTATTTTCTCCTTACGCATCTGTGCGGT
ATTTCACACCGCATATATGGTGCACTCTCAGTACAATCTGCTCTGATGCCGCATAGT
TAAGCCAGTATACACTCCGCTATCGCTACGTGACTGGGTCATGGCTGCGCCCCGAC
ACCCGCCAACACCCGCTGACGCGCCCTGACGGGCTTGTCTGCTCCCGGCATCCGCT
TACAGACAAGCTGTGACCGTCTCCGGGAGCTGCATGTGTCAGAGGTTTTCACCGTC
ATCACCGAAACGCGCGAGGCAGCTGCGGTAAAGCTCATCAGCGTGGTCGTGAAGC
GATTCACAGATGTCTGCCTGTTCATCCGCGTCCAGCTCGTTGAGTTTCTCCAGAAGC
GTTAATGTCTGGCTTCTGATAAAGCGGGCCATGTTAAGGGCGGTTTTTTCCTGTTTG
GTCACTGATGCCTCCGTGTAAGGGGGATTTCTGTTCATGGGGGTAATGATACCGAT
GAAACGAGAGAGGATGCTCACGATACGGGTTACTGATGATGAACATGCCCGGTTA
CTGGAACGTTGTGAGGGTAAACAACTGGCGGTATGGATGCGGCGGGACCAGAGAA
AAATCACTCAGGGTCAATGCCAGCGCTTCGTTAATACAGATGTAGGTGTTCCACAG
GGTAGCCAGCAGCATCCTGCGATGCAGATCCGGAACATAATGGTGCAGGGCGCTG
ACTTCCGCGTTTCCAGACTTTACGAAACACGGAAACCGAAGACCATTCATGTTGTT
GCTCAGGTCGCAGACGTTTTGCAGCAGCAGTCGCTTCACGTTCGCTCGCGTATCGG
TGATTCATTCTGCTAACCAGTAAGGCAACCCCGCCAGCCTAGCCGGGTCCTCAACG
ACAGGAGCACGATCATGCGCACCCGTGGGGCCGCCATGCCGGCGATAATGGCCTG
CTTCTCGCCGAAACGTTTGGTGGCGGGACCAGTGACGAAGGCTTGAGCGAGGGCG
TGCAAGATTCCGAATACCGCAAGCGACAGGCCGATCATCGTCGCGCTCCAGCGAA
AGCGGTCCTCGCCGAAAATGACCCAGAGCGCTGCCGGCACCTGTCCTACGAGTTGC
ATGATAAAGAAGACAGTCATAAGTGCGGCGACGATAGTCATGCCCCGCGCCCACC
GGAAGGAGCTGACTGGGTTGAAGGCTCTCAAGGGCATCGGTCGAGATCCCGGTGC
CTAATGAGTGAGCTAACTTACATTAATTGCGTT
pFUGW-GFP
(SEQ ID NO: 92)
GTCGACGGATCGGGAGATCTCCCGATCCCCTATGGTGCACTCTCAGTACAATCTGC
TCTGATGCCGCATAGTTAAGCCAGTATCTGCTCCCTGCTTGTGTGTTGGAGGTCGCT
GAGTAGTGCGCGAGCAAAATTTAAGCTACAACAAGGCAAGGCTTGACCGACAATT
GCATGAAGAATCTGCTTAGGGTTAGGCGTTTTGCGCTGCTTCGCGATGTACGGGCC
AGATATACGCGTTGACATTGATTATTGACTAGTTATTAATAGTAATCAATTACGGG
GTCATTAGTTCATAGCCCATATATGGAGTTCCGCGTTACATAACTTACGGTAAATG
GCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGG
ATTTCCAAGTCTCCACCCCATTGACGTCAATGGGAGTTTGTTTTGGCACCAAAATCA
ACGGGACTTTCCAAAATGTCGTAACAACTCCGCCCCATTGACGCAAATGGGCGGTA
GGCGTGTACGGTGGGAGGTCTATATAAGCAGCGCGTTTTGCCTGTACTGGGTCTCT
CTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCT
AGCAGTGGCGCCCGAACAGGGACTTGAAAGCGAAAGGGAAACCAGAGGAGCTCTC
TCGACGCAGGACTCGGCTTGCTGAAGCGCGCACGGCAAGAGGCGAGGGGCGGCGA
CTGGTGAGTACGCCAAAAATTTTGACTAGCGGAGGCTAGAAGGAGAGAGATGGGT
GCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGC
AGGGAGCTAGAACGATTCGCAGTTAATCCTGGCCTGTTAGAAACATCAGAAGGCT
GTAGACAAATACTGGGACAGCTACAACCATCCCTTCAGACAGGATCAGAAGAACT
TAGATCATTATATAATACAGTAGCAACCCTCTATTGTGTGCATCAAAGGATAGAGA
TAAAAGACACCAAGGAAGCTTTAGACAAGATAGAGGAAGAGCAAAACAAAAGTA
AGACCACCGCACAGCAAGCGGCCGCTGATCTTCAGACCTGGAGGAGGAGATATGA
GGGACAATTGGAGAAGTGAATTATATAAATATAAAGTAGTAAAAATTGAACCATT
AGGAGTAGCACCCACCAAGGCAAAGAGAAGAGTGGTGCAGAGAGAAAAAAGAGC
AGTGGGAATAGGAGCTTTGTTCCTTGGGTTCTTGGGAGCAGCAGGAAGCACTATGG
GCGCAGCGTCAATGACGCTGACGGTACAGGCCAGACAATTATTGTCTGGTATAGTG
CAGCAGCAGAACAATTTGCTGAGGGCTATTGAGGCGCAACAGCATCTGTTGCAACT
CACAGTCTGGGGCATCAAGCAGCTCCAGGCAAGAATCCTGGCTGTGGAAAGATAC
CTAAAGGATCAACAGCTCCTGGGGATTTGGGGTTGCTCTGGAAAACTCATTTGCAC
CACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAAT
ACACTCCTTAATTGAAGAATCGCAAAACCAGCAAGAAAAGAATGAACAAGAATTA
TTGGAATTAGATAAATGGGCAAGTTTGTGGAATTGGTTTAACATAACAAATTGGCT
GTGGTATATAAAATTATTCATAATGATAGTAGGAGGCTTGGTAGGTTTAAGAATAG
TTTTTGCTGTACTTTCTATAGTGAATAGAGTTAGGCAGGGATATTCACCATTATCGT
TTCAGACCCACCTCCCAACCCCGAGGGGACCCGACAGGCCCGAAGGAATAGAAGA
AGAAGGTGGAGAGAGAGACAGAGACAGATCCATTCGATTAGTGAACGGATCGGCA
CTGCGTGCGCCAATTCTGCAGACAAATGGCAGTATTCATCCACAATTTTAAAAGAA
AAGGGGGGATTGGGGGGTACAGTGCAGGGGAAAGAATAGTAGACATAATAGCAA
CAGACATACAAACTAAAGAATTACAAAAACAAATTACAAAAATTCAAAATTTTCG
GGTTTATTACAGGGACAGCAGAGATCCAGTTTGGTTAATTAAgggtgcagcggcctccgcgcc
gggttttggcgcctcccgcgggcgcccccctcctcacggcgagcgctgccacgtcagacgaagggcgcaggagcgttcctgatccttc
cgcccggacgctcaggacagcggcccgctgctcataagactcggccttagaaccccagtatcagcagaaggacattttaggacgggac
ttgggtgactctagggcactggttttctttccagagagcggaacaggcgaggaaaagtagtcccttctcggcgattctgcggagggatctc
cgtggggcggtgaacgccgatgattatataaggacgcgccgggtgtggcacagctagttccgtcgcagccgggatttgggtcgcggttc
ttgtttgtggatcgctgtgatcgtcacttggtgagttgcgggctgctgggctggccggggctttcgtggccgccgggccgctcggtgggac
ggaagcgtgtggagagaccgccaagggctgtagtctgggtccgcgagcaaggttgccctgaactgggggttggggggagcgcacaa
aatggcggctgttcccgagtcttgaatggaagacgcttgtaaggcgggctgtgaggtcgttgaaacaaggtggggggcatggtgggcg
gcaagaacccaaggtcttgaggccttcgctaatgcgggaaagctcttattcgggtgagatgggctggggcaccatctggggaccctgac
gtgaagtttgtcactgactggagaactcgggtttgtcgtctggttgcgggggcggcagttatgcggtgccgttgggcagtgcacccgtacc
tttgggagcgcgcgcctcgtcgtgtcgtgacgtcacccgttctgttggcttataatgcagggtggggccacctgccggtaggtgtgcggta
ggcttttctccgtcgcaggacgcagggttcgggcctagggtaggctctcctgaatcgacaggcgccggacctctggtgaggggaggga
taagtgaggcgtcagtttctttggtcggttttatgtacctatcttcttaagtagctgaagctccggttttgaactatgcgctcggggttggcgagt
gtgttttgtgaagttttttaggcaccttttgaaatgtaatcatttgggtcaatatgtaattttcagtgttagactagtaaagcttctgcaggtcgact
ctagaaaattgtccgctaaattctggccgtttttggcttttttgttagacaGGATCCCCGGGTACCGGTCGCCACCAT
GGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATG
CCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTA
CCCCGACCACATGAAGCAGCACGACTTCTTCAAGTCCGCCATGCCCGAAGGCTACG
TCCAGGAGCGCACCATCTTCTTCAAGGACGACGGCAACTACAAGACCCGCGCCGA
GGTGAAGTTCGAGGGCGACACCCTGGTGAACCGCATCGAGCTGAAGGGCATCGAC
TTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAA
GATCCGCCACAACATCGAGGACGGCAGCGTGCAGCTCGCCGACCACTACCAGCAG
AACACCCCCATCGGCGACGGCCCCGTGCTGCTGCCCGACAACCACTACCTGAGCAC
CCAGTCCGCCCTGAGCAAAGACCCCAACGAGAAGCGCGATCACATGGTCCTGCTG
GAGTTCGTGACCGCCGCCGGGATCACTCTCGGCATGGACGAGCTGTACAAGTAAA
GCGGCCGCGACTCTAGAATTCGATATCAAGCTTATCGATAATCAACCTCTGGATTA
CAAAATTTGTGAAAGATTGACTGGTATTCTTAACTATGTTGCTCCTTTTACGCTATG
TGGATACGCTGCTTTAATGCCTTTGTATCATGCTATTGCTTCCCGTATGGCTTTCATT
TTCTCCTCCTTGTATAAATCCTGGTTGCTGTCTCTTTATGAGGAGTTGTGGCCCGTT
GTCAGGCAACGTGGCGTGGTGTGCACTGTGTTTGCTGACGCAACCCCCACTGGTTG
GGGCATTGCCACCACCTGTCAGCTCCTTTCCGGGACTTTCGCTTTCCCCCTCCCTAT
TGCCACGGCGGAACTCATCGCCGCCTGCCTTGCCCGCTGCTGGACAGGGGCTCGGC
TGTTGGGCACTGACAATTCCGTGGTGTTGTCGGGGAAATCATCGTCCTTTCCTTGGC
TGCTCGCCTGTGTTGCCACCTGGATTCTGCGCGGGACGTCCTTCTGCTACGTCCCTT
CGGCCCTCAATCCAGCGGACCTTCCTTCCCGCGGCCTGCTGCCGGCTCTGCGGCCTC
TTCCGCGTCTTCGCCTTCGCCCTCAGACGAGTCGGATCTCCCTTTGGGCCGCCTCCC
CGCATCGATACCGTCGACCTCGAGACCTAGAAAAACATGGAGCAATCACAAGTAG
CAATACAGCAGCTACCAATGCTGATTGTGCCTGGCTAGAAGCACAAGAGGAGGAG
GAGGTGGGTTTTCCAGTCACACCTCAGGTACCTTTAAGACCAATGACTTACAAGGC
AGCTGTAGATCTTAGCCACTTTTTAAAAGAAAAGGGGGGACTGGAAGGGCTAATTC
ACTCCCAACGAAGACAAGATATCCTTGATCTGTGGATCTACCACACACAAGGCTAC
TTCCCTGATTGGCAGAACTACACACCAGGGCCAGGGATCAGATATCCACTGACCTT
TGGATGGTGCTACAAGCTAGTACCAGTTGAGCAAGAGAAGGTAGAAGAAGCCAAT
GAAGGAGAGAACACCCGCTTGTTACACCCTGTGAGCCTGCATGGGATGGATGACC
CGGAGAGAGAAGTATTAGAGTGGAGGTTTGACAGCCGCCTAGCATTTCATCACATG
GCCCGAGAGCTGCATCCGGACTGTACTGGGTCTCTCTGGTTAGACCAGATCTGAGC
CTGGGAGCTCTCTGGCTAACTAGGGAACCCACTGCTTAAGCCTCAATAAAGCTTGC
CTTGAGTGCTTCAAGTAGTGTGTGCCCGTCTGTTGTGTGACTCTGGTAACTAGAGAT
CCCTCAGACCCTTTTAGTCAGTGTGGAAAATCTCTAGCAGGGCCCGTTTAAACCCG
CTGATCAGCCTCGACTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGA
GGAAATTGCATCGCATTGTCTGAGTAGGTGTCATTCTATTCTGGGGGGTGGGGTGG
GGCAGGACAGCAAGGGGGAGGATTGGGAAGACAATAGCAGGCATGCTGGGGATG
CGGTGGGCTCTATGGCTTCTGAGGCGGAAAGAACCAGCTGGGGCTCTAGGGGGTA
TCCCCACGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTCTTCCCTT
CCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGT
GATGGTTCACGTAGTGGGCCATCGCCCTGATAGACGGTTTTTCGCCCTTTGACGTTG
GAGTCCACGTTCTTTAATAGTGGACTCTTGTTCCAAACTGGAACAACACTCAACCC
TATCTCGGTCTATTCTTTTGATTTATAAGGGATTTTGCCGATTTCGGCCTATTGGTTA
AAAAATGAGCTGATTTAACAAAAATTTAACGCGAATTAATTCTGTGGAATGTGTGT
CAGTTAGGGTGTGGAAAGTCCCCAGGCTCCCCAGCAGGCAGAAGTATGCAAAGCA
TGCATCTCAATTAGTCAGCAACCAGGTGTGGAAAGTCCCCAGGCTCCCCAGCAGGC
AGAAGTATGCAAAGCATGCATCTCAATTAGTCAGCAACCATAGTCCCGCCCCTAAC
TCCGCCCATCCCGCCCCTAACTCCGCCCAGTTCCGCCCATTCTCCGCCCCATGGCTG
ACTAATTTTTTTTATTTATGCAGAGGCCGAGGCCGCCTCTGCCTCTGAGCTATTCCA
GAAGTAGTGAGGAGGCTTTTTTGGAGGCCTAGGCTTTTGCAAAAAGCTCCCGGGAG
CTTGTATATCCATTTTCGGATCTGATCAGCACGTGTTGACAATTAATCATCGGCATA
GTATATCGGCATAGTATAATACGACAAGGTGAGGAACTAAACCATGGCCAAGTTG
ACCAGTGCCGTTCCGGTGCTCACCGCGCGCGACGTCGCCGGAGCGGTCGAGTTCTG
GACCGACCGGCTCGGGTTCTCCCGGGACTTCGTGGAGGACGACTTCGCCGGTGTGG
TCCGGGACGACGTGACCCTGTTCATCAGCGCGGTCCAGGACCAGGTGGTGCCGGAC
AACACCCTGGCCTGGGTGTGGGTGCGCGGCCTGGACGAGCTGTACGCCGAGTGGTC
GGAGGTCGTGTCCACGAACTTCCGGGACGCCTCCGGGCCGGCCATGACCGAGATC
GGCGAGCAGCCGTGGGGGCGGGAGTTCGCCCTGCGCGACCCGGCCGGCAACTGCG
TGCACTTCGTGGCCGAGGAGCAGGACTGACACGTGCTACGAGATTTCGATTCCACC
GCCGCCTTCTATGAAAGGTTGGGCTTCGGAATCGTTTTCCGGGACGCCGGCTGGAT
GATCCTCCAGCGCGGGGATCTCATGCTGGAGTTCTTCGCCCACCCCAACTTGTTTAT
TGCAGCTTATAATGGTTACAAATAAAGCAATAGCATCACAAATTTCACAAATAAAG
CATTTTTTTCACTGCATTCTAGTTGTGGTTTGTCCAAACTCATCAATGTATCTTATCA
TGTCTGTATACCGTCGACCTCTAGCTAGAGCTTGGCGTAATCATGGTCATAGCTGTT
TCCTGTGTGAAATTGTTATCCGCTCACAATTCCACACAACATACGAGCCGGAAGCA
TAAAGTGTAAAGCCTGGGGTGCCTAATGAGTGAGCTAACTCACATTAATTGCGTTG
CGCTCACTGCCCGCTTTCCAGTCGGGAAACCTGTCGTGCCAGCTGCATTAATGAAT
CGGCCAACGCGCGGGGAGAGGCGGTTTGCGTATTGGGCGCTCTTCCGCTTCCTCGC
TCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTT
TTTCCATAGGCTCCGCCCCCCTGACGAGCATCACAAAAATCGACGCTCAAGTCAGA
GGTGGCGAAACCCGACAGGACTATAAAGATACCAGGCGTTTCCCCCTGGAAGCTC
CCTCGTGCGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCAGTTCGGT
GTAGGTCGTTCGCTCCAAGCTGGGCTGTGTGCACGAACCCCCCGTTCAGCCCGACC
GCTGCGCCTTATCCGGTAACTATCGTCTTGAGTCCAACCCGGTAAGACACGACTTA
TCGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGAACAGTA
TTTGGTATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCAAGCAGC
AGATTACGCGCAGAAAAAAAGGATCTCAAGAAGATCCTTTGATCTTTTCTACGGGG
TCTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAATCT
AAAGTATATATGAGTAAACTTGGTCTGACAGTTACCAATGCTTAATCAGTGAGGCA
CCTATCTCAGCGATCTGTCTATTTCGTTCATCCATAGTTGCCTGACTCCCCGTCGTG
TAGATAACTACGATACGGGAGGGCTTACCATCTGGCCCCAGTGCTGCAATGATACC
GCGAGACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTCTATTAA
TTGTTGCCGGGAAGCTAGAGTAAGTAGTTCGCCAGTTAATAGTTTGCGCAACGTTG
TTGCCATTGCTACAGGCATCGTGGTGTCACGCTCGTCGTTTGGTATGGCTTCATTCA
GCTCCGGTTCCCAACGATCAAGGCGAGTTACATGATCCCCCATGTTGTGCAAAAAA
GCGGTTAGCTCCTTCGGTCCTCCGATCGTTGTCAGAAGTAAGTTGGCCGCAGTGTT
ATCACTCATGGTTATGGCAGCACTGCATAATTCTCTTACTGTCATGCCATCCGTAAG
ATGCTTTTCTGTGACTGGTGAGTACTCAACCAAGTCATTCTGAGAATAGTGTATGC
GGCGACCGAGTTGCTCTTGCCCGGCGTCAATACGGGATAATACCGCGCCACATAGC
AGAACTTTAAAAGTGCTCATCATTGGAAAACGTTCTTCGGGGCGAAAACTCTCAAG
GATCTTACCGCTGTTGAGATCCAGTTCGATGTAACCCACTCGTGCACCCAACTGAT
CTTCAGCATCTTTTACTTTCACCAGCGTTTCTGGGTGAGCAAAAACAGGAAGGCAA
AATGCCGCAAAAAAGGGAATAAGGGCGACACGGAAATGTTGAATACTCATACTCT
TCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGA
AAAGTGCCACCTGAC
pFUGW-GFP-MATR3
(SEQ ID NO: 93)
GTCGACGGATCGGGAGATCTCCCGATCCCCTATGGTGCACTCTCAGTACAATCTGC
TCTGATGCCGCATAGTTAAGCCAGTATCTGCTCCCTGCTTGTGTGTTGGAGGTCGCT
GAGTAGTGCGCGAGCAAAATTTAAGCTACAACAAGGCAAGGCTTGACCGACAATT
GCATGAAGAATCTGCTTAGGGTTAGGCGTTTTGCGCTGCTTCGCGATGTACGGGCC
AGATATACGCGTTGACATTGATTATTGACTAGTTATTAATAGTAATCAATTACGGG
GTCATTAGTTCATAGCCCATATATGGAGTTCCGCGTTACATAACTTACGGTAAATG
GCCCGCCTGGCTGACCGCCCAACGACCCCCGCCCATTGACGTCAATAATGACGTAT
GTTCCCATAGTAACGCCAATAGGGACTTTCCATTGACGTCAATGGGTGGAGTATTT
ACGGTAAACTGCCCACTTGGCAGTACATCAAGTGTATCATATGCCAAGTACGCCCC
CTATTGACGTCAATGACGGTAAATGGCCCGCCTGGCATTATGCCCAGTACATGACC
TTATGGGACTTTCCTACTTGGCAGTACATCTACGTATTAGTCATCGCTATTACCATG
GTGATGCGGTTTTGGCAGTACATCAATGGGCGTGGATAGCGGTTTGACTCACGGGG
ATTTCCAAGTCTCCACCCCATTGACGTCAATGGGAGTTTGTTTTGGCACCAAAATCA
ACGGGACTTTCCAAAATGTCGTAACAACTCCGCCCCATTGACGCAAATGGGCGGTA
GGCGTGTACGGTGGGAGGTCTATATAAGCAGCGCGTTTTGCCTGTACTGGGTCTCT
CTGGTTAGACCAGATCTGAGCCTGGGAGCTCTCTGGCTAACTAGGGAACCCACTGC
TTAAGCCTCAATAAAGCTTGCCTTGAGTGCTTCAAGTAGTGTGTGCCCGTCTGTTGT
GTGACTCTGGTAACTAGAGATCCCTCAGACCCTTTTAGTCAGTGTGGAAAATCTCT
AGCAGTGGCGCCCGAACAGGGACTTGAAAGCGAAAGGGAAACCAGAGGAGCTCTC
TCGACGCAGGACTCGGCTTGCTGAAGCGCGCACGGCAAGAGGCGAGGGGCGGCGA
CTGGTGAGTACGCCAAAAATTTTGACTAGCGGAGGCTAGAAGGAGAGAGATGGGT
GCGAGAGCGTCAGTATTAAGCGGGGGAGAATTAGATCGCGATGGGAAAAAATTCG
GTTAAGGCCAGGGGGAAAGAAAAAATATAAATTAAAACATATAGTATGGGCAAGC
AGGGAGCTAGAACGATTCGCAGTTAATCCTGGCCTGTTAGAAACATCAGAAGGCT
GTAGACAAATACTGGGACAGCTACAACCATCCCTTCAGACAGGATCAGAAGAACT
TAGATCATTATATAATACAGTAGCAACCCTCTATTGTGTGCATCAAAGGATAGAGA
TAAAAGACACCAAGGAAGCTTTAGACAAGATAGAGGAAGAGCAAAACAAAAGTA
AGACCACCGCACAGCAAGCGGCCGCTGATCTTCAGACCTGGAGGAGGAGATATGA
GGGACAATTGGAGAAGTGAATTATATAAATATAAAGTAGTAAAAATTGAACCATT
AGGAGTAGCACCCACCAAGGCAAAGAGAAGAGTGGTGCAGAGAGAAAAAAGAGC
AGTGGGAATAGGAGCTTTGTTCCTTGGGTTCTTGGGAGCAGCAGGAAGCACTATGG
GCGCAGCGTCAATGACGCTGACGGTACAGGCCAGACAATTATTGTCTGGTATAGTG
CAGCAGCAGAACAATTTGCTGAGGGCTATTGAGGCGCAACAGCATCTGTTGCAACT
CACAGTCTGGGGCATCAAGCAGCTCCAGGCAAGAATCCTGGCTGTGGAAAGATAC
CTAAAGGATCAACAGCTCCTGGGGATTTGGGGTTGCTCTGGAAAACTCATTTGCAC
CACTGCTGTGCCTTGGAATGCTAGTTGGAGTAATAAATCTCTGGAACAGATTTGGA
ATCACACGACCTGGATGGAGTGGGACAGAGAAATTAACAATTACACAAGCTTAAT
ACACTCCTTAATTGAAGAATCGCAAAACCAGCAAGAAAAGAATGAACAAGAATTA
TTGGAATTAGATAAATGGGCAAGTTTGTGGAATTGGTTTAACATAACAAATTGGCT
GTGGTATATAAAATTATTCATAATGATAGTAGGAGGCTTGGTAGGTTTAAGAATAG
TTTTTGCTGTACTTTCTATAGTGAATAGAGTTAGGCAGGGATATTCACCATTATCGT
TTCAGACCCACCTCCCAACCCCGAGGGGACCCGACAGGCCCGAAGGAATAGAAGA
AGAAGGTGGAGAGAGAGACAGAGACAGATCCATTCGATTAGTGAACGGATCGGCA
CTGCGTGCGCCAATTCTGCAGACAAATGGCAGTATTCATCCACAATTTTAAAAGAA
AAGGGGGGATTGGGGGGTACAGTGCAGGGGAAAGAATAGTAGACATAATAGCAA
CAGACATACAAACTAAAGAATTACAAAAACAAATTACAAAAATTCAAAATTTTCG
GGTTTATTACAGGGACAGCAGAGATCCAGTTTGGTTAATTAAgggtgcagcggcctccgcgcc
gggttttggcgcctcccgcgggcgcccccctcctcacggcgagcgctgccacgtcagacgaagggcgcaggagcgttcctgatccttc
cgcccggacgctcaggacagcggcccgctgctcataagactcggccttagaaccccagtatcagcagaaggacattttaggacgggac
ttgggtgactctagggcactggttttctttccagagagcggaacaggcgaggaaaagtagtcccttctcggcgattctgcggagggatctc
cgtggggcggtgaacgccgatgattatataaggacgcgccgggtgtggcacagctagttccgtcgcagccgggatttgggtcgcggttc
ttgtttgtggatcgctgtgatcgtcacttggtgagttgcgggctgctgggctggccggggctttcgtggccgccgggccgctcggtgggac
ggaagcgtgtggagagaccgccaagggctgtagtctgggtccgcgagcaaggttgccctgaactgggggttggggggagcgcacaa
aatggcggctgttcccgagtcttgaatggaagacgcttgtaaggcgggctgtgaggtcgttgaaacaaggtggggggcatggtgggcg
gcaagaacccaaggtcttgaggccttcgctaatgcgggaaagctcttattcgggtgagatgggctggggcaccatctggggaccctgac
gtgaagtttgtcactgactggagaactcgggtttgtcgtctggttgcgggggcggcagttatgcggtgccgttgggcagtgcacccgtacc
tttgggagcgcgcgcctcgtcgtgtcgtgacgtcacccgttctgttggcttataatgcagggtggggccacctgccggtaggtgtgcggta
ggcttttctccgtcgcaggacgcagggttcgggcctagggtaggctctcctgaatcgacaggcgccggacctctggtgaggggaggga
taagtgaggcgtcagtttctttggtcggttttatgtacctatcttcttaagtagctgaagctccggttttgaactatgcgctcggggttggcgagt
gtgttttgtgaagttttttaggcaccttttgaaatgtaatcatttgggtcaatatgtaattttcagtgttagactagtaaagcttctgcaggtcgact
ctagaaaattgtccgctaaattctggccgtttttggcttttttgttagacaGGATCCCCGGGTACCGGTCGCCACCAT
GGTGAGCAAGGGCGAGGAGCTGTTCACCGGGGTGGTGCCCATCCTGGTCGAGCTG
GACGGCGACGTAAACGGCCACAAGTTCAGCGTGTCCGGCGAGGGCGAGGGCGATG
CCACCTACGGCAAGCTGACCCTGAAGTTCATCTGCACCACCGGCAAGCTGCCCGTG
CCCTGGCCCACCCTCGTGACCACCCTGACCTACGGCGTGCAGTGCTTCAGCCGCTA
CCCCGACCACATGAAGCAGCACGACTTCTTCAAGTCCGCCATGCCCGAAGGCTACG
TCCAGGAGCGCACCATCTTCTTCAAGGACGACGGCAACTACAAGACCCGCGCCGA
GGTGAAGTTCGAGGGCGACACCCTGGTGAACCGCATCGAGCTGAAGGGCATCGAC
TTCAAGGAGGACGGCAACATCCTGGGGCACAAGCTGGAGTACAACTACAACAGCC
ACAACGTCTATATCATGGCCGACAAGCAGAAGAACGGCATCAAGGTGAACTTCAA
GATCCGCCACAACATCGAGGACGGCAGCGTGCAGCTCGCCGACCACTACCAGCAG
AACACCCCCATCGGCGACGGCCCCGTGCTGCTGCCCGACAACCACTACCTGAGCAC
CCAGTCCGCCCTGAGCAAAGACCCCAACGAGAAGCGCGATCACATGGTCCTGCTG
GAGTTCGTGACCGCCGCCGGGATCACTCTCGGCATGGACGAGCTGTACATGTCCAA
GTCATTCCAGCAGTCATCTCTCAGTAGGGACTCACAGGGTCATGGGCGTGACCTGT
CTGCGGCAGGAATAGGCCTTCTTGCTGCTGCTACCCAGTCTTTAAGTATGCCAGCA
TCTCTTGGAAGGATGAACCAGGGTACTGCACGCCTTGCTAGTTTAATGAATCTTGG
AATGAGTTCTTCATTGAATCAACAAGGAGCTCATAGTGCACTGTCTTCTGCTAGTA
CTTCTTCCCATAATTTGCAGTCTATATTTAACATTGGAAGTAGAGGTCCACTCCCTT
TATCTTCTCAACACCGTGGAGATGCAGACCAGGCCAGTAACATTTTGGCCAGCTTT
GGTCTGTCTGCTAGAGACTTAGATGAACTGAGTCGTTATCCAGAGGACAAGATTAC
TCCTGAGAATTTGCCCCAAATCCTTCTACAGCTTAAAAGGAGGAGAACTGAAGAAG
GCCCTACCTTGAGTTATGGTAGAGATGGCAGATCTGCTACACGGGAGCCACCATAC
AGAGTACCTAGGGATGATTGGGAAGAAAAAAGGCACTTTAGAAGAGATAGTTTTG
ATGATCGTGGTCCTAGTCTCAACCCAGTGCTTGATTATGACCATGGAAGTCGTTCTC
AAGAATCTGGTTATTATGACAGAATGGATTATGAAGATGACAGATTAAGAGATGG
AGAAAGGTGTAGGGATGATTCTTTTTTTGGTGAGACCTCGCATAACTATCATAAAT
TTGACAGTGAGTATGAGAGAATGGGACGTGGTCCTGGCCCCTTACAAGAGAGATCT
CTCTTTGAGAAAAAGAGAGGCGCTCCTCCAAGTAGCAATATTGAAGACTTCCATGG
ACTCTTACCGAAGGGTTATCCCCATCTGTGCTCTATATGTGATTTGCCAGTTCATTC
TAATAAGGAGTGGAGTCAACATATCAATGGAGCAAGTCACAGTCGTCGATGCCAG
CTTCTTCTTGAAATCTACCCAGAATGGAATCCTGACAATGATACAGGACACACAAT
GGGTGATCCATTCATGTTGCAGCAGTCTACAAATCCAGCACCAGGAATTCTGGGAC
CTCCACCTCCCTCATTTCATCTTGGGGGACCAGCAGTTGGACCAAGAGGAAATCTG
GGTGCTGGAAATGGAAACCTGCAAGGACCTAGACACATGCAGAAAGGCAGAGTGG
AAACTAGCAGAGTTGTTCACATCATGGATTTTCAACGAGGGAAAAACTTGAGATAC
CAGCTATTACAGCTGGTAGAACCATTTGGAGTCATTTCAAATCATCTGATTCTAAAT
AAAATTAATGAGGCATTTATTGAAATGGCAACCACAGAGGATGCTCAGGCCGCAG
TGGATTATTACACAACCACACCAGCGTTAGTATTTGGCAAGCCAGTGAGAGTTCAT
TTATCCCAGAAGTATAAAAGAATAAAGAAACCTGAAGGAAAGCCAGATCAGAAGT
TTGATCAAAAGCAAGAGCTTGGACGTGTGATACATCTCAGCAATTTGCCGCATTCT
GGCTATTCTGATAGTGCTGTTCTCAAGCTTGCTGAGCCTTATGGGAAAATAAAGAA
TTACATATTGATGAGGATGAAAAGTCAGGCTTTTATTGAGATGGAGACAAGAGAA
GATGCAATGGCAATGGTTGACCATTGTTTGAAAAAAGCCCTTTGGTTTCAGGGGAG
ATGTGTGAAGGTTGACCTGTCTGAGAAATATAAAAAACTGGTTCTGAGGATTCCAA
ACAGAGGCATTGATTTACTGAAAAAAGATAAATCCCGAAAAAGATCTTACTCTCCA
GATGGCAAAGAATCTCCAAGTGATAAGAAATCCAAAACTGATGGTTCCCAGAAGA
CTGAGAGTTCAACCGAAGGTAAAGAACAAGAAGAGAAGTCCGGTGAAGATGGTGA
GAAAGACACAAAGGATGACCAGACAGAGCAGGAACCTAATATGCTTCTTGAATCT
GAAGATGAGCTACTTGTAGATGAAGAAGAAGCAGCAGCACTGCTAGAAAGTGGCA
GTTCAGTGGGAGACGAGACCGATCTTGCTAATTTAGGTGATGTGGCTTCTGATGGG
AAAAAGGAACCATCAGATAAAGCTGTGAAAAAAGATGGAAGTGCTTCAGCAGCAG
CAAAGAAAAAGCTTAAAAAGGTGGACAAGATCGAGGAACTTGATCAAGAAAACG
AAGCAGCGTTGGAAAATGGAATTAAAAATGAGGAAAACACAGAACCAGGTGCTGA
ATCTTCTGAGAACGCTGATGATCCCAACAAAGATACAAGTGAAAACGCAGATGGT
CAAAGTGATGAGAACAAGGACGACTATACAATCCCAGATGAGTATAGAATTGGAC
CATATCAGCCCAATGTTCCTGTTGGTATAGACTATGTGATACCTAAAACAGGGTTTT
ACTGTAAGCTGTGTTCACTCTTTTATACAAATGAAGAAGTTGCAAAGAATACTCAT
TGCAGCAGCCTTCCTCATTATCAGAAATTAAAGAAATTTCTGAATAAATTGGCAGA
AGAACGCAGACAGAAGAAGGAAACTTAAAGTAAAGCGGCCGCGACTCTAGAATTC
GATATCAAGCTTATCGATAATCAACCTCTGGATTACAAAATTTGTGAAAGATTGAC
TGGTATTCTTAACTATGTTGCTCCTTTTACGCTATGTGGATACGCTGCTTTAATGCCT
TTGTATCATGCTATTGCTTCCCGTATGGCTTTCATTTTCTCCTCCTTGTATAAATCCT
GGTTGCTGTCTCTTTATGAGGAGTTGTGGCCCGTTGTCAGGCAACGTGGCGTGGTG
TGCACTGTGTTTGCTGACGCAACCCCCACTGGTTGGGGCATTGCCACCACCTGTCA
GCTCCTTTCCGGGACTTTCGCTTTCCCCCTCCCTATTGCCACGGCGGAACTCATCGC
CGCCTGCCTTGCCCGCTGCTGGACAGGGGCTCGGCTGTTGGGCACTGACAATTCCG
TGGTGTTGTCGGGGAAATCATCGTCCTTTCCTTGGCTGCTCGCCTGTGTTGCCACCT
GGATTCTGCGCGGGACGTCCTTCTGCTACGTCCCTTCGGCCCTCAATCCAGCGGAC
CTTCCTTCCCGCGGCCTGCTGCCGGCTCTGCGGCCTCTTCCGCGTCTTCGCCTTCGC
CCTCAGACGAGTCGGATCTCCCTTTGGGCCGCCTCCCCGCATCGATACCGTCGACC
TCGAGACCTAGAAAAACATGGAGCAATCACAAGTAGCAATACAGCAGCTACCAAT
GCTGATTGTGCCTGGCTAGAAGCACAAGAGGAGGAGGAGGTGGGTTTTCCAGTCA
CACCTCAGGTACCTTTAAGACCAATGACTTACAAGGCAGCTGTAGATCTTAGCCAC
TTTTTAAAAGAAAAGGGGGGACTGGAAGGGCTAATTCACTCCCAACGAAGACAAG
ATATCCTTGATCTGTGGATCTACCACACACAAGGCTACTTCCCTGATTGGCAGAAC
TACACACCAGGGCCAGGGATCAGATATCCACTGACCTTTGGATGGTGCTACAAGCT
AGTACCAGTTGAGCAAGAGAAGGTAGAAGAAGCCAATGAAGGAGAGAACACCCG
CTTGTTACACCCTGTGAGCCTGCATGGGATGGATGACCCGGAGAGAGAAGTATTAG
AGTGGAGGTTTGACAGCCGCCTAGCATTTCATCACATGGCCCGAGAGCTGCATCCG
GACTGTACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTCAAGTAG
TGTGTGCCCGTCTGTTGTGTGACTCTGGTAACTAGAGATCCCTCAGACCCTTTTAGT
CAGTGTGGAAAATCTCTAGCAGGGCCCGTTTAAACCCGCTGATCAGCCTCGACTGT
GCCTTCTAGTTGCCAGCCATCTGTTGTTTGCCCCTCCCCCGTGCCTTCCTTGACCCTG
GAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGGCTT
CTGAGGCGGAAAGAACCAGCTGGGGCTCTAGGGGGTATCCCCACGCGCCCTGTAG
CGGCGCATTAAGCGCGGCGGGTGTGGTGGTTACGCGCAGCGTGACCGCTACACTTG
CCAGCGCCCTAGCGCCCGCTCCTTTCGCTTTCTTCCCTTCCTTTCTCGCCACGTTCGC
CGGCTTTCCCCGTCAAGCTCTAAATCGGGGGCTCCCTTTAGGGTTCCGATTTAGTGC
TTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATA
GTGGACTCTTGTTCCAAACTGGAACAACACTCAACCCTATCTCGGTCTATTCTTTTG
ATTTATAAGGGATTTTGCCGATTTCGGCCTATTGGTTAAAAAATGAGCTGATTTAAC
AAAAATTTAACGCGAATTAATTCTGTGGAATGTGTGTCAGTTAGGGTGTGGAAAGT
CCCCAGGCTCCCCAGCAGGCAGAAGTATGCAAAGCATGCATCTCAATTAGTCAGCA
ACCAGGTGTGGAAAGTCCCCAGGCTCCCCAGCAGGCAGAAGTATGCAAAGCATGC
ATCTCAATTAGTCAGCAACCATAGTCCCGCCCCTAACTCCGCCCATCCCGCCCCTA
ACTCCGCCCAGTTCCGCCCATTCTCCGCCCCATGGCTGACTAATTTTTTTTATTTATG
CAGAGGCCGAGGCCGCCTCTGCCTCTGAGCTATTCCAGAAGTAGTGAGGAGGCTTT
TTTGGAGGCCTAGGCTTTTGCAAAAAGCTCCCGGGAGCTTGTATATCCATTTTCGG
ATCTGATCAGCACGTGTTGACAATTAATCATCGGCATAGTATATCGGCATAGTATA
ATACGACAAGGTGAGGAACTAAACCATGGCCAAGTTGACCAGTGCCGTTCCGGTG
CTCACCGCGCGCGACGTCGCCGGAGCGGTCGAGTTCTGGACCGACCGGCTCGGGTT
CTCCCGGGACTTCGTGGAGGACGACTTCGCCGGTGTGGTCCGGGACGACGTGACCC
TGTTCATCAGCGCGGTCCAGGACCAGGTGGTGCCGGACAACACCCTGGCCTGGGTG
TGGGTGCGCGGCCTGGACGAGCTGTACGCCGAGTGGTCGGAGGTCGTGTCCACGA
ACTTCCGGGACGCCTCCGGGCCGGCCATGACCGAGATCGGCGAGCAGCCGTGGGG
GCGGGAGTTCGCCCTGCGCGACCCGGCCGGCAACTGCGTGCACTTCGTGGCCGAGG
AGCAGGACTGACACGTGCTACGAGATTTCGATTCCACCGCCGCCTTCTATGAAAGG
TTGGGCTTCGGAATCGTTTTCCGGGACGCCGGCTGGATGATCCTCCAGCGCGGGGA
TCTCATGCTGGAGTTCTTCGCCCACCCCAACTTGTTTATTGCAGCTTATAATGGTTA
CAAATAAAGCAATAGCATCACAAATTTCACAAATAAAGCATTTTTTTCACTGCATT
CTAGTTGTGGTTTGTCCAAACTCATCAATGTATCTTATCATGTCTGTATACCGTCGA
CCTCTAGCTAGAGCTTGGCGTAATCATGGTCATAGCTGTTTCCTGTGTGAAATTGTT
ATCCGCTCACAATTCCACACAACATACGAGCCGGAAGCATAAAGTGTAAAGCCTG
GGGTGCCTAATGAGTGAGCTAACTCACATTAATTGCGTTGCGCTCACTGCCCGCTTT
CCAGTCGGGAAACCTGTCGTGCCAGCTGCATTAATGAATCGGCCAACGCGCGGGG
AGAGGCGGTTTGCGTATTGGGCGCTCTTCCGCTTCCTCGCTCACTGACTCGCTGCGC
TCGGTCGTTCGGCTGCGGCGAGCGGTATCAGCTCACTCAAAGGCGGTAATACGGTT
ATCCACAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGGCTCCGCC
CCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTG
TTCCGACCCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGT
AACTATCGTCTTGAGTCCAACCCGGTAAGACACGACTTATCGCCACTGGCAGCAGC
CACTGGTAACAGGATTAGCAGAGCGAGGTATGTAGGCGGTGCTACAGAGTTCTTG
AAGTGGTGGCCTAACTACGGCTACACTAGAAGAACAGTATTTGGTATCTGCGCTCT
GCTGAAGCCAGTTACCTTCGGAAAAAGAGTTGGTAGCTCTTGATCCGGCAAACAAA
CCACCGCTGGTAGCGGTGGTTTTTTTGTTTGCAAGCAGCAGATTACGCGCAGAAAA
AAAGGATCTCAAGAAGATCCTTTGATCTTTTCTACGGGGTCTGACGCTCAGTGGAA
CGAAAACTCACGTTAAGGGATTTTGGTCATGAGATTATCAAAAAGGATCTTCACCT
AGATCCTTTTAAATTAAAAATGAAGTTTTAAATCAATCTAAAGTATATATGAGTAA
ACTTGGTCTGACAGTTACCAATGCTTAATCAGTGAGGCACCTATCTCAGCGATCTG
TCTATTTCGTTCATCCATAGTTGCCTGACTCCCCGTCGTGTAGATAACTACGATACG
GGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGGAAGCTAG
AGTAAGTAGTTCGCCAGTTAATAGTTTGCGCAACGTTGTTGCCATTGCTACAGGCA
TCGTGGTGTCACGCTCGTCGTTTGGTATGGCTTCATTCAGCTCCGGTTCCCAACGAT
CAAGGCGAGTTACATGATCCCCCATGTTGTGCAAAAAAGCGGTTAGCTCCTTCGGT
CCTCCGATCGTTGTCAGAAGTAAGTTGGCCGCAGTGTTATCACTCATGGTTATGGC
AGCACTGCATAATTCTCTTACTGTCATGCCATCCGTAAGATGCTTTTCTGTGACTGG
TGAGTACTCAACCAAGTCATTCTGAGAATAGTGTATGCGGCGACCGAGTTGCTCTT
GCCCGGCGTCAATACGGGATAATACCGCGCCACATAGCAGAACTTTAAAAGTGCTC
ATCATTGGAAAACGTTCTTCGGGGCGAAAACTCTCAAGGATCTTACCGCTGTTGAG
ATCCAGTTCGATGTAACCCACTCGTGCACCCAACTGATCTTCAGCATCTTTTACTTT
CACCAGCGTTTCTGGGTGAGCAAAAACAGGAAGGCAAAATGCCGCAAAAAAGGGA
ATAAGGGCGACACGGAAATGTTGAATACTCATACTCTTCCTTTTTCAATATTATTGA
AGCATTTATCAGGGTTATTGTCTCATGAGCGGATACATATTTGAATGTATTTAGAA
AAATAAACAAATAGGGGTTCCGCGCACATTTCCCCGAAAAGTGCCACCTGAC

Total Proteins Extraction and Immunoblotting

HEK293 cells were harvested and lysed in IP buffer [50 mM Tris-HCl pH 7.5; 150 mM NaCl; 1% NP-40; 5 mM EDTA; 5 mM EGTA; protease inhibitors]. Cell extracts were resolved on a 10% or 6% SDS-PAGE acrylamide gels and transferred to nitrocellulose blotting membrane (GE Healthcare Life Sciences) using a wet transfer method. The membranes were blotted with the antibodies indicated in each figure, and bands were visualized using the ECL Western blotting substrate (Thermo Fisher Scientific). Membranes were incubated with the following primary antibodies: anti-FLAG M2 (Sigma-Aldrich F1804), anti-HA.11 (Covance, MMS-101R), anti-MATR3 (Thermo Fisher Scientific, PAS-57720), anti-6xHis (Clontech, 631212), anti-GST (Sigma-Aldrich G1160), anti-tubulin (Sigma, T9026). Secondary antibodies conjugated to horseradish peroxidase (Jackson Immunoresearch) were used at 1:10,000 dilutions (anti-mouse IgG HRP 715-035-150, anti-rabbit IgG HRP, 711-035-152).

Cell Culture

HEK293 and HEK293T cells were grown in DMEM high glucose medium with L-Glutamine and Sodium Pyruvate (EuroClone) supplemented with 10% Fetal Bovine Serum (FBS, Thermo Fisher Scientific) and 1% penicillin/streptomycin (Pen/Strep, Thermo Fisher Scientific).

FSHD and control primary human myoblasts were kindly provided by Dr. Rabi Tawil, the Richard Fields Center for FSHD Research biobank, Department of Neurology, University of Rochester, N.Y., USA (https://www.urmc.rochester.edu/neurology/fields-center.aspx).

Myoblasts were grown in F10 medium (Sigma-Aldrich), supplemented with 20% FBS, 1% Pen-Strep, 10 ng/ml bFGF (Tebu-bio), and 1 μM dexamethasone (Sigma-Aldrich). To induce differentiation, myoblasts were plated at a confluence of 50,000 cells/cm2 and 24 h after seeding, growth medium was replaced by DMEM:F12 (1:1, Sigma-Aldrich) supplemented with 20% knockout serum replacer (KOSR, Thermo Fisher Scientific), 3.151 g/L glucose, 10 mM MEM non-essential amino acids (Thermo Fisher Scientific), 100 mM sodium pyruvate (Thermo Fisher Scientific). Differentiation was carried out for 96 h.

Generation of DUX4 Flp-In T-REx 293 Cell Line

DUX4 coding sequence (NP_001292997.1) (SEQ ID NO:80) was cloned in frame into a pCDNA5/FRT/STREP-HA backbone using the Gateway gene cloning strategy. The plasmid was then co-transfected together with the pOG44 Flp-Recombinase Expression vector (ThermoFisher) into Flp-In T-REx 293 cells (Thermo Fisher Scientific) to generate a STREP-HA-DUX4-inducible cell line, according to the vendor protocol. Cells were then selected using both Blasticidin (Thermo Fisher Scientific) and Hygromycin B (Thermo Fisher Scientific) and resistant clones expanded and used for the experiments. STREP-HA DUX4 Flp-In T-REx 293 cells were growth in DMEM 10% Tetracycline-Free FBS (GIBCO) supplemented with 1% Pen/Strep (Thermo Fisher Scientific) and STREP-HA DUX4 expression was induced upon doxycycline (MERCK) administration. The parental Flp-In T-REx 293 cells were grown in parallel and used as negative control.

Affinity Purification from Nuclear Extracts

For the STREP-HA affinity purifications of parental and STREP-HA DUX4 Flp-In T-REx 293 cells, 1 μg/mL doxycycline was added to the cell culture media for 8 h prior to cell harvesting. Nuclear protein extraction and STREP-HA affinity purification was conducted as previously described (83). Briefly, cells were lysed in buffer N (300 mM sucrose, 10 mM HEPES pH 7.9, 10 mM KCl, 0.1 mM EDTA, 0.1 mM EGTA, 0.1 mM DTT, 0.75 mM spermidine, 0.15 mM spermine, 0.1% Nonidet P-40, 50 mM NaF, protease inhibitors) for 5 min on ice and then centrifuged (500 g for 5 min) to separate the nuclear pellet from the supernatant containing the cytoplasmic fraction. The nuclear pellet was then washed with buffer N and resuspended in buffer C420 (20 mM HEPES pH 7.9, 420 mM NaCl, 25% glycerol, 1 mM EDTA, 1 mM EGTA, 0.1 mM DTT, 50 mM NaF, protease inhibitors), vortexed briefly, and shaken vigorously for 30 min. Samples were then centrifuged for 1 h at 100000 g and the supernatant containing the soluble nuclear proteins were collected and quantified by Bradford assay. Hundred milligrams of nuclear extracts were used for the two-step affinity purifications. Prior to purification, nuclear extracts were adjusted to 150 mM NaCl with HEPES buffer (20 mM HEPES, 50 mM NaF, protease inhibitors) and brought to the final volume of 7.5 mL with TNN-HS buffer (50 mM HEPES pH 8.0, 150 mM NaCl, 5 mM EDTA, 0.5% NP-40, 50 mM NaF, protease inhibitors). Samples were then incubated for 20 min at 4° C. on a rotating wheel with RNase A, Benzonase and avidin to remove nucleic acids and to saturate endogenously biotinylated proteins, respectively. Next, the nuclear extracts were incubated overnight at 4° C. on a rotating wheel and the day after the flow-through was removed, beads were washed 3 times with TNN-HS and proteins bound to the beads were eluted with 3 consecutive incubations with 300 μl of 2.5 mM biotin in TNN-HS buffer. The biotin eluate was subsequently incubated with anti-HA-agarose beads (MERCK) for 2 h at 4° C. on a rotating wheel. Samples were centrifuged for 3 min at 300 g and the beads were washed 3 times with TNN-HS buffer. Another two washing steps with TNN-HS buffer without detergent and inhibitors were performed to remove traces of detergent that are detrimental to LC-MS analysis. Finally, proteins were eluted in 50 μl 2% SDS buffer, boiled 5 min at 95° C. and centrifuged 3 min at 300 g. The supernatant containing the eluted proteins were processed according to the Filter Aided Sample Preparation (FASP) protocol (84) to remove the SDS prior trypsin digestion using EMD Millipore Amicon Ultra-0.5 Centrifugal Filter Units (Thermo Fisher Scientific). Within the procedure, samples were reduced with Dithiothreitol (DTT), alkylated with Iodocetamide and digested with Trypsin sequencing grade (MERCK), as previously described (85).

MS Analysis and Protein Identification

Tryptic peptides were desalted using StageTip C18 (Thermo Fisher Scientific) and analyzed by nLC-MS/MS using a Q-Exactive mass spectrometer (Thermo Fisher Scientific) equipped with a nano-electrospray ion source (Proxeon Biosystems) and a nanoUPLC Easy nLC 1000 (Proxeon Biosystems). Peptide separations occurred on a homemade (75 μm i.d., 12 cm long) reverse phase silica capillary column, packed with 1.9-μm ReproSil-Pur 120 C18-AQ (Dr. Maisch GmbH, Germany). A gradient of eluents A (distilled water with 0.1% v/v formic acid) and B (acetonitrile with 0.1% v/v formic acid) was used to achieve separation (300 nl/min flow rate), from 5% B to 50% B in 88 minutes. Full scan spectra were acquired with the lock-mass option, resolution set to 70,000 and mass range from m/z 300 to 2000 Da. The ten most intense doubly and triply charged ions were selected and fragmented.

To quantify proteins, the raw data were loaded into the MaxQuant (86) software version 1.5.2.8 to search the human_proteome 20180425 (93,606 sequences; 37,037,628 residues). Searches were performed with the following settings: trypsin as proteolytic enzyme; 3 missed cleavages allowed; carbamidomethylation on cysteine as fixed modification; protein N-terminus-acetylation, methionine oxidation as variable modifications. Peptides and proteins were accepted with a FDR less than 1%. Label-free protein quantification was based on the spectral counts considering only proteins identified with minimum two peptides in any STREP-HA DUX4 purification. The following filtering criterion was used to discriminate the specific interactors of STREP-HA DUX4: the protein must be detected in all the three STREP-HA DUX4 biological replicates with spectral counts fold enrichment >4 with respect of the control affinity purifications performed on parental cells.

Transfection of siRNA and Plasmids

Transfection of siRNA was performed by using Lipofectamine 3000 Transfection Reagent (Thermo Fisher Scientific), following the manufacturer's instructions. For FSHD muscle cells, siRNAs were delivered 24 h after induction of differentiation. Medium was replaced the day after transfection and myotubes were harvested 96 h after induction of differentiation. List of siRNAs used in this study is provided in Table S2.

Plasmids were delivered by using Lipofectamine LTX Reagent with PLU Reagent (Thermo Fisher Scientific), following the manufacturer's instructions.

When transfection of both siRNA and plasmid was required, HEK293 cells were reverse-transfected with siRNA by using Lipofectamine 3000 Transfection Reagent and the day after they were transfected with DUX4 construct by using Lipofectamine LTX Reagent with PLUS Reagent, following manufacturer's instructions. Cells were harvested 48 h after the last transfection.

Lentiviral Production and Transduction

For MATR3 overexpression experiments in FSHD muscle cells, lentiviral particles were produced in HEK293T cells. Briefly, 4×10⁶ HEK293T cells were seeded in 10 cm dish plate and the day after transfected with 6.5 μg of lentiviral vectors carrying either GFP alone (pFUGW:GFP, a kind gift from Shanahan CM lab) or MATR3 cDNA (NM_199189.2) (SEQ ID NO:94)

| | |
|---|---|
| 779 | at |
| 781 | gtccaagtca ttccagcagt catctctcag tagggactca |
| | cagggtcatg ggcgtgacct |
| 841 | gtctgcggca ggaataggcc ttcttgctgc tgctacccag |
| | tctttaagta tgccagcatc |
| 901 | tcttggaagg atgaaccagg gtactgcacg ccttgctagt |
| | ttaatgaatc ttggaatgag |
| 961 | ttcttcattg aatcaacaag gagctcatag tgcactgtct |
| | tctgctagta cttcttccca |
| 1021 | taatttgcag tctatattta acattggaag tagaggtcca |
| | ctccctttat cttctcaaca |
| 1081 | ccgtggagat gcagaccagg ccagtaacat tttggccagc |
| | tttggtctgt ctgctagaga |
| 1141 | cttagatgaa ctgagtcgtt atccagagga caagattact |
| | cctgagaatt tgccccaaat |
| 1201 | ccttctacag cttaaaagga ggagaactga agaaggccct |
| | accttgagtt atggtagaga |
| 1261 | tggcagatct gctacacggg agccaccata cagagtacct |
| | agggatgatt gggaagaaaa |
| 1321 | aaggcacttt agaagagata gttttgatga tcgtggtcct |
| | agtctcaacc cagtgcttga |
| 1381 | ttatgaccat ggaagtcgtt ctcaagaatc tggttattat |
| | gacagaatgg attatgaaga |
| 1441 | tgacagatta agagatggag aaaggtgtag ggatgattct |
| | ttttttggtg agacctcgca |
| 1501 | taactatcat aaatttgaca gtgagtatga gagaatggga |
| | cgtggtcctg gccccttaca |
| 1561 | agagagatct ctctttgaga aaaagagagg cgctcctcca |
| | agtagcaata ttgaagactt |
| 1621 | ccatggactc ttaccgaagg gttatcccca tctgtgctct |
| | atatgtgatt tgccagttca |
| 1681 | ttctaataag gagtggagtc aacatatcaa tggagcaagt |
| | cacagtcgtc gatgccagct |
| 1741 | tcttcttgaa atctacccag aatggaatcc tgacaatgat |
| | acaggacaca caatgggtga |
| 1801 | tccattcatg ttgcagcagt ctacaaatcc agcaccagga |
| | attctgggac ctccacctcc |
| 1861 | ctcatttcat cttgggggac cagcagttgg accaagagga |
| | aatctgggtg ctggaaatgg |
| 1921 | aaacctgcaa ggacctagac acatgcagaa aggcagagtg |
| | gaaactagca gagttgttca |
| 1981 | catcatggat tttcaacgag ggaaaaactt gagataccag |
| | ctattacagc tggtagaacc |
| 2041 | atttggagtc atttcaaatc atctgattct aaataaaatt |
| | aatgaggcat ttattgaaat |
| 2101 | ggcaaccaca gaggatgctc aggccgcagt ggattattac |
| | acaaccacac cagcgttagt |
| 2161 | atttggcaag ccagtgagag ttcatttatc ccagaagtat |
| | aaaagaataa agaaacctga |
| 2221 | aggaaagcca gatcagaagt ttgatcaaaa gcaagagctt |
| | ggacgtgtga tacatctcag |
| 2281 | caatttgccg cattctggct attctgatag tgctgttctc |
| | aagcttgctg agccttatgg |
| 2341 | gaaaataaag aattacatat tgatgaggat gaaaagtcag |
| | gcttttattg agatggagac |
| 2401 | aagagaagat gcaatggcaa tggttgacca ttgtttgaaa |
| | aaagcccttt ggtttcaggg |
| 2461 | gagatgtgtg aaggttgacc tgtctgagaa atataaaaaa |
| | ctggttctga ggattccaaa |
| 2521 | cagaggcatt gatttactga aaaaagataa atcccgaaaa |
| | agatcttact ctccagatgg |
| 2581 | caaagaatct ccaagtgata agaaatccaa aactgatggt |
| | tcccagaaga ctgagagttc |
| 2641 | aaccgaaggt aaagaacaag aagagaagtc cggtgaagat |
| | ggtgagaaag acacaaagga |
| 2701 | tgaccagaca gagcaggaac ctaatatgct tcttgaatct |
| | gaagatgagc tacttgtaga |
| 2761 | tgaagaagaa gcagcagcac tgctagaaag tggcagttca |
| | gtgggagacg agaccgatct |
| 2821 | tgctaattta ggtgatgtgg cttctgatgg gaaaaaggaa |
| | ccatcagata aagctgtgaa |
| 2881 | aaaagatgga agtgcttcag cagcagcaaa gaaaaagctt |
| | aaaaaggtgg acaagatcga |
| 2941 | ggaacttgat caagaaaacg aagcagcgtt ggaaaatgga |
| | attaaaaatg aggaaaacac |
| 3001 | agaaccaggt gctgaatctt ctgagaacgc tgatgatccc |
| | aacaaagata caagtgaaaa |
| 3061 | cgcagatggt caaagtgatg agaacaagga cgactataca |
| | atcccagatg agtatagaat |
| 3121 | tggaccatat cagcccaatg ttcctgttgg tatagactat |
| | gtgataccta aaacagggtt |
| 3181 | ttactgtaag ctgtgttcac tcttttatac aaatgaagaa |
| | gttgcaaaga atactcattg |
| 3241 | cagcagcctt cctcattatc agaaattaaa gaaatttctg |
| | aataaattgg cagaagaacg |
| 3301 | cagacagaag aaggaaactt aa |

fused to GFP (pFUGW:GFP-MATR3), 6 μg of pCMV-dR8.91 plasmid and 0.65 μg of pCMV-VSV-G plasmid. Three virus collections were performed for each construct. Viral preparation was concentrated of 100 fold by ultra-centrifuging the suspension at 20,000 rpm for 2 h at 4° C. and then resuspended in 250 μl of Opti-MEM Reduced Serum Medium (Thermo Fisher Scientific) and stored at −80° C.

FSHD muscle cells in 12 wells plates, 24 h after induction of differentiation, were transduced with 65 μl of concentrated virus in differentiation medium containing 8 μg/ml polybrene and harvested 72 h after infection.

RNA Extraction, Reverse Transcription and Quantitative Real-Time PCR

Total RNA was extracted from HEK293 cells, healthy or FSHD myotubes using PureLink RNA Mini Kit (Thermo Fisher Scientific), following the manufacturer's instructions. Briefly, cells were lysed in Lysis buffer supplemented with 2-mercaptoethanol and homogenized by passing the lysate 5-10 times through a 21-gauge syringe needle. After adding one volume of 70% ethanol, lysate was loaded onto the spin cartridge provided by the kit, washed, treated with DNAseI (PureLink DNase Set, Thermo Fisher Scientific) and eluted in RNAse-free water. cDNA synthesis was performed using SuperScript III First-Strand Synthesis System (Thermo Fisher Scientific), following the manufacturer's instructions.

Quantitative real-time PCR (qPCR) was performed with SYBR GreenER qPCR SuperMix Universal (Thermo Fisher Scientific) using CFX96 Real-Time PCR Detection System (Bio-Rad). Primers used for RT-qPCR are listed in Table S2. Relative quantification was performed using the ΔΔCt method. Specific details of each data set are provided in the Figure legends.

Immunofluorescence of DUX4

FSHD muscle cells were plated on coverslips and differentiated for 96 h. Myotubes were fixed in 4% paraformaldehyde (Societa Italiana Chimici) in PBS for 10 min at room temperature. After 3 washes in PBS, cells were permeabilized in 1% Triton X-100 (Sigma-Aldrich) in PBS for 15 min at room temperature and blocked in 2% goat serum, 2% horse serum, 2% BSA, 0.1% Triton-X100 in PBS, for 45 min at room temperature. Cells were then incubated with 1:100 anti-DUX4 E5-5 antibody (rabbit; Abcam ab124699) at 37° C. in a humid chamber overnight. After 3 washes in PBS, cells were incubated with fluorescent-conjugated Alexa 555 goat anti-rabbit secondary antibody (Molecular Probes A-27039) for 45 min at RT and rinsed again in PBS. Counterstaining with Hoechst 33342 was performed for 10 min at RT and after 3 washes in PBS coverslips were mounted and imaged by a fluorescence microscope.

Myotube Morphology Analysis

For myotube morphology analysis, cells were fixed in 4% PFA, permeabilized with 1% TritonX-100 in PBS and immunostained with mouse MF20 antibody (Developmental Studies Hybridoma Bank; dilution 1:2) followed by Alexa Fluor 488 goat anti-mouse (Molecular Probes; dilution 1:500) and Hoechst (1 mg/ml, Sigma-Aldrich; dilution 1:2.000). Cells were imaged using fluorescence microscope (Observer.Z1, Zeiss). Fusion index analysis was performed with ImageJ software by counting the number of nuclei included or not into myotubes (myosin positive syncytia containing at least 3 nuclei). Three independent differentiation experiments were performed and 5 fields per well were analyzed.

Cell Viability and Apoptotic Assay

Cell viability in HEK293 and STREP-HA DUX4 Flp-In T-REx 293 cells was measured using the CellTiter-Glo luminescent assay (Promega), based on quantitation of ATP, following the manufacturer's instructions. Apoptosis was measured through Caspase-Glo 3/7 luminescent assay (Promega), based on quantification of caspase-3 and -7 activity, following the manufacturer's instructions. Briefly, 100 μl of CellTiter-Glo or Caspase-Glo 3/7 Reagent respectively was added to 100 μl of cell suspension in a white 96-well plate. The plate was incubated for 40 minutes at room temperature in the dark and then luminescence was quantified by Wallac 1420 multilabel Victor3 microplate reader (Perkin Elmer). These assays were performed in STREP-HA DUX4 Flp-In T-REx 293 cells 24 h after doxycycline administration, and in HEK293 cells 48 h after transfection.

For Staurosporine treatment, HEK293 cells were transfected at 90% confluence with FLAG pCMV vector, empty or carrying MATR3 full-length using Lipofectamine LTX with PLUS Reagent (Thermo Fisher Scientific). 404 DMSO (Dimethyl Sulfoxide; Sigma-Aldrich) or 40 μM Staurosporine (Sigma-Aldrich) were added to the cells and incubated for 6 hours at 37° C. Then, cells were collected and the Caspase 3/7 Glo assay was performed as previously described. Apoptotic levels in primary human myotubes were determined using the live imaging system IncuCyte (Essen BioScience). To this end, 50000 cells/cm² were plated in a 12 well plate, differentiation and transduction with lentiviral vectors carrying GFP only or GFP-MATR3 were performed as described above. 24 h after transduction, the differentiation medium was refreshed adding 1 μl/well of Incucyte Caspase 3/7 green Apoptosis assay reagent (Essen BioScience). The plate was placed on an IncuCyte S3 Live-Cell Analysis System and followed for the entire incubation period (72 h). Every 3 hours the system acquired images and confluency and caspase signal were measured using the IncuCyte software (Essen BioScience). Results are expressed as % of apoptotic cells, normalizing the caspase signal over cell confluency.

Strep-Tactin Pull-Down Assays

HEK293T cells were plated on 10 cm cell culture plates and at 90% confluence they were transfected with pTO STREP-HA vector empty or carrying DUX4 full-length or DUX4 dbd using PolyFect Transfection Reagent (QIAGEN).

24 hours after transfection, cells were harvested and nuclear extracts were prepared by lysing the cell membrane with buffer N (300 mM sucrose; 10 mM Hepes pH 7.9; 10 mM KCl; 0.1 mM EDTA; 0.1 mM EGTA; 0.1 mM DTT; 0.75 mM spermidine; 0.15 mM spermine; 0.1% NP-40 substitute; protease inhibitors) followed by extraction of nuclear proteins using buffer C420 (20 mM Hepes pH 7.9, 420 mM NaCl, 25% glycerol, 1 mM EDTA, 1 mM EGTA, 0.1 mM DTT, protease inhibitors). Nuclear extracts were cleared by ultracentrifugation at 100000 g, 4° C. for 1 hour.

The pull-down was performed using 600 μg of nuclear proteins, adding 2 volumes of HEPES buffer (20 mM Hepes pH 8; 50 mM NaF; protease inhibitors) and TNN buffer [50 mM Hepes pH 8.0; 150 mM NaCl; 5 mM EDTA; 0.5% NP-40 substitute; 50 mM NaF; protease inhibitors] to reduce the NaCl concentration. Nuclear extracts were incubated for 1 h at 4° C. with Avidin, Benzonase (Sigma Aldrich) and RNase A (Thermo Fisher) and precleared with Protein G sepharose beads (GE Healthcare Life Sciences) for 1 h at 4° C. with rotation. Protein complexes were obtained by incubation of nuclear extracts with 40 μl of Strep-Tactin sepharose beads (IBA Lifesciences) overnight at 4° C. in gentle rotation. After 3 washes with IP-buffer (50 mM Tris-HCl pH 7.5, 150 mM NaCl, 1% NP-40, 1 mM EDTA, 0.5 mM EGTA), proteins were specifically eluted adding 2.5 mM D-Biotin (Sigma-Aldrich). Input (10% or 0,5%) and bound fractions (10%) of the pull down were analyzed by immunoblotting.

Proximity Ligation Assay

Proximity ligation assay of MATR3 and DUX4 was performed in muscle cells plated on coverslips and differentiated for 96 h, by using Duolink in situ Red kit (Sigma-Aldrich). Myotubes were fixed in 4% paraformaldehyde (Societa Italiana Chimici) in PBS for 10 min at 4° C. After 3 washes in PBS, cells were permeabilized in 1% TritonX-100 (Sigma-Aldrich) in PBS for 15 min at room temperature and blocked in 2% goat serum, 2% horse serum, 2% BSA, 0.1% Triton-X100 in PBS, for 45 min at room temperature. Coverslips were then incubated with primary antibodies, diluted in the antibody diluent provided by the kit, at 37° C. in a humid chamber overnight. Antibodies used are the following: α-MATR3 1:200 (PAS-57720, Thermo Fisher Scientific), α-DUX4 1:50 (P2B1, Sigma-Aldrich). Incubation with PLA probes, Ligation and Polymerase reactions were carried out following the manufacturer's instructions. Coverslips were mounted using Duolink In situ Mounting Medium with DAPI (Sigma-Aldrich) and imaged using a fluorescence microscope.

Recombinant Protein Purification

6xHis-DUX4 dbd,
GST (AAA57092.1)
(SEQ ID NO: 95)

| | |
|---|---|
| 1 | mspilgywki kglvgptrll leyleekyee hlyerdegdk |
| | wrnkkfelgl efpnlpyyid |
| 61 | gdvkltgsma iiryiadkhn mlggcpkera eismlegavl |
| | dirygvsria yskdfetlkv |
| 121 | dflsklpeml kmfedrlchk tylngdhvth pdfmlydald |
| | vvlymdpmcl dafpklvcfk |
| 181 | krieaipqid kylksskyia wplqgwqatf gggdhppksd |
| | lvprgsrras vgspgihrd |

and GST-MATR3 1-287 proteins were expressed in Rosetta2 (DE3) pLys E. coli (Novagen). Bacteria were grown in LB medium supplemented with antibiotics and the induction was made with 1 mM IPTG (Biosciences) for 2 hours at 37° C. for GST-MATR3 1-287 and GST or 20 hours at 18° C. for 6xHis-DUX4 dbd.

Bacterial pellets were resuspended in Lysis Buffer 1 [PBS; 1 mM PMSF; 5 mM 2-mercaptoethanol] or Lysis Buffer 2 [50 mM NaH2PO4, 1M NaCl, pH 8.0, plus protease inhibitors] for GST- and His-tagged proteins, respectively.

Bacteria were sonicated using a Bandelin-sonoplus HD3100 (probe MS73) sonicator [10 cycles of 30 sec on and 30 sec off 80% amplitude], incubated by gentle rotation for 15 minutes at 4° C. after adding Triton X-100 (1%; Sigma), and centrifuged at 15000 rpm at 4° C. for 20 minutes. Supernatants were incubated 1 hour at 4° C. with Glutathione-Agarose beads (Sigma) or His-Select Nickel Affinity gel beads (Sigma), for GST- and His-tagged proteins, respectively. Beads were washed with Lysis Buffer 1 or Lysis Buffer 2 plus 10 mM imidazole (Fluka), for GST- and His-tagged proteins respectively.

Proteins were eluted with elution solution 1 [20 mM glutathione, 100 mM Tris-HCl pH8.0, 120 mM NaCl] for GST-tagged protein and with elution solution 2 [50 mM NaH2PO4, 1M NaCl, 250 mM imidazole (pH 8.0)] for His-tagged proteins.

Proteins were dialyzed overnight at 4° C. in Slide-A-Lyzer dialysis cassettes (Thermo scientific) in Lysis solution. The purification steps and the obtained proteins were analyzed by Coomassie Blue staining loading samples on 10% polyacrylamide gels.

The obtained proteins were supplemented with 50% glycerol and stored at −20° C.

GST Pull Down Assays

For GST pulldown assays, 40 pmol of purified GST only or GST-MATR3 1-287 were immobilized on Glutathione-Agarose beads (Sigma) and incubated with 40 pmol of purified soluble His-DUX4 dbd in 1 ml IPP100 buffer [10 mM Tris-HCl, pH 8, 100 mM NaCl, 0.1% NP-40] supplemented with 2 mM DTT and 1 mM PMSF for 2 hours at 4° C. Beads were washed three times with IPP100 buffer and boiled in Laemmli buffer. Bounded fractions (10%) were loaded on 10% acrylamide gel and immunoblotted using α-GST (Sigma Aldrich, #G1160) and α-6xHis antibody (Clontech, #631212).

Electromobility Shift Assays (EMSA)

For EMSA, 3′ end biotin-labelled oligonucleotides (Table S2) were employed. Complementary oligonucleotides were annealed by mixing together at a 1:1 molar ratio and incubated in boiling water for 5 min. Then, they were slowly cooled to RT.

Twenty femtomoles of probes were incubated with 15 pmol of in vitro purified 6xHis DUX4 dbd and/or GST-MATR3 1-287 proteins in the presence of 1 μg of poly(dI:dC) and 5 μg of salmon sperm DNA in a buffer containing 10 mM Tris, pH 7.5, 0.1 mM EDTA, 1 mM DTT, 100 mM KCl, 3 mM MgCl2, 12% glycerol.

After 2 h incubation at RT, DNA-protein complexes were separated by electrophoresis in 8% (w/v) acrylamide gels formed in 0.5×TBE (Sigma-Aldrich) and run in the same buffer at 5 mA at 4° C. Then, the gels were transferred on a Biodyne nylon membrane (Thermo Scientific) at 380 mA for 45 min at 4° C. The membrane was crosslinked at 120 mJ/cm2 with an UV-Stratalinker and the biotin-labelled DNA was detected by chemiluminescence using the LightShift Chemiluminescent EMSA Kit (Thermo Scientific) following the manufacturer's protocol.

Statistical Analysis

All statistical analyses were performed using GraphPad Prism 5.0a (GraphPad Software, San Diego, USA). Statistical significance was calculated by Student's t-test on at least three independent experiments. P-value: *p<0.05; **p<0.01; ***p<0.001; ****p<0.0001. Details of each dataset are provided in the corresponding figure legend.

### EXAMPLES

### Example 1: Proteomics to Identify the DUX4 Nuclear Interactome

A “guilt by association” approach to characterizing the biological function of a protein based on the identity of its associated factors is widely used in proteomics. To this aim, the inventors fused DUX4 to a streptavidin-binding peptide and a hemagglutinin tag (SH-tag) and generated dox-inducible SH-tagged DUX4 (iSH-DUX4) HEK293 cells. In iSH-DUX4 cells, expression of DUX4 protein is detectable as soon as 4 h after dox administration, DUX4 target genes are upregulated by 8 h, and significant apoptosis is detectable within 24 h of dox treatment (FIG. 1), similar to what has been observed in human muscle cells (24). The inventors performed tandem affinity purification using nuclease-treated and pre-cleared nuclear extracts from dox-induced control and iSH-DUX4 cells under high stringency prior to mass spectrometry (TAP-MS) (25) (26) (27) (28) (29), to reduce nonspecific background binding and identify tight DUX4 interactors. Importantly, in order to avoid possible side effects due to apoptosis, the inventors collected the cells after just 8 h of dox treatment, the minimum time to observe induction of DUX4 targets but far from any sign of detectable cell death. The inventors performed TAP purifications from three independent sets of nuclear extracts. Using stringent statistical evaluation, the inventors found 11 proteins that reproducibly and selectively associate with DUX4 (FIG. 2 and Table 3).

TABLE 3
Proteins associated with DUX4

| | | |
|---|---|---|
| ANAPC7 | Anaphase-promoting | J. Neurosci. 25, |
| | complex subunit 7 | 8115-21 (2005). |
| C1QBP | Complement component | Nucleic Acids Res. 32, |
| | 1 Q subcomponent-binding | 3632-3641 (2004). |
| | protein, mitochondrial | |
| CDC23 | Cell division cycle | Nature 438, |
| | protein 23 homolog | 690-695 (2005). |
| CDC27 | Cell division cycle | Nature 438, |
| | protein 27 homolog | 690-695 (2005). |
| DUX4 | Double homeobox protein 4 | |
| ILF2 | Interleukin enhancer- | EMBO J. 29, |
| | binding factor 2 | 3260-3271 (2010). |
| PRKDC | DNA-dependent protein | EMBO J. 21, |
| | kinase catalytic subunit | 3000-3008 (2002). |
| SMARCC2 | SWI/SNF complex | Cell Rep. 13, |
| | subunit SMARCC2 | 1842-1854 (2015). |
| MATR3 | Matrin-3 | Trends Genet. 2018 |
| | | Jun; 34(6): 404-423 |
| RUVBL1 | RuvB-like 1 | Oncogene 21, 5835- |
| | | 5843 (2002). |
| KPNB1 | Importin subunit beta-1 | Curr. Opin. Struct. Biol. |
| | | 11, 703-15 (2001) |
| SLC25A5 | ADP/ATP translocase 2; | Cancer Res. 66, |
| | ADP/ATP translocase 2, | 9143-52 (2006). |
| | N-terminally processed | |

Beside karyopherin beta 1 (KPNB1), that is likely simply responsible for DUX4 nuclear localization, the remaining proteins have all been involved in regulation of gene expression (Table 3).

### Example 2: Matrin 3 Inhibits DUX4-Induced Toxicity in HEK293 Cells

To determine the relevance of the identified interactors on DUX4-medited cell toxicity, knockdown studies were performed. Despite the inventors' ability to achieve efficient knockdown for all 10 selected interactors individually (FIG. 3A), none showed significant effects on cell viability when depleted in the absence of DUX4 (FIG. 3B). However, to the inventors' surprise, they found that loss of the novel interacting protein, MATR3, significantly increased apoptosis due to DUX4 ectopic expression in HEK293 cells (FIG. 3C). Conversely, MATR3 expression significantly protected cells from DUX4-induced apoptosis (FIG. 3D). No other interactor tested was able to significantly and reproducibly affect DUX4 toxicity (FIG. 3C-D). Importantly, the inventors found that MATR3 did not prevent apoptosis caused by Staurosporine treatment (FIG. 4) indicating that MATR3 is not a general inhibitor of apoptosis. Thus, while the other novel DUX4 interactors here identified could be involved in other aspects of DUX4 biology, MATR3 stood out as the only factor playing a key role in specifically modulating DUX4-induced toxicity.

### Example 3: MATR3 Blocks Induction of DUX4 Targets in HEK293 Cells

MATR3 is a multifunctional protein regulating gene expression at multiple levels (30) (31) (32) (33). Since the ability of DUX4 to activate gene expression is strictly required to execute its toxicity (9) (10) (22) (11) (23) (34), the inventors analyzed the expression of previously identified DUX4 target genes upon MATR3 manipulation. Intriguingly, in HEK293 cells ectopically expressing DUX4 the inventors found a significant increase in the expression of known DUX4 targets following MATR3 knockdown (FIG. 5A). Conversely, DUX4 targets were significantly downregulated in cells expressing MATR3 (FIG. 5B). Notably, DUX4 levels were not affected by MATR3 manipulation in these settings (FIG. 5A-C).

### Example 4: MATR3 Interacts with the DNA-Binding Domain of DUX4

To confirm the interaction between DUX4 and MATR3, the inventors initially performed semi-endogenous Strep-Tactin pull-down experiments using nuclease-treated nuclear extracts. As shown in FIG. 6A, ectopically expressed DUX4 was able to specifically interact with the endogenous MATR3 in HEK293 cells. Intriguingly, a fragment retaining just the DNA-binding domain as the only known DUX4 functional domain was sufficient to interact with MATR3 (FIG. 6A), suggesting that MATR3 interacts with DUX4 DNA-binding domain (DUX4 dbd). Given the fact that the endogenous DUX4 protein is expressed in a minority of FSHD myonuclei (35) (36), co-IP experiments with the endogenous DUX4 in FSHD muscle cells cannot be performed. Therefore, to investigate the interaction between the endogenous DUX4 and MATR3 proteins the inventors used in situ Proximity Ligation Assay (PLA), which allows to visualize protein-protein interaction at single cell level (37). The inventors performed PLA in primary human FSHD muscle cells using antibodies specific for the endogenous DUX4 and MATR3. As shown in FIG. 6B, a positive PLA signal was detectable in FSHD muscle cells (which express endogenous DUX4, FIG. 7). A number of controls support the specificity of the result. For example, the PLA signal requires the presence of DUX4 since it was abolished by DUX4 knockdown (FIG. 6B). Moreover, the PLA signal was absent when omitting the DUX4 and/or MATR3 antibodies (FIG. 8). Collectively, these results demonstrate that the endogenous DUX4 and MATR3 interact in FSHD muscle cells and suggest that MATR3 binds to DUX4 dbd.

### Example 5: MATR3 Inhibits DUX4 Directly by Blocking its Ability to Bind DNA

To map the portion of MATR3 involved in the interaction with DUX4, the inventors compared the activity of full-length MATR3 to that of various MATR3 deletion mutants (FIG. 9A). Intriguingly, the inventors found that a 287-amino acid N-terminal fragment devoid of any known MATR3 functional domain is sufficient to inhibit DUX4-induced cells death to the same extent as full-length MATR3 (FIG. 9B-C). Notably, a MATR3 fragment lacking the first 287 amino acids is unable to significantly protect from DUX4-induced apoptosis (FIG. 9B-C). Hence, these data indicate that the first 287 amino acids of MATR3 are required for the interaction with DUX4. To determine if MATR3 directly binds to DUX4, the inventors performed pull-down experiments using purified, recombinant versions of the above identified DUX4 and MATR3 fragments. FIG. 9D shows that MATR3 fragment 1-287 directly binds to DUX4 dbd. Based on this and because the inventors found that MATR3 blocks the activation of DUX4 targets, the inventors surmised that MATR3 could interfere with the ability of DUX4 to associate with its genomic targets. To test their hypothesis, the inventors performed electrophoretic mobility shift assays with recombinant DUX4 dbd and a labeled oligonucleotide containing DUX4 binding sites in the presence or absence of recombinant MATR3 fragment 1-287. FIG. 9E shows that MATR3 inhibits DNA binding by DUX4. Since MATR3 fragment 1-287 lacks nucleic acids binding domains and is unable to bind DNA (FIG. 9E), present results strongly support a model in which MATR3 acts by blocking DUX4 access to its genomic sites.

### Example 6: MATR3 Inhibits the Expression of DUX4 and DUX4 Targets in FSHD Muscle Cells

The above functional data have been obtained by inducing DUX4 target genes and cell toxicity through DUX4 ectopic expression in HEK293 cells. To support the biological relevance of the present findings, the inventors analyzed the expression of DUX4 targets upon MATR3 loss- and gain-of-function in primary FSHD muscle cells. Notably, MATR3 knockdown caused a significant increase in the expression of DUX4 targets (FIG. 10A). On the contrary, DUX4 targets were downregulated in cells overexpressing MATR3 (FIG. 10B). Surprisingly, the inventors found that the expression of the endogenous DUX4 gene was significantly increased by MATR3 loss-of-function, while MATR3 gain-of-function led to a significant decrease of DUX4 expression in FSHD muscle cells (FIG. 10A-B). In contrast, the inventors found that MATR3 manipulation did not cause any significant alteration in the expression of critical muscle genes such as DYSTROPHIN and MYOGENIN (FIG. 10A-B).

### Example 7: MATR3 Rescues Viability and Myogenic Differentiation of FSHD Muscle Cells

Molecules directly able to protect muscle cells of FSHD patients from DUX4-induced cell death have never been reported. In primary muscle cells of FSHD patients, DUX4 is expressed by a minority of FSHD nuclei (38) (39) (FIG. 7). Hence, only a fraction of FSHD muscle cells undergoes DUX4-induced cell death making it difficult to monitor the efficacy of possible therapeutic treatments. To address this issue, the inventors performed live, real-time, single cell apoptosis assays in large numbers of cells from each culture in an automated and unbiased manner. This approach allows to correlate apoptotic signals with high definition phase contrast images to provide additional biological insight and morphological validation of apoptotic cell death (e.g. cell shrinkage, membrane blebbing, nuclear condensation). Importantly, the inventors used primary FSHD muscle cells which have been reported to display higher than 10% DUX4 positive myonuclei (40) to facilitate the detection of cell death. To test the ability of MATR3 to protect from endogenous DUX4-induced cell death, the inventors transduced primary FSHD muscle cells with a control lentivirus or a lentivirus expressing MATR3 and monitored cell death over time. As shown in FIG. 11A-C, MATR3 expression leads to a significant decrease in FSHD muscle cell death with respect to control infected cells.

DUX4 expression has been shown to interfere with muscle differentiation (41) and muscle cells from FSHD patients display impaired myogenesis (42) (43). The inventors hence wondered if MATR3, by allowing survival of DUX4 expressing cells, is able to rescue the myogenic defects of FSHD muscle cells. To test this, the inventors transduced primary FSHD muscle cells with control or MATR3 lentiviruses and measured their ability to differentiate into myotubes. As shown in FIG. 11D-E, the inventors found that MATR3 expression significantly rescues the myogenic and fusion indexes of FSHD muscle cells allowing for the production of myotubes with a significantly increased number of myonuclei with respect to control infected cells. Collectively, the present results strongly indicate that MATR3 is a natural regulator of DUX4 activity that binds to DUX4 DNA-binding domain preventing activation of its targets and induction of apoptosis.

DUX4 is a homeodomain-containing transcription factor and an important regulator of early human development as it plays an essential role in activating the embryonic genome during the 2- to 8-cell stage of development (Nat. Genet. 49, 925-934 (2017); Nat. Genet. 49, 935-940 (2017); Nat. Genet. 49, 941-945 (2017). As such, it is not typically expressed in healthy somatic cells, and importantly it is silent in healthy skeletal muscle or B-cells.
Facioscapulohumeral muscular dystrophy (FSHD) is one of the most prevalent neuromuscular disorders (Neurology 83, 1056-9 (2014) and leads to significant lifetime morbidity, with up to 25% of patients requiring wheelchair. The disease is characterized by rostro-caudal progressive and asymmetric weakness in a specific subset of muscles. Symptoms typically appear as asymmetric weakness of the facial (facio), shoulder (scapulo), and upper arm (humeral) muscles, and progress to affect nearly all skeletal muscle groups. Extra-muscular manifestations can occur in severe cases, including retinal vasculopathy, hearing loss, respiratory defects, cardiac involvement, mental retardation and epilepsy (Curr. Neurol. Neurosci. Rep. 16, 66 (2016). FSHD is not caused by a classical form of gene mutation that results in loss or altered protein function. Likewise, it differs from typical muscular dystrophies by the absence of sarcolemma defects (J. Cell Biol. 191, 1049-1060 (2010). Instead, FSHD is linked to epigenetic alterations affecting the D4Z4 macrosatellite repeat array in 4q35 and causing chromatin relaxation leading to inappropriate gain of expression of the D4Z4-embedded double homeobox 4 (DUX4) gene (Curr. Neurol. Neurosci. Rep. 16, 66 (2016).
Acute lymphoblastic leukemia (ALL) is the most common cancer among children and the most frequent cause of death from cancer before 20 years of age. Approximately 80-85% of pediatric ALL is of B cell origin and results from arrest at an immature B-precursor cell stage (N. Engl. J. Med. 373, 1541-52 (2015). The underlying etiology of most cases of childhood ALL remains largely unknown. Nevertheless, sentinel chromosomal translocations occur frequently and recurrent ALL-associated translocations can be initiating events that drive leukemogenesis (J. Clin. Oncol. 33, 2938-48 (2015). Importantly, the characterization of gene expression, biochemical and functional consequences of these mutations may provide a window of therapeutic opportunity. Indeed, therapeutic strategies tailored to target ALL-associated driver lesions and pathways may increase anti-leukemia efficacy and decrease relapse, as well as reduce undesirable off-target toxicities (J. Clin. Oncol. 33, 2938-48 (2015). Recently, recurrent DUX4 rearrangements were reported in up to 7% of B-ALL patients (Nat. Genet. 48, 569-74 (2016); EBioMedicine 8, 173-83 (2016); Nat. Commun. 7, 11790 (2016); Nat. Genet. 48, 1481-1489 (2016). Nearly all cases exhibit rearrangement of DUX4 to the immunoglobulin heavy chain (IGH) enhancer region resulting in truncation of DUX4 C terminus and addition of amino acids from read-through into the IGH locus. The rearrangement has two functional consequences. First, the translocation hijacks the IGH enhancer resulting in overexpression of DUX4 in the B cell lineage. Second, the truncation of DUX4 C terminus and the appendage of amino acids encoded by the IGH locus changes the biology of the resulting DUX4-IGH fusion protein. While DUX4 is pro-apoptotic, DUX4-IGH induces transformation in NIH-3T3 fibroblasts and is required for the proliferation of DUX4-IGH expressing NALM6 B-ALL cells (Nat. Genet. 48, 569-74 (2016); Nat. Genet. 48, 1481-1489 (2016). Moreover, expression of DUX4-IGH in mouse pro-B cells is sufficient to give rise to leukemia. In contrast, mouse pro-B cells expressing wild-type DUX4 undergo cell death (Nat. Genet. 48, 569-74 (2016). The DUX4 rearrangement is a clonal event acquired early in leukemogenesis and the expression of DUX4-IGH is maintained in leukemias at relapse (Nat. Genet. 48, 569-74 (2016); Nat. Genet. 48, 1481-1489 (2016), strongly supporting DUX4-IGH as an oncogenic driver.

Despite the genetic defect underlying FSHD being known for 25 years, no therapeutic option is currently available. Consensus amongst researchers in the FSHD field points to the aberrant expression of DUX4 as the main driver of the dystrophic pathology. Envisaging potential therapeutic avenues to treat FSHD, several approaches are possible, including: i) re-establish silencing at the D4Z4 locus; ii) prevent translation of the DUX4 RNA; or iii) block the toxic activity of DUX4. While intriguing proof of principle studies have been published assessing the possibility to inhibit DUX4 transcription or degrade its RNA (44) (45) (46) (47) (48) (49) (50) (51) (52) (53), currently their major limitations are poor specificity or inefficient in vivo delivery. Instead, development of rational therapeutic approaches to specifically counteract DUX4-induced toxicity has been hampered by limited understanding of the molecular mechanism through which DUX4 activity is regulated. While inhibitors of DUX4 activity have been previously reported (54) (55) (56) (57), they act non-specifically, indirectly and/or have not been tested in FSHD muscle cells. Instead, the present results point to MATR3 as a physiological inhibitor of DUX4 activity. The inventors propose that MATR3 protects from DUX4-induced toxicity by directly inhibiting DUX4 binding to target loci with the end result of preventing transcriptional activation of genes toxic to muscle cells. As a result, MATR3 not only promotes survival of FSHD muscle cells but also allows their myogenic differentiation. Thus, MATR3 rescues two key features of DUX4-induced toxicity.

In addition to directly blocking DUX4 activity, the inventors found that MATR3 is also able to inhibit DUX4 expression. Intriguingly, this effect is restricted to the expression of the endogenous DUX4 gene since MATR3 is unable to affect levels of transfected DUX4. Recently, a positive feed-forward mechanism involving the DUX4 target MBD3L2 and necessary for the full induction of DUX4 transcription in FSHD muscle cells has been reported (58). MBD3L2 works by counteracting DUX4 transcriptional repression mediated by the Nucleosome Remodeling Deacetylase (NuRD) complex. MDB3L2 is selectively expressed in FSHD muscle cells, while it is normally silent in healthy muscle cells. Importantly, MDB3L2 knockdown significantly decreases DUX4 expression in FSHD muscle cells suggesting that DUX4-induced MBD3L2 contributes to the amplification of DUX4 transcription in FSHD muscle cells (58). Since the inventors found that MATR3 expression is associated with MBD3L2 downregulation, it is tempting to speculate that MATR3 decreases DUX4 expression in FSHD muscle cells indirectly by blocking the induction of MDB3L2 by DUX4.

Notwithstanding their distinct clinical definitions, FSHD shares intriguing molecular features with amyotrophic lateral sclerosis (ALS), the most common motor neuron disease. Overlapping aspects include altered proteostasis, aberrant RNA metabolism, activation of human endogenous retroviruses, increased oxidative stress, aggregates of TDP-43 and cell death (8) (59) (60) (61) (62) (63) (64) (22) (65) (66) (67) (40) (68) (69) (70). A molecular explanation for these similarities is still lacking. Intriguingly, MATR3 interacts with TDP-43 (71), forms aggregates with TDP-43 in ALS (72) and MATR3 mutations cause ALS (71), indicating that MATR3 dysfunction is integrally linked to ALS pathogenesis. ALS is linked to different forms of muscular disorders. In this regard, a recurrent mutation in MATR3 causes asymmetric progressive autosomal-dominant distal myopathy (73), which also shows clinical manifestations overlapping with FSHD. The inventors found that a MATR3 N-terminal fragment of 287 amino acids is sufficient to bind DUX4 and inhibit its activity. While this region does not display any known functional domain, it contains a mutation hotspot in ALS (74) and the only amino acid mutated in distal myopathy (73). These disorders may be considered different phenotypes of the same spectrum, which could help to identify common pathological pathways and therapeutic targets. FSHD is characterized by an extensive intrafamilial variability in clinical severity and disease progression, with ˜20% of affected individuals becoming wheelchair-dependent, while an equal proportion of same genetic defect carriers family relatives remaining asymptomatic throughout their lives (75) (2). This variability is only in part explained by currently known FSHD disease modifiers (76). Hence, MATR3 may act as an additional modifier of disease severity in families with FSHD. For example, a MATR3 mutation decreasing its ability to bind DUX4 could be associated with a more severe FSHD phenotype. On the contrary, a MATR3 mutation increasing its ability to bind DUX4 would be protective.

Using a rat primary neuron model, toxicity upon MATR3 overexpression was recently reported (77). Intriguingly, the toxicity was dose-dependent being mostly evident with high levels of MATR3 overexpression. Notably, deletion of MATR3 zinc finger 2 rescued MATR3 overexpression toxicity (77). The inventors found no evidence of toxicity by MATR3 overexpression in HEK293 or FSHD muscle cells. On the contrary, MATR3 overexpression promoted survival and myogenic differentiation of FSHD muscle cells. This could be in part due to the fact that, in the present settings, the level of MATR3 overexpression is relatively modest. It is also possible that the toxicity associated with MATR3 overexpression is restricted to neurons. The inventors found that a MATR3 fragment lacking all known functional domains (including zinc finger 2) is as effective as the full-length MATR3 in directly blocking DUX4 activity. To the best of inventors' knowledge, the other known MATR3 interactors described thus far bind to MATR3 domains located away from the MATR3 region responsible for binding to DUX4 and/or associate to MATR3 indirectly through nucleic acid bridges (78) (79) (71) (80) (81). Hence, overexpression of the minimal MATR3 fragment binding DUX4 would not interfere with the interaction of MATR3 with its other partners further decreasing the possibility to observe toxic effects.

In summary, the present results show that MATR3 is a natural inhibitor of DUX4 activity that binds to DUX4 DNA-binding domain and prevents activation of its targets and induction of apoptosis. As the first identified protein able to control both DUX4 expression and activity, MATR3 is an intriguing target for the development of novel therapeutic strategies to effectively treat a condition associated with aberrant expression and/or function of DUX4 such as FSHD or ALL.

### Example 8: MATR3 Interacts with the DUX4-IGH Oncogene, Inhibits DUX4-IGH Activity and Blocks Production of the Leukemia Driver ERGalt

Material and Methods (FIG. 12)

HEK293T cells were plated on 10 cm cell culture plates and when 90% confluence they were transfected with 3 μg of pLBC2-BS-RFCA-BCVIII vector carrying DUX4 or DUX4-IGH and/or 6 μg of pCMV-Tag2B vector carrying FLAG-tagged MATR3, using Lipofectamine LTX with Plus Reagent (Thermo Fisher Scientific), following manufacturer's instructions. 24 hours after transfection, cells were harvested and nuclear extracts were prepared by lysing the cell membrane with buffer N (300 mM sucrose; 10 mM Hepes pH 7.9; 10 mM KCl; 0.1 mM EDTA; 0.1 mM EGTA; 0.1 mM DTT; 0.75 mM spermidine; 0.15 mM spermine; 0.1% NP-40 substitute; protease inhibitors) followed by extraction of nuclear proteins using buffer C420 (20 mM Hepes pH 7.9, 420 mM NaCl, 25% glycerol, 1 mM EDTA, 1 mM EGTA, 0.1 mM DTT, protease inhibitors). Nuclear extracts were cleared by ultracentrifugation at 100000 g, 4° C. for 1 hour. The pull-down was performed using 400 μg of nuclear proteins, adding 2 volumes of HEPES buffer (20 mM Hepes pH 8; 50 mM NaF; protease inhibitors) and TNN buffer [50 mM Hepes pH 8.0; 150 mM NaCl; 5 mM EDTA; 0.5% NP-40 substitute; 50 mM NaF; protease inhibitors] to reduce the NaCl concentration. Nuclear extracts were incubated for 1 h at 4° C. with Avidin, Benzonase (Sigma Aldrich) and RNase A (Thermo Fisher Scientific). Protein complexes were obtained by incubation of nuclear extracts with 30 μl of anti-FLAG M2 Affinity Gel beads (Sigma Aldrich) overnight at 4° C. in gentle rotation. After 3 washes with TNN buffer, proteins were eluted by adding 20 μl of 4× Laemmli buffer. 10 μl of 10% Input and pulldown proteins were analyzed by immunoblotting using antibodies specific for DUX4 (anti-Dux4 E5-5, Abcam ab124699) or FLAG (anti-FLAG M2, Sigma-Aldrich F1804).

Material and Methods (FIG. 13)

HEK293 cells were transfected with a DUX4-IGH-dependent GFP reporter (previously described in doi: 10.1093/hmg/ddv315) in combination with a DUX4-IGH expressing vector and MATR3 N-term FLAG vector (or empty vectors, EV) in a ratio of 0.25:1:2 using Lipofectamine LTX, according to manufacturer instruction. The cells were assayed after 24 hours for activation of the GFP-reporter by fluorescence microscopy using Zeiss Observer.Z1 and the AxioVision software.

Material and Methods (FIG. 14)

NALM6 B-ALL cells were transduced by 2 rounds of spin-infection for 90 minutes at 1290×g with FUGW GFP or FUGW GFP-MATR3 lentiviral vectors, where the GFP is fused to the N-terminus of the MATR3 ORF. Lentiviral production was carried out in HEK293T packaging cells using the calcium phosphate method, as previously described (doi: 10.1093/hmg/ddu536). For the Western blot analysis, whole cell lysates were obtained using the IP buffer [50 mM Tris-HCl pH 7.5; 150 mM NaCl; 1% NP-40; 5 mM EDTA; 5 mM EGTA; plus protease inhibitors (Sigma)]. Total protein concentration was measured using the Bradford reagent (Bio-Rad) and the spectrophotometer GeneQuant1300 (GE Healthcare Life Sciences). Equal amount of total proteins were loaded on 10% SDS-Page and Actin used as protein loading control. Antibodies were purchased from Abcam [Anti-Dux4 antibody (E5-5) ab124699; Anti-ERG antibody (EPR3864(2)) ab133264; Anti-beta Actin antibody (mAbcam 8226)—Loading Control (ab8226)].

MATR3 Interacts with the DUX4-IGH Oncogene

As described above, MATR3 directly binds to the DUX4 DNA-binding domain, a region which is maintained DUX4-IGH. MATR3 is thus predicted to bind also DUX4-IGH. To directly confirm the interaction between MATR3 and DUX4-IGH, inventors performed nuclear pulldowns using a FLAG-tagged form of MATR3, followed by immunoblotting for DUX4-IGH (DUX4 was used as positive control). Importantly, MATR3 is able to bind DUX4-IGH (FIG. 12).

MATR3 Inhibits DUX4-IGH Activity

To compare side-by-side the ability of MATR3 to regulate the activity of DUX4 and DUX4-IGH, inventors took advantage of a reporter system carrying DUX4/DUX4-IGH binding sites upstream of a GFP reporter gene (described in doi: 10.1093/hmg/ddv315). Intriguingly, the inventors found that MATR3 is able to strongly inhibit both DUX4 and DUX4-IGH activity (FIG. 13).

MATR3 Blocks Production of the Leukemia Driver ERGalt

Transcriptional disregulation of the ETS transcription factor gene ERG is a hallmark of the subtype of B-progenitor ALL caused by DUX4-IGH, with expression of the novel coding ERG transcript ERGalt. ERGalt is directly induced by DUX4, and is present in all cases of DUX4-IGH leukemia, but rarely in any other tumor. Importantly, ERGalt ectopic expression induces leukemia, in line with the possibility that ERGalt is required in the pathogenesis of human DUX4-IGH ALL (doi: 10.1038/ng.3691).

To evaluate the ability of MATR3 to regulate ERGalt production, inventors used NALM6 cells, a B-ALL line endogenously expressing DUX4-IGH and requiring DUX4-IGH for proliferation (doi: 10.1038/ng.3691). Notably, the inventors found that MATR3 delivery blunt the expression of the DUX4-IGH target gene ERGalt in leukemic cells.

Collectively, the present data strongly support that MATR3 binds to DUX4-IGH DNA-binding domain, preventing the activation of a key target gene for leukemogenesis.

DUX4 encodes for a transcription factor with increasingly important roles in normal physiology and in disease.

DUX4 is a key gene responsible for genome activation at the cleavage stage of embryonic development (doi: 10.1038/ng.3844; doi: 10.1038/ng.3846; doi: 10.1038/ng.3858). It is silent in adult tissues except testis and thymus, possibly mediating elimination of pre-T cells that fail (3-selection in the latter (doi: 10.1084/jem.20181444).

Regarding human diseases, DUX4 was first reported to be ectopically reactivated in skeletal muscle causing FSHD muscular dystrophy, one of the most common neuromuscular disorders (doi: 10.1093/hmg/ddy162).

DUX4 has been also reported to be overexpressed in several solid tumors where it mediates immune evasion (doi: 10.1016/j.stem.2018.10.011; doi: 10.1016/j.devcel.2019.06.011).

Finally, translocations of DUX4 in the immunoglobulin heavy chain (IGH) locus occurs in 7% of acute lymphoblastic leukemia (ALL), the most common pediatric cancer and the major cause of cancer-related death before the age of 20 (doi: 10.1038/ng.3535; doi: 10.1038/ng.3691; doi: 10.1016/j.ebiom.2016.04.038; doi: 10.1038/ncomms11790).

Present data point to MATR3 as a natural inhibitor of DUX4 activity. Based on this, MATR3-based compounds are useful to block DUX4 and DUX4-IGH aberrant activity in muscular dystrophy and cancer.

### REFERENCES

1. J. C. W. Deenen, et al., Neurology 83, 1056-9 (2014).
2. L. H. Wang, et al., Curr. Neurol. Neurosci. Rep. 16, 66 (2016).
3. D. S. S. et al., J. Cell Biol. 191, 1049-1060 (2010).
4. P. G. Hendrickson, et al., Nat. Genet. 49, 925-934 (2017).
5. J. L. Whiddon, et al., Nat. Genet. 49, 935-940 (2017).
6. A. De Iaco, et al., Nat. Genet. 49, 941-945 (2017).
7. V. Kowaljow, et al., Neuromuscul. Disord. 17, 611-23 (2007).
8. D. Bosnakovski, et al., EMBO J. 27, 2766-79 (2008).
9. L. M. Wallace, et al., Ann. Neurol. 69, 540-52 (2011).
10. L. N. Geng, et al., Dev. Cell 22, 38-51 (2012).
11. Z. Yao, et al., Hum. Mol. Genet. 23, 5342-52 (2014).
12. A. M. Rickard, et al., Hum. Mol. Genet. 24, 5901-14 (2015).
13. M. Sandri, et al., J. Neuropathol. Exp. Neurol. 60, 302-12 (2001).
14. G. J. Block, et al., Hum. Mol. Genet. 22, 4661-72 (2013).
15. J. M. Statland, et al., J. Neuromuscul. Dis. 2, 291-299 (2015).
16. A. M. Rickard, et al., Hum. Mol. Genet. 24, 5901-14 (2015).
17. R. Tawil, et al., Neurology 48, 46-9 (1997).
18. E. Passerieux, et al., Free Radic. Biol. Med. 81, 158-69 (2015).
19. J. T. Kissel, et al., Neurology 57, 1434-40 (2001).
20. B. H. Elsheikh, et al., Neurology 68, 1428-9 (2007).
21. K. R. Wagner, et al., Ann. Neurol. 63, 561-71 (2008).
22. S. Homma, et al., Ann. Clin. Transl. Neurol. 2, 151-66 (2015).
23. S. Jagannathan, et al., Hum. Mol. Genet. 25, 4419-4431 (2016).
24. S. H. Choi, et al., Nucleic Acids Res. 44, 5161-5173 (2016).
25. G. Rigaut, et al., Nat. Biotechnol. 17, 1030-1032 (1999).
26. T. Kocher, et al., Nat. Methods 4, 807-815 (2007).
27. T. Glatter, et al., Mol. Syst. Biol. 5, 237 (2009).
28. Y. Li, Biotechnol. Lett. 33, 1487-99 (2011).
29. M. Varjosalo, et al., Nat. Methods 10, 307-314 (2013).
30. P. Belgrader, et al., J. Biol. Chem. 266, 9893-9 (1991).
31. H. Nakayasu, et al., Proc. Natl. Acad. Sci. U.S.A 88, 10312-6 (1991).
32. Y. Hibino, et al., Biochem. Biophys. Res. Commun. 279, 282-7 (2000).
33. Z. Zhang, et al., Cell 106, 465-75 (2001).
34. E. D. Corona, et al., PLoS One 8, e75614 (2013).
35. L. Snider, et al., PLoS Genet. 6, e1001181 (2010).
36. A. Tassin, et al., J. Cell. Mol. Med. 17, 76-89 (2013).
37. O. Soderberg, et al., Methods 45, 227-32 (2008).
38. L. Snider, et al., PLoS Genet. 6, e1001181 (2010).
39. T. I. Jones, et al., Hum. Mol. Genet. 21, 4419-30 (2012).
40. G. J. Block, et al., Hum. Mol. Genet. 22, 4661-72 (2013).
41. D. Bosnakovski, et al., EMBO J. 27, 2766-2779 (2008).
42. C. Vanderplanck, et al., PLoS One 6, e26820 (2011).
43. L. Caron, et al., Stem Cells Transl. Med. 5, 1145-61 (2016).
44. L. M. Wallace, et al., Mol. Ther. 20, 1417-23 (2012).
45. J.-W. Lim, et al., Hum. Mol. Genet. 24, 4817-28 (2015).
46. E. Ansseau, et al., Genes (Basel). 8, 93 (2017).
47. E. Ansseau, et al., Genes (Basel). 8, 93 (2017).
48. A. E. Campbell, et al., Skelet. Muscle 7, 16 (2017).
49. G. Golshirazi, et al., Methods Mol. Biol. 1828, 91-124 (2018).
50. C. L. Himeda, T et al., Mol. Ther. 26, 1797-1807 (2018).
51. J. M. Cruz, et al., J. Biol. Chem. 293, 11837-11849 (2018).
52. C. L. Himeda, et al., Mol. Ther. 24, 527-535 (2016).
53. J. C. Chen, et al., Mol. Ther. 24, 1405-11 (2016).
54. D. Bosnakovski, et al., Skelet. Muscle 4, 4 (2014).
55. L. A. Moyle, et al., Elife 5 (2016), doi:10.7554/eLife.11405.
56. S. C. Shadle, et al., PLoS Genet. 13, e1006658 (2017).
57. E. Teveroni, et al., J. Clin. Invest. 127, 1531-1545 (2017).
58. A. E. Campbell, et al., Elife 7 (2018), doi:10.7554/eLife.31023.
59. I. R. Mackenzie, et al., Lancet. Neurol. 9, 995-1007 (2010).
60. R. Douville, et al., Ann. Neurol. 69, 141-51 (2011).
61. W. Li, et al., Sci. Transl. Med. 7, 307ra153 (2015).
62. L. N. Bowen, et al., Neurology 87, 1756-1762 (2016).
63. L. Krug, et al., PLoS Genet. 13, e1006635 (2017).
64. J. P. Taylor, et al., Nature 539, 197-206 (2016).
65. J. M. Young, et al., PLoS Genet. 9, e1003947 (2013).
66. S. T. Winokur, et al., Neuromuscul. Disord. 13, 322-33 (2003).
67. M. Barro, et al., J. Cell. Mol. Med. 14, 275-89 (2010).
68. A. Turki, et al., Free Radic. Biol. Med. 53, 1068-79 (2012).
69. A. Dandapat, et al., Cell Rep. 8, 1484-1496 (2014).
70. L. Caron, et al., Stem Cells Transl. Med. 5 (2016), doi:10.5966/sctm.2015-0224.
71. J. O. Johnson, et al., Nat. Neurosci. 17, 664-666 (2014).
72. M. Tada, H. et al., Am. J. Pathol. 188, 507-514 (2018).
73. J. Senderek, et al., Am. J. Hum. Genet. 84, 511-8 (2009).
74. A. Boehringer, et al., Sci. Rep. 7, 14529 (2017).
75. M. V. Neguembor, et al., in The Online Metabolic and Molecular Bases of Inherited Disease, FACMG, Editor, Grant Mitch, Ed. (McGraw-Hill Medical, 2015).
76. K. Mul, et al., Clin. Genet. (2018), doi:10.1111/cge.13446.
77. A. M. Malik, et al., Elife 7 (2018), doi:10.7554/eLife.35977.
78. M. Salton, et al., PLoS One 6, e23882 (2011).
79. M. B. Coelho, et al., EMBO J. 34, 653-68 (2015).
80. A. Kula, et al., Retrovirology 8, 60 (2011).
81. S. Tenzer, et al., J. Proteome Res. 12, 2869-2884 (2013).
82. P. Zhou, et al., J. Biomol. NMR 46, 23-31 (2010).
83. R. Giambruno, et al., J. Proteome Res. 12, 4018-27 (2013).
84. J. R. Wi§ niewski, et al., Nat. Methods 6, 359-62 (2009).
85. M. L. Huber, et al., J. Proteome Res. 13, 1147-55 (2014).
86. J. Cox, et al., J. Proteome Res. 10, 1794-805 (2011).

1. A method of treating a condition associated with an aberrant expression and/or function of a DUX4 protein and/or of a DUX4 fusion protein, comprising administering an amount of MATRIN-3 (MATR3), fragment, variant, fusion, or conjugate thereof to a patient in need of such treatment.

2. The method of claim 1 wherein the MATRIN-3 (MATR3) variant is selected from Table 1.

3. The method of claim 1 wherein the MATRIN-3 (MATR3) or a fragment thereof is an MCPP-MATRIN-3 (MATR3) fusion protein or an MCPP-Degrader-MATRIN-3 (MATR3) fusion protein.

4. The method of claim 1 wherein the MATRIN-3 (MATR3) or a fragment thereof is a fatty acid-MATRIN-3 (MATR3) conjugate or a PEG-MATRIN-3 (MATR3) conjugate.

5. (canceled)

6. The method of claim 1, wherein said method further comprises administering a therapeutic agent.

7. A method of treating a condition associated with aberrant expression and/or function of a DUX4 protein and/or of a DUX4 fusion protein, comprising administering an amount of a nucleic acid construct encoding the MATRIN-3 (MATR3), fragment, variant, fusion, or conjugate thereof to a patient in need of such treatment.

8. The method of claim 7, wherein the nucleic acid construct is part of an expression vector, and wherein the expression vector optionally comprises a promoter operatively linked to the nucleic acid construct.

9. The method of claim 8 wherein said expression vector is an AVV vector.

10. The method of claim 8 wherein the promoter is a muscle-specific promoter.

11. The method of claim 8, wherein the expression vector is part of a transformed cell and the cell is either a eukaryotic cell selected from the group consisting of a mammalian cell, an insect cell, a plant cell, a yeast cell and a protozoa cell, or the cell is a bacterial cell.

12. The method of claim 1 wherein the condition associated with aberrant expression and/or function of DUX4 protein and/or of DUX4 fusion proteins is selected from the group consisting of: muscular dystrophy, infection, and cancer.

13. The method according to claim 12 wherein the cancer is selected from the group consisting of: acute lymphoblastic leukemia, undifferentiated small round blue cell sarcoma, rhabdomyosarcoma, breast, testis, kidney, stomach, lung, thymus, liver, uterus, larynx, esophagus, tongue, heart, connective, mouth, colon, mesothelioma, bladder, ovary, brain, tonsil, pancreas, peritoneum, prostatic or thyroid cancer.

14. The method according to claim 12 wherein the infection is a herpes virus infection or wherein the muscular dystrophy is facioscapulohumeral muscular dystrophy (FSHD).

15. The method of claim 13, wherein the cancer is acute lymphoblastic leukemia.
